(19) United States
(12) Patent Application Publication
(10) Pub. No.: US 2026/0217672 A1
(43) Pub. Date: Jul. 30, 2026

(54) BENZAMIDE COMPOUNDS FOR TREATMENT OF BACTERIAL INFECTIONS

(71) Applicant: Hoffmann-La Roche Inc., Little Falls, NJ (US)

(72) Inventors: Fabian Dey, Zurich (CH); Xiao Ding, Shanghai (CN); Houguang Shi, Shanghai (CN); Xuefei Tan, Shanghai (CN); Jun Wu, Shanghai (CN); Jiamin Zheng, Shanghai (CN); Mingwei Zhou, Shanghai (CN)

(73) Assignee: Hoffmann-La Roche Inc., Little Falls, NJ (US)

(21) Appl. No.: 18/823,634

(22) PCT Filed: Mar. 2, 2023
(86) PCT No.: PCT/EP2023/055251

§ 371 Date: Sep. 3, 2024

(30) Foreign Application Priority Data
Mar. 4, 2022 (WO) PCT/CN2022/079326

(51) Int. Cl. C07D 401/12; A61K 31/496; A61K 31/4985; A61K 31/519; A61K 31/5377; A61K 31/635; A61P 31/04; C07D 213/74; C07D 213/79; C07D 213/84; C07D 239/42; C07D 239/48; C07D 295/26; C07D 401/04; C07D 401/14; C07D 413/12; C07D 471/04; C07D 487/04; C07D 487/08; C07D 498/10

(52) U.S. Cl. CPC C07D 401/12; A61K 31/496; A61K 31/4985; A61K 31/519; A61K 31/5377; A61K 31/635; A61P 31/04; C07D 213/74; C07D 213/79; C07D 213/84; C07D 239/42; C07D 239/48; C07D 295/26; C07D 401/04; C07D 401/14; C07D 413/12; C07D 471/04; C07D 487/04; C07D 487/08; C07D 498/10

(57) ABSTRACT

The present invention relates to compounds of formula (I), wherein R¹ to R⁸, Y, A, Q, X and M are as described herein, and their pharmaceutically acceptable salt thereof, and compositions including the compounds and methods of using the compounds.

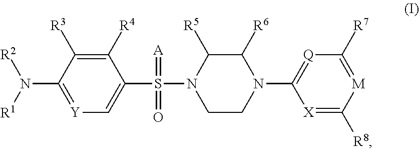

The present invention relates to organic compounds useful for the treatment and/or prevention of bacterial infections in a mammal. Specifically these molecules can inhibit the LPS synthesis pathway, in particular to inhibit LpxH, and are useful for treating bacterial infections.

### BACKGROUND OF THE INVENTION

The intensive use of antibiotics has exerted a selective evolutionary pressure on microorganisms to produce genetically based resistance mechanisms. Modern medicine and socio-economic behaviour exacerbate the problem of resistance development by creating slow growth situations for pathogenic microbes, e.g. in artificial joints, and by supporting long-term host reservoirs, e.g. in immune-compromised patients. In hospital settings, an increasing number of strains of Staphylococcus aureus, Streptococcus pneumoniae, Enterococcus spp., Enterobacteriaceae such as Klebsiella pneumonia, Acinetobacter baumannii and Pseudomonas aeruginosa, major sources of infections, are becoming multi-drug resistant and therefore difficult to treat. This is particularly the case for Gram-negative organisms where the situation is getting worrisome since no novel agents with a differentiated mechanism of action have been approved for decades. Therefore, there is an important medical need for new antibacterial compounds addressing Gram-negative resistant bacteria, in particular third generation cephalosporins- and carbapenem-resistant Enterobacteriaceae and multi-drug-resistant Pseudomonas aeruginosa and Acinetobacter baumannii. One way to tackle the problem of cross-resistance to established classes of antibiotics is to inhibit an essential protein or function not targeted by current antibiotics.

Gram-negative bacteria are unique in that their outer membrane contains Lipopolysaccha-ride (LPS), which is crucial for maintaining membrane integrity, and is essential for bacterial viability (reviewed in Ann. Rev. Biochem 76: 295-329, 2007). The major lipid component of LPS is Lipid A, and inhibition of Lipid A biosynthesis is lethal to bacteria. Lipid A is synthesized on the cytoplasmic surface of the bacterial inner membrane via a pathway that consists of nine different enzymes. These enzymes are highly conserved in most Gram-negative bacteria. LpxH, a calcineurin-like phosphatase (CLP), catalyzes the hydrolysis of UDP-2,3-diacyl-glucosamine (UDP-DAGn) to yield Lipid X and UMP (22, 24, 25). LpxH has no mammalian homologue, making it a good target for the development of novel antibiotics targeting Gram-negative bacteria.

### SUMMARY OF THE INVENTION

The present invention relates to novel compounds of formula (I),

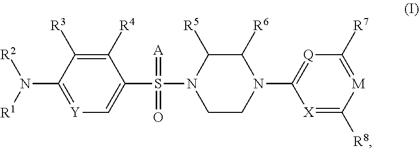

wherein
R¹ is COR⁹; wherein
   R⁹ is ((hydroxyC₁₋₆alkyl)amino)C₁₋₆alkyl,
      1-oxa-2,9-diazaspiro[4.5]dec-2-enyl,
      4,5,6,7-tetrahydropyrazolo[1,5-a]pyrazinyl,
      4,6-dihydropyrrolo[3,4-c]pyrazolyl substituted by C₁₋₆alkyl,
      6,7-dihydro-5H-pyrrolo[3,4-b]pyridinyl,
      indanyl substituted by amino or aminoC₁₋₆alkylamino,
      isoindolinyl,
      piperazinylC₁₋₆alkyl,
      pyrazolo[1,5-a]pyrimidinyl,
      pyrazolyl substituted once or twice by the substituents independently selected from aminoC₁₋₆alkyl, azetidinyl, C₁₋₆alkyl and carboxy,
      pyridinyl substituted by (aminoC₁₋₆alkylamino)C₁₋₆alkyl, or
      phenyl substituted once or twice by the substituents independently selected from ((amino(carboxy)C₁₋₆alkyl)amino)C₁₋₆alkyl, ((aminoC₁₋₆alkyl)-3-azabicyclo[3.1.0]hexanyl)C₁₋₆alkyl, ((aminoC₁₋₆alkyl)pyrrolidinyl)C₁₋₆alkyl, ((aminoC₃₋₇cycloalkyl)C₁₋₆alkylamino)C₁₋₆alkyl, ((aminoC₃-7cycloalkyl)pyrrolidinyl)C₁₋₆alkyl, ((oxooxazolidinyl)C₁₋₆alkylamino)C₁₋₆alkyl, ((oxopyrrolidinyl)C₁₋₆alkylamino)C₁₋₆alkyl, ((piperidinyl)C₁₋₆alkylamino)C₁₋₆alkyl, (3,3a,4,5,6,6a-hexahydro-2H-pyrrolo[3,4-b]pyrrolyl)C₁₋₆alkyl, (3,6-diazabicyclo[3.1.1]heptanyl)C₁₋₆alkyl, (amino-2-azaspiro[3.3]heptanyl)C₁₋₆alkyl, (amino-5-azaspiro[2.4]heptanyl)C₁₋₆alkyl, (aminoC₁₋₆alkyl)piperazinyl, (aminoC₁₋₆alkyl)pyrrolidinyl, (aminoC₁₋₆alkylamino)C₁₋₆alkyl, (aminopyrrolidinyl)C₁₋₆alkyl, (C₁₋₆alkyl-2,3,3a,4,6,6a-hexahydropyrrolo[2,3-c]pyrrolyl)C₁₋₆alkyl, (diaminoC₁₋₆alkylcarbonyl)amino, (morpholinylC₁₋₆alkylamino)C₁₋₆alkyl, (piperazinylC₁₋₆alkylamino)C₁₋₆alkyl, (piperidinylCl-6alkylamino)C₁₋₆alkyl, [((C₁₋₆alkyl)₂aminocarbonylC₁₋₆alkyl)amino]C₁₋₆alkyl, [((hydroxyC₁₋₆alkyl)₂amino)C₁₋₆alkylamino]C₁₋₆alkyl, 2,5-diazabicyclo[2.2.1]heptanyl, 3,6-diazabicyclo[3.2.0]heptanyl, aminoC₁₋₆alkoxy, aminoC₁₋₆alkyl, aminopyrrolidinyl, azetidinylC₁₋₆alkoxy, azetidinylC₁₋₆alkylamino, C₁₋₆alkylcarbonyloxy, C₁₋₆alkoxy, carboxy, hydroxy and piperazinyl;
R² is H;
R³ is H, hydroxy or C₁₋₆alkoxy; or
R² and R³ together with the atoms they are attached to form a pyrrolidinyl;
R⁴ is H, hydroxy or C₁₋₆alkoxy;
R⁵ is H or C₁₋₆alkyl;
R⁶ is H or C₁₋₆alkyl; or
R⁵ and R⁶ together with the atoms they are attached to form a C₃₋₇cycloalkyl;
R⁷ is halogen, cyano, haloC₁₋₆alkyl or C₁₋₆alkoxycarbonyl;
R⁸ is H, halogen, cyano, amino, C₃₋₇cycloalkoxy, C₁₋₆alkyl, C₁₋₆alkoxy or haloC₁₋₆alkyl;
Y is CH or N;
A is O, N(CN), or N(C₁₋₆alkyl);
Q is CH or N;
X is CH or N;
M is N or CR¹⁰; wherein R¹⁰ is H, halogen, C₁₋₆alkyl or C₃₋₇cycloalkyl;
with the proviso that Q, X and M are not N simultaneously;
or a pharmaceutically acceptable salt thereof.

### DETAILED DESCRIPTION OF THE INVENTION

### Definitions

The term “C₁₋₆alkyl” denotes a saturated, linear or branched chain alkyl group containing 1 to 6, particularly 1 to 4 carbon atoms, for example methyl, ethyl, n-propyl, isopropyl, n-butyl, isobutyl, tert-butyl and the like. Particular “C₁₋₆alkyl” groups are methyl, ethyl and n-propyl.

The term “C₁₋₆alkylene” denotes a linear or branched saturated divalent hydrocarbon group of 1 to 6 carbon atoms or a divalent branched saturated divalent hydrocarbon group of 3 to 6 carbon atoms. Examples of C₁₋₆alkylene groups include methylene, ethylene, propylene, 2-methylpropylene, butylene, 2-ethylbutylene, pentylene, hexylene.

The term “halogen” and “halo” are used interchangeably herein and denote fluoro, chloro, bromo, or iodo.

The term “haloC₁₋₆alkyl” denotes a C₁₋₆alkyl group wherein at least one of the hydrogen atoms of the C₁₋₆alkyl group has been replaced by same or different halogen atoms, particularly fluoro atoms. Examples of haloC₁₋₆alkyl include monofluoro-, difluoro- or trifluoro-methyl, -ethyl or -propyl, for example 3,3,3-trifluoropropyl, 2-fluoroethyl, trifluoroethyl, fluoromethyl, difluoromethyl, difluoroethyl or trifluoromethyl.

The term “C₃₋₇cycloalkyl” denotes a monovalent saturated monocyclic or bicyclic hydrocarbon group of 3 to 7 ring carbon atoms. Bicyclic means consisting of two saturated carbocycles having one or more carbon atoms in common. Examples for monocyclic cycloalkyl are cyclopropyl, cyclobutyl, cyclopentyl, cyclohexyl or cycloheptyl. Examples for bicyclic cycloalkyl are bicyclo[1.1.0]butyl, bicyclo[2.2.1]heptanyl, or bicyclo[2.2.2]octanyl.

The term “C₃₋₇cycloalkoxy” denotes C₃₋₇cycloalkyl-O—.

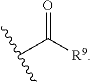

The term “COR⁹” denotes

The term “oxo” denotes a divalent oxygen atom ═O.

The term “phenylene” denotes divalent phenyl group.

The term “pyrazolylene” denotes divalent pyrazolyl group.

The term “pharmaceutically acceptable salts” denotes salts which are not biologically or otherwise undesirable. Pharmaceutically acceptable salts include both acid and base addition salts.

The term “pharmaceutically acceptable acid addition salt” denotes those pharmaceutically acceptable salts formed with inorganic acids such as hydrochloric acid, hydrobromic acid, sulfuric acid, nitric acid, carbonic acid, phosphoric acid, and organic acids selected from aliphatic, cycloaliphatic, aromatic, araliphatic, heterocyclic, carboxylic, and sulfonic classes of organic acids such as formic acid, acetic acid, propionic acid, glycolic acid, gluconic acid, lactic acid, pyruvic acid, oxalic acid, malic acid, maleic acid, maloneic acid, succinic acid, fumaric acid, tartaric acid, citric acid, aspartic acid, ascorbic acid, glutamic acid, anthranilic acid, benzoic acid, cinnamic acid, mandelic acid, embonic acid, phenylacetic acid, methanesulfonic acid, ethanesulfonic acid, p-toluenesulfonic acid, and salicyclic acid.

The term “pharmaceutically acceptable base addition salt” denotes those pharmaceutically acceptable salts formed with an organic or inorganic base. Examples of acceptable inorganic bases include sodium, potassium, ammonium, calcium, magnesium, iron, zinc, copper, manganese, and aluminum salts. Salts derived from pharmaceutically acceptable organic nontoxic bases includes salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-diethylaminoethanol, trimethamine, dicyclohexylamine, lysine, arginine, histidine, caffeine, procaine, hydrabamine, choline, betaine, ethylenediamine, glucosamine, methylglucamine, theobromine, purines, piperizine, piperidine, N-ethylpiperidine, and polyamine resins.

The term “A pharmaceutically active metabolite” denotes a pharmacologically active product produced through metabolism in the body of a specified compound or salt thereof. After entry into the body, most drugs are substrates for chemical reactions that may change their physical properties and biologic effects. These metabolic conversions, which usually affect the polarity of the compounds of the invention, alter the way in which drugs are distributed in and excreted from the body. However, in some cases, metabolism of a drug is required for therapeutic effect.

The term “therapeutically effective amount” denotes an amount of a compound or molecule of the present invention that, when administered to a subject, (i) treats or prevents the particular disease, condition or disorder, (ii) attenuates, ameliorates or eliminates one or more symptoms of the particular disease, condition, or disorder, or (iii) prevents or delays the onset of one or more symptoms of the particular disease, condition or disorder described herein. The therapeutically effective amount will vary depending on the compound, the disease state being treated, the severity of the disease treated, the age and relative health of the subject, the route and form of administration, the judgement of the attending medical or veterinary practitioner, and other factors.

The term “pharmaceutical composition” denotes a mixture or solution comprising a therapeutically effective amount of an active pharmaceutical ingredient together with pharmaceutically acceptable excipients to be administered to a mammal, e.g., a human in need thereof.

### Inhibitor of Udp-2,3-Diacylglucosamine Hydrolase (Lpxh)

The present invention relates to (i) a compound of formula (I),

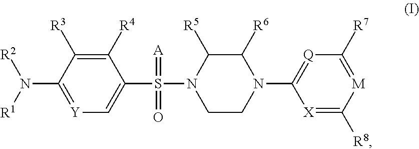

wherein
R¹ is COR⁹; wherein
   R⁹ is ((hydroxyC₁₋₆alkyl)amino)C₁₋₆alkyl,
      1-oxa-2,9-diazaspiro[4.5]dec-2-enyl,
      4,5,6,7-tetrahydropyrazolo[1,5-a]pyrazinyl,
      4,6-dihydropyrrolo[3,4-c]pyrazolyl substituted by C₁₋₆alkyl,
      6,7-dihydro-5H-pyrrolo[3,4-b]pyridinyl,
      indanyl substituted by amino or aminoC₁₋₆alkylamino,
      isoindolinyl,
      piperazinylC₁₋₆alkyl,
      pyrazolo[1,5-a]pyrimidinyl,
      pyrazolyl substituted once or twice by the substituents independently selected from aminoC₁₋₆alkyl, azetidinyl, C₁₋₆alkyl and carboxy,
      pyridinyl substituted by (aminoC₁₋₆alkylamino)C₁₋₆alkyl, or
      phenyl substituted once or twice by the substituents independently selected from ((amino(carboxy)C₁₋₆alkyl)amino)C₁₋₆alkyl, ((aminoC₁₋₆alkyl)-3-azabicyclo[3.1.0]hexanyl)C₁₋₆alkyl, ((aminoC₁₋₆alkyl)pyrrolidinyl)C₁₋₆alkyl, ((aminoC₃₋₇cycloalkyl)C₁₋₆alkylamino)C₁₋₆alkyl, ((aminoC₃-7cycloalkyl)pyrrolidinyl)C₁₋₆alkyl, ((oxooxazolidinyl)C₁₋₆alkylamino)C₁₋₆alkyl, ((oxopyrrolidinyl)C₁₋₆alkylamino)C₁₋₆alkyl, ((piperidinyl)C₁₋₆alkylamino)C₁₋₆alkyl, (3,3a,4,5,6,6a-hexahydro-2H-pyrrolo[3,4-b]pyrrolyl)C₁₋₆alkyl, (3,6-diazabicyclo[3.1.1]heptanyl)C₁₋₆alkyl, (amino-2-azaspiro[3.3]heptanyl)C₁₋₆alkyl, (amino-5-azaspiro[2.4]heptanyl)C₁₋₆alkyl, (aminoC₁₋₆alkyl)piperazinyl, (aminoC₁₋₆alkyl)pyrrolidinyl, (aminoC₁₋₆alkylamino)C₁₋₆alkyl, (aminopyrrolidinyl)C₁₋₆alkyl, (C₁₋₆alkyl-2,3,3a,4,6,6a-hexahydropyrrolo[2,3-c]pyrrolyl)C₁₋₆alkyl, (diaminoC₁₋₆alkylcarbonyl)amino, (morpholinylC₁₋₆alkylamino)C₁₋₆alkyl, (piperazinylC₁₋₆alkylamino)C₁₋₆alkyl, (piperidinylC₁₋₆alkylamino)C₁₋₆alkyl, [((C₁₋₆alkyl)₂aminocarbonylC₁₋₆alkyl)amino]C₁₋₆alkyl, [((hydroxyC₁₋₆alkyl)₂amino)C₁₋₆alkylamino]C₁₋₆alkyl, 2,5-diazabicyclo[2.2.1]heptanyl, 3,6-diazabicyclo[3.2.0]heptanyl, aminoC₁₋₆alkoxy, aminoC₁₋₆alkyl, aminopyrrolidinyl, azetidinylC₁₋₆alkoxy, azetidinylC₁₋₆alkylamino, C₁₋₆alkylcarbonyloxy, C₁₋₆alkoxy, carboxy, hydroxy and piperazinyl;
R² is H;
R³ is H, hydroxy or C₁₋₆alkoxy; or
R² and R³ together with the atoms they are attached to form a pyrrolidinyl;
R⁴ is H, hydroxy or C₁₋₆alkoxy;
R⁵ is H or C₁₋₆alkyl;
R⁶ is H or C₁₋₆alkyl; or
R⁵ and R⁶ together with the atoms they are attached to form a C₃₋₇cycloalkyl;
R⁷ is halogen, cyano, haloC₁₋₆alkyl or C₁₋₆alkoxycarbonyl;
R⁸ is H, halogen, cyano, amino, C₃₋₇cycloalkoxy, C₁₋₆alkyl, C₁₋₆alkoxy or haloC₁₋₆alkyl; Y is CH or N;
A is O, N(CN), or N(C₁₋₆alkyl);
Q is CH or N;
X is CH or N;
M is N or CR¹⁰; wherein R¹⁰ is H, halogen, C₁₋₆alkyl or C₃₋₇cycloalkyl;
with the proviso that Q, X and M are not N simultaneously;
or a pharmaceutically acceptable salt thereof.

A further embodiment of present invention is (ii) a compound of formula (I), wherein
R¹ is COR⁹; wherein
   R⁹ is 6,7-dihydro-5H-pyrrolo[3,4-b]pyridinyl,
      indanyl substituted by amino or aminoC₁₋₆alkylamino,
      isoindolinyl,
      pyrazolo[1,5-a]pyrimidinyl,
      pyridinyl substituted by (aminoC₁₋₆alkylamino)C₁₋₆alkyl, or
      phenyl substituted once or twice by the substituents independently selected from ((aminoC₁₋₆alkyl)-3-azabicyclo[3.1.0]hexanyl)C₁₋₆alkyl, (aminoC₁₋₆alkyl)pyrrolidinyl, (aminoC₁₋₆alkylamino)C₁₋₆alkyl, aminoC₁₋₆alkoxy, aminoC₁₋₆alkyl, aminopyrrolidinyl, azetidinylC₁₋₆alkoxy, carboxy and piperazinyl.

A further embodiment of present invention is (iii) a compound of formula (I) according to (i) or (ii), or a pharmaceutically acceptable salt thereof, wherein R¹ is COR⁹; wherein R⁹ is 6,7-dihydro-5H-pyrrolo[3,4-b]pyridinyl, aminoindanyl, (3-aminopropylamino)indanyl, isoindolinyl, pyrazolo[1,5-a]pyrimidinyl, ((3-aminopropylamino)methyl)pyridinyl, ((3-aminopropylamino)methyl)phenyl, ((3-aminopropylamino)ethyl)phenyl, carboxyphenyl, (aminoethoxy)phenyl, (azetidin-3-ylmethoxy)phenyl, (aminopyrrolidin-1-yl)phenyl, (aminoethyl)phenyl, (piperazin-1-yl)phenyl, (3-aminopropoxy)₂phenyl, (1-(3-aminopropyl)pyrrolidin-2-yl)phenyl, (1-(3-aminopropyl)pyrrolidin-3-yl)phenyl or [[1-(aminomethyl)-3-azabicyclo[3.1.0]hexan-3-yl)methyl]phenyl.

A further embodiment of present invention is (iv) a compound of formula (I), according to any one of (i) to (iii), or a pharmaceutically acceptable salt thereof, wherein R² is H; R³ is H; or R² and R³ together with the atoms they are attached to form a pyrrolidinyl.

A further embodiment of present invention is (v) a compound of formula (I), according to any one of (i) to (iv), wherein R⁴ is H; R⁵ is H; and R⁶ is H.

A further embodiment of present invention is (vi) a compound of formula (I), or a pharmaceutically acceptable salt thereof, according to any one of (i) to (v), wherein R⁷ is halogen or haloC₁₋₆alkyl.

A further embodiment of present invention is (vii) a compound of formula (I), or a pharmaceutically acceptable salt thereof, according to any one of (i) to (vi), wherein R⁷ is bromo, trifluoromethyl or difluoroethyl.

A further embodiment of present invention is (viii) a compound of formula (I), or a pharmaceutically acceptable salt thereof, according to any one of (i) to (vii), wherein R⁸ is halogen or C₁₋₆alkyl.

A further embodiment of present invention is (ix) a compound of formula (I), or a pharmaceutically acceptable salt thereof, according to any one of (i) to (viii), wherein R⁸ is chloro or methyl.

A further embodiment of present invention is (x) a compound of formula (I), or a pharmaceutically acceptable salt thereof, according to any one of (i) to (ix), wherein Y is CH.

A further embodiment of present invention is (xi) a compound of formula (I), or a pharmaceutically acceptable salt thereof, according to any one of (i) to (x), wherein A is O.

A further embodiment of present invention is (xii) a compound of formula (I), or a pharmaceutically acceptable salt thereof, according to any one of (i) to (xi), wherein X is N.

A further embodiment of present invention is (xiii) a compound of formula (I), or a pharmaceutically acceptable salt thereof, according to any one of (i) to (xii), wherein M is CH.

A further embodiment of present invention is (xiv) a compound of formula (I),
R¹ is COR⁹; wherein
   R⁹ is 6,7-dihydro-5H-pyrrolo[3,4-b]pyridinyl,
      indanyl substituted by amino or aminoC₁₋₆alkylamino,
      isoindolinyl,
      pyrazolo[1,5-a]pyrimidinyl,
      pyridinyl substituted by (aminoC₁₋₆alkylamino)C₁₋₆alkyl, or
      phenyl substituted once or twice by the substituents independently selected from ((aminoC₁₋₆alkyl)-3-azabicyclo[3.1.0]hexanyl)C₁₋₆alkyl, (aminoC₁₋₆alkyl)pyrrolidinyl, (aminoC₁₋₆alkylamino)C₁₋₆alkyl, aminoC₁₋₆alkoxy, aminoC₁₋₆alkyl, aminopyrrolidinyl, azetidinylC₁₋₆alkoxy, carboxy and piperazinyl;
R² is H;
R³ is H; or
R² and R³ together with the atoms they are attached to form a pyrrolidinyl;
R⁴ is H;
R⁵ is H;
R⁶ is H;
R⁷ is halogen or haloC₁₋₆alkyl;
R⁸ is halogen or C₁₋₆alkyl;
Y is CH;
A is O;
Qis CH or N;
X is N;
M is CH;
or a pharmaceutically acceptable salt thereof.

A further embodiment of present invention is (xv) a compound of formula (I),
R¹ is COR⁹; wherein R⁹ is 6,7-dihydro-5H-pyrrolo[3,4-b]pyridinyl, aminoindanyl, (3-aminopropylamino)indanyl, isoindolinyl, pyrazolo[1,5-a]pyrimidinyl, ((3-aminopropylamino)methyl)pyridinyl, ((3-aminopropylamino)methyl)phenyl, ((3-aminopropylamino)ethyl)phenyl, carboxyphenyl, (aminoethoxy)phenyl, (azetidin-3-ylmethoxy)phenyl, (aminopyrrolidin-1-yl)phenyl, (aminoethyl)phenyl, (piperazin-1-yl)phenyl, (3-aminopropoxy)₂phenyl, (1-(3-aminopropyl)pyrrolidin-2-yl)phenyl, (1-(3-aminopropyl)pyrrolidin-3-yl)phenyl or [[1-(aminomethyl)-3-azabicyclo[3.1.0]hexan-3-yl)methyl]phenyl;
R² is H;
R³ is H; or
R² and R³ together with the atoms they are attached to form a pyrrolidinyl;
R⁴ is H;
R⁵ is H;
R⁶ is H;
R⁷ is bromo, trifluoromethyl or difluoroethyl;
R⁸ is chloro or methyl;
Y is CH;
A is O;
Qis CH or N;
X is N;
M is CH;
or a pharmaceutically acceptable salt thereof.

Another embodiment of present invention is a compound of formula (I) or (Ia) selected from the following:
4-[(3-Aminopropylamino)methyl]-N-[4-[4-[6-chloro-4-(trifluoromethyl)-2-pyridyl]piperazin-1-yl]sulfonylphenyl]benzamide;
4-[(3-Aminopropylamino)methyl]-N-[4-[4-[4-(trifluoromethyl)-2-pyridyl]piperazin-1-yl]sulfonylphenyl]benzamide;
4-[(3-Aminopropylamino)methyl]-N-[4-[4-[5-chloro-4-(trifluoromethyl)-2-pyridyl]piperazin-1-yl]sulfonylphenyl]benzamide;
4-[(3-Aminopropylamino)methyl]-N-[4-[4-[3-chloro-5-(trifluoromethyl) phenyl]piperazin-1-yl]sulfonylphenyl]benzamide;
4-[(3-Aminopropylamino)methyl]-N-[4-[4-[2-chloro-6-(trifluoromethyl)pyrimidin-4-yl]piperazin-1-yl]sulfonylphenyl]benzamide;
4-[(3-Aminopropylamino)methyl]-N-[4-[4-[2-amino-6-(trifluoromethyl)pyrimidin-4-yl]piperazin-1-yl]sulfonylphenyl]benzamide;
4-[(3-Aminopropylamino)methyl]-N-[4-[4-(4-bromo-6-chloro-2-pyridyl)piperazin-1-yl]sulfonylphenyl]benzamide;
4-[(3-Aminopropylamino)methyl]-N-[4-[4-[5-cyclopropyl-4-(trifluoromethyl)-2-pyridyl]piperazin-1-yl]sulfonylphenyl]benzamide;
4-[(3-Aminopropylamino)methyl]-N-[4-[4-[6-(cyclopropoxy)-4-(trifluoromethyl)-2-pyridyl]piperazin-1-yl]sulfonylphenyl]benzamide;
4-[(3-Aminopropylamino)methyl]-N-[4-[4-[6-ethoxy-4-(trifluoromethyl)-2-pyridyl]piperazin-1-yl]sulfonylphenyl]benzamide;
4-[(3-Aminopropylamino)methyl]-N-[4-[4-[6-ethyl-4-(trifluoromethyl)-2-pyridyl]piperazin-1-yl]sulfonylphenyl]benzamide;
4-[(3-Aminopropylamino)methyl]-N-[4-[4-[4-(trifluoromethyl)pyrimidin-2-yl]piperazin-1-yl]sulfonylphenyl]benzamide;
4-[2-(3-Aminopropylamino)ethyl]-N-[4-[4-[5-methyl-4-(trifluoromethyl)-2-pyridyl]piperazin-1-yl]sulfonylphenyl]benzamide;
4-[2-(3-Aminopropylamino)ethyl]-N-[4-[4-(4,5-dichloro-2-pyridyl)piperazin-1-yl]sulfonylphenyl]benzamide;
4-[2-(3-Aminopropylamino)ethyl]-N-[4-[4-[4-cyano-6-(trifluoromethyl)-2-pyridyl]piperazin-1-yl]sulfonylphenyl]benzamide;
4-[2-(3-Aminopropylamino)ethyl]-N-[4-[4-[6-cyano-4-(trifluoromethyl)-2-pyridyl]piperazin-1-yl]sulfonylphenyl]benzamide;
4-[2-(3-Aminopropylamino)ethyl]-N-[4-[4-[6-chloro-4-(1,1-difluoroethyl)-2-pyridyl]piperazin-1-yl]sulfonylphenyl]benzamide;
3-[(3-Aminopropylamino)methyl]-N-[4-[4-[6-methyl-4-(trifluoromethyl)-2-pyridyl]piperazin-1-yl]sulfonylphenyl]benzamide;
Methyl 2-[4-[4-[[3-[(3-aminopropylamino)methyl]benzoyl]amino]phenyl]sulfonylpiperazin-1-yl]-6-chloro-pyridine-4-carboxylate;
3-[(3-Aminopropylamino)methyl]-N-[4-[4-(6-chloro-4-cyano-2-pyridyl)piperazin-1-yl]sulfonylphenyl]benzamide;
3-[(3-Aminopropylamino)methyl]-N-[4-[4-[6-chloro-4-(trifluoromethyl)-2-pyridyl]-2-methyl-piperazin-1-yl]sulfonylphenyl]benzamide;
3-[(3-Aminopropylamino)methyl]-N-[4-[4-[6-chloro-4-(trifluoromethyl)-2-pyridyl]-3-methyl-piperazin-1-yl]sulfonylphenyl]benzamide;
3-[(3-Aminopropylamino)methyl]-N-[4-[[5-[6-chloro-4-(trifluoromethyl)-2-pyridyl]-2,5-diazabicyclo[4.2.0]octan-2-yl]sulfonyl]phenyl]benzamide;
3-[(3-Aminopropylamino)methyl]-N-[4-[4-[6-chloro-4-(trifluoromethyl)-2-pyridyl]piperazin-1-yl]sulfonylphenyl]benzamide;
3-[2-(3-Aminopropylamino)ethyl]-N-[4-[S-[4-[6-chloro-4-(trifluoromethyl)-2-pyridyl]piperazin-1-yl]-N-cyano-sulfonimidoyl]phenyl]benzamide;
3-[(3-aminopropylamino)methyl]-N-[4-[S-[4-[6-chloro-4-(trifluoromethyl)-2-pyridyl]piperazin-1-yl]-N-methyl-sulfonimidoyl]phenyl]benzamide;
3-[(3-Aminopropylamino)methyl]-N-[4-[4-[6-chloro-4-(trifluoromethyl)-2-pyridyl]piperazin-1-yl]sulfonyl-3-methoxy-phenyl]benzamide;
3-[(3-Aminopropylamino)methyl]-N-[4-[4-[6-chloro-4-(trifluoromethyl)-2-pyridyl]piperazin-1-yl]sulfonyl-3-hydroxy-phenyl]benzamide;
4-[(3-Aminopropylamino)methyl]-N-[5-[4-[6-chloro-4-(trifluoromethyl)-2-pyridyl]piperazin-1-yl]sulfonyl-2-pyridyl]benzamide;
3-[(3-Aminopropylamino)methyl]-N-[4-[4-[6-chloro-4-(trifluoromethyl)-2-pyridyl]piperazin-1-yl]sulfonyl-2-methoxy-phenyl]benzamide;
3-[(3-Aminopropylamino)methyl]-N-[4-[4-[6-chloro-4-(trifluoromethyl)-2-pyridyl]piperazin-1-yl]sulfonyl-2-hydroxy-phenyl]benzamide;
N-[4-[4-[6-chloro-4-(trifluoromethyl)-2-pyridyl]piperazin-1-yl]sulfonylphenyl]-2-methyl-4,6-dihydropyrrolo[3,4-c]pyrazole-5-carboxamide;
N-[4-[4-[6-chloro-4-(trifluoromethyl)-2-pyridyl]piperazin-1-yl]sulfonylphenyl]-1-methyl-4,6-dihydropyrrolo[3,4-c]pyrazole-5-carboxamide;
[5-[4-[6-Chloro-4-(trifluoromethyl)-2-pyridyl]piperazin-1-yl]sulfonylindolin-1-yl]-pyrazolo[1,5-a]pyrimidin-3-yl-methanone;
4-[5-[4-[6-Chloro-4-(trifluoromethyl)-2-pyridyl]piperazin-1-yl]sulfonylindoline-1-carbonyl]-2-methyl-pyrazole-3-carboxylic acid;
[1-(Azetidin-3-yl)pyrazol-4-yl]-[5-[4-[6-chloro-4-(trifluoromethyl)-2-pyridyl]piperazin-1-yl]sulfonylindolin-1-yl]methanone;
[1-(3-Aminopropyl)pyrazol-4-yl]-[5-[4-[6-chloro-4-(trifluoromethyl)-2-pyridyl]piperazin-1-yl]sulfonylindolin-1-yl]methanone;
[5-[4-[6-Chloro-4-(trifluoromethyl)-2-pyridyl]piperazin-1-yl]sulfonylindolin-1-yl]-(4,5,6,7-tetrahydropyrazolo[1,5-a]pyrazin-3-yl)methanone;
[5-[4-[6-Chloro-4-(trifluoromethyl)-2-pyridyl]piperazin-1-yl]sulfonylindolin-1-yl]-(1-oxa-2,9-diazaspiro[4.5]dec-2-en-3-yl)methanone;
N-[4-[4-[6-chloro-4-(trifluoromethyl)-2-pyridyl]piperazin-1-yl]sulfonylphenyl]-2-piperazin-1-yl-acetamide;
1-[5-[4-[6-Chloro-4-(trifluoromethyl)-2-pyridyl]piperazin-1-yl]sulfonylindolin-1-yl]-2-(2-hydroxyethylamino)ethanone;
2-[5-[4-[6-Chloro-4-(trifluoromethyl)-2-pyridyl]piperazin-1-yl]sulfonylindoline-1-carbonyl]benzoic acid;
[2-Acetoxy-4-[5-[4-[6-chloro-4-(trifluoromethyl)-2-pyridyl]piperazin-1-yl]sulfonylindoline-1-carbonyl]phenyl]acetate;
N-[4-[4-[4-methyl-6-(trifluoromethyl)pyrimidin-2-yl]piperazin-1-yl]sulfonylphenyl]-6,7-dihydro-5H-pyrrolo[3,4-b]pyridine-3-carboxamide;
N-[4-[4-[6-methyl-4-(trifluoromethyl)-2-pyridyl]piperazin-1-yl]sulfonylphenyl]-6,7-dihydro-5H-pyrrolo[3,4-b]pyridine-3-carboxamide;
N-[4-[4-[6-chloro-4-(trifluoromethyl)-2-pyridyl]piperazin-1-yl]sulfonylphenyl]isoindoline-5-carboxamide;
N-[4-[4-[6-chloro-4-(trifluoromethyl)-2-pyridyl]piperazin-1-yl]sulfonylphenyl]-3-(2,5-diazabicyclo[2.2.1]heptan-2-yl)benzamide;
3-(2-Aminoethoxy)-N-[4-[4-[6-chloro-4-(trifluoromethyl)-2-pyridyl]piperazin-1-yl]sulfonylphenyl]benzamide;
3-(Azetidin-3-ylmethylamino)-N-[4-[4-[6-chloro-4-(trifluoromethyl)-2-pyridyl]piperazin-1-yl]sulfonylphenyl]benzamide;
3-(Azetidin-3-ylmethoxy)-N-[4-[4-[6-chloro-4-(trifluoromethyl)-2-pyridyl]piperazin-1-yl]sulfonylphenyl]benzamide;
N-[4-[4-[6-chloro-4-(trifluoromethyl)-2-pyridyl]piperazin-1-yl]sulfonylphenyl]-3-[(3R)-3-aminopyrrolidin-1-yl]benzamide;
N-[4-[4-[6-Chloro-4-(trifluoromethyl)-2-pyridyl]piperazin-1-yl]sulfonylphenyl]-3-[3,6-diazabicyclo[3.2.0]heptan-3-yl]benzamide;
3-(2-Aminoethyl)-N-[4-[4-[6-chloro-4-(trifluoromethyl)-2-pyridyl]piperazin-1-yl]sulfonylphenyl]benzamide;
N-[4-[4-[6-chloro-4-(trifluoromethyl)-2-pyridyl]piperazin-1-yl]sulfonylphenyl]-3-piperazin-1-yl-benzamide;
2-Amino-N-[4-[4-[6-chloro-4-(trifluoromethyl)-2-pyridyl]piperazin-1-yl]sulfonylphenyl]indane-5-carboxamide;
5-(Aminomethyl)-N-[4-[4-[6-chloro-4-(trifluoromethyl)-2-pyridyl]piperazin-1-yl]sulfonylphenyl]-2-hydroxy-benzamide;
N-[4-[4-[6-chloro-4-(trifluoromethyl)-2-pyridyl]piperazin-1-yl]sulfonylphenyl]-3-(2,4-diaminobutanoylamino)benzamide;
4-[(3-Aminopropylamino)methyl]-N-[4-[4-[6-chloro-4-(trifluoromethyl)-2-pyridyl]piperazin-1-yl]sulfonylphenyl]pyridine-2-carboxamide;
6-[(3-Aminopropylamino)methyl]-N-[4-[4-[6-chloro-4-(trifluoromethyl)-2-pyridyl]piperazin-1-yl]sulfonylphenyl]pyridine-2-carboxamide;
5-[(3-Aminopropylamino)methyl]-N-[4-[4-[6-chloro-4-(trifluoromethyl)-2-pyridyl]piperazin-1-yl]sulfonylphenyl]pyridine-3-carboxamide;
2-(3-Aminopropylamino)-N-[4-[4-[6-chloro-4-(trifluoromethyl)-2-pyridyl]piperazin-1-yl]sulfonylphenyl]indane-5-carboxamide;
3-(3-Aminopropylamino)-N-[4-[4-[6-chloro-4-(trifluoromethyl)-2-pyridyl]piperazin-1-yl]sulfonylphenyl]indane-5-carboxamide;
N-[4-[4-[6-chloro-4-(trifluoromethyl)-2-pyridyl]piperazin-1-yl]sulfonylphenyl]-2-methoxy-5-[(5-methyl-2,3,3a,4,6,6a-hexahydropyrrolo[2,3-c]pyrrol-1-yl)methyl]benzamide;
1-(3-Aminopropylamino)-N-[4-[4-[6-chloro-4-(trifluoromethyl)-2-pyridyl]piperazin-1-yl]sulfonylphenyl]indane-5-carboxamide;
3,4-Bis(3-Aminopropoxy)-N-[4-[4-[6-chloro-4-(trifluoromethyl)-2-pyridyl]piperazin-1-yl]sulfonylphenyl]benzamide;
3-[1-(3-Aminopropyl)pyrrolidin-2-yl]-N-[4-[4-[6-chloro-4-(trifluoromethyl)-2-pyridyl]piperazin-1-yl]sulfonylphenyl]benzamide;
3-[1-(3-Aminopropyl)pyrrolidin-3-yl]-N-[4-[4-[6-chloro-4-(trifluoromethyl)-2-pyridyl]piperazin-1-yl]sulfonylphenyl]benzamide;
3-[4-(3-Aminopropyl)piperazin-1-yl]-N-[4-[4-[6-chloro-4-(trifluoromethyl)-2-pyridyl]piperazin-1-yl]sulfonylphenyl]benzamide;
4-[2-(3-Aminopyrrolidin-1-yl)ethyl]-N-[4-[4-[6-chloro-4-(trifluoromethyl)-2-pyridyl]piperazin-1-yl]sulfonylphenyl]benzamide;
4-[2-(7-Amino-2-azaspiro[3.3]heptan-2-yl)ethyl]-N-[4-[4-[6-chloro-4-(trifluoromethyl)-2-pyridyl]piperazin-1-yl]sulfonylphenyl]benzamide;
N-[4-[4-[6-chloro-4-(trifluoromethyl)-2-pyridyl]piperazin-1-yl]sulfonylphenyl]-4-[2-(5-methyl-2,3,3a,4,6,6a-hexahydropyrrolo[2,3-c]pyrrol-1-yl)ethyl]benzamide;
N-[4-[4-[6-chloro-4-(trifluoromethyl)-2-pyridyl]piperazin-1-yl]sulfonylphenyl]-4-[[(2S)-2-(2-aminoethyl)pyrrolidin-1-yl]methyl]benzamide;
N-[4-[4-[6-chloro-4-(trifluoromethyl)-2-pyridyl]piperazin-1-yl]sulfonylphenyl]-4-[2-(3-morpholinopropylamino)ethyl]benzamide;
4-[2-(2-Amino-5-azaspiro[2.4]heptan-5-yl)ethyl]-N-[4-[4-[6-chloro-4-(trifluoromethyl)-2-pyridyl]piperazin-1-yl]sulfonylphenyl]benzamide;
N-[4-[4-[6-chloro-4-(trifluoromethyl)-2-pyridyl]piperazin-1-yl]sulfonylphenyl]-4-[2-(3,6-diazabicyclo[3.1.1]heptan-6-yl)ethyl]benzamide;
N-[4-[4-[6-chloro-4-(trifluoromethyl)-2-pyridyl]piperazin-1-yl]sulfonylphenyl]-3-[(4-piperidylmethylamino)methyl]benzamide;
3-[[(3-Aminocyclobutyl)methylamino]methyl]-N-[4-[4-[6-chloro-4-(trifluoromethyl)-2-pyridyl]piperazin-1-yl]sulfonylphenyl]benzamide;
N-[4-[4-[6-chloro-4-(trifluoromethyl)-2-pyridyl]piperazin-1-yl]sulfonylphenyl]-3-[(3-piperazin-1-ylpropylamino)methyl]benzamide;
3-[[3-(1-Aminocyclopropyl)pyrrolidin-1-yl]methyl]-N-[4-[4-[6-chloro-4-(trifluoromethyl)-2-pyridyl]piperazin-1-yl]sulfonylphenyl]benzamide;
3-[[3-[Bis(2-hydroxyethyl)amino]propylamino]methyl]-N-[4-[4-[6-chloro-4-(trifluoromethyl)-2-pyridyl]piperazin-1-yl]sulfonylphenyl]benzamide;
N-[4-[4-[6-chloro-4-(trifluoromethyl)-2-pyridyl]piperazin-1-yl]sulfonylphenyl]-2-methoxy-5-[[3-(4-piperidyl)propylamino]methyl]benzamide;
3-[[1-(Aminomethyl)-3-azabicyclo[3.1.0]hexan-3-yl]methyl]-N-[4-[4-[6-chloro-4-(trifluoromethyl)-2-pyridyl]piperazin-1-yl]sulfonylphenyl]benzamide;
(2R)-2-Amino-4-[[3-[[4-[4-[6-chloro-4-(trifluoromethyl)-2-pyridyl]piperazin-1-yl]sulfonylphenyl]carbamoyl]phenyl]methylamino]butanoic acid;
N-[4-[4-[6-chloro-4-(trifluoromethyl)-2-pyridyl]piperazin-1-yl]sulfonylphenyl]-3-[[(2-oxooxazolidin-4-yl)methylamino]methyl]benzamide;
N-[4-[4-[6-chloro-4-(trifluoromethyl)-2-pyridyl]piperazin-1-yl]sulfonylphenyl]-3-[[(5-oxopyrrolidin-3-yl)methylamino]methyl]benzamide;
N-[4-[4-[6-chloro-4-(trifluoromethyl)-2-pyridyl]piperazin-1-yl]sulfonylphenyl]-3-[[[3-(dimethylamino)-3-oxo-propyl]amino]methyl]benzamide;
5-(3,3a,4,5,6,6a-Hexahydro-2H-pyrrolo[3,4-b]pyrrol-1-ylmethyl)-N-[4-[4-[6-chloro-4-(trifluoromethyl)-2-pyridyl]piperazin-1-yl]sulfonylphenyl]-2-methoxy-benzamide; and
2-[(3-Aminopropylamino)methyl]-N-[4-[4-[6-chloro-4-(trifluoromethyl)-2-pyridyl]piperazin-1-yl]sulfonylphenyl]benzamide;
   or a pharmaceutically acceptable salt thereof.

A further embodiment of present invention is (xviii) a compound or pharmaceutically acceptable salt according to any one of (i) to (xvi) for use as therapeutically active substance.

A further embodiment of present invention is (xix) a pharmaceutical composition comprising a compound in accordance with any one of claims (i) to (xvi) and a therapeutically inert carrier.

A further embodiment of present invention is (xx) the use of a compound according to any one of (i) to (xvi) for the inhibition of LpxH.

A further embodiment of present invention is (xxi) the use of a compound according to any one of (i) to (xvi) for the treatment or prophylaxis of bacterial infection, particularly the bacteria is gram-negative bacteria.

A further embodiment of present invention is (xxii) the use of a compound according to any one of (i) to (xvi) for the preparation of a medicament for the treatment or prophylaxis of bacterial infection, particularly the bacteria is gram-negative bacteria.

A further embodiment of present invention is (xxiii) the use of a compound according to (xxi) or (xxii), wherein the gram-negative bacteria is selected from Enterobacteriaceae, Neisseria gonorrhoeae, Haemophilus influenzae, Helicobacter pylorus, Acinetobacter baumannii and Pseudomonas aeruginosa.

A further embodiment of present invention is (xxiv) the use of a compound according to (xxiii), wherein the gram-negative bacteria is Enterobacteriaceae, wherein Enterobacteriaceae is Klebsiella pneumoniae or Escherichia coli.

A further embodiment of present invention is (xxv) a compound or pharmaceutically acceptable salt according to any one of (i) to (xvi) for use in the treatment or prophylaxis of bacterial infection, particularly the bacteria is gram-negative bacteria.

A further embodiment of present invention is (xxvi) a compound according to (xxv), wherein the gram-negative bacteria is selected from Enterobacteriaceae, Neisseria gonorrhoeae, Haemophilus influenzae, Helicobacter pylorus, Acinetobacter baumannii and Pseudomonas aeruginosa.

A further embodiment of present invention is (xxvii) a compound according to (xxvi), wherein the gram-negative bacteria is Enterobacteriaceae, wherein Enterobacteriaceae is Klebsiella pneumoniae or Escherichia coli.

A further embodiment of present invention is (xxviii) a compound or pharmaceutically acceptable salt according to any one of (i) to (xvi), when manufactured according to a process of (xvii).

A further embodiment of present invention is (xxix) a method for the treatment or prophylaxis of bacterial infection, particularly the bacteria is gram-negative bacteria, which method comprises administering a therapeutically effective amount of a compound as defined in any one of (i) to (xvi), to a patient in need thereof.

### Pharmaceutical Compositions and Administration

Another embodiment provides pharmaceutical compositions or medicaments containing the compounds of the invention and a therapeutically inert carrier, diluent or excipient, as well as methods of using the compounds of the invention to prepare such compositions and medicaments. In one example, compounds of formula (I) may be formulated by mixing at ambient temperature at the appropriate pH, and at the desired degree of purity, with physiologically acceptable carriers, i.e., carriers that are non-toxic to recipients at the dosages and concentrations employed into a galenical administration form. The pH of the formulation depends mainly on the particular use and the concentration of compound, but preferably ranges anywhere from about 3 to about 8. In one example, a compound of formula (I) is formulated in an acetate buffer, at pH 5. In another embodiment, the compounds of formula (I) are sterile. The compound may be stored, for example, as a solid or amorphous composition, as a lyophilized formulation or as an aqueous solution.

Compositions are formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “effective amount” of the compound to be administered will be governed by such considerations, and is the minimum amount necessary to reduced bacterial load or improve host survival through the inhibition of Lipid A biosynthesis by targeting LpxH enzyme. For example, such amount may be below the amount that is toxic to normal cells, or the mammal as a whole.

In one example, the pharmaceutically effective amount of the compound of the invention administered parenterally per dose will be in the range of about 0.1 to 1000 mg/kg, alternatively about 1 to 100 mg/kg of patient body weight per day, with the typical initial range of compound used being 0.3 to 15 mg/kg/day. In another embodiment, oral unit dosage forms, such as tablets and capsules, preferably contain from about 5 to about 5000 mg of the compound of the invention.

The compounds of the invention may be administered by any suitable means, including oral, topical (including buccal and sublingual), rectal, vaginal, transdermal, parenteral, subcutaneous, intraperitoneal, intrapulmonary, intradermal, intrathecal and epidural and intranasal, and, if desired for local treatment, intralesional administration. Parenteral infusions include intramuscular, intravenous, intraarterial, intraperitoneal, or subcutaneous administration.

The compounds of the present invention may be administered in any convenient administrative form, e.g., tablets, powders, capsules, solutions, dispersions, suspensions, syrups, sprays, suppositories, gels, emulsions, patches, etc. Such compositions may contain components conventional in pharmaceutical preparations, e.g., diluents, carriers, pH modifiers, sweeteners, bulking agents, and further active agents.

A typical formulation is prepared by mixing a compound of the present invention and a carrier or excipient. Suitable carriers and excipients are well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., a compound of the present invention or pharmaceutical composition thereof) or aid in the manufacturing of the pharmaceutical product (i.e., medicament).

An example of a suitable oral dosage form is a tablet containing about 10 to 500 mg of the compound of the invention compounded with about 40 to 400 mg anhydrous lactose, about 5 to 50 mg sodium croscarmellose, about 5 to 50 mg polyvinylpyrrolidone (PVP) K30, and about 1 to 10 mg magnesium stearate. The powdered ingredients are first mixed together and then mixed with a solution of the PVP. The resulting composition can be dried, granulated, mixed with the magnesium stearate and compressed to tablet form using conventional equipment. An example of an aerosol formulation can be prepared by dissolving the compound, for example 5 to 1000 mg) of the invention in a suitable buffer solution, e.g. a phosphate buffer, adding a tonicifier, e.g. a salt such sodium chloride, if desired. The solution may be filtered, e.g., using a 0.2 micron filter, to remove impurities and contaminants.

An embodiment, therefore, includes a pharmaceutical composition comprising a compound of Formula (I), or a stereoisomer or pharmaceutically acceptable salt thereof. In a further embodiment includes a pharmaceutical composition comprising a compound of Formula (I), or a stereoisomer or pharmaceutically acceptable salt thereof, together with a pharmaceutically acceptable carrier or excipient.

Another embodiment includes a pharmaceutical composition comprising a compound of formula (I) for use in the treatment and/or prevention of bacterial infections.

The following composition A and B illustrate typical compositions of the present invention, but serve merely as representative thereof.

### Composition A

A compound of the present invention can be used in a manner known per se as the active ingredient for the production of tablets of the following composition:

| | Per tablet | |
|---|---|---|
| Active ingredient | 200 | mg |
| Microcrystalline cellulose | 155 | mg |
| Corn starch | 25 | mg |
| Talc | 25 | mg |
| Hydroxypropylmethylcellulose | 20 | mg |
| | 425 | mg |

### Composition B

A compound of the present invention can be used in a manner known per se as the active ingredient for the production of capsules of the following composition:

Per capsule

| | | |
|---|---|---|
| Active ingredient | 100.0 | mg |
| Corn starch | 20.0 | mg |
| Lactose | 95.0 | mg |
| Talc | 4.5 | mg |
| Magnesium stearate | 0.5 | mg |
| | 220.0 | mg |

### Indications and Methods of Treatment

The compounds of the invention are inhibitors of the LpxH enzyme, a key enzyme of the LPS synthesis pathway that is essential in most gram-negative bacteria. Accordingly, the compounds of the invention can prevent bacterial growth of susceptible organisms and are useful for: preventing or treating a bacterial infection, preferably a Gram-negative bacterial infection (all claimed) e.g. nosocomial pneumonia, urinary tract infections, systemic infections (bacteraemia and sepsis), skin and soft tissue infections, surgical infections, eye infections, intraabdominal infections, lung infections and diabetic foot infections caused by Gram-negative bacteria e.g. third generation cephalosporins- and carbapenem-resistant Enterobacteriaeceae (e.g. Klebsiella pneumoniae, Escherichia coli) and multi-drug-resistant Pseudomonas aeruginosa and Acinetobacter baumannii or Acinetobacter spp., e.g. Neisseria gonorrhoeae, Haemophilus influenzae, Helicobacter pylorus e.g. Bacteroides spp. e.g. Bacteroides fragilis, Bacteroides thetaiotaomicron, Bacteroides distasonis, Campylobacter jejuni, Campylobacter fetus or Campylobacter coil, Francisella tularensis and Providencia spp. e.g. Providencia stuartii, Providencia rettgeri or Providencia alcalifaciens and Pseudomonas spp.; and for cleaning purposes e.g. to remove pathogenic microbes and bacteria from surgical instruments, catheters and artificial implants or to make a room or an area aseptic.

The products of the invention can be administered, for example, parenterally e.g. by injection, or administered orally, perorally, such as in the form of tablets, coated tablets, dragees, hard and soft gelatin capsules, solutions, emulsions or suspensions, or rectally, such as in the form of suppositories. Pharmaceutical compositions containing these compounds can be prepared using conventional procedures familiar to those skilled in the art, such as by combining the ingredients into a dosage form together with suitable, non-toxic, inert, therapeutically compatible solid or liquid carrier materials and, if desired, the usual pharmaceutical adjuvants. It is contemplated that the compounds are ultimately embodied into compositions of suitable oral, parenteral or topical dosage forms. The compositions of this invention can contain, as optional ingredients, any of the various adjuvants, which are used ordinarily in the production of pharmaceutical preparations. Thus, for example, in formulating the present compositions into the desired oral dosage forms, one may use, as optional ingredients, fillers, such as co-precipitated aluminum hydroxide-calcium carbonate, di-calcium phosphate or lactose; disintegrating agents such as maize starch; and lubricating agents, such as talc, calcium stearate, and the like. It should be fully understood, however, that the optional ingredients herein named are given by way of example only and that the invention is not restricted to the use hereof. Other such adjuvants, which are well known in the art, can be employed in carrying out this invention. Suitable as such carrier materials are not only inorganic, but also organic carrier materials. Thus, for tablets, coated tablets, dragees and hard gelatin capsules there can be used, for example, lactose, maize starch or derivatives thereof, talc, stearic acid or its salts. Suitable carriers for soft gelatin capsules are, for example, vegetable oils, waxes, fats and semi-solid and liquid polyols (depending on the nature of the active substance; no carriers are, however, required in the case of soft gelatin capsules). Suitable carrier materials for the preparation of solutions and syrups are, for example, water, polyols, saccharose, invert sugar and glucose. Suitable carrier materials for suppositories are, for example, natural or hardened oils, waxes, fats and semi-liquid or liquid polyols. As pharmaceutical adjuvants there are contemplated the usual preservatives, solubilizers, stabilizers, wetting agents, emulsifiers, sweeteners, colorants, flavorings, salts for varying the osmotic pressure, buffers, coating agents and antioxidants.

### Synthesis

The compounds of the present invention can be prepared by any conventional means. Suitable processes for synthesizing these compounds as well as their starting materials are provided in the schemes below and in the examples. All substituents, in particular, R¹, R², R³, R⁴, ²⁵Y, Q¹, Q², Q³, Q⁴ and Q⁵ are defined above. Furthermore, and unless explicitly otherwise stated, all reactions, reaction conditions, abbreviations and symbols have the meanings well known to a person of ordinary skill in organic chemistry.

General synthetic routes for preparing the compound of formula (I) are shown below.

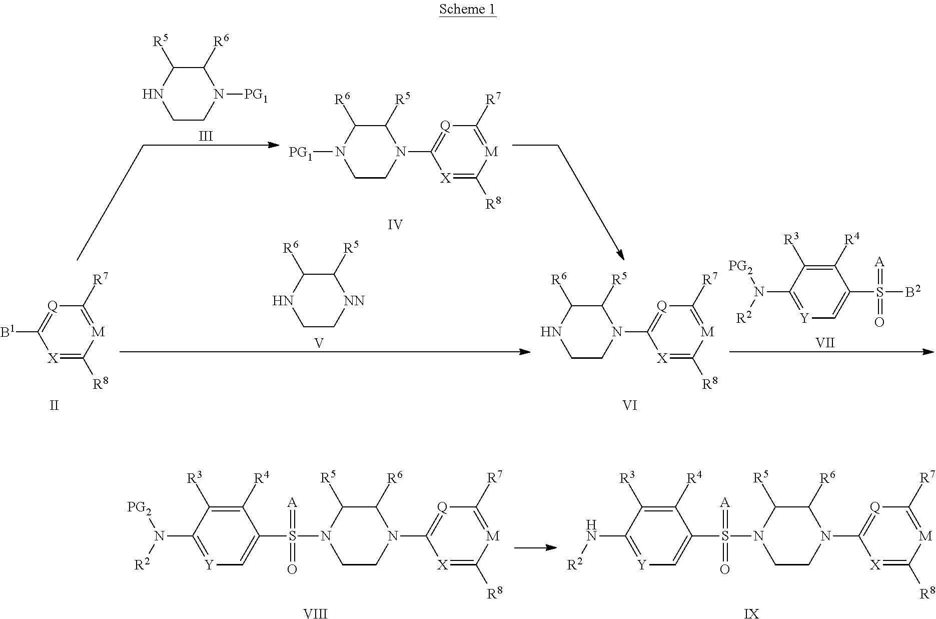

wherein B¹ and B² are halogen; PG¹ is a protecting group, such as tert-butoxycarbonyl or benzyloxycarbonyl group; PG² is a protecting group, such as acetyl group.

Nucleophilic substitution reaction between compound of formula (II) and compound of formula (III) affords compound of formula (IV), which is deprotected with a suitable acid, such as TFA, or reduction reagents, such as palladium on carbon under H₂, to afford compound of formula (VI). Alternatively, compound of formula (VI) could be obtained directly using nucleophilic substitution reaction between compound of formula (II) and compound of formula (V) in the presence a suitable base, such as TEA. Compound of formula (VIII) could be obtained from a sulfonating reaction between halogenated phenyl sulfonyl chlorides (VII) and compound of formula (VI). The formation of compound (IX) can be achieved by treating compound of formula (VIII) with a suitable acid, such as HCl, or a base, such as K₂CO₃.

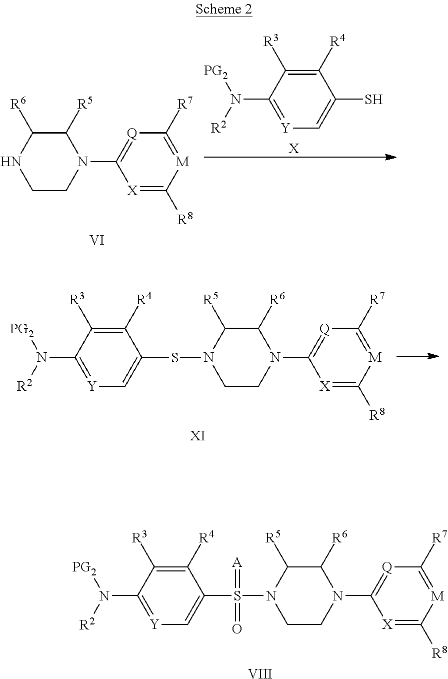

wherein PG² is a protecting group, such as acetyl group.

Alternatively, compound of formula (VIII) can also be prepared in the process illustrated in the scheme 2. Compound of formula (XI) can be obtained by coupling reaction between compound of formula (VI) and compound of formula (X) in the presence of catalyst such as CuO. Oxidation of compound of formula (XI) will give compound of formula (VIII) in the presence of suitable oxidant reagent, such as PIDA.

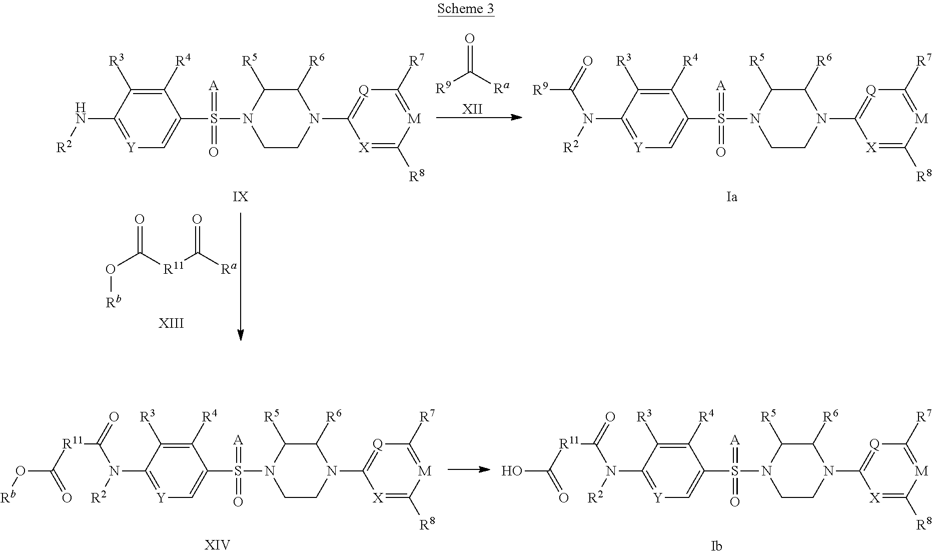

wherein R^{a} is halogen or OH; R^{b} is C₁₋₆alkyl, such as methyl or ethyl, R¹¹ is pyrazolylene, phenylene or aminoC₁₋₆alkylaminoC₁₋₆alkylene.

The compound of formula (Ia) can be prepared by coupling reaction between compound of formula (IX) and compound of formula (XII) in the presence of condensation reagent such as TCFH or acylation reaction in the presence of base such as TEA.

The compound of formula (Ib) can be prepared by starting with coupling reaction or acylation reaction between compound of formula (IX) and compound of formula (XIII) to afford compound of formula (XIV). Hydrolysis of compound of formula (XIV) in the presence a suitable base, such as LiGH, affords compound of formula of (Ib).

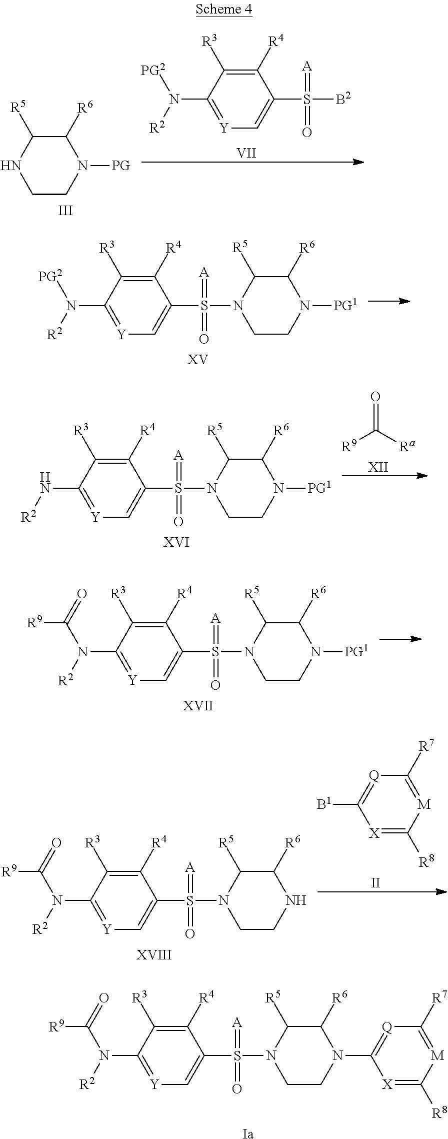

Compound of formula (XV) can be obtained from a nucleophilic substitution reaction between compound of formula (III) and compound of formula (VII) in the presence a suitable base such as DIPEA or TEA. Deprotection of compound of formula (XV) affords compound of formula (XVI) in the presence a suitable acid, such as HCl, or base, such as K₂CO₃. Compound of formula (XVII) can be obtained by acylation reaction between compound of formula (XVI) and compound of formula (XII) in the presence a suitable base such as TEA. In some cases, compound of formula (XVII) needs to be further protected by another PG¹. Deprotection of compound of formula (XVII) affords compound of formula (XVIII) using a suitable acid such as TFA or reduction reagents such as palladium on carbon under H₂ atmosphere. The nucleophilic substitution between compound of formula (II) and compound of formula (XVIII) in the presence of suitable base such as Cs₂CO₃ affords compound of formula (Ia). In some cases, compound of formula (Ia) needs to be further deprotected to remove PG¹.

Compounds of this invention can be obtained as mixtures of diastereomers or enantiomers, which can be separated by methods well known in the art, e.g. (chiral) HPLC or SFC.

This invention also relates to a process for the preparation of a compound of formula (I) comprising any of the following steps:
a) formation of compound of formula (Ia),

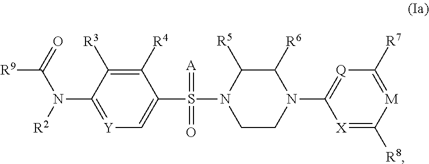

via coupling reaction or acylation reaction between compound of formula (IX),

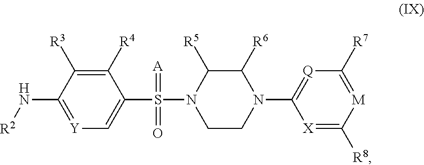

and compound of formula (XII),

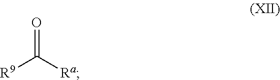

b) formation of compound of formula (Ib),

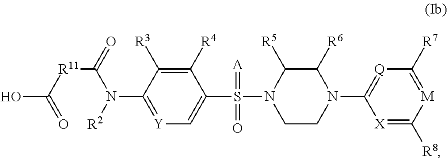

via hydrolysis of compound of formula (XIV),

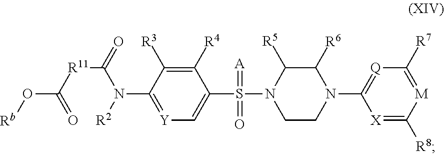

c) formation of compound of formula (Ia),

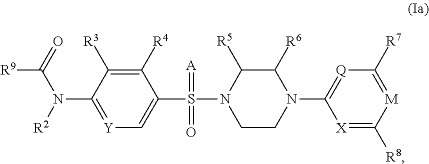

via nucleophilic substitution between compound of formula (XVIII),

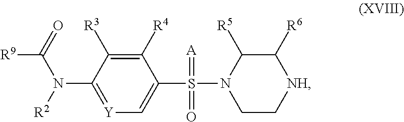

and compound of formula (II),

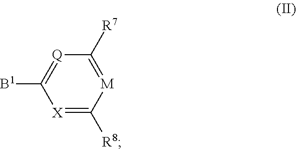

wherein B¹ is halogen; PG¹ is a protecting group, such as tert-butoxycarbonyl or benzyloxycarbonyl group; PG² is a protecting group, such as acetyl group; R^{a} is halogen or OH; R^{b} is C₁₋₆alkyl, such as methyl or ethyl, R¹¹ is pyrazolylene, phenylene or aminoC₁₋₆alkylaminoC₁₋₆alkylene.

A compound of formula (I) when manufactured according to the above process is also an object of the invention.

### EXAMPLES

The invention will be more fully understood by reference to the following examples. They should not, however, be construed as limiting the scope of the invention.

### Abbreviations

The invention will be more fully understood by reference to the following examples. They should not, however, be construed as limiting the scope of the invention.

Abbreviations used herein are as follows:
aq.: aqueous
DAST: diethylaminosulfur trifluoride
DBU: 1,8-diazabicyclo[5.4.0]undec-7-ene
DCM: dichloromethane
DIPEA: N,N-Diisopropylethylamine
DMAP: 4-dimethylaminopyridine
DMEDA: 1,2-dimethylethylenediamine
DMP: Dess-Martin periodinane
FA: formic acid
FBS: fetal bovine serum
HATU: 1-[bis(dimethylamino)methylene]-1H-1,2,3-triazolo[4,5-b]pyridinium 3-oxide
hexafluorophosphate
IC₅₀: the molar concentration of an inhibitor, which produces 50% of the maximum
possible response for that inhibitor.
HPLC: high performance liquid chromatography
MS (ESI): mass spectroscopy (electron spray ionization)
MeCN: acetonitrile
MsCl: methylsulfonyl chloride
MTBE: methyl tert-butyl ether
obsd: observed
PE: petroleum ether
PIDA: phenyliodo(III)diacetate
TCFH: Chloro-N,N,N′,N′-tetramethylformamidinium hexafluorophosphate
TFA: trifluoroacetic acid
TEA: triethylamine
δ: chemical shift
SFC: supercritical fluid chromatography
TLC: thin layer chromatography

### General Experimental Conditions

Intermediates and final compounds were purified by flash chromatography using one of the following instruments: i) Biotage SP1 system and the Quad 12/25 Cartridge module. ii) ISCO combi-flash chromatography instrument. Silica gel brand and pore size: i) KP-STL 60 A, particle size: 40-60 μm; ii) CAS registry NO: Silica Gel: 63231-67-4, particle size: 47-60 micron silica gel; iii) ZCX from Qingdao Haiyang Chemical Co., Ltd, pore: 200-300 or 300-400.

Intermediates and final compounds were purified by preparative HPLC on reversed phase column using XBridge™ Prep-C18 (5 μm, OBD™ 30×100 mm) column, SunFire™ Prep-C18 (5 μm, OBD™ 30×100 mm) column, Phenomenex Synergi-C18 (10 μm, 25×150 mm) or Phenomenex Gemini-C18 (10 μm, 25×150 mm). Waters AutoP purification System (Sample Manager 2767, Pump 2525, Detector: Micromass ZQ and UV 2487, solvent system: acetonitrile and 0.1% ammonium hydroxide in water; acetonitrile and 0.1% FA in water or acetonitrile and 0.1% TFA in water). Or Gilson-281 purification System (Pump 322, Detector: UV 156, solvent system: acetonitrile and 0.05% ammonium hydroxide in water; acetonitrile and 0.225% FA in water; acetonitrile and 0.05% HCl in water; acetonitrile and 0.075% TFA in water; or acetonitrile and water).

For SFC chiral separation, intermediates were separated by chiral column (Daicel chiralpak IC, 5 μm, 30×250 mm), AS (10 μm, 30×250 mm) or AD (10 μm, 30×250 mm) using Mettler Toledo Multigram III system SFC, Waters 80Q preparative SFC or Thar 80 preparative SFC, solvent system: CO₂ and IPA (0.5% TEA in IPA) or CO₂ and MeOH (0.1% NH₃—H₂O in MeOH), back pressure 100bar, detection UV@254 or 220 nm.

LC/MS spectra of compounds were obtained using a LC/MS (Waters™ Alliance 2795-Micromass ZQ, Shimadzu Alliance 2020-Micromass ZQ or Agilent Alliance 6110-Micromass ZQ), LC/MS conditions were as follows (running time 3 or 1.5 mins):
Acidic condition I: A: 0.1% TFA in H₂O; B: 0.1% TFA in acetonitrile;
Acidic condition II: A: 0.0375% TFA in H₂O; B: 0.01875% TFA in acetonitrile;
Basic condition I: A: 0.1% NH₃—H₂O in H₂O; B: acetonitrile;
Basic condition II: A: 0.025% NH₃—H₂O in H₂O; B: acetonitrile;
Neutral condition: A: H₂O; B: acetonitrile.

Mass spectra (MS): generally only ions which indicate the parent mass are reported, and unless otherwise stated the mass ion quoted is the positive mass ion (MH)*.

NMR Spectra were obtained using Bruker Avance 400 MHz.

The microwave assisted reactions were carried out in a Biotage Initiator Sixty microwave synthesizer. All reactions involving air-sensitive reagents were performed under an argon or nitrogen atmosphere. Reagents were used as received from commercial suppliers without further purification unless otherwise noted.

### Preparative Examples

The following examples are intended to illustrate the meaning of the present invention but should by no means represent a limitation within the meaning of the present invention:

Intermediate 1: 4-[4-[6-Chloro-4-(trifluoromethyl)-2-pyridyl]piperazin-1-yl]sulfonylaniline

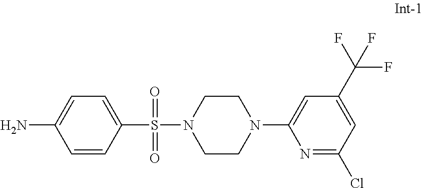 [SMILES: Nc1ccc(S(=O)(=O)N2CCN(c3cc(C(F)(F)F)cc(Cl)n3)CC2)cc1]

Step 1: 1-[6-Chloro-4-(trifluoromethyl)-2-pyridyl]piperazine

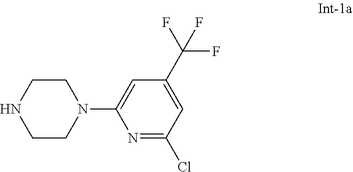 [SMILES: FC(F)(F)c1cc(Cl)nc(N2CCNCC2)c1]

To a solution of piperazine (47.86 g, 555.58 mmol) in 1,4-dioxane (150 mL) were added 2,6-dichloro-4-(trifluoromethyl)pyridine (15.0 g, 69.45 mmol) slowly. The mixture was stirred at 60° C. for 1 hour. After completion, the mixture was diluted with water (300 mL) and extracted with EA (200 mL×3). The combined organic layer was washed with brine (300 mL×4), dried over anhydrous Na₂SO₄ and concentrated to give compound Int-1a (18 g, 67.76 mmol, 97.6% yield) as a white solid. MS obsd.: 265.8 (MH+).

Step 2: N-[4-[4-[6-chloro-4-(trifluoromethyl)-2-pyridyl]piperazin-1-yl]sulfonylphenyl]acetamide

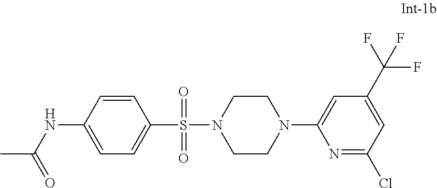 [SMILES: CC(=O)Nc1ccc(S(=O)(=O)N2CCN(c3cc(C(F)(F)F)cc(Cl)n3)CC2)cc1]

To a solution of compound Int-1a (500.0 mg, 1.88 mmol) and DIPEA (0.81 mL, 4.63 mmol) in DCM (15 mL) was added 4-acetamidobenzenesulfonyl chloride (468.0 mg, 2.0 mmol) at 0° C. The mixture was slowly warmed up to rt and stirred at rt for 1 hour. After completion, the mixture was poured into water (20 mL) and extracted with DCM (20 mL×3). The combined organic layer was concentrated under vacuum to give crude product, which was purified by silica-gel chromatography (elute with EtOAc:PE=20%) to afford compound Int-1b as a white solid (800 mg, 92% yield). MS obsd. (ESI⁺) [(M+H)⁺]: 463.2.

Step 2: 4-[4-[6-Chloro-4-(trifluoromethyl)-2-pyridyl]piperazin-1-yl]sulfonylaniline

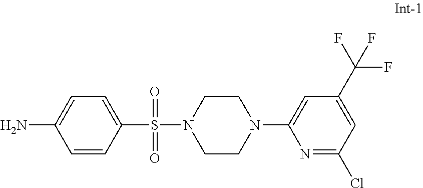 [SMILES: Nc1ccc(S(=O)(=O)N2CCN(c3cc(C(F)(F)F)cc(Cl)n3)CC2)cc1]

To a solution of compound Int-1a (970.0 mg, 2.1 mmol) in THF (10 mL) was added hydrochloric acid (12 M, 5 mL) at rt. The mixture was heated to 40° C. for 1 hour. After completion, the mixture was concentrate in vacuo to give a residue, which was diluted with sat. NaHCO₃(aq, 30 mL) and extracted with EtOAc (20 mL×3). The combined organic layer was washed with brine (50 mL), dried over anhydrous Na₂SO₄ and concentrated under vacuum to give compound Int-1 (830 mg, 94.11% yield) as yellow oil. MS obsd. (ESI⁺) [(M+H)⁺]: 421.2.

Intermediate 2: 4-(Chloromethyl)-N-[4-[4-[6-chloro-4-(trifluoromethyl)-2-pyridyl]piperazin-1-yl]sulfonylphenyl]benzamide

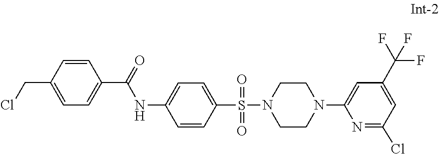 [SMILES: O=C(Nc1ccc(S(=O)(=O)N2CCN(c3cc(C(F)(F)F)cc(Cl)n3)CC2)cc1)c1ccc(CCl)cc1]

To a solution of compound Int-1 (820.0 mg, 1.95 mmol) and TEA (0.81 mL, 5.85 mmol) in DCM (20 mL) was added 4-(chloromethyl)benzoyl chloride (736.68 mg, 3.9 mmol) at rt and stirred for 1 hour. After completion, the mixture was quenched with water (30 mL) and extracted with DCM (20 mL×3). The combined organic layer was washed with brine (50 mL), dried over anhydrous Na₂SO₄ and concentrated under vacuum to give crude product, which was purified by prep-HPLC (TFA as additive) to give compound Int-2 (600 mg, 1.05 mmol, 53.7% yield) as yellow solid. MS obsd. (ESI⁺) [(M+H)⁺]: 573.2.

Intermediate 3: 3-(Chloromethyl)-N-[4-[4-[6-chloro-4-(trifluoromethyl)-2-pyridyl]piperazin-1-yl]sulfonylphenyl]benzamide

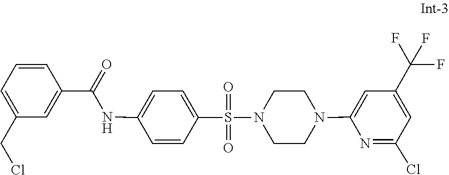 [SMILES: O=C(Nc1ccc(S(=O)(=O)N2CCN(c3cc(C(F)(F)F)cc(Cl)n3)CC2)cc1)c1cccc(CCl)c1]

To a solution of compound Int-1 (2.0 g, 4.75 mmol) and TEA (1.6 mL, 11.7 mmol) in DCM (40 mL) was added 3-(chloromethyl)benzoyl chloride (1.08 g, 5.7 mmol) at rt and the reaction mixture was stirred for 1 h. After completion, the mixture was quenched with water (50 mL) and extracted with DCM (30 mL×3). The combined organic layer was washed with brine (50 mL), dried over anhydrous Na₂SO₄ and concentrated under vacuum to give crude product, which was purified by silica-gel chromatography (elute with EtOAc:PE=20%) to give Int-3 (2.0 g, 3.49 mmol, 73.4% yield) as white solid. MS obsd. (ESI⁺) [(M+H)⁺]: 573.2.

Intermediate 4: 5-[4-[6-Chloro-4-(trifluoromethyl)-2-pyridyl]piperazin-1-yl]sulfonylindoline

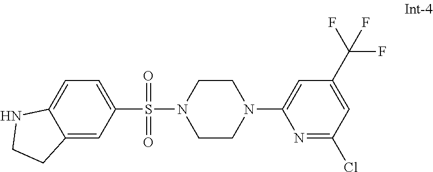 [SMILES: O=S(=O)(c1ccc2c(c1)CCN2)N1CCN(c2cc(C(F)(F)F)cc(Cl)n2)CC1]

Step 1: 1-Indolin-1-ylethanone

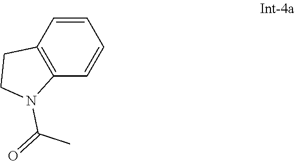

A solution of indoline (6.5 g, 54.5 mmol) in acetic anhydride (20 mL) was heated at 80° C. for 2 hours. And then, the reaction mixture was concentrated to afford crude Int-4a as a yellow gum (8.9 g, 54.6 mmol, 100% yield), which was used in the next step directly. MS obsd. (ESI⁺) [(M+H)⁺]: 162.1.

Step 2: 1-Acetylindoline-5-sulfonyl chloride

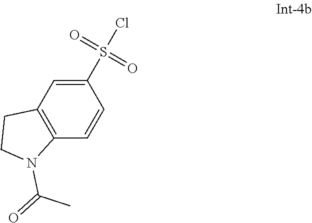 [SMILES: CC(=O)N1CCc2cc(S(=O)(=O)Cl)ccc21]

A solution of compound Int-4a (8.2 g, 50.9 mmol) in sulfurochloridic acid (237 g, 2.03 mol) was stirred at rt for 2 hours. And then, the mixture was poured onto ice, extracted with DCM (100 mL×3). The combined organic layer was dried over Na₂SO₄ and concentrated under vacuum to give crude compound Int-4b (3.0 g, 22.7% yield), which was used in the next step directly.

Step 3: 1-[6-[4-[6-Chloro-4-(trifluoromethyl)-2-pyridyl]piperazin-1-yl]sulfonylindolin-1-yl]ethanone

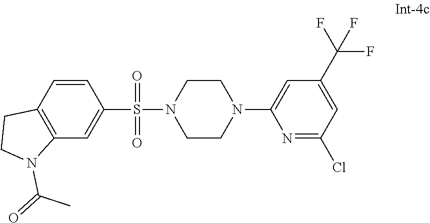 [SMILES: CC(=O)N1CCc2ccc(S(=O)(=O)N3CCN(c4cc(C(F)(F)F)cc(Cl)n4)CC3)cc21]

To a solution of compound Int-1a (1.8 g, 6.78 mmol) and TEA (1.8 g, 17.3 mmol) in DCM (100 mL) was added compound Int-4b (1.5 g, 5.8 mmol) portion-wise at 0° C. The mixture was stirred at rt for 2 hours. And then, the mixture was quenched with water (50 mL), extracted with DCM (100 mL×3). The combined organic layer was concentrated under vacuum to give a crude product, which was purified by silica-gel chromatography (elute with EtOAc:PE=20%) to afford compound Int-4c (2.5 g, 75.3%) as light yellow solid. MS obsd. (ESI⁺) [(M+H)⁺]: 489.3.

Step 4: 6-[4-[6-chloro-4-(rifluoromethyl)-2-pyridyl]piperazin-1-yl]sulfonylindoline

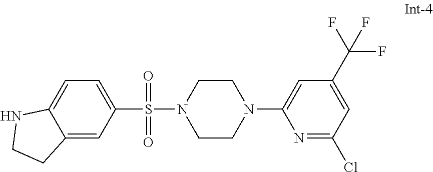 [SMILES: O=S(=O)(c1ccc2c(c1)CCN2)N1CCN(c2cc(C(F)(F)F)cc(Cl)n2)CC1]

To a solution of compound Int-4c (2.0 g, 4.09 mmol) in THF (20 mL) was added hydrochloric acid (12 M, 5 mL) at rt. The mixture was heated to 70° C. for 1 hour. After completion, the mixture was concentrate under vacuum to give a residue, which was diluted with sat. NaHCO₃ (30 mL) and extracted with EtOAc (20 mL×3). The combined organic layer was washed with brine (50 mL), dried over anhydrous Na₂SO₄ and concentrated under vacuum to give crude compound Int-4 (1.0 g, 54.7% yield) as a yellow solid. MS obsd. (ESI⁺) [(M+H)⁺]: 447.2.

Intermediate 5: Tert-butyl N-[3-(tert-butoxycarbonylamino)propyl]-N-[[4-[(4-piperazin-1-ylsulfonylphenyl)carbamoyl]phenyl]methyl]carbamate

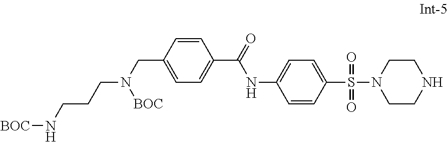 [SMILES: CC(C)(C)OC(=O)NCCCN(Cc1ccc(C(=O)Nc2ccc(S(=O)(=O)N3CCNCC3)cc2)cc1)C(=O)OC(C)(C)C]

Step 1: Benzyl 4-(4-acetamidophenyl)sulfonylpiperazine-1-carboxylate

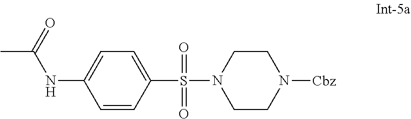 [SMILES: CC(=O)Nc1ccc(S(=O)(=O)N2CCN(C(=O)OCc3ccccc3)CC2)cc1]

To a solution of benzyl piperazine-1-carboxylate (5 g, 22.7 mmol) and 4-acetamidobenzenesulfonyl chloride (5.3 g, 22.7 mmol) in DCM (100 mL) was added TEA (6.88 g, 68.1 mmol) dropwise at rt. The mixture was stirred at rt for 2 hours. And then, the mixture was quenched with NaHCO₃ aq. (100 mL), extracted with DCM (100 mL×3). The combined organic layer was concentrated under vacuum to give crude, which was rinsed with hexane to afford desired product Int-5a (9.2 g, 97.1% yield) as a white solid. MS obsd. (ESI⁺) [(M+H)⁺]: 418.2.

Step 2: Benzyl 4-(4-aminophenyl)sulfonylpiperazine-1-carboxylate

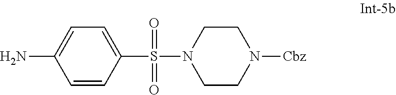 [SMILES: Nc1ccc(S(=O)(=O)N2CCN(C(=O)OCc3ccccc3)CC2)cc1]

To a mixture of Int-5a (9.2 g, 22 mmol) in MeOH (100 mL) was added AcCi (8 mL, 22 mmol) dropwise at rt. The mixture was heated at 60° C. for 4 hours. And then, the reaction mixture was concentrated to afford crude compound Int-5b (8.0 g, 96.7% yield) as a white solid. MS obsd. (ESI⁺) [(M+H)⁺]: 376.2.

Step 3: Benzyl 4-[4-[[4-(chloromethyl)benzoyl]amino]phenyl]sulfonylpiperazine-1-carboxylate

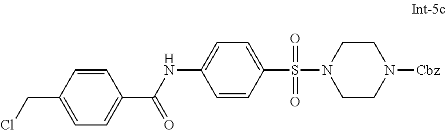 [SMILES: O=C(Nc1ccc(S(=O)(=O)N2CCN(C(=O)OCc3ccccc3)CC2)cc1)c1ccc(CCl)cc1]

To a solution of compound Int-5b (2.0 g, 5.32 mmol) and TEA (1.6 mL, 11.7 mmol) in DCM (40 mL) was added 4-(chloromethyl)benzoyl chloride (1.08 g, 5.7 mmol) at rt and stirred for 1 hour. After completion, the mixture was quenched with water (50 mL) and extracted with DCM (30 mL×3). The combined organic layer was washed with brine (50 mL), dried over anhydrous Na₂SO₄ and concentrated under vacuum to give crude product, which was purified by silica-gel chromatography (elute with EtOAc:PE=20%) to give compound Int-5c (2.0 g, 73.4% yield) as a white solid. MS obsd. (ESI⁺) [(M+H)⁺]: 528.2.

Step 4: Benzyl 4-[4-[[4-[[3-(tert-butoxycarbonylamino)propylamino]methyl]benzoyl]amino]phenyl]sulfonylpiperazine-1-carboxylate

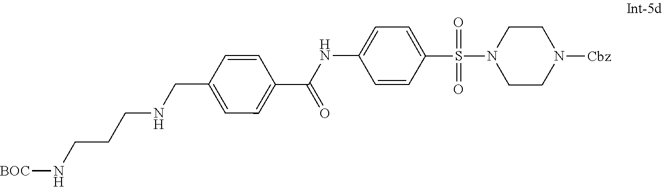 [SMILES: CC(C)(C)OC(=O)NCCCNCc1ccc(C(=O)Nc2ccc(S(=O)(=O)N3CCN(C(=O)OCc4ccccc4)CC3)cc2)cc1]

A mixture of compound Int-5c (2.0 g, 3.78 mmol), DIPEA (2.7 mL, 15.3 mmol) and tert-butyl N-(3-aminopropyl)carbamate (1.2 mL, 5.6 mmol) in THF (100 mL) was stirred at 70° C. for 16 hours. After completion, the solvent was removed under vacuum and the residue was purified by prep-HPLC (TFA as additive) to afford compound Int-5d (1.5 g, 59.5% yield). MS obsd. (ESI⁺) [(M+H)⁺]: 666.3.

Step 5: Benzyl 4-[4-[[4-[[tert-butoxycarbonyl-[3-(tert-butoxycarbonylamino)propyl]amino]methyl]benzoyl]aminolphenyl]sulfonylpiperazine-1-carboxylate

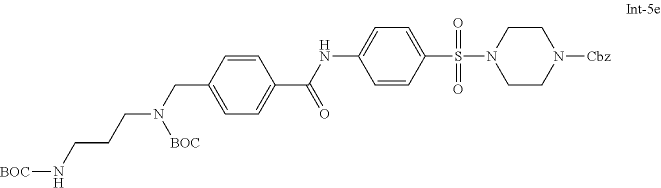 [SMILES: CC(C)(C)OC(=O)NCCCN(Cc1ccc(C(=O)Nc2ccc(S(=O)(=O)N3CCN(C(=O)OCc4ccccc4)CC3)cc2)cc1)C(=O)OC(C)(C)C]

To a mixture of compound Int-5d (1.5 g, 2.25 mmol) and DIPEA (1.0 mL, 5.7 mmol) in DCM (40 mL) was added Boc-anhydride (1.0 g, 4.58 mmol) portion-wise at rt and stirred at rt for 1 hour. After completion, the mixture was quenched with saturated NaHCO₃ aq. (40 mL), extracted with DCM (40 mL×3). The combined organic layer was concentrated under vacuum to give a crude product, which was purified by silica-gel chromatography (elute with MeOH:DCM=10%) to afford compound Int-5e (1.6 g, 2.08 mmol, 92.8% yield). MS obsd. (ESI⁺) [(M+H)⁺]: 766.3.

Step 7: Tert-butyl N-[3-(tert-butoxycarbonylamino)propyl]-N-[[4-[(4-piperazin-1-ylsulfonylphenyl)carbamoyl]phenyl]methyl]carbamate

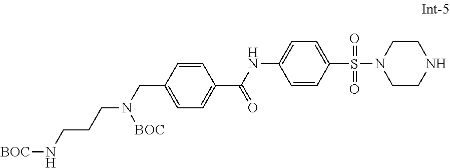 [SMILES: CC(C)(C)OC(=O)NCCCN(Cc1ccc(C(=O)Nc2ccc(S(=O)(=O)N3CCNCC3)cc2)cc1)C(=O)OC(C)(C)C]

The mixture of compound Int-5e (1.6 g, 2.08 mmol) and Pd/C (200 mg, 10% Pd on Carbon) under H₂ balloon was stirred at rt for 8 hours. After completion, the mixture was filtered and the filtrate was concentrated under vacuum to give crude Int-5 (1.2 g, 91.3%), which was used in the next step directly. MS obsd. (ESI⁺) [(M+H)⁺]: 632.3.

Intermediate 6: Tert-butyl N-[3-(tert-butoxycarbonylamino)propyl]-N-[[3-[(4-piperazin-1-ylsulfonylphenyl)carbamoyl]phenyl]methyl]carbamate

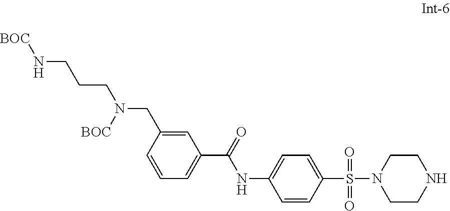 [SMILES: CC(C)(C)OC(=O)NCCCN(Cc1cccc(C(=O)Nc2ccc(S(=O)(=O)N3CCNCC3)cc2)c1)C(=O)OC(C)(C)C]

The title compound was prepared in analogy to the preparation of compound Int-5 by replacing 4-(chloromethyl)benzoyl chloride with 3-(chloromethyl)benzoyl chloride in Step 3. Compound Int-6 (1.0 g) was obtained. MS obsd. (ESI⁺) [(M+H)⁺]: 623.3.

Intermediate 7: Tert-butyl N-[3-(tert-butoxycarbonylamino)propyl]-N-[2-[4-[(4-piperazin-1-ylsulfonylphenyl)carbamoyl]phenyl]ethyl]carbamate

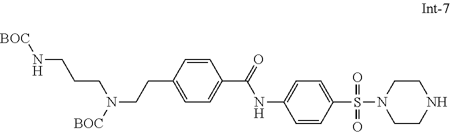 [SMILES: CC(C)(C)OC(=O)NCCCN(CCc1ccc(C(=O)Nc2ccc(S(=O)(=O)N3CCNCC3)cc2)cc1)C(=O)OC(C)(C)C]

The title compound was prepared in analogy to the preparation of compound Int-5 by replacing 4-(chloromethyl)benzoyl chloride with 4-(2-bromoethyl)benzoyl chloride in Step 3. Compound Int-7 (1.1 g) was obtained. MS obsd. (ESI⁺) [(M+H)⁺]: 646.3.

Intermediate 8: 2,6-Dichloro-4-(1,1-difluoroethyl)pyridine

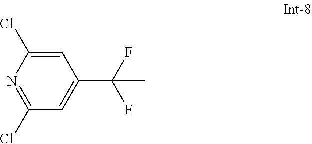

1-(2,6-Dichloro-4-pyridyl)ethanone (300.0 mg, 1.58 mmol, 1 eq) was dissolved in DAST (2.0 mL) and the reaction was stirred at rt for 6 hours. After completion, the mixture was poured into ice-water (40 mL), extracted with EtOAc (20 mL×3). The combined organic layer was dried over Na₂SO₄, concentrated under vacuum to give residue, which was purified by prep-HPLC (TFA buffer) to afford compound Int-8 (167 mg, 44.90% yield). MS obsd. (ESI⁺) [(M+H)⁺]: 212.1.

Intermediate 9: 3-[[Tert-butoxycarbonyl-[3-(tert-butoxycarbonylamino)propyl]amino]methyl]benzoic acid

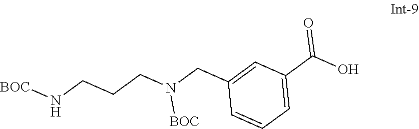 [SMILES: CC(C)(C)OC(=O)NCCCN(Cc1cccc(C(=O)O)c1)C(=O)OC(C)(C)C]

Step 1: Methyl 3-[[3-(tert-butoxycarbonylamino)propylamino]methyl]benzoate

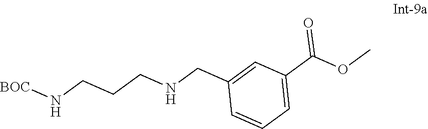 [SMILES: COC(=O)c1cccc(CNCCCNC(=O)OC(C)(C)C)c1]

A mixture of methyl 3-(bromomethyl)benzoate (2.0 g, 8.73 mmol), tert-butyl N-(3-aminopropyl)carbamate (2.28 g, 13.1 mmol), K₂CO₃ (12.1 g, 8.73 mmol,) and KI (0.05 mL, 0.870 mmol) in MeCN (20 mL) was heated to 60° C. for 1 hour. After completion, the mixture was concentrated under vacuum to give a crude product, which was diluted with water (20 mL). The aqueous phase was extracted with EtOAc (20 mL×3). The combined organic layer was washed with brine (50 mL), dried with anhydrous Na₂SO₄ and concentrated under vacuum to give compound Int-9a (2.8 g, 49.74% yield) as a colorless oil, which was used in the next step directly. MS obsd. (ESI⁺) [(M+H)⁺]: 323.1.

Step 2: Methyl 3-[[tert-butoxycarbonyl-[3-(tert-butoxycarbonylamino)propyl]amino]methyl]benzoate

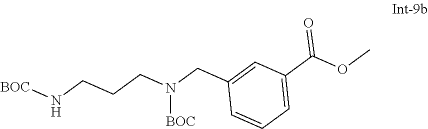 [SMILES: COC(=O)c1cccc(CN(CCCNC(=O)OC(C)(C)C)C(=O)OC(C)(C)C)c1]

To a solution of methyl Int-9a (2.8 g, 8.68 mmol) in THF (10 mL) and water (10 mL) were added Na₂CO₃ (1.84 g, 17.37 mmol) and BOC anhydride (1.90 g, 8.68 mmol). The reaction mixture was stirred at 20° C. for 1 hour. After completion, the reaction mixture was diluted with water (50 mL), extracted with EtOAc (20 mL×3). The combined organic layer was washed with brine (50 mL), dried over anhydrous Na₂SO₄, concentrated under vacuum to give compound Int-9b (3.5 g, 47.69% yield) as a colorless oil, which was used directly in the next step. MS obsd. (ESI⁺) [(M+H)⁺]: 423.2.

Step 3: 3-[[Tert-butoxycarbonyl-[3-(tert-butoxycarbonylamino)propyl]amino]methyl]benzoic acid

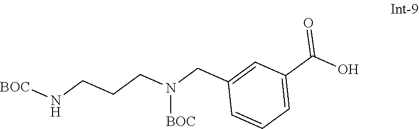 [SMILES: CC(C)(C)OC(=O)NCCCN(Cc1cccc(C(=O)O)c1)C(=O)OC(C)(C)C]

To a solution of Int-9b (3.5 g, 8.28 mmol) in EtOH (20 mL) and water (10 mL) was added NaOH (1.66 g, 41.42 mmol) in one portion. The reaction mixture was stirred at 20° C. for 2 hours. After completion, the mixture was concentrated under vacuum to remove EtOH. The residue was diluted with water (20 mL) and then adjusted the pH to 5-6 with HCl (aq. 2 mol/L). The aqueous phase was extracted with EtOAc (20 mL×3). The combined organic layer was dried with anhydrous Na₂SO₄, concentrated under vacuum to give a crude, which was purified by prep-HPLC (TFA as additive) to give compound Int-9 (1.4 g, 41.37% yield) as a colorless oil. MS obsd. (ESI⁺) [(M+H)⁺]: 409.2.

Intermediate 10: 3-[2-[Tert-butoxycarbonyl-[3-(tert-butoxycarbonylamino)propyl]amino]ethyl]benzoic acid

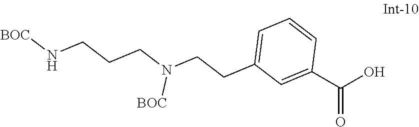 [SMILES: CC(C)(C)OC(=O)NCCCN(CCc1cccc(C(=O)O)c1)C(=O)OC(C)(C)C]

The title compound was prepared in analogy to the preparation of compound Int-9 by replacing methyl 3-(bromomethyl)benzoate with methyl 3-(2-bromoethyl)benzoate in step 1. Compound Int-10 (1.2 g) was obtained. MS obsd. (ESI⁺) [(M+H)⁺]: 423.1.

Intermediate 11: N-(4-methylsulfinamoylphenyl)acetamide

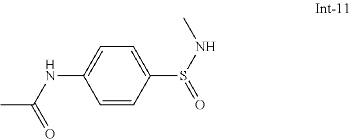

Step 1: 4-Acetamidobenzenesulfinic acid

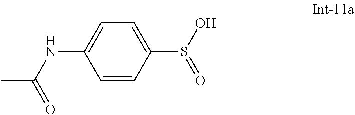

To a solution of sodium sulfite (12.84 g, 101.85 mmol) and NaHCO₃ (10.27 g, 122.22 mmol) in water (55 mL) was added N-acetylsulfanilyl chloride (11.9 g, 50.93 mmol) slowly at 70° C. After the addition, the mixture was stirred at 70° C. for 2 hours. After completion, the mixture was diluted with water (100 mL) and acidified with 60% H₂SO₄ (50 mL) at 0° C. and the resulting precipitate was collected by filtration. The filter cake was washed by 20% aq. H₂SO₄(100 mL) and dried under vacuum to give compound Int-11a (8 g, 78.85% yield) as a white solid. MS obsd. (ESI⁺) [(M+H)⁺]: 200.0.

Step 2: 4-Acetamidobenzenesulfinyl chloride

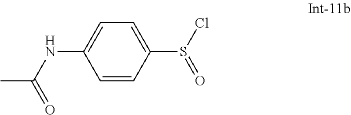

To a solution of compound Int-11a (7.0 g, 35.14 mmol) in DCM (30 mL) and THF (30 mL) were added SOCl₂ (16.72 g, 140.54 mmol) in one portion. The mixture was stirred at 15° C. for 4 hours. The mixture was concentrated under vacuum to give crude compound Int-11b (7 g, 91.53% yield) as a yellow solid, which was used in the next step directly.

Step 3: N-(4-methylsulfinamoylphenyl)acetamide

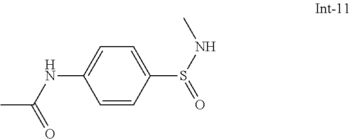

To a solution of monomethylamine in THF (27.56 mL, 55.13 mmol) in THF (20 mL) was added 4-acetamidobenzenesulfinyl chloride (1.5 g, 6.89 mmol) in DCM (5.0 mL) dropwise. The mixture was stirred at 15° C. for 1 hour. After completion, the mixture was diluted with water (50 mL) and extracted with EtOAc (100 mL×3). The combined organic layer was washed with brine (50 mL), dried over anhydrous Na₂SO₄ and concentrated under vacuum to give compound Int-11 (1.2 g, 82.04% yield) as a yellow solid, which was used in the next step directly. MS obsd. (ESI⁺) [(M+H)⁺]: 212.8.

Intermediate 12A and 12B: 2-Methyl-5,6-dihydro-4H-pyrrolo[3,4-c]pyrazole (Int-12A) and 1-Methyl-5,6-dihydro-4H-pyrrolo[3,4-c]pyrazole (Int-12B)

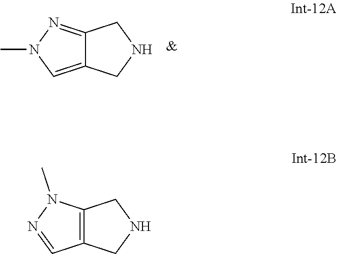

Step1: Tert-butyl 2-methyl-4,6-dihydropyrrolo[3,4-c]pyrazole-5-carboxylate (Int-11Aa) and Tert-butyl 1-methyl-4,6-dihydropyrrolo[3,4-c]pyrazole-5-carboxylate (Int-11Ba)

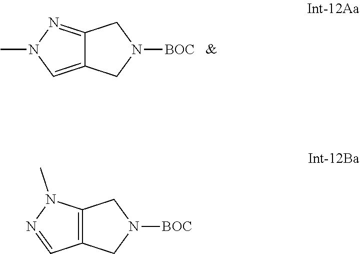 [SMILES: Cn1cc2c(n1)CN(C(=O)OC(C)(C)C)C2 ; Cn1ncc2c1CN(C(=O)OC(C)(C)C)C2]

To a solution of tert-butyl 4,6-dihydro-2H-pyrrolo[3,4-c]pyrazole-5-carboxylate (600 mg, 2.87 mmol) in DMF (30 mL) was added NaH (229.4 mg, 5.74 mmol) at 0° C. The resulting suspension mixture was stirred at rt for 15 minutes. And then, iodomethane (1221 mg, 8.6 mmol) was added to the mixture and the mixture was stirred at 25° C. for 4 hours. After completion, the reaction was quenched with ice-water (30 mL), extracted with EtOAc (30 mL×2). The combined organic layer was concentrated under vacuum to give a crude product, which was purified by silica-gel chromatography (elute with EtOAc:PE=50%) to give the mixture of Int-12Aa&12Ba (400 mg, 37.48% yield) as a brown oil. MS obsd. (ESI⁺) [(M+H)⁺]: 224.1.

Step 2: 2-Methyl-5,6-dihydro-4H-pyrrolo[3,4-c]pyrazole (Int-12A) and 1-Methyl-5,6-dihydro-4H-pyrrolo[3,4-c]pyrazole (Int-12B)

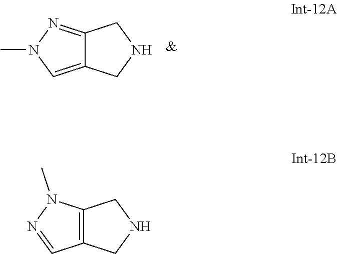

The mixture of compounds Int-12Aa&12Ba (400 mg, 1.792 mmol) in DCM (10 mL) and TFA (2 mL) was stirred at rt for 4 hours. After completion, the mixture was concentrated under vacuum to give the TFA salt of Int-12A&12B (200 mg, 56.48% yield) as a brown oil. MS obsd. (ESI⁺) [(M+H)⁺]: 124.1.

Intermediate 13: 4-[4-[4-Methyl-6-(trifluoromethyl)pyrimidin-2-yl]piperazin-1-yl]sulfonylaniline

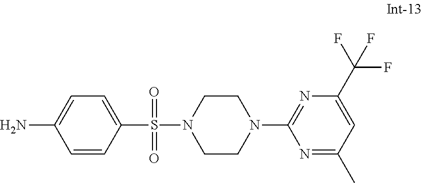 [SMILES: Cc1cc(C(F)(F)F)nc(N2CCN(S(=O)(=O)c3ccc(N)cc3)CC2)n1]

The title compound was prepared in analogy to the preparation of compound Int-1 by replacing 2,6-dichloro-4-(trifluoromethyl)pyridine with 2-chloro-4-methyl-6-(trifluoromethyl)pyrimidine in step1. Compound Int-13 (1.2 g) was obtained. MS obsd. (ESI⁺) [(M+H)⁺]: 402.1.

Intermediate 14: 2-(Tert-butoxycarbonyl)isoindoline-5-carboxylic acid

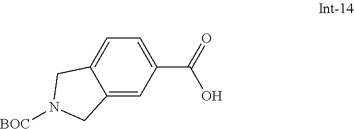 [SMILES: CC(C)(C)OC(=O)N1Cc2ccc(C(=O)O)cc2C1]

Step 1: 2-Tert-butyl 5-(2,4,6-trichlorophenyl) isoindoline-2,5-dicarboxylate

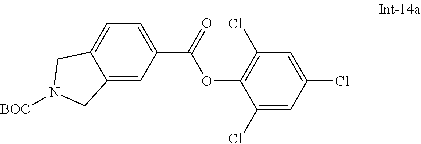 [SMILES: CC(C)(C)OC(=O)N1Cc2ccc(C(=O)Oc3c(Cl)cc(Cl)cc3Cl)cc2C1]

To a solution of tert-butyl 5-bromoisoindoline-2-carboxylate (238.0 mg, 0.800 mmol) in toluene (4 mL) were added 9,9-dimethyl-4,5-bis(diphenylphosphino)xanthene (92.37 mg, 0.160 mmol), palladium (II) acetate (17.92 mg, 0.080 mmol), TEA (0.22 mL, 1.6 mmol, 2 eq) and (2,4,6-trichlorophenyl) formate (215.95 mg, 0.960 mmol) in sequence. The reaction mixture was heated to 90° C. for 15 hours under N₂. After completion, the reaction mixture was concentrated to give a residue, which was purified by prep-HPLC (FA as additive) to give compound Int-14a (260 mg, 73.58% yield) as a white solid. MS obsd. (ESI⁺) [(M+H)⁺]: 442.0.

Step 2: 2-(Tert-butoxycarbonyl)isoindoline-5-carboxylic acid

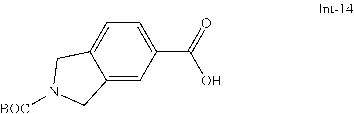 [SMILES: CC(C)(C)OC(=O)N1Cc2ccc(C(=O)O)cc2C1]

A solution of compound Int-14a (240.0 mg, 0.540 mmol) and NaOH (216.84 mg, 5.42 mmol) in THF (1.02 mL) and water (0.102 mL) was stirred for 2 hours at 20° C. After completion, the mixture was acidified with 1N HCl to pH=6. The solution was extracted with DCM (50 mL×3). The combined organic layer was washed with brine (100 mL), dried over Na₂SO₄, concentrated under reduced pressure to give compound Int-14 (130 mg, 91.08% yield) as a yellow oil. MS obsd. (ESI⁺) [(M+H)⁺]: 264.0.

Intermediate 15: 3-[2-(Tert-butoxycarbonylamino)ethyl]benzoic acid

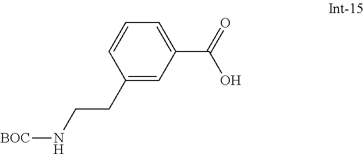 [SMILES: CC(C)(C)OC(=O)NCCc1cccc(C(=O)O)c1]

The title compound was prepared in analogy to the preparation of compound Int-14 by replacing tert-butyl 5-bromoisoindoline-2-carboxylate with tert-butyl N-[2-(3-bromophenyl)ethyl]carbamate in step1. Compound Int-15 (1.3 g) was obtained. MS obsd. (ESI⁺) [(M+H)⁺]: 266.0.

Intermediate 16: 2-(Tert-butoxycarbonylamino)indane-5-carboxylic acid

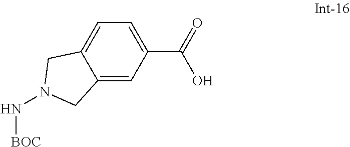 [SMILES: CC(C)(C)OC(=O)NN1Cc2ccc(C(=O)O)cc2C1]

The title compound was prepared in analogy to the preparation of compound Int-14 by replacing tert-butyl 5-bromoisoindoline-2-carboxylate with tert-butyl N-(5-bromoindan-2-yl)carbamate in step1. Compound Int-16 (1.1 g) was obtained. MS obsd. (ESI⁺) [(M+H)⁺]: 278.1.

Intermediate 17: 6-[[Tert-butoxycarbonyl-[3-(tert-butoxycarbonylamino)propyl]amino]methyl]pyridine-2-carboxylic acid

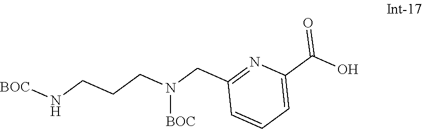 [SMILES: CC(C)(C)OC(=O)NCCCN(Cc1cccc(C(=O)O)n1)C(=O)OC(C)(C)C]

The title compound was prepared in analogy to the preparation of compound Int-9 by replacing methyl 3-(bromomethyl)benzoate with methyl 6-(bromomethyl)pyridine-2-carboxylate in step 1. Compound Int-17 (1.0 g) was obtained. MS obsd. (ESI⁺) [(M+H)⁺]: 410.2

Intermediate 18: 2-[Tert-butoxycarbonyl-[3-(tert-butoxycarbonylamino)propyl]amino]indane-5-carboxylic acid

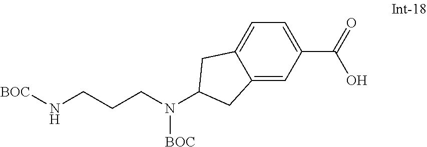 [SMILES: CC(C)(C)OC(=O)NCCCN(C(=O)OC(C)(C)C)C1Cc2ccc(C(=O)O)cc2C1]

Step 1: Tert-butyl N-[3-[(5-bromoindan-2-yl)amino]propyl]carbamate

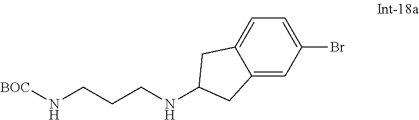 [SMILES: CC(C)(C)OC(=O)NCCCNC1Cc2ccc(Br)cc2C1]

A mixture of 5-bromoindan-2-one (600.0 mg, 2.84 mmol), tert-butyl N-(3-aminopropyl)carbamate (0.99 g, 5.69 mmol) and anhydrous Na₂SO₄ (400 mg, 2.84 mmol) in MeOH (5 mL) and THF (5 mL) was stirred at 25° C. for 3 hours. Then sodium cyanoborohydride (893.21 mg, 14.21 mmol) was added to the mixture and stirred at 25° C. for another 12 hours. After completion, the mixture was poured into ice-water (100 mL) and extracted with EtOAc (150 mL×3). The combined organic layer was washed with brine (100 mL), dried over anhydrous Na₂SO₄ and concentrated under vacuum to give crude compound Int-18a (800 mg, 76.2% yield) as a light yellow oil, which was used in next step directly. MS obsd. (ESI⁺) [(M+H)⁺]: 369.1.

Step 2: Tert-butyl N-(5-bromoindan-2-yl)-N-[3-(tert-butoxycarbonylamino)propyl]carbamate

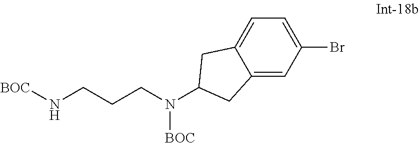 [SMILES: CC(C)(C)OC(=O)NCCCN(C(=O)OC(C)(C)C)C1Cc2ccc(Br)cc2C1]

To a solution of compound Int-18a (800.0 mg, 2.17 mmol) and Boc anhydride (0.57 g, 2.6 mmol) in THF (20 mL) was added TEA (0.3 mL, 2.17 mmol) dropwise. The mixture was stirred at 15° C. for 12 hours. After completion, the mixture was poured into water (50 mL) and extracted with EtOAc (50 mL×3). The combined organic layer was washed with brine (30 mL), dried over anhydrous Na₂SO₄ and concentrated under vacuum to give residue, which was purified by silica-gel chromatography (elute with EtOAc:PE=20%) to give compound Int-18b (900 mg, 88.51% yield) as a yellow oil. MS obsd. (ESI⁺) [(M+H)⁺]: 469.3.

Step 3: (2,4,6-Trichlorophenyl) 2-[tert-butoxycarbonyl-[3-(tert-butoxycarbonylamino)propyl]amino]indane-5-carboxylate

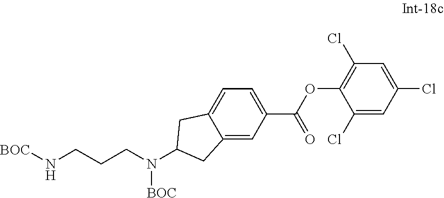 [SMILES: CC(C)(C)OC(=O)NCCCN(C(=O)OC(C)(C)C)C1Cc2ccc(C(=O)Oc3c(Cl)cc(Cl)cc3Cl)cc2C1]

The title compound was prepared in analogy to the preparation of compound Int-14a by replacing tert-butyl 5-bromoisoindoline-2-carboxylate with compound 18b in step1. Compound Int-18c (800 mg) was obtained as yellow solid. MS obsd. (ESI⁺) [(M+H)⁺]: 613.0.

Step 4: 2-[Tert-butoxycarbonyl-[3-(tert-butoxycarbonylamino)propyl]amino]indane-5-carboxylic acid

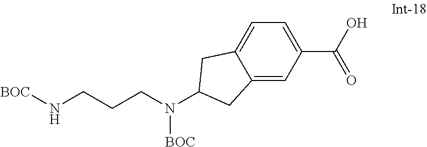 [SMILES: CC(C)(C)OC(=O)NCCCN(C(=O)OC(C)(C)C)C1Cc2ccc(C(=O)O)cc2C1]

The title compound was prepared in analogy to the preparation of compound Int-14 by replacing compound Int-14a with compound Int-18c in step 2. Compound Int-18 (300 mg) was obtained as a white solid. MS obsd. (ESI⁺) [(M+H)⁺]: 435.2.

Intermediate 19: 3-[1-[3-(Tert-butoxycarbonylamino)propyl]pyrrolidin-2-yl]benzoic acid

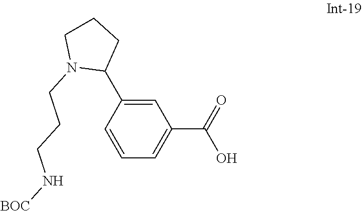 [SMILES: CC(C)(C)OC(=O)NCCCN1CCCC1c1cccc(C(=O)O)c1]

Step 1: Tert-butyl N-[3-[2-(3-bromophenyl)pyrrolidin-1-yl]propyl]carbamate

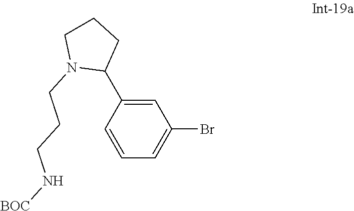 [SMILES: CC(C)(C)OC(=O)NCCCN1CCCC1c1cccc(Br)c1]

The mixture of 2-(3-bromophenyl) pyrrolidine (2.0 g, 8.85 mmol), K₂CO₃ (2.44 g, 17.69 mmol), KI (0.23 mL, 4.42 mmol) and tert-butyl N-(3-chloropropyl) carbamate (2.56 g, 13.27 mmol) in MeCN (10 mL) was stirred at 65° C. for 2 hours. After completion, the mixture was poured into water (100 mL) and extracted with EtOAc (100 mL×3). The combined organic layer was washed by brine, dried over Na₂SO₄ and concentrated under vacuum to give crude product, which was purified by and prep-HPLC (FA as additive) to afford compound Int-18a (2.5 g, 73.73% yield) as yellow oil. MS obsd. (ESI⁺) [(M+H)⁺]: 383.2.

Step 2: Methyl 3-[1-[3-(tert-butoxycarbonylamino)propyl]pyrrolidin-2-yl]benzoate

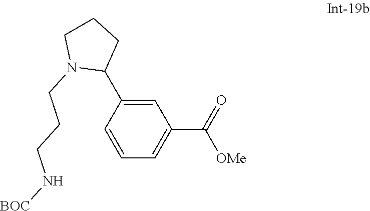 [SMILES: COC(=O)c1cccc(C2CCCN2CCCNC(=O)OC(C)(C)C)c1]

The mixture of Int-19a (1000.0 mg, 2.61 mmol), TEA (0.65 mL, 4.7 mmol) and [1,1′-bis(diphenylphosphino)ferrocene]dichloropalladium(II) (114.53 mg, 0.160 mmol) was purged with carbon oxide and heated to 80° C. for 16 hours under CO atmosphere. After completion, the mixture was concentrated under vacuum to give a residue, which was purified by silica-gel chromatography (elute with EtOAc:PE=10%) to give compound int-19b (500 mg, 1.38 mmol) as yellow oil. MS obsd. (ESI⁺) [(M+H)⁺]: 363.3.

Step 3: 3-[1-[3-(Tert-butoxycarbonylamino)propyl]pyrrolidin-2-yl]benzoic acid

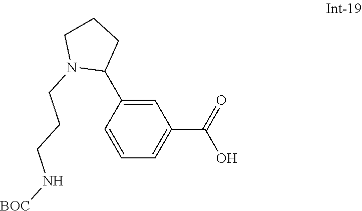 [SMILES: CC(C)(C)OC(=O)NCCCN1CCCC1c1cccc(C(=O)O)c1]

The mixture of compound Int-19b (400.0 mg, 1.1 mmol) and LiGH (264.31 mg, 11.04 mmol) in THF (9.35 mL) and water (9.35 mL) was stirred at 60° C. for 2 hours. After completion, the mixture was acidified to PH=5-6 using 1N HCl solution. The mixture was extracted with EA:MeOH=10:1(100 mL×3). The combined organic layer was dried over Na₂SO₄ and concentrated under vacuum to give crude product which was further purified by silica-gel chromatography (elute with MeOH:DCM=20%) to provide compound Int-19 (100 mg, 26.01% yield) as yellow oil. MS obsd. (ESI⁺) [(M+H)⁺]: 349.1.

Intermediate 20: 3-[1-[3-(Tert-butoxycarbonylamino)propyl]pyrrolidin-3-yl]benzoic acid

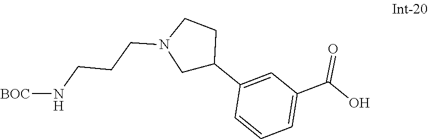 [SMILES: CC(C)(C)OC(=O)NCCCN1CCC(c2cccc(C(=O)O)c2)C1]

The title compound was prepared in analogy to the preparation of compound Int-19 by replacing 2-(3-bromophenyl) pyrrolidine with 3-(3-bromophenyl) pyrrolidine in step 1. Compound Int-20 (300 mg) was obtained as a white solid. MS obsd. (ESI⁺) [(M+H)⁺]: 349.1.

### Example 001: 4-[(3-Aminopropylamino)methyl]-N-[4-[4-[6-chloro-4-(trifluoromethyl)-2-pyridyl]piperazin-1-yl]sulfonylphenyl]benzamide

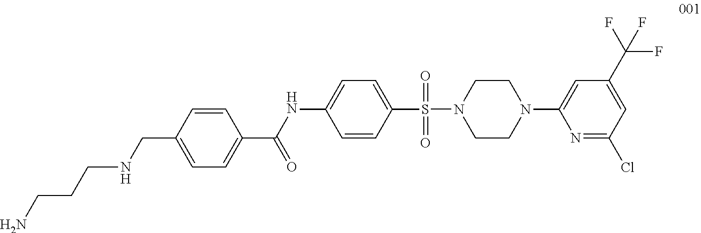 [SMILES: NCCCNCc1ccc(C(=O)Nc2ccc(S(=O)(=O)N3CCN(c4cc(C(F)(F)F)cc(Cl)n4)CC3)cc2)cc1]

Step 1: Tert-butyl N-[3-[[4-[[4-[4-[6-chloro-4-(trifluoromethyl)-2-pyridyl]piperazin-1-yl]sulfonylphenyl]carbamoyl]phenyl]methylamino]propyl]carbamate

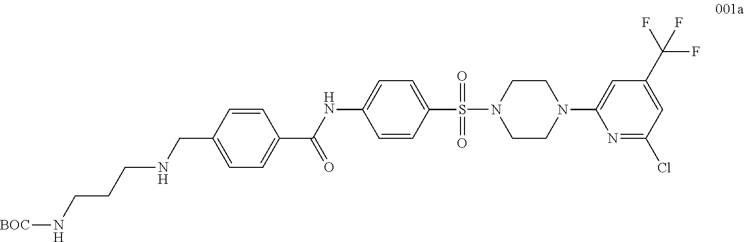 [SMILES: CC(C)(C)OC(=O)NCCCNCc1ccc(C(=O)Nc2ccc(S(=O)(=O)N3CCN(c4cc(C(F)(F)F)cc(Cl)n4)CC3)cc2)cc1]

To a solution of compound Int-2 (60.0 mg, 0.100 mmol) in MeCN (10 mL) were added tert-butyl N-(3-aminopropyl)carbamate (36.46 mg, 0.210 mmol), K₂CO₃ (28.92 mg, 0.210 mmol) and KI (8.3 mg, 0.050 mmol) in one portion. The mixture was heated to 60° C. for 2 hours. After completion, the mixture was concentrated in vacuo to give a residue, which was diluted with water (20 mL). The aqueous phase was extracted with DCM (10 mL×3). The combined organic layer was washed with brine (20 mL), dried with anhydrous Na₂SO₄ and concentrated under vacuum to give crude product, which was purified by prep-TLC (DCM/MeOH=10/1) to afford compound 001a (55 mg, 73.91% yield) as a yellow solid. MS obsd. (ESI⁺) [(M+H)]⁺: 711.3.

Step 2: 4-[(3-Aminopropylamino)methyl]-N-[4-[4-[6-chloro-4-(trifluoromethyl)-2-pyridyl]piperazin-1-yl]sulfonylphenyl]benzamide

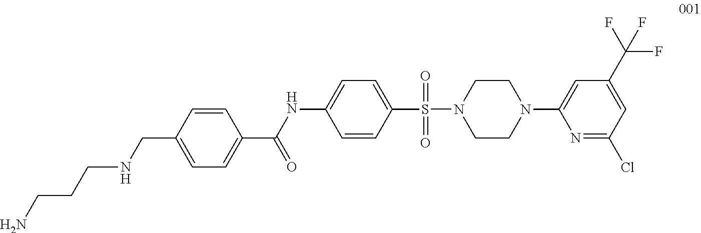 [SMILES: NCCCNCc1ccc(C(=O)Nc2ccc(S(=O)(=O)N3CCN(c4cc(C(F)(F)F)cc(Cl)n4)CC3)cc2)cc1]

To a solution of compound 001a (55.0 mg, 0.080 mmol) in DCM (1 mL) was added TFA (0.6 mL, 7.73 mmol) in one portion. The mixture was stirred at 25° C. for 1 hour. After completion, the mixture was concentrated under vacuum to give a residue, which was purified by prep-HPLC (HCl as additive) to afford Example 001 (25 mg, 48.07% yield) was obtained as white solid. MS obsd. (ESI⁺) [(M+H)]⁺: 611.3.

### Example 001: ¹H NMR (400 MHz, DMSO-d6) δ ppm 10.78 (s, 1H), 9.68-9.53 (m, 2H), 8.15-8.01 (m, 7H), 7.80-7.73 (m, 4H), 7.13-7.06 (m, 1H), 6.96 (s, 1H), 4.26-4.18 (m, 2H), 3.76-3.68 (m, 4H), 3.06-2.95 (m, 6H), 2.94-2.85 (m, 2H), 2.08-1.99 (m, 2H)

### Example 002: 4-[(3-Aminopropylamino)methyl]-N-[4-[4-[4-(trifluoromethyl)-2-pyridyl]piperazin-1-yl]sulfonylphenyl]benzamide

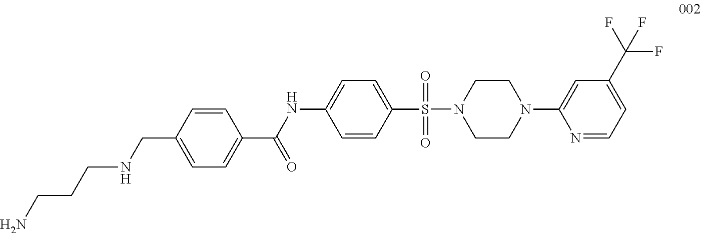 [SMILES: NCCCNCc1ccc(C(=O)Nc2ccc(S(=O)(=O)N3CCN(c4cc(C(F)(F)F)ccn4)CC3)cc2)cc1]

The title compound was prepared in analogy to the preparation of Example 001, replacing compound 2,6-dichloro-4-(trifluoromethyl)pyridine with 2-chloro-4-(trifluoromethyl)pyridine in Step 1 of preparing Int-1. Example 002 (100 mg) was obtained as a white solid. MS obsd. (ESI⁺) [(M+H)⁺]: 577.2.

### Example 002: ¹H NMR (400 MHz, DMSO-d6) δ ppm 10.71 (s, 1H), 8.30 (d, J=4.9 Hz, 1H), 8.08 (d, J=8.3 Hz, 2H), 7.94 (d, J=7.3 Hz, 2H), 7.75 (d, J=8.3 Hz, 2H), 7.52 (d, J=7.1 Hz, 2H), 7.09 (s, 1H), 6.89 (d, J=5.0 Hz, 1H), 3.82 (s, 2H), 3.71 (s, 4H), 2.97 (s, 4H), 2.86 (s, 2H), 2.62 (s, 2H), 1.74 (s, 2H). ¹⁹F NMR (376 MHz, DMSO-d6) δ ppm −63.57 (s, 3F)

### Example 003: 4-[(3-Aminopropylamino)methyl]-N-[4-[4-[5-chloro-4-(trifluoromethyl)-2-pyridyl]piperazin-1-yl]sulfonylphenyl]benzamide

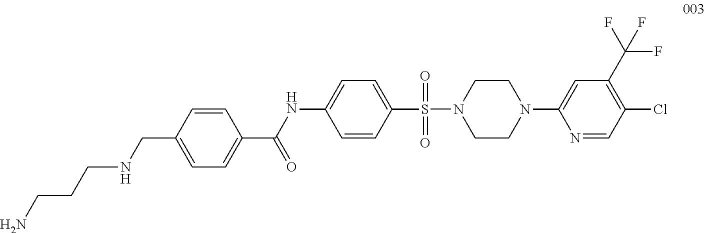 [SMILES: NCCCNCc1ccc(C(=O)Nc2ccc(S(=O)(=O)N3CCN(c4cc(C(F)(F)F)c(Cl)cn4)CC3)cc2)cc1]

Step 1: Tert-butyl N-[3-(tert-butoxycarbonylamino)propyl]-N-[[4-[[4-[4-[5-chloro-4-(trifluoromethyl)-2-pyridyl]piperazin-1-yl]sulfonylphenyl]carbamoyl]phenyl]methyl]carbamate

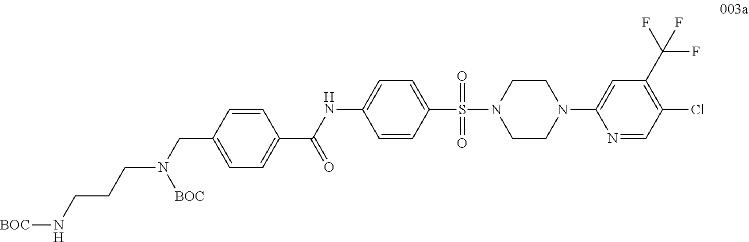 [SMILES: CC(C)(C)OC(=O)NCCCN(Cc1ccc(C(=O)Nc2ccc(S(=O)(=O)N3CCN(c4cc(C(F)(F)F)c(Cl)cn4)CC3)cc2)cc1)C(=O)OC(C)(C)C]

A mixture of 2,5-dichloro-4-(trifluoromethyl)pyridine (38.83 mg, 0.180 mmol), compound Int-5 (80.0 mg, 0.180 mmol) and Cs₂CO₃ (mg, 0.180 mmol) in NMP (5 mL) was heated at 100° C. for 1 hour. After completion, the mixture was poured into 50 mL water and extracted with EtOAc (25 mL×2). The combined organic layer was washed with brine (25 mL×2), dried over Na₂SO₄ and concentrated to give a residue, which was purified by silica-gel chromatography (elute with EtOAc:PE=30%) to afford the desired product compound 003a (65 mg, 40.11% yield) as a white solid. MS obsd. (ESI⁺) [(M+H)⁺]: 811.2.

Step 2: 4-[(3-Aminopropylamino)methyl]-N-[4-[4-[5-chloro-4-(trifluoromethyl)-2-pyridyl]piperazin-1-yl]sulfonylphenyl]benzamide

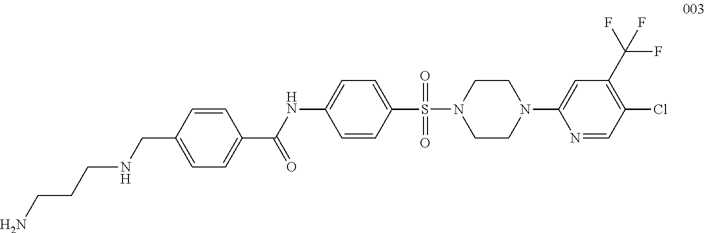 [SMILES: NCCCNCc1ccc(C(=O)Nc2ccc(S(=O)(=O)N3CCN(c4cc(C(F)(F)F)c(Cl)cn4)CC3)cc2)cc1]

A solution of compound 003a (55.0 mg, 0.070 mmol) in TFA/dioxane (4 M, 5 mL) was stirred at rt for 4 hours. After completion, the solvent was removed under vacuum to give the crude product which was purified by prep-HPLC (TFA as additive) to afford Example 003 (35 mg, 78.2% yield) as a white solid. MS obsd. (ESI⁺) [(M+H)⁺]: 611.2.

### Example 003: ¹H NMR (400 MHz, DMSO-d6) δ ppm 10.70 (s, 1H), 8.31 (s, 1H), 8.08 (d, J=8.8 Hz, 2H), 7.94 (d, J=8.1 Hz, 2H), 7.75 (d, J=8.8 Hz, 2H), 7.51 (d, J=8.0 Hz, 2H), 7.18 (s, 1H), 3.80 (s, 2H), 3.71 (s, 4H), 2.98 (s, 4H), 2.85 (t, J=6.9 Hz, 2H), 2.60 (t, J=6.3 Hz, 2H), 1.65-1.79 (m, 2H). ¹⁹F NMR (376 MHz, DMSO-d6) δ ppm −63.43 (s, 3F)

### Example 004: 4-[(3-Aminopropylamino)methyl]-N-[4-[4-[3-chloro-5-(trifluoromethyl) phenyl]piperazin-1-yl]sulfonylphenyl]benzamide

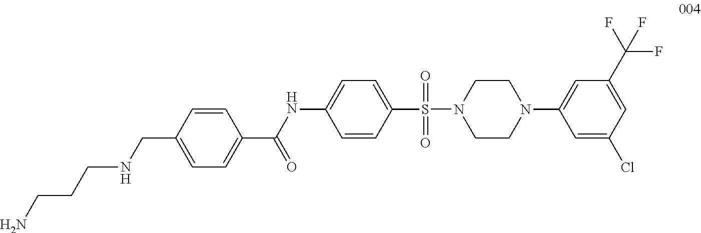 [SMILES: NCCCNCc1ccc(C(=O)Nc2ccc(S(=O)(=O)N3CCN(c4cc(Cl)cc(C(F)(F)F)c4)CC3)cc2)cc1]

Step 1: Tert-butyl 4-[3-chloro-5-(trifluoromethyl)phenyl]piperazine-1-carboxylate

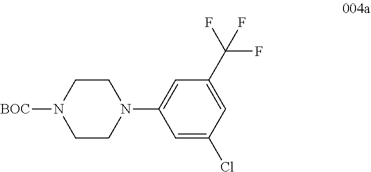 [SMILES: CC(C)(C)OC(=O)N1CCN(c2cc(Cl)cc(C(F)(F)F)c2)CC1]

A mixture of 1-chloro-3-iodo-5-(trifluoromethyl)benzene (2.0 g, 6.53 mmol), tert-butyl piperazine-1-carboxylate (1.82 g, 9.79 mmol), copper(I) iodide (1.24 g, 6.53 mmol), K₂CO₃(1.8 g, 13.1 mmol), proline (225 mg, 1.96 mmol) in DMF (30 mL) was stirred for 3 hours at 130° C. in a microwave reactor. After completion, the mixture was diluted with EtOAc (150 mL), and washed with water (50 mL×3). The organic layer was dried over Na₂SO₄ and concentrated under vacuum to give a residue, which was purified by silica-gel chromatography (elute with EtOAc:PE=20%) to afford compound 004a (1.0 g, 42.0% yield) as a white solid. MS obsd. (ESI⁺) [(M+H)⁺]: 365.1.

### Step 2: 1-[3-Chloro-5-(trifluoromethyl)phenyl]piperazine

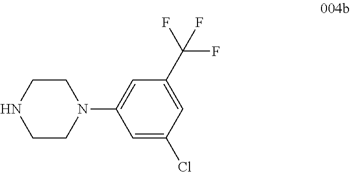 [SMILES: FC(F)(F)c1cc(Cl)cc(N2CCNCC2)c1]

A mixture of compound 004a (800 mg, 2.19 mmol) and TFA (2.5 g, 21.9 mmol) in DCM (15 mL) was stirred for 2 hours at rt. And then, the reaction mixture was concentrated to afford crude compound 004b (580 mg, 100% yield) as yellow gum, which was used in the next step directly. MS obsd. (ESI⁺) [(M+H)⁺]: 265.1.

Step 3: N-[4-[4-[3-chloro-5-(trifluoromethyl)phenyl]piperazin-1-yl]sulfonylphenyl]acetamide

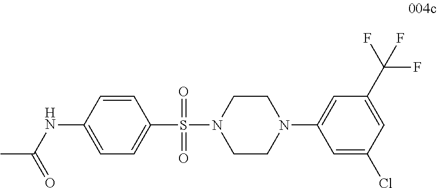 [SMILES: CC(=O)Nc1ccc(S(=O)(=O)N2CCN(c3cc(Cl)cc(C(F)(F)F)c3)CC2)cc1]

A mixture of compound 004b (580 mg, 2.18 mmol), 4-acetamidobenzenesulfonyl chloride (510 mg, 2.18 mmol) and DIPEA (500 mg, 3.87 mmol) in DCM (20 mL) was stirred for 1 hour at rt. Then the mixture was washed with saturated NaHCO₃ aqueous solution (20 mL×3). The organic layer was dried over Na₂SO₄ and concentrated under vacuum to give a crude residue, which was purified by silica-gel chromatography (elute with EtOAc:PE=15%) to afford compound 004c (1.0 g, 42.0% yield) as white solid. MS obsd. (ESI⁺) [(M+H)⁺]: 462.1.

### Step 4: 4-[4-[3-Chloro-5-(trifluoromethyl)phenyl]piperazin-1-yl]sulfonylaniline

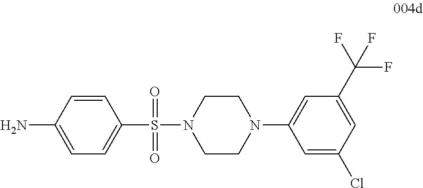 [SMILES: Nc1ccc(S(=O)(=O)N2CCN(c3cc(Cl)cc(C(F)(F)F)c3)CC2)cc1]

A mixture of compound 004c (400 mg, 866 μmol), HCl aqueous solution (1 mL, 866 μmol) in MeOH (3 mL) was heated for 10 minutes at 100° C. in a microwave reactor. After removal of solvent, the residue was dissolved in DCM (20 mL), washed with saturated NaHCO₃ solution (20 mL×2) and brine (20 mL×2). The organic layer was dried over Na₂SO₄, concentrated under vacuum to give crude compound 004d (363.6 mg, 100%) as a white solid, which was used in next step directly. MS obsd. (ESI⁺) [(M+H)⁺]: 419.9.

Step 5: 4-(Chloromethyl)-N-[4-[4-[3-chloro-5-(trifluoromethyl)phenyl]piperazin-1-yl]sulfonylphenyl]benzamide

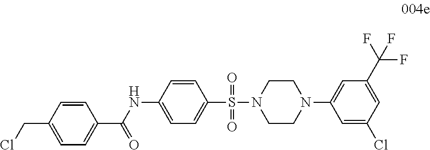 [SMILES: O=C(Nc1ccc(S(=O)(=O)N2CCN(c3cc(Cl)cc(C(F)(F)F)c3)CC2)cc1)c1ccc(CCl)cc1]

A mixture of compound 004d (190 mg, 453 μmol), 4-(chloromethyl)benzoyl chloride (128 mg, 679 μmol) and pyridine (107 mg, 1.36 mmol) in DCM (3 mL) was stirred for 4 hours at rt. After completion, the reaction mixture was concentrated to afford residue, which was purified by silica-gel chromatography (elute with EtOAc:PE=20%) to afford compound 004e (36.1 mg, 30.0% yield) as a white solid. MS obsd. (ESI⁺) [(M+H)⁺]: 572.1.

Step 6: 4-[(3-Aminopropylamino)methyl]-N-[4-[4-[3-chloro-5-(trifluoromethyl) phenyl]piperazin-1-yl]sulfonylphenyl]benzamide

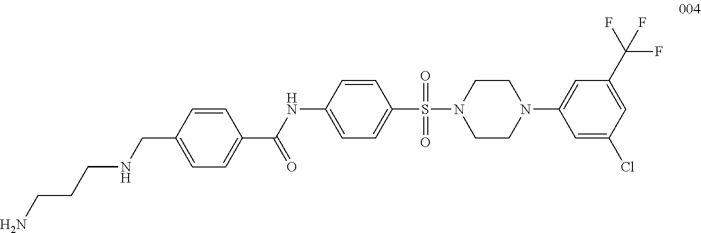 [SMILES: NCCCNCc1ccc(C(=O)Nc2ccc(S(=O)(=O)N3CCN(c4cc(Cl)cc(C(F)(F)F)c4)CC3)cc2)cc1]

A mixture of compound 004d (80 mg, 140 μmol), propane-1,3-diamine (51.8 mg, 699 μmol) in DMF (5 mL) was stirred at 100° C. for 3 h. After completion, the reaction mixture was concentrated and residue was purified by prep-HPLC (TFA as additive) to afford Example 004 (29.1 mg, 32.94%). MS obsd. (ESI⁺) [(M+H)⁺]: 610.3.

### Example 004: ¹H NMR (400 MHz, DMSO-d6) δ ppm 8.35 (s, 1H), 8.08 (d, 2H, J=8.8 Hz), 7.93 (d, 2H, J=8.3 Hz), 7.77 (d, 2H, J=8.9 Hz), 7.49 (d, 2H, J=8.2 Hz), 7.23 (s, 1H), 7.15 (s, 1H), 7.11 (s, 1H), 3.76 (s, 2H), 3.3-3.4 (m, 4H), 3.0-3.0 (m, 4H), 2.8-2.9 (m, 2H), 2.56 (s, 2H), 1.6-1.7 (m, 2H). ¹⁹F NMR (376 MHz, DMSO-d6) δ ppm −61.38 (s, 3F)

### Example 005: 4-[(3-Aminopropylamino)methyl]-N-[4-[4-[2-chloro-6-(trifluoromethyl)pyrimidin-4-yl]piperazin-1-yl]sulfonylphenyl]benzamide

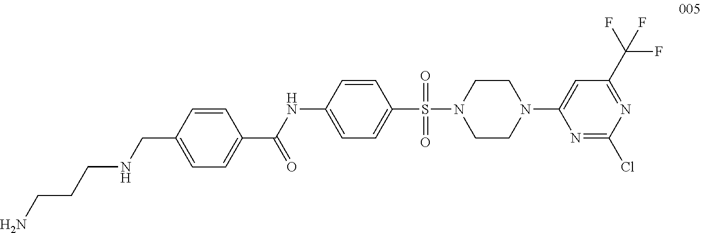 [SMILES: NCCCNCc1ccc(C(=O)Nc2ccc(S(=O)(=O)N3CCN(c4cc(C(F)(F)F)nc(Cl)n4)CC3)cc2)cc1]

The title compound was prepared in analogy to the preparation of Example 003 by replacing compound 2,5-dichloro-4-(trifluoromethyl)pyridine with compound 2,4-dichloro-6-(trifluoromethyl)pyrimidine in step 1 and the reaction was run at rt. Example 005 (32 mg) was obtained as white solid. MS obsd. (ESI⁺) [(M+H)⁺]: 612.1.

### Example 005: ¹H NMR (400 MHz, DMSO-d6) δ ppm 10.70 (s, 1H), 8.67 (s, 1H), 8.07 (d, J=8.1 Hz, 2H), 7.94 (s, 2H), 7.74 (d, J=8.2 Hz, 2H), 7.51 (s, 2H), 3.88 (s, 4H), 3.80 (s, 2H), 2.99 (s, 4H), 2.86 (s, 2H), 2.57 (d, J=20.2 Hz, 2H), 1.73 (s, 2H). ¹⁹F NMR (376 MHz, DMSO-d6) δ ppm −69.48 (s, 3F)

### Example 006: 4-[(3-Aminopropylamino)methyl]-N-[4-[4-[2-amino-6-(trifluoromethyl)pyrimidin-4-yl]piperazin-1-yl]sulfonylphenyl]benzamide

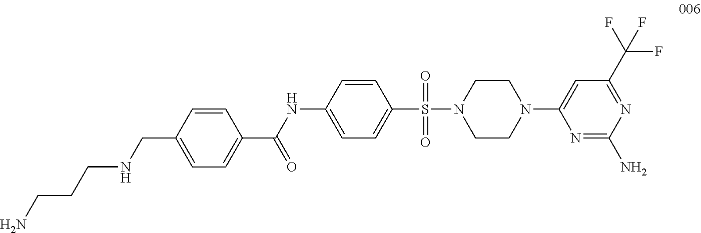 [SMILES: NCCCNCc1ccc(C(=O)Nc2ccc(S(=O)(=O)N3CCN(c4cc(C(F)(F)F)nc(N)n4)CC3)cc2)cc1]

The title compound was prepared in analogy to the preparation of Example 003 by replacing compound 2,5-dichloro-4-(trifluoromethyl)pyridine with compound 2-chloro-6-(trifluoromethyl)pyrimidin-4-amine in Step 1 and the reaction was heated at 160° C. for 1 hour in a microwave reactor. Example 006 (10 mg) was obtained as white solid. MS obsd. (ESI⁺) [(M+H)⁺]: 593.2.

### Example 006: ¹H NMR (400 MHz, DMSO-d6) δ ppm 10.70 (s, 1H), 8.09 (d, J=8.8 Hz, 2H), 7.96 (d, J=8.0 Hz, 2H), 7.73 (d, J=8.8 Hz, 2H), 7.54 (d, J=8.0 Hz, 2H), 7.12 (s, 2H), 6.10 (s, 1H), 3.84 (s, 2H), 3.78 (s, 4H), 2.80-2.97 (m, 6H), 2.65 (s, 2H), 1.77 (s, 2H). ¹⁹F NMR (376 MHz, DMSO-d6) δ ppm −63.43 (s, 3F)

### Example 007: 4-[(3-Aminopropylamino)methyl]-N-[4-[4-(4-bromo-6-chloro-2-pyridyl)piperazin-1-yl]sulfonylphenyl]benzamide

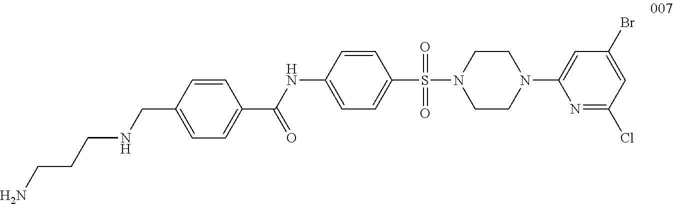 [SMILES: NCCCNCc1ccc(C(=O)Nc2ccc(S(=O)(=O)N3CCN(c4cc(Br)cc(Cl)n4)CC3)cc2)cc1]

The title compound was prepared in analogy to the preparation of Example 003 by replacing compound 2,5-dichloro-4-(trifluoromethyl)pyridine with 4-bromo-2,6-dichloro-pyridine in Step 1. Example 007 (8 mg) was obtained as an off-white solid. MS obsd. (ESI⁺) [(M+H)⁺]: 621.1.

### Example 007: ¹H NMR (400 MHz, DMSO-d6) δ ppm 10.65 (s, 1H), 8.07 (d, J=8.8 Hz, 2H), 7.93 (d, J=8.0 Hz, 2H), 7.75 (d, J=8.8 Hz, 2H), 7.50 (d, J=7.9 Hz, 2H), 7.04 (s, 1H), 6.93 (s, 1H), 3.77 (s, 2H), 3.63 (s, 4H), 2.96 (d, J=4.3 Hz, 4H), 2.85 (s, 2H), 2.56 (d, J=6.3 Hz, 2H), 1.70 (s, 2H)

### Example 008: 4-[(3-Aminopropylamino)methyl]-N-[4-[4-[5-cyclopropyl-4-(trifluoromethyl)-2-pyridyl]piperazin-1-yl]sulfonylphenyl]benzamide

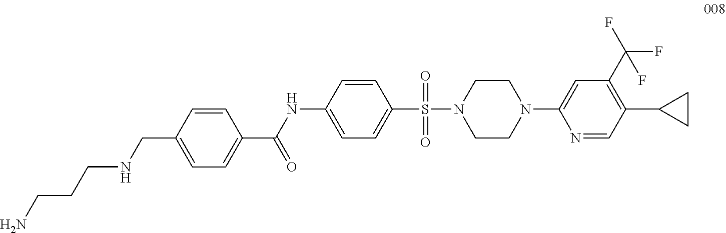 [SMILES: NCCCNCc1ccc(C(=O)Nc2ccc(S(=O)(=O)N3CCN(c4cc(C(F)(F)F)c(C5CC5)cn4)CC3)cc2)cc1]

Step 1: Tert-butyl N-[[4-[[4-[4-[5-bromo-4-(trifluoromethyl)-2-pyridyl]piperazin-1-yl]sulfonylphenyl]carbamoyl]phenyl]methyl]-N-[3-(tert-butoxycarbonylamino)propyl]carbamate

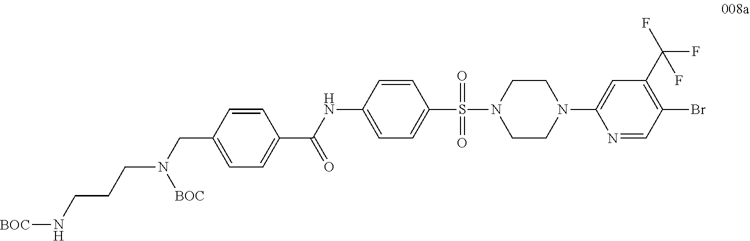 [SMILES: CC(C)(C)OC(=O)NCCCN(Cc1ccc(C(=O)Nc2ccc(S(=O)(=O)N3CCN(c4cc(C(F)(F)F)c(Br)cn4)CC3)cc2)cc1)C(=O)OC(C)(C)C]

Compound 008a was prepared in analogy to the preparation of compound 003a by replacing 2,5-dichloro-4-(trifluoromethyl)pyridine with 5-bromo-2-chloro-4-(trifluoromethyl)pyridine in step 1. Compound 008a (48 mg) was obtained. MS obsd. (ESI⁺) [(M+H)⁺1: 855.1.

Step 2: Tert-butyl N-[3-(tert-butoxycarbonylamino)propyl]-N-[[4-[[4-[4-[5-cyclopropyl-4-(trifluoromethyl)-2-pyridyl]piperazin-1-yl]sulfonylphenyl]carbamoyl]phenyl]methyl]carbamate

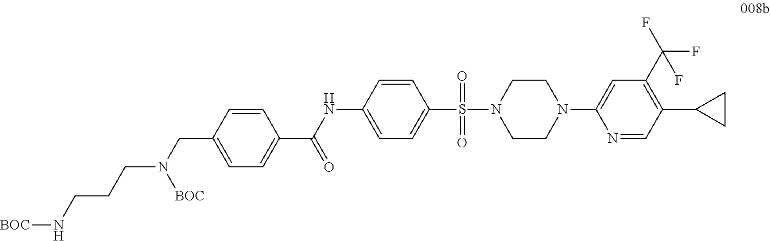 [SMILES: CC(C)(C)OC(=O)NCCCN(Cc1ccc(C(=O)Nc2ccc(S(=O)(=O)N3CCN(c4cc(C(F)(F)F)c(C5CC5)cn4)CC3)cc2)cc1)C(=O)OC(C)(C)C]

A mixture of Pd(OAc)₂ (4.72 mg, 0.020 mmol), compound 008a (72.0 mg, 0.080 mmol), potassium cyclopropyltrifluoroborate (3.11 mg, 0.020 mmol), 2-dicyclohexylphosphino-2′,6′-dimethoxybiphenyl (0.86 mg) in toluene (8.17 mL) and water (0.817 mL) was bubbled with N₂ and stirred at 90° C. for 12 hours. After completion, the reaction was filtrated with Buchner funnel and the filtrate was concentrated under vacuum to give residue, which was purified by prep-HPLC (FA as additive) to give compound 008b (32 mg, 41.9% yield) as a white solid. MS obsd. (ESI⁺) [(M+H)⁺]: 817.1.

Step 3: 4-[(3-Aminopropylamino)methyl]-N-[4-[4-[5-cyclopropyl-4-(trifluoromethyl)-2-pyridyl]piperazin-1-yl]sulfonylphenyl]benzamide

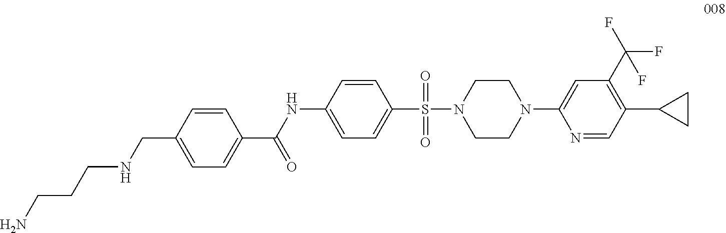 [SMILES: NCCCNCc1ccc(C(=O)Nc2ccc(S(=O)(=O)N3CCN(c4cc(C(F)(F)F)c(C5CC5)cn4)CC3)cc2)cc1]

Compound 008b (30.0 mg, 0.040 mmol) in 4M TFA/dioxane (5 mL) was stirred at rt for 4 hours. After completion, the solvent was removed under vacuum to give the crude product, which was purified by prep-HPLC to afford Example 008 (8 mg, 23.53% yield). MS obsd. (ESI⁺) [(M+H)⁺]: 617.1.

### Example 008: ¹H NMR (400 MHz, DMSO-d6) δ 10.69 (s, 1H), 8.39 (s, 2H), 8.07 (d, J=8.5 Hz, 2H), 8.01 (s, 1H), 7.93 (d, J=7.5 Hz, 2H), 7.75 (d, J=8.5 Hz, 2H), 7.50 (d, J=7.4 Hz, 2H), 7.01 (s, 1H), 3.78 (s, 2H), 3.71 (s, 1H), 3.64 (s, 4H), 2.97 (s, 4H), 2.85 (s, 2H), 2.58 (s, 2H), 1.85 (s, 1H), 1.71 (s, 2H), 0.87 (d, J=7.6 Hz, 2H), 0.69 (d, J=4.7 Hz, 2H). ¹⁹F NMR (376 MHz, DMSO-d6) δ −61.59 (s, 3F)

### Example 009: 4-[(3-Aminopropylamino)methyl]-N-[4-[4-[6-(cyclopropoxy)-4-(trifluoromethyl)-2-pyridyl]piperazin-1-yl]sulfonylphenyl]benzamide

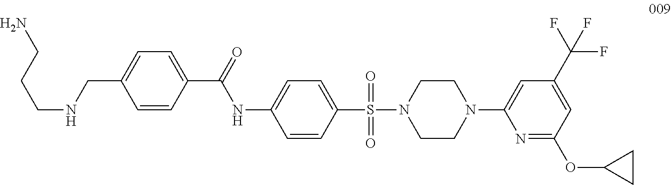 [SMILES: NCCCNCc1ccc(C(=O)Nc2ccc(S(=O)(=O)N3CCN(c4cc(C(F)(F)F)cc(OC5CC5)n4)CC3)cc2)cc1]

### Step 1: 2,6-Dibromo-4-(trifluoromethyl)pyridine

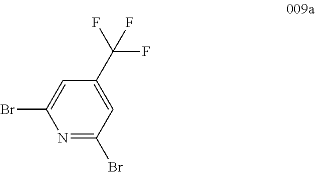 [SMILES: FC(F)(F)c1cc(Br)nc(Br)c1]

Into the flask charged with 2,6-dichloro-4-(trifluoromethyl)pyridine (2.65 mL, 18.1 mmol) was added HBr in AcOH (30 mL, 166 mmol). The reaction was heated at 85° C. and stirred overnight. After completion, the reaction was extracted with EtOAc (100 mL×3). The combined organic layer was washed with brine (150 mL), dried over anhydrous Na₂SO₄, filtered and concentrated under vacuum to give crude compound 009a (4.31 g, 78% yield) as yellow oil, which was used directly in the next step.

### Step 2: 2-Bromo-6-(cyclopropoxy)-4-(trifluoromethyl)pyridine

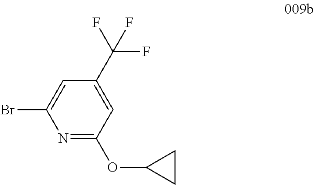 [SMILES: FC(F)(F)c1cc(Br)nc(OC2CC2)c1]

Compound 009a (0.4 g, 1.31 mmol) was dissolved in THF (5 mL), followed by adding cyclopropanol (100 mg, 1.72 mmol) and then NaH (78.7 mg, 1.97 mmol) in the sealed tube. The reaction was heated at 90° C. and kept stirring at the same temperature overnight. After completion, the reaction was cooled to room temperature and quenched by adding water (50 mL). The resulting residue was extracted with EtOAc (50 mL×3). The combined organic layer was washed with brine (100 mL), dried over anhydrous Na₂SO₄, filtered and concentrated under vacuum to afford compound 009b (366 mg, 100% yield) as yellow oil. MS obsd. (ESI⁺) [(M+H)⁺]: 282.0.

Step 3: Tert-butyl 4-[6-(cyclopropoxy)-4-(trifluoromethyl)-2-pyridyl]piperazine-1-carboxylate

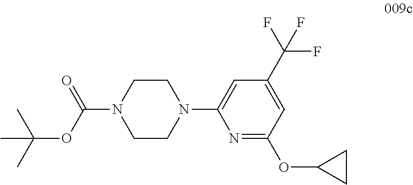 [SMILES: CC(C)(C)OC(=O)N1CCN(c2cc(C(F)(F)F)cc(OC3CC3)n2)CC1]

Compound 009b (800 mg, 2.84 mmol), tert-butyl piperazine-1-carboxylate (1.06 g, 5.67 mmol) and K₂CO₃ (1.18 g, 8.51 mmol) were dissolved in DMSO (20 mL). The reaction was heated at 100° C. and stirred overnight. After the starting material was consumed, the reaction was cooled to rt and quenched by adding water (50 mL). The reaction mixture was extracted with EtOAc (50 mL×3). The combined organic layer was washed with brine (100 mL), dried over anhydrous Na₂SO₄, filtered and concentrated under vacuum to afford compound 009c (1.0 g, 91% yield) as yellow oil. MS obsd. (ESI⁺) [(M+H)⁺]: 388.4.

Step 4: 1-[6-(Cyclopropoxy)-4-(trifluoromethyl)-2-pyridyl]piperazine

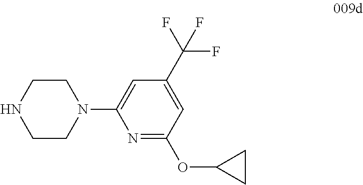 [SMILES: FC(F)(F)c1cc(OC2CC2)nc(N2CCNCC2)c1]

Compound 009c (1.0 g, 2.58 mmol) was dissolved in the DCM (2.5 mL), followed by adding TFA (2.5 mL, 1.86 mmol). The reaction was stirred at rt and went completed after 1 hour. Then the reaction mixture was concentrated in vacuo and the residue formed an azeotrope with toluene 2 times to afford compound 009d (0.6 g, 80% yield) as a yellow solid. MS obsd. (ESI⁺) [(M+H)⁺]: 288.4.

Step 5: N-[4-[4-[6-(cyclopropoxy)-4-(trifluoromethyl)-2-pyridyl]piperazin-1-yl]sulfonylphenyl]acetamide

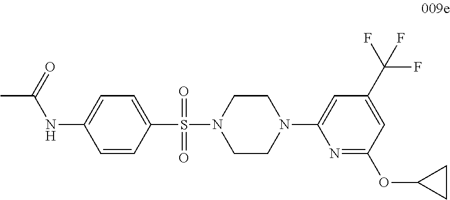 [SMILES: CC(=O)Nc1ccc(S(=O)(=O)N2CCN(c3cc(C(F)(F)F)cc(OC4CC4)n3)CC2)cc1]

A mixture of compound 009d (74 mg, 258 μmol), pyridine (249 μL, 3.09 mmol) and 4-acetamidobenzenesulfonyl chloride (300 mg, 1.28 mmol) in DCM (12.5 mL) was stirred at 0° C. for 30 minutes, then allowed to warm to rt. After competition, the reaction was extracted with EtOAc (10 mL×3). The combined organic layer was washed with brine (15 mL), dried over anhydrous Na₂SO₄ and concentrated under vacuum to give crude compound 009e (100 mg, 80.6% yield) as a yellow solid. MS obsd. (ESI⁺) [(M+H)⁺]: 485.2.

Step 5: 4-[4-[6-(Cyclopropoxy)-4-(trifluoromethyl)-2-pyridyl]piperazin-1-yl]sulfonylaniline

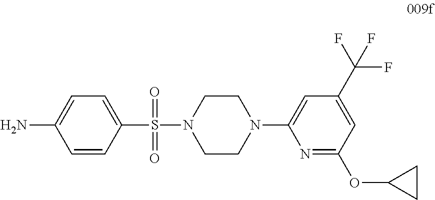 [SMILES: Nc1ccc(S(=O)(=O)N2CCN(c3cc(C(F)(F)F)cc(OC4CC4)n3)CC2)cc1]

Compound 009e (100 mg, 206 μmol) was dissolved in the MeOH (5 mL). Into the stirring solution was added HCl (2 mL, 8 mmol) dropwise. The mixture was then heated at 70° C. and kept stirring overnight. After competition, the reaction was extracted with EtOAc (10 mL×3). The combined organic layer was washed with brine (15 mL), dried over anhydrous sodium sulfate, filtered and concentrated in vacuo to give crude compound 009f (90 mg, 98.6% yield) as a yellow solid. MS obsd. (ESI⁺) [(M+H)⁺]: 443.2.

Step 6: 4-(Chloromethyl)-N-[4-[4-[6-(cyclopropoxy)-4-(trifluoromethyl)-2-pyridyl]piperazin-1-yl]sulfonylphenyl]benzamide

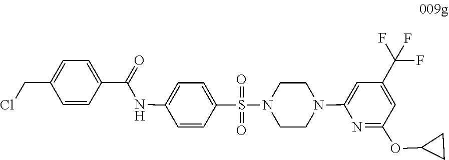 [SMILES: O=C(Nc1ccc(S(=O)(=O)N2CCN(c3cc(C(F)(F)F)cc(OC4CC4)n3)CC2)cc1)c1ccc(CCl)cc1]

Compound 009f (100 mg, 226 μmol), 4-(chloromethyl)benzoyl chloride (128 mg, 678 μmol) and TEA (94.5 μL, 678 μmol) were stirred at rt overnight. After completion, the mixture was quenched with water (50 mL), diluted with DCM (30 mL). The combined organic layer was washed with brine (50 mL), dried over anhydrous Na₂SO₄ and concentrated under vacuum to give curde product, which was purified by silica-gel chromatography (eluent with EtOAc:PE=25%) to afford compound 009g (120 mg, 89.2% yield) as a white solid. MS obsd. (ESI⁺) [(M+H)⁺]: 595.0.

Step 7: Tert-butyl N-[3-[[4-[[4-[4-[6-(cyclopropoxy)-4-(trifluoromethyl)-2-pyridyl]piperazin-1-yl]sulfonylphenyl]carbamoyl]phenyl]methylamino]propyl]carbamate

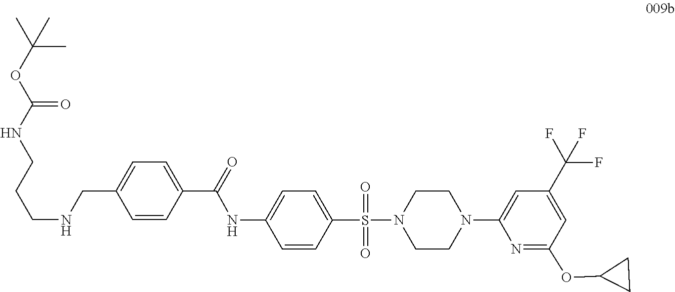 [SMILES: CC(C)(C)OC(=O)NCCCNCc1ccc(C(=O)Nc2ccc(S(=O)(=O)N3CCN(c4cc(C(F)(F)F)cc(OC5CC5)n4)CC3)cc2)cc1]

Compound 009g (0.1 g, 168 μmol) and tert-butyl (3-aminopropyl)carbamate (58.6 mg, 336 μmol) were dissolved in the MeCN (1.68 mL), followed by adding potassium carbonate (69.7 mg, 504 μmol). The reaction was stirred at 70° C. for 3 hours. After completion, the reaction was cooled to rt and extracted with EtOAc (10 mL×3). The combined organic layer was washed with brine (15 mL), dried over anhydrous sodium sulfate, filtered and concentrated in vacuo to give crude compound 009h (105 mg, 85.7% yield) as yellow oil. MS obsd. (ESI⁺) [(M+H)⁺]: 733.3.

Step 8: 4-[(3-Aminopropylamino)methyl]-N-[4-[4-[6-(cyclopropoxy)-4-(trifluoromethyl)-2-pyridyl]piperazin-1-yl]sulfonylphenyl]benzamide

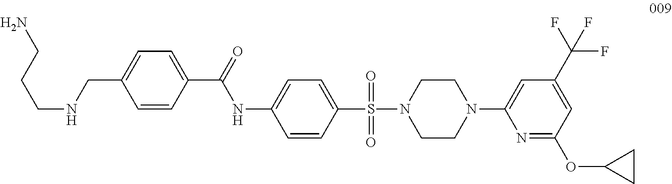 [SMILES: NCCCNCc1ccc(C(=O)Nc2ccc(S(=O)(=O)N3CCN(c4cc(C(F)(F)F)cc(OC5CC5)n4)CC3)cc2)cc1]

Compound 009h (0.2 g, 273 μmol) was dissolved in the DCM (10 mL), followed by adding TFA (2.5 mL, 1.86 mmol). The reaction mixture was stirred at rt for 1 hour. The reaction mixture was concentrated under vacuum and purified by prep-HPLC (TFA as additive) to afford Example 009 (20 mg, 7.3% yield) as a white solid. MS obsd. (ESI⁺) [(M+H)⁺]: 633.1.

### Example 009: ¹H NMR (400 MHz, DMSO-d₆) δ ppm 8.91-9.08 (m, 1H), 8.00-8.15 (m, 3H), 7.73-7.79 (m, 3H), 7.56-7.69 (m, 2H), 6.54-6.71 (m, 1H), 6.22-6.37 (m, 1H), 4.04-4.32 (m, 3H), 3.60-3.83 (m, 4H), 2.92-3.07 (m, 6H), 2.75-2.92 (m, 2H), 2.02-2.15 (m, 1H), 1.80-2.00 (m, 2H), 0.70-0.82 (m, 2H), 0.55-0.70 (m, 2H). ¹⁹F NMR (376 MHz, DMSO-d₆) δ ppm −63.48 (m, 3F)

### Example 010: 4-[(3-Aminopropylamino)methyl]-N-[4-[4-[6-ethoxy-4-(trifluoromethyl)-2-pyridyl]piperazin-1-yl]sulfonylphenyl]benzamide

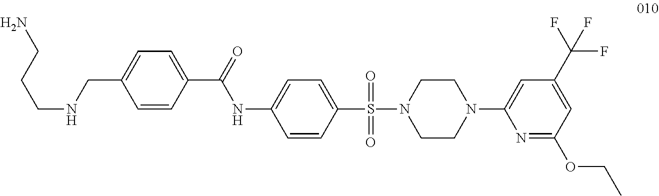 [SMILES: CCOc1cc(C(F)(F)F)cc(N2CCN(S(=O)(=O)c3ccc(NC(=O)c4ccc(CNCCCN)cc4)cc3)CC2)n1]

The title compound was prepared in analogy to the preparation of Example 009 by using ethanol instead of cyclopropanol in Step 2. Example 010 (15 mg) was obtained as a white solid. MS obsd. (ESI⁺) [(M+H)⁺]: 621.1.

### Example 010: ¹H NMR (400 MHz, DMSO-d₆) δ ppm 7.98-8.17 (m, 3H), 7.73-7.87 (m, 4H), 7.59-7.70 (m, 2H), 6.48-6.61 (m, 1H), 6.17-6.31 (m, 1H), 4.15-4.36 (m, 4H), 3.62-3.76 (m, 4H), 2.93-3.09 (m, 6H), 2.79-2.93 (m, 2H), 1.78-1.98 (m, 2H), 1.18-1.34 (m, 3H). ¹⁹F NMR (376 MHz, DMSO-d₆) δ ppm −63.55 (m, 3F)

### Example 011: 4-[(3-Aminopropylamino)methyl]-N-[4-[4-[6-ethyl-4-(trifluoromethyl)-2-pyridyl]piperazin-1-yl]sulfonylphenyl]benzamide

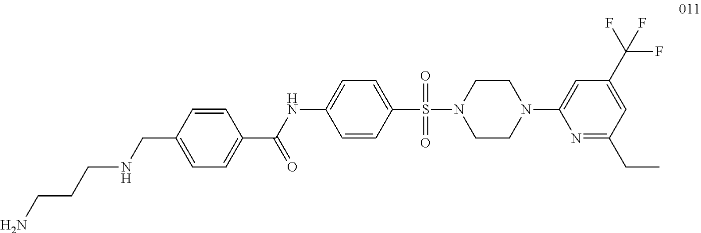 [SMILES: CCc1cc(C(F)(F)F)cc(N2CCN(S(=O)(=O)c3ccc(NC(=O)c4ccc(CNCCCN)cc4)cc3)CC2)n1]

Step 1: Tert-butyl N-[3-(tert-butoxycarbonylamino)propyl]-N-[[4-[[4-[4-[6-chloro-4-(trifluoromethyl)-2-pyridyl]piperazin-1-yl]sulfonylphenyl]carbamoyl]phenyl]methyl]carbamate

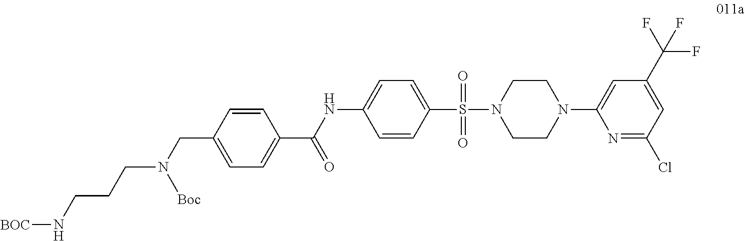 [SMILES: CC(C)(C)OC(=O)NCCCN(Cc1ccc(C(=O)Nc2ccc(S(=O)(=O)N3CCN(c4cc(C(F)(F)F)cc(Cl)n4)CC3)cc2)cc1)C(=O)OC(C)(C)C]

The title compound was prepared in analogy to step 1 of the preparation of compound 003a by replacing compound 2,5-dichloro-4-(trifluoromethyl)pyridine with compound 2,6-dichloro-4-(trifluoromethyl)pyridine. Compound Olla (1.0 g) was obtained. MS obsd. (ESI⁺) [(M+H)⁺]: 811.3.

Step 2: Tert-butyl N-[3-(tert-butoxycarbonylamino)propyl]-N-[[4-[[4-[4-[6-ethyl-4-(trifluoromethyl)-2-pyridyl]piperazin-1-yl]sulfonylphenyl]carbamoyl]phenyl]methyl]carbamate

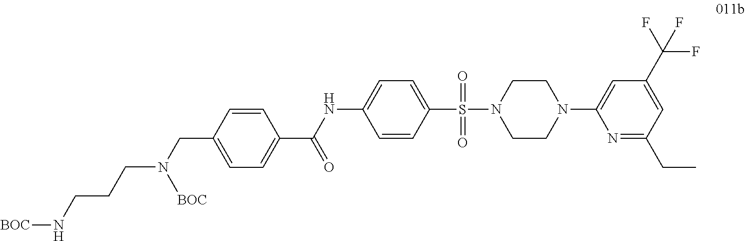 [SMILES: CCc1cc(C(F)(F)F)cc(N2CCN(S(=O)(=O)c3ccc(NC(=O)c4ccc(CN(CCCNC(=O)OC(C)(C)C)C(=O)OC(C)(C)C)cc4)cc3)CC2)n1]

Compound 011a (40.0 mg, 0.050 mmol) and bis(tri-tert-butylphosphine)palladium(0) (50.0 mg) was dissolved in DCM (5 mL). Then Al(Et)₃ (11.26 mg, 0.10 mmol) was added and the reaction was stirred at rt for 12 hours. The solid was filtered and the filtrate was concentrated under vacuum to give crude, which was purified by silica-gel chromatography (elute with EtOAc:PE=40%) to afford compound 011b (35 mg, 79.38% yield). MS obsd. (ESI⁺) [(M+H)⁺]: 805.3.

Step 3: 4-[(3-Aminopropylamino)methyl]-N-[4-[4-[6-ethyl-4-(trifluoromethyl)-2-pyridyl]piperazin-1-yl]sulfonylphenyl]benzamide

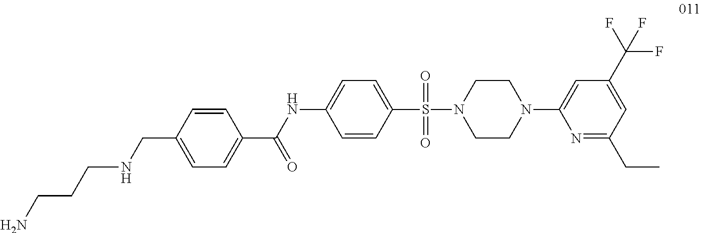 [SMILES: CCc1cc(C(F)(F)F)cc(N2CCN(S(=O)(=O)c3ccc(NC(=O)c4ccc(CNCCCN)cc4)cc3)CC2)n1]

The title compound was prepared in analogy to the preparation of Example 003 by replacing compound 003a with compound 011b in step 2. Example 011 (14 mg) was obtained. MS obsd. (ESI⁺) [(M+H)⁺]: 605.2.

### Example 011: ¹H NMR (400 MHz, DMSO-d6) δ ppm 10.68 (s, 1H), 8.08 (d, J=8.4 Hz, 2H), 7.94 (d, J=7.2 Hz, 2H), 7.75 (d, J=8.4 Hz, 2H), 7.50 (d, J=6.3 Hz, 2H), 6.88 (s, 1H), 6.77 (s, 1H), 3.81 (d, J=17.2 Hz, 2H), 3.70 (s, 4H), 2.98 (s, 4H), 2.85 (s, 2H), 2.70-2.54 (m, 4H), 1.72 (s, 2H), 1.17 (t, J=7.5 Hz, 3H). ¹⁹F NMR (376 MHz, DMSO-d6) δ ppm −63.43 (s, 3F)

### Example 012: 4-[(3-Aminopropylamino)methyl]-N-[4-[4-[4-(trifluoromethyl)pyrimidin-2-yl]piperazin-1-yl]sulfonylphenyl]benzamide

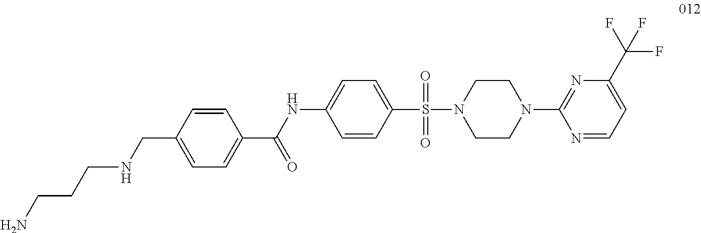 [SMILES: NCCCNCc1ccc(C(=O)Nc2ccc(S(=O)(=O)N3CCN(c4nccc(C(F)(F)F)n4)CC3)cc2)cc1]

The title compound was prepared in analogy to the preparation of Example 003 by replacing 2,5-dichloro-4-(trifluoromethyl)pyridine with 2-chloro-4-(trifluoromethyl)pyrimidine in step 1. Example 012 (14 mg) was obtained. MS obsd. (ESI⁺) [(M+H)⁺]: 578.3.

### Example 012: ¹H NMR (400 MHz, DMSO-d6) δ ppm 10.70 (s, 1H), 8.67 (d, J=4.6 Hz, 1H), 8.07 (d, J=8.1 Hz, 2H), 7.94 (s, 2H), 7.74 (d, J=8.2 Hz, 2H), 7.51 (s, 2H), 7.04 (d, J=4.7 Hz, 1H), 3.88 (s, 4H), 3.80 (s, 2H), 2.99 (s, 4H), 2.86 (s, 2H), 2.57 (d, J=20.2 Hz, 2H), 1.73 (s, 2H). 1⁹F NMR (376 MHz, DMSO-d6) δ ppm −69.48 (s, 3F)

### Example 013: 4-[2-(3-Aminopropylamino)ethyl]-N-[4-[4-[5-methyl-4-(trifluoromethyl)-2-pyridyl]piperazin-1-yl]sulfonylphenyl]benzamide

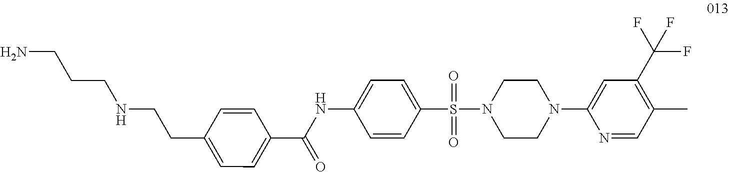 [SMILES: Cc1cnc(N2CCN(S(=O)(=O)c3ccc(NC(=O)c4ccc(CCNCCCN)cc4)cc3)CC2)cc1C(F)(F)F]

Step 1: Tert-butyl N-[2-[4-[[4-[4-[5-bromo-4-(trifluoromethyl)-2-pyridyl]piperazin-1-yl]sulfonylphenyl]carbamoyl]phenyl]ethyl]-N-[3-(tert-butoxycarbonylamino)propyl]carbamate

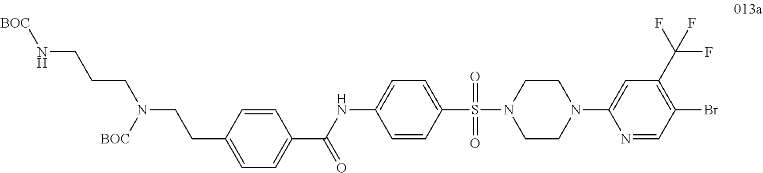 [SMILES: CC(C)(C)OC(=O)NCCCN(CCc1ccc(C(=O)Nc2ccc(S(=O)(=O)N3CCN(c4cc(C(F)(F)F)c(Br)cn4)CC3)cc2)cc1)C(=O)OC(C)(C)C]

The title compound was prepared in analogy to the preparation of compound 003a in step 1 by replacing 2,5-dichloro-4-(trifluoromethyl)pyridine with 5-bromo-2-chloro-4-(trifluoromethyl)pyridine and compound Int-5 with compound Int-7. Compound 013a (31 mg) was obtained. MS obsd. (ESI⁺) [(M+H)⁺]: 869.3.

Step 2: Tert-butyl N-[3-(tert-butoxycarbonylamino)propyl]-N-[2-[4-[[4-[4-[5-methyl-4-(trifluoromethyl)-2-pyridyl]piperazin-1-yl]sulfonylphenyl]carbamoyl]phenyl]ethyl]carbamate

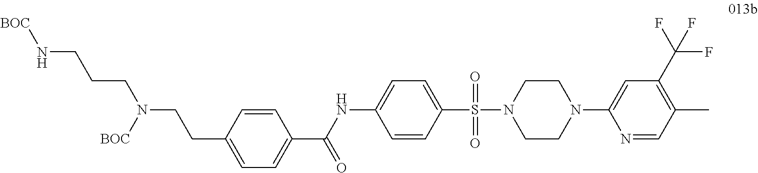 [SMILES: Cc1cnc(N2CCN(S(=O)(=O)c3ccc(NC(=O)c4ccc(CCN(CCCNC(=O)OC(C)(C)C)C(=O)OC(C)(C)C)cc4)cc3)CC2)cc1C(F)(F)F]

A mixture of [1,1′-bis(diphenylphosphino)ferrocene]dichloropalladium(II) (52.16 mg, 0.070 mmol), compound 013a (31.0 mg, 0.040 mmol) and 2,4,6-trimethyl-1,3,5,2,4,6-trioxatriborinane (0.1 mmol) in 1,4-dioxane (2.31 mL) was bubbled with N₂ and then heated at 110° C. for 1 hour in a microwave reactor. The mixture was poured into water (10 mL), extracted with EtOAc (10 mL×3). The combined organic layer were dried over Na₂SO₄ and concentrated under vacuum to give crude product, which was purified by silica-gel chromatography (elute with EtOAc:PE=50%) to give compound 013b as a white solid (21 mg, 65.88% yield). MS obsd. (ESI⁺) [(M+H)⁺]: 805.1.

Step 3: 4-[2-(3-Aminopropylamino)ethyl]-N-[4-[4-[5-methyl-4-(trifluoromethyl)-2-pyridyl]piperazin-1-yl]sulfonylphenyl]benzamide

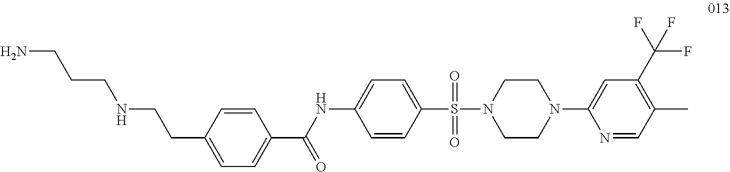 [SMILES: Cc1cnc(N2CCN(S(=O)(=O)c3ccc(NC(=O)c4ccc(CCNCCCN)cc4)cc3)CC2)cc1C(F)(F)F]

The title compound was prepared in analogy to the preparation of Example 003 in step 2 by replacing compound 003a with compound 013b. Example 013 (6 mg) was obtained as white solid. MS obsd. (ESI⁺) [(M+H)⁺]: 605.3.

### Example 013: ¹H NMR (400 MHz, DMSO-d6) δ ppm 10.71 (s, 1H), 8.14 (s, 1H), 8.07 (d, J=8.5 Hz, 2H), 7.90 (d, J=7.5 Hz, 2H), 7.74 (d, J=8.4 Hz, 2H), 7.39 (d, J=7.4 Hz, 2H), 7.02 (s, 1H), 3.64 (s, 4H), 2.97 (s, 4H), 2.82 (s, 6H), 2.67 (s, 2H), 2.22 (s, 3H), 1.68 (s, 2H). ¹⁹F NMR (376 MHz, DMSO-d6) δ ppm −62.89 (s, 3F)

### Example 014: 4-[2-(3-Aminopropylamino)ethyl]-N-[4-[4-(4,5-dichloro-2-pyridyl)piperazin-1-yl]sulfonylphenyl]benzamide

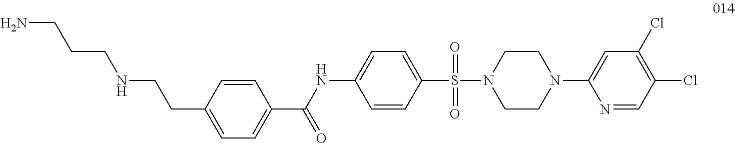 [SMILES: NCCCNCCc1ccc(C(=O)Nc2ccc(S(=O)(=O)N3CCN(c4cc(Cl)c(Cl)cn4)CC3)cc2)cc1]

The title compound was prepared in analogy to the preparation of Example 003 by replacing compound 2,5-dichloro-4-(trifluoromethyl)pyridine with 2,4,5-trichloropyridine and compound Int-5 with compound Int-7 in step 1. Example 014 (31 mg) was obtained. MS obsd. (ESI⁺) [(M+H)⁺]: 591.2.

### Example 014: ¹H NMR (400 MHz, CD₃OD) δ ppm 8.04 (s, 1H), 7.92 (d, J=8.2 Hz, 2H), 7.85 (d, J=7.6 Hz, 2H), 7.71 (d, J=8.1 Hz, 2H), 7.36 (d, J=7.7 Hz, 2H), 6.97 (s, 1H), 3.23 (d, J=4.0 Hz, 4H), 3.10 (d, J=15.0 Hz, 6H), 2.98 (s, 6H), 1.93 (s, 2H)

### Example 015: 4-[2-(3-Aminopropylamino)ethyl]-N-[4-[4-[4-cyano-6-(trifluoromethyl)-2-pyridyl]piperazin-1-yl]sulfonylphenyl]benzamide

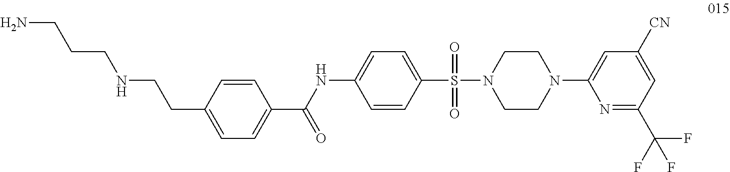 [SMILES: N#Cc1cc(N2CCN(S(=O)(=O)c3ccc(NC(=O)c4ccc(CCNCCCN)cc4)cc3)CC2)nc(C(F)(F)F)c1]

The title compound was prepared in analogy to the preparation of Example 003 by replacing compound 2,5-dichloro-4-(trifluoromethyl)pyridine with 2-chloro-6-(trifluoromethyl)pyridine-4-carbonitrile and compound Int-5 with compound Int-7 in step 1. Example 015 (10 mg) was obtained as a white solid. MS obsd. (ESI⁺) [(M+H)⁺]: 616.3.

### Example 015: ¹H NMR (400 M Hz, DMSO-d6) δ ppm 10.68 (s, 1H), 8.06 (d, J=8.7 Hz, 2H), 7.90 (d, J=8.0 Hz, 2H), 7.75 (d, J=8.7 Hz, 2H), 7.67 (s, 1H), 7.44 (s, 1H), 7.39 (d, J=7.9 Hz, 2H), 3.76 (s, 5H), 3.00 (s, 4H), 2.81 (s, 6H), 2.66 (s, 2H), 1.67 (s, 2H). ¹⁹F NMR (376 MHz, DMSO-d6) δ ppm −67.72 (s, 3F)

### Example 016: 4-[2-(3-Aminopropylamino)ethyl]-N-[4-[4-[6-cyano-4-(trifluoromethyl)-2-pyridyl]piperazin-1-yl]sulfonylphenyl]benzamide

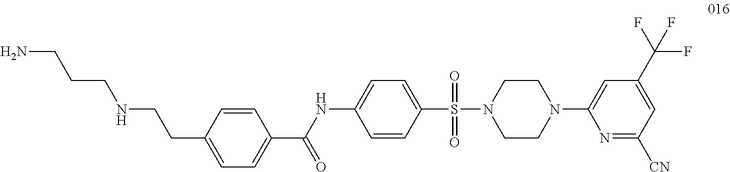 [SMILES: N#Cc1cc(C(F)(F)F)cc(N2CCN(S(=O)(=O)c3ccc(NC(=O)c4ccc(CCNCCCN)cc4)cc3)CC2)n1]

The title compound was prepared in analogy to the preparation of Example 003 by replacing compound 2,5-dichloro-4-(trifluoromethyl)pyridine with 6-chloro-4-(trifluoromethyl)pyridine-2-carbonitrile and compound Int-5 with compound Int-7 in step 1. Example 016 (10 mg) was obtained as a white solid. MS obsd. (ESI⁺) [(M+H)⁺]: 616.2.

### Example 016: ¹H NMR (400 MHz, DMSO-d6) δ ppm 10.68 (s, 1H), 8.36 (s, 2H), 8.04 (d, J=8.7 Hz, 2H), 7.87 (d, J=7.9 Hz, 2H), 7.72 (d, J=8.7 Hz, 2H), 7.56 (s, 1H), 7.45 (s, 1H), 7.37 (d, J=7.8 Hz, 2H), 3.74 (s, 4H), 2.97 (s, 4H), 2.79 (s, 6H), 2.64 (s, 2H), 1.65 (s, 2H). ¹⁹F NMR (376 MHz, DMSO-d6) δ ppm −63.71 (s, 3F)

### Example 017: 4-[2-(3-Aminopropylamino)ethyl]-N-[4-[4-[6-chloro-4-(1,1-difluoroethyl)-2-pyridyl]piperazin-1-yl]sulfonylphenyl]benzamide

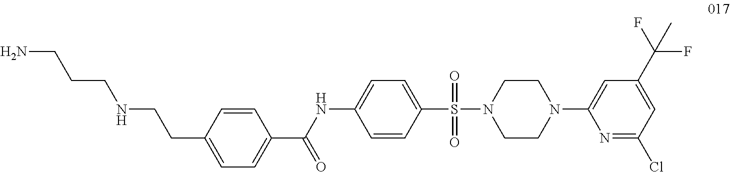 [SMILES: CC(F)(F)c1cc(Cl)nc(N2CCN(S(=O)(=O)c3ccc(NC(=O)c4ccc(CCNCCCN)cc4)cc3)CC2)c1]

The title compound was prepared in analogy to the preparation of Example 003 by replacing compound 2,5-dichloro-4-(trifluoromethyl)pyridine with compound Int-8 and compound Int-5 with compound Int-7 in step 1. Example 015 (8 mg) was obtained as a white solid. MS obsd. (ESI⁺) [(M+H)⁺]: 621.1.

### Example 017: ¹H NMR (400 MHz, DMSO-d6) δ ppm 10.70 (s, 1H), 8.37 (s, 2H), 8.08 (d, J=8.6 Hz, 2H), 7.91 (d, J=7.6 Hz, 2H), 7.75 (d, J=8.6 Hz, 2H), 7.39 (d, J=7.6 Hz, 2H), 6.87 (s, 1H), 6.79 (s, 1H), 3.67 (s, 4H), 2.98 (s, 4H), 2.83 (s, 6H), 2.69 (s, 2H), 1.91 (t, J=19.2 Hz, 3H), 1.70 (s, 2H). ¹⁹F NMR (376 MHz, DMSO) δ ppm −87.87 (s, 2F)

### Example 018: 3-[(3-Aminopropylamino)methyl]-N-[4-[4-[6-methyl-4-(trifluoromethyl)-2-pyridyl]piperazin-1-yl]sulfonylphenyl]benzamide

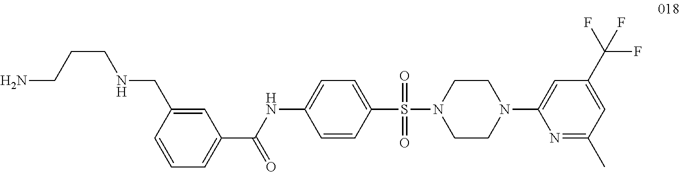 [SMILES: Cc1cc(C(F)(F)F)cc(N2CCN(S(=O)(=O)c3ccc(NC(=O)c4cccc(CNCCCN)c4)cc3)CC2)n1]

The title compound was prepared in analogy to the preparation of Example 003 by replacing 2,5-dichloro-4-(trifluoromethyl)pyridine with 2-chloro-6-methyl-4-(trifluoromethyl)pyridine and compound Int-5 with compound Int-6 in step 1. Example 018 (8 mg) was obtained as a white solid. MS obsd. (ESI⁺) [(M+H)⁺]: 591.1.

### Example 018: ¹H NMR (CD₃OD, 400 MHz) δ ppm 7.99 (s, 1H), 7.9-8.0 (m, 3H), 7.7-7.7 (m, 2H), 7.6-7.7 (m, 1H), 7.5-7.6 (m, 1H), 6.67 (s, 1H), 6.62 (s, 1H), 4.2-4.3 (m, 2H), 3.6-3.6 (m, 4H), 3.1-3.1 (m, 2H), 2.9-3.0 (m, 6H), 2.3-2.3 (s, 3H), 2.0-2.1 (m, 2H). ¹⁹F NMR (376 MHz, DMSO-d6) δ ppm −66.52 (s, 3F)

### Example 019: Methyl 2-[4-[4-[[3-[(3-aminopropylamino)methyl]benzoyl]amino]phenyl]sulfonylpiperazin-1-yl]-6-chloro-pyridine-4-carboxylate

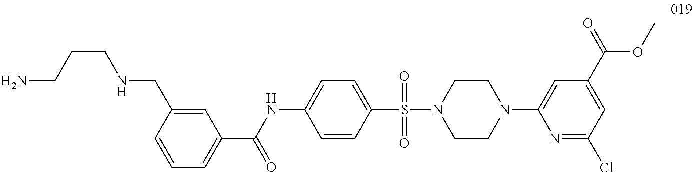 [SMILES: COC(=O)c1cc(Cl)nc(N2CCN(S(=O)(=O)c3ccc(NC(=O)c4cccc(CNCCCN)c4)cc3)CC2)c1]

The title compound was prepared in analogy to the preparation of Example 003 by replacing 2,5-dichloro-4-(trifluoromethyl)pyridine with methyl 2,6-dichloropyridine-4-carboxylate and compound Int-5 with compound Int-6 in step 1. Example 019 (12 mg) was obtained as a white solid. MS obsd. (ESI⁺) [(M+H)⁺]: 601.1.

### Example 019: ¹H NMR (CD₃OD, 400 MHz) δ ppm 7.98 (s, 1H), 7.9-8.0 (m, 3H), 7.7-7.7 (m, 2H), 7.62 (d, 1H, J=7.6 Hz), 7.5-7.6 (m, 1H), 7.09 (d, 1H, J=0.7 Hz), 6.96 (d, 1H, J=0.7 Hz), 4.19 (s, 2H), 3.79 (s, 3H), 3.6-3.6 (m, 4H), 2.9-3.1 (m, 8H), 1.98 (quin, 2H, J=7.7 Hz)

### Example 020: 3-[(3-Aminopropylamino)methyl]-N-[4-[4-(6-chloro-4-cyano-2-pyridyl)piperazin-1-yl]sulfonylphenyl]benzamide

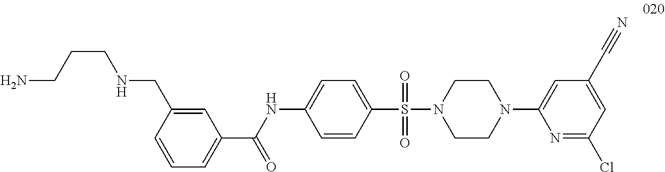 [SMILES: N#Cc1cc(Cl)nc(N2CCN(S(=O)(=O)c3ccc(NC(=O)c4cccc(CNCCCN)c4)cc3)CC2)c1]

The title compound was prepared in analogy to the preparation of Example 003 by replacing 2,5-dichloro-4-(trifluoromethyl)pyridine with 2,6-dichloropyridine-4-carbonitrile and compound Int-5 with compound Int-6 in step 1. Example 020 (15 mg) was obtained as a white solid. MS obsd. (ESI⁺) [(M+H)⁺]: 568.2.

### Example 020: ¹H NMR (CD₃OD, 400 MHz) δ ppm 8.0 (s, 1H), 7.91 (d, 3H, J=8.8 Hz), 7.70 (d, 2H, J=8.8 Hz), 7.6-7.7 (m, 1H), 7.5-7.6 (m, 1H), 6.94 (s, 1H), 6.77 (s, 1H), 4.23 (s, 2H), 3.6-3.7 (m, 4H), 3.1-3.1 (m, 2H), 2.9-3.0 (m, 6H), 2.01 (t, 2H, J=7.7 Hz)

### Example 021: 3-[(3-Aminopropylamino)methyl]-N-[4-[4-[6-chloro-4-(trifluoromethyl)-2-pyridyl]-2-methyl-piperazin-1-yl]sulfonylphenyl]benzamide

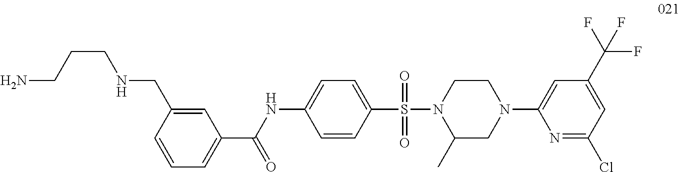 [SMILES: CC1CN(c2cc(C(F)(F)F)cc(Cl)n2)CCN1S(=O)(=O)c1ccc(NC(=O)c2cccc(CNCCCN)c2)cc1]

Step 1: Tert-butyl 4-(4-acetamidophenyl)sulfonyl-3-methyl-piperazine-1-carboxylate

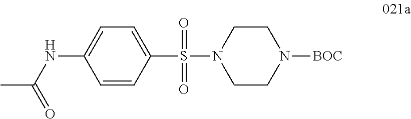 [SMILES: CC(=O)Nc1ccc(S(=O)(=O)N2CCN(C(=O)OC(C)(C)C)CC2)cc1]

To a solution of 4-N-BOC-2-methylpiperazine (857.11 mg, 4.28 mmol), TEA (1.19 mL, 8.56 mmol) in DCM (10 mL) was added N-acetylsulfanilyl chloride (1.0 g, 4.28 mmol) in one portion and the reaction was at 25° C. for 1 hour. After completion, the mixture was diluted with DCM (20 mL), washed with water (20 mL) and brine (20 mL). The organic layer was dried over anhydrous Na₂SO₄, concentrated under vacuum to afford compound 021a (1.5 g, 88.18% yield) as yellow solid, which was used in the next step directly. MS obsd. (ESI⁺) [(M+Na)⁺]: 420.3.

Step 2: N-[4-(2-methylpiperazin-1-yl)sulfonylphenyl]acetamide

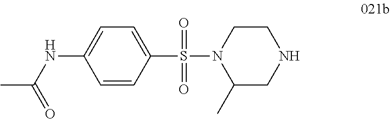 [SMILES: CC(=O)Nc1ccc(S(=O)(=O)N2CCNCC2C)cc1]

A mixture of compound 021a (1.5 g, 3.77 mmol) in DCM (15 mL) and TFA (14.54 mL, 188.68 mmol) was stirred at 20° C. for 1 hour. After completion, the mixture was concentrated under vacuum to give compound 021b (1.5 g, 96.62% yield) as a yellow oil, which was used in the next step directly. MS obsd. (ESI⁺) [(M+H)⁺]: 297.9.

Step 3: N-[4-[4-[6-chloro-4-(trifluoromethyl)-2-pyridyl]-2-methyl-piperazin-1-yl]sulfonylphenyl]acetamide

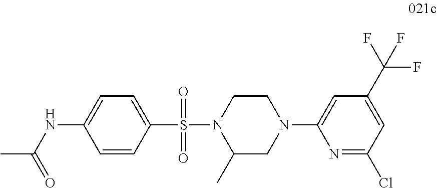 [SMILES: CC(=O)Nc1ccc(S(=O)(=O)N2CCN(c3cc(C(F)(F)F)cc(Cl)n3)CC2C)cc1]

The mixture of compound 021b (1.5 g, 3.65 mmol), DIPEA (1.27 mL, 7.29 mmol), 2,6-dichloro-4-(trifluoromethyl)pyridine (787.52 mg, 3.65 mmol) in DMSO (20 mL) was heated at 90° C. for 24 hours. After completion, the reaction mixture was diluted with water (50 mL), extracted with EtOAc (30 mL×3). The combined organic layer was washed with brine (50 mL), dried over anhydrous Na₂SO₄, concentrated under vacuum to give crude product, which was purified by prep-HPLC (TFA as additive) to give compound 021c (400 mg, 23% yield) as a yellow oil. MS obsd. (ESI⁺) [(M+H)⁺]: 477.1.

Step 4: 4-[4-[6-Chloro-4-(trifluoromethyl)-2-pyridyl]-2-methyl-piperazin-1-yl]sulfonylaniline

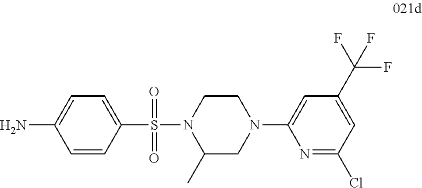 [SMILES: CC1CN(c2cc(C(F)(F)F)cc(Cl)n2)CCN1S(=O)(=O)c1ccc(N)cc1]

Compound 021d was prepared in analogy to the preparation of compound 004d by replacing compound 004c with compound 021c. Compound 021d (180 mg) was obtained as a white solid. MS obsd. (ESI⁺) [(M+Na)⁺]: 435.2.

Step 5: Tert-butyl N-[3-(tert-butoxycarbonylamino)propyl]-N-[[3-[[4-[4-[6-chloro-4-(trifluoromethyl)-2-pyridyl]-2-methyl-piperazin-1-yl]sulfonylphenyl]carbamoyl]phenyl]methyl]carbamate

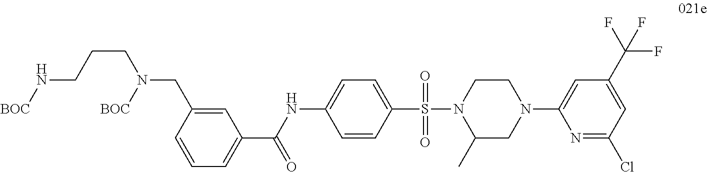 [SMILES: CC1CN(c2cc(C(F)(F)F)cc(Cl)n2)CCN1S(=O)(=O)c1ccc(NC(=O)c2cccc(CN(CCCNC(=O)OC(C)(C)C)C(=O)OC(C)(C)C)c2)cc1]

To a solution of compound Int-9 (100.0 mg, 0.240 mmol) in MeCN (2 mL) was added 3-methylpyridine (45.6 mg, 0.490 mmol), methanesulfonyl chloride (0.03 mL, 0.370 mmol) in one portion and the mixture was stirred at rt for 1 hour. And then, compound 021d (117.1 mg, 0.270 mmol) was added to the mixture and the reaction was stirred at rt for 2 hours. After completion, the mixture was concentrated under vacuum to give a residue, which was purified by prep-HPLC (TFA as additive) to give compound 021e (78 mg, 37.96% yield) as a yellow oil. MS obsd. (ESI⁺) [(M+H)⁺]: 825.3.

Step 6: 3-[(3-Aminopropylamino)methyl]-N-[4-[4-[6-chloro-4-(trifluoromethyl)-2-pyridyl]-2-methyl-piperazin-1-yl]sulfonylphenyl]benzamide

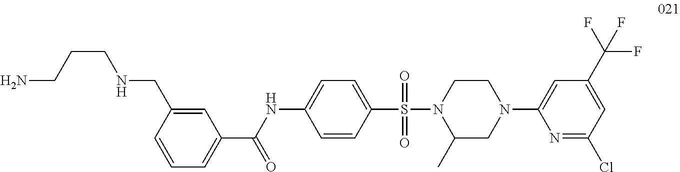 [SMILES: CC1CN(c2cc(C(F)(F)F)cc(Cl)n2)CCN1S(=O)(=O)c1ccc(NC(=O)c2cccc(CNCCCN)c2)cc1]

The mixture of compound 021e (78.0 mg, 0.090 mmol) in DCM (1 mL) and TFA (0.36 mL) was stirred at 20° C. for 1 hour. After completion, the mixture was concentrated under vacuum to give a residue, which was purified by prep-HPLC (NH₄HCO₃ as additive) to afford Example 021 (28 mg, 47.03% yield) as a white solid. MS obsd. (ESI⁺) [(M+H)⁺]: 625.5.

### Example 021: ¹H NMR (400 MHz, DMSO-d₆) δ ppm 8.10-7.97 (m, 2H), 7.93-7.88 (m, 1H), 7.86-7.76 (m, 3H), 7.63-7.54 (m, 1H), 7.51-7.44 (m, 1H), 7.03 (s, 1H), 6.92 (s, 1H), 4.17-4.07 (m, 2H), 4.06-3.98 (m, 1H), 3.77 (s, 2H), 3.71-3.63 (m, 1H), 3.20-3.13 (m, 2H), 3.03-2.92 (m, 2H), 1.57-1.45 (m, 2H), 1.02 (d, 3H)

### Example 022: 3-[(3-Aminopropylamino)methyl]-N-[4-[4-[6-chloro-4-(trifluoromethyl)-2-pyridyl]-3-methyl-piperazin-1-yl]sulfonylphenyl]benzamide

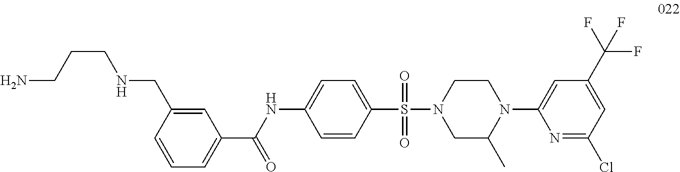 [SMILES: CC1CN(S(=O)(=O)c2ccc(NC(=O)c3cccc(CNCCCN)c3)cc2)CCN1c1cc(C(F)(F)F)cc(Cl)n1]

The title compound was prepared in analogy to the preparation of Example 021 by replacing 4-N-BOC-3-methylpiperazine with 4-N-BOC-2-methylpiperazine in step 1. Example 022 (18 mg) was obtained as a white solid. MS obsd. (ESI⁺) [(M+H)⁺]: 625.1.

### Example 022: ¹H NMR (400 MHz, DMSO-d6) δ ppm 8.12-8.04 (m, 2H), 7.95-7.89 (m, 1H), 7.86-7.79 (m, 1H), 7.77-7.71 (m, 2H), 7.60-7.52 (m, 1H), 7.52-7.43 (m, 1H), 7.04 (s, 1H), 7.00-6.94 (m, 1H), 4.74-4.63 (m, 1H), 4.28-4.16 (m, 1H), 3.76 (s, 2H), 3.72-3.66 (m, 1H), 3.54-3.47 (m, 2H), 3.27-3.16 (m, 2H), 2.91-2.72 (m, 2H), 2.46-2.29 (m, 2H), 1.70-1.50 (m, 2H), 1.19 (d, 3H)

### Example 023: 3-[(3-Aminopropylamino)methyl]-N-[4-[[5-[6-chloro-4-(trifluoromethyl)-2-pyridyl]-2,5-diazabicyclo[4.2.0]octan-2-yl]sulfonyl]phenyl]benzamide

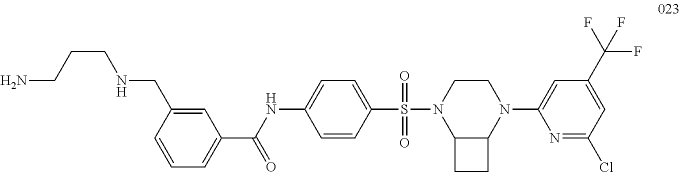 [SMILES: NCCCNCc1cccc(C(=O)Nc2ccc(S(=O)(=O)N3CCN(c4cc(C(F)(F)F)cc(Cl)n4)C4CCC43)cc2)c1]

The title compound was prepared in analogy to the preparation of Example 021 by replacing 4-N-BOC-2-methylpiperazine with tert-butyl 2,5-diazabicyclo[4.2.0]octane-2-carboxylate in step 1. Example 023 (10 mg) was obtained as a white solid. MS obsd. (ESI⁺) [(M+H)⁺]: 637.2.

### Example 023: ¹H NMR (400 MHz, DMSO-d6) δ 11.01-10.70 (m, 1H), 9.75-9.44 (m, 2H), 8.33-8.26 (m, 1H), 8.20-8.08 (m, 5H), 8.05-7.96 (m, 1H), 7.86-7.75 (m, 3H), 7.65-7.54 (m, 1H), 7.07-6.98 (m, 1H), 6.85-6.73 (m, 1H), 4.52-4.40 (m, 1H), 4.32-4.18 (m, 2H), 4.10-3.98 (m, 1H), 3.85-3.73 (m, 1H), 3.65-3.51 (m, 2H), 3.10-2.99 (m, 2H), 2.98-2.87 (m, 3H), 2.22-2.10 (m, 2H), 2.07-1.92 (m, 4H)

### Example 024: 3-[(3-Aminopropylamino)methyl]-N-[4-[4-[6-chloro-4-(trifluoromethyl)-2-pyridyl]piperazin-1-yl]sulfonylphenyl]benzamide

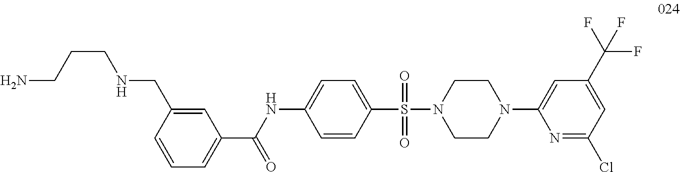 [SMILES: NCCCNCc1cccc(C(=O)Nc2ccc(S(=O)(=O)N3CCN(c4cc(C(F)(F)F)cc(Cl)n4)CC3)cc2)c1]

The title compound was prepared in analogy to the preparation of Example 003 by replacing compound Int-5 with compound Int-6 in step 1. Example 024 (10 mg) was obtained as a white solid. MS obsd. (ESI⁺) [(M+H)⁺]: 611.1.

### Example 024: ¹H NMR (CD₃OD, 400 MHz) δ ppm 8.01 (s, 1H), 7.96 (d, 3H, J=8.7 Hz), 7.8-7.8 (m, 2H), 7.65 (d, 1H, J=7.8 Hz), 7.5-7.6 (m, 1H), 6.83 (s, 1H), 6.70 (s, 1H), 4.41 (d, 2H, J=13.7 Hz), 4.24 (s, 2H), 3.1-3.1 (m, 2H), 2.9-3.0 (m, 2H), 2.8-2.9 (m, 2H), 1.9-2.1 (m, 4H), 1.57 (dq, 2H, J=4.3, 12.5 Hz)

### Example 025: 3-[2-(3-Aminopropylamino)ethyl]-N-[4-[S-[4-[6-chloro-4-(trifluoromethyl)-2-pyridyl]piperazin-1-yl]-N-cyano-sulfonimidoyl]phenyl]benzamide

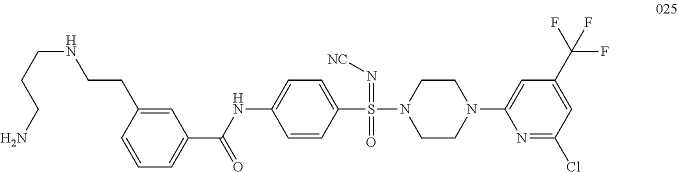 [SMILES: N#CN=S(=O)(c1ccc(NC(=O)c2cccc(CCNCCCN)c2)cc1)N1CCN(c2cc(C(F)(F)F)cc(Cl)n2)CC1]

Step 1: N-[4-[4-[6-chloro-4-(trifluoromethyl)-2-pyridyl]piperazin-1-yl]sulfanylphenyl]acetamide

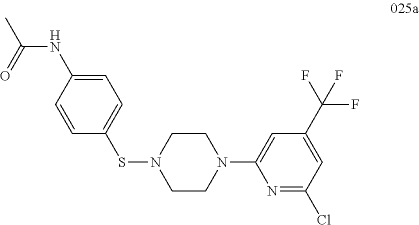 [SMILES: CC(=O)Nc1ccc(SN2CCN(c3cc(C(F)(F)F)cc(Cl)n3)CC2)cc1]

The mixture of 4-acetamidothiophenol (2.64 g, 15.81 mmol), compound Int-1a (3.5 g, 13.17 mmol), 1,10-Phenanthroline (474.84 mg, 2.63 mmol) and copper oxide (210.8 mg, 2.63 mmol) in DMSO (25 mL) was stirred at 60° C. for 24 hours under oxygen (1.5 L, 0.750 mmol) (15 Psi, one balloon). After completion, the mixture was diluted with water (100 mL) and extracted with EtOAc (200 mL×3). The combined organic layer was washed with brine (200 mL), dried over anhydrous Na₂SO₄, concentrated under vacuum to give residue, which was purified by silica-gel column chromatography (elute with EtOAc:PE=20%) to give compound 025a (3 g, 52.85% yield) as a yellow solid. MS obsd. (ESI⁺) [(M+H)⁺]: 430.9.

Step 2: N-[4-[[4-[6-chloro-4-(trifluoromethyl)-2-pyridyl]piperazin-1-yl]sulfonimidoyl]phenyl]acetamide

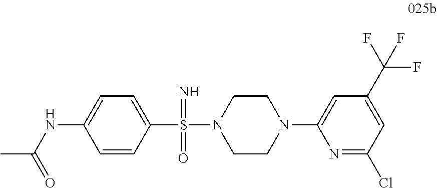 [SMILES: CC(=O)Nc1ccc(S(=N)(=O)N2CCN(c3cc(C(F)(F)F)cc(Cl)n3)CC2)cc1]

To a solution of compound 025a (800.0 mg, 1.86 mmol) and ammonia in THF (2.79 mL, 2.79 mmol) in methanol (15 mL) were added PIDA (1.79 g, 5.57 mmol) portion-wise at rt. The reaction mixture was stirred at 15° C. for 12 hours. After completion, the mixture was diluted with water (100 mL), extracted with EtOAc (100 mL×3). The combined organic layer was washed with brine (100 mL×3), dried over anhydrous Na₂SO₄, concentrated under vacuum to give residue, which was purified by silica-gel chromatography (elute with EtOAc:PE=10%) to give compound 025b (600 mg, 69.96% yield) as white solid. MS obsd. (ESI⁺) [(M+H)⁺]: 461.9.

Step 3: N-[4-[S-[4-[6-chloro-4-(trifluoromethyl)-2-pyridyl]piperazin-1-yl]-N-cyano-sulfonimidoyl]phenyl]acetamide

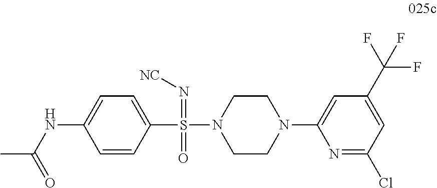 [SMILES: CC(=O)Nc1ccc(S(=O)(=NC#N)N2CCN(c3cc(C(F)(F)F)cc(Cl)n3)CC2)cc1]

To a solution of compound 025b (550.0 mg, 1.19 mmol) in MeCN (20 mL) were added 2,2′-azobis(2-methylpropionitrile) (293.3 mg, 1.79 mmol), potassium carbonate (329.14 mg, 2.38 mmol) and copper iodide (45.36 mg, 0.240 mmol). The reaction mixture was stirred at 75° C. for 12 hours under oxygen (1.5 L, 1.19 mmol, 1 eq) (one balloon pressure). After completion, the mixture was diluted with EA (200 mL) and filtered. The filtrate was concentrated to give residue, which was purified by silica-gel chromatography (15% EA in PE) to give compound 025c (300 mg, 50.32% yield) as white solid. MS obsd. (ESI⁺) [(M+H)⁺]: 487.0.

Step 4: [(4-Aminophenyl)-[4-[6-chloro-4-(trifluoromethyl)-2-pyridyl]piperazin-1-yl]-oxo-λ\hat{ }{6}-sulfanylidene]cyanamide

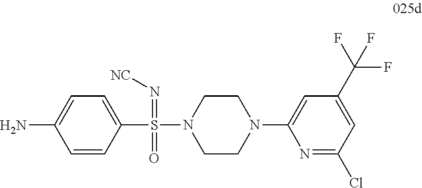 [SMILES: N#CN=S(=O)(c1ccc(N)cc1)N1CCN(c2cc(C(F)(F)F)cc(Cl)n2)CC1]

To a solution of compound 025c (120.0 mg, 0.250 mmol) in isopropyl alcohol (4 mL) were added concentrated hydrochloric acid (1.0 mL, 12 mmol) in one portion. The mixture was stirred at 25° C. for 12 hours. After completion, the mixture was concentrated under vacuum to give residue, which was purified by prep-TLC (elute with EtOAc:PE=50%) to afford compound 025d (70 mg, 63.85% yield) as a white solid. MS obsd. (ESI⁺) [(M+H)⁺]: 444.9.

Step 5: Tert-butyl N-[3-(tert-butoxycarbonylamino)propyl]-N-[2-[3-[[4-[S-[4-[6-chloro-4-(trifluoromethyl)-2-pyridyl]piperazin-1-yl]-N-cyano-sulfonimidoyl]phenyl]carbamoyl]phenyl]ethyl]carbamate

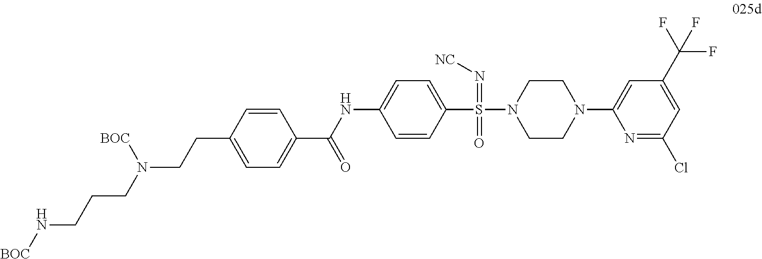 [SMILES: CC(C)(C)OC(=O)NCCCN(CCc1ccc(C(=O)Nc2ccc(S(=O)(=NC#N)N3CCN(c4cc(C(F)(F)F)cc(Cl)n4)CC3)cc2)cc1)C(=O)OC(C)(C)C]

To a solution of compound Int-9 (79.78 mg, 0.190 mmol) and 3-methylpyridine (43.96 mg, 0.470 mmol, 3 eq) in MeCN (1.5 mL) was added MsCl (0.02 mL, 0.240 mmol) and the mixture was stirred at 15° C. for 3 h. And then, compound 025c (70.0 mg, 0.160 mmol) was added to the mixture and the mixture was stirred at 30° C. for 12 h. After completion, the mixture was concentrated under vacuum and the residue was purified by prep-HPLC (FA as additive) to give compound 025d (80 mg, 59.86% yield) as a white solid. MS obsd. (ESI⁺) [(M+H)⁺]: 849.3.

Step 6: 3-[2-(3-Aminopropylamino)ethyl]-N-[4-[S-[4-[6-chloro-4-(trifluoromethyl)-2-pyridyl]piperazin-1-yl]-N-cyano-sulfonimidoyl]phenyl]benzamide

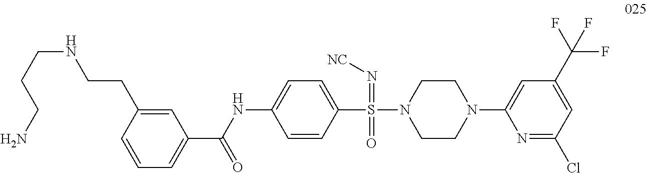 [SMILES: N#CN=S(=O)(c1ccc(NC(=O)c2cccc(CCNCCCN)c2)cc1)N1CCN(c2cc(C(F)(F)F)cc(Cl)n2)CC1]

The mixture of compound 025d (70.0 mg, 0.080 mmol) in DCM (2 mL) and TFA (0.3 mL, 0.080 mmol) was stirred at 15° C. for 2 hours. After completion, the mixture was concentrated under vacuum to give a residue, which was purified by prep-HPLC (TFA as additive) to give example 025 (20.5 mg, 29.92% yield) as a white solid. MS obsd. (ESI⁺) [(M+H)⁺]: 649.3.

### Example 025: ¹H NMR (400 MHz, DMSO-d6) δ ppm 8.33-8.43 (m, 1H), 8.15-8.24 (m, 2H), 7.97-8.07 (m, 2H), 7.83-7.91 (m, 2H), 7.49-7.58 (m, 2H), 7.15-7.18 (m, 1H), 7.01-7.06 (m, 1H), 3.80-3.84 (m, 4H), 3.23-3.32 (m, 4H), 2.86-2.95 (m, 6H), 2.73-2.81 (m, 2H), 1.70-1.81 (m, 2H)

### Example 026: 3-[(3-aminopropylamino)methyl]-N-[4-[S-[4-[6-chloro-4-(trifluoromethyl)-2-pyridyl]piperazin-1-yl]-N-methyl-sulfonimidoyl]phenyl]benzamide

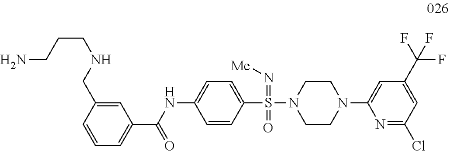 [SMILES: CN=S(=O)(c1ccc(NC(=O)c2cccc(CNCCCN)c2)cc1)N1CCN(c2cc(C(F)(F)F)cc(Cl)n2)CC1]

Step 1: N-[4-[S-[4-[6-chloro-4-(trifluoromethyl)-2-pyridyl]piperazin-1-yl]-N-methyl-sulfonimidoyl]phenyl]acetamide

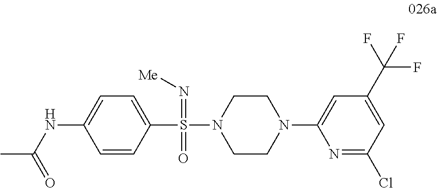 [SMILES: CN=S(=O)(c1ccc(NC(C)=O)cc1)N1CCN(c2cc(C(F)(F)F)cc(Cl)n2)CC1]

To a solution of compound Int-1a (1.0 g, 3.77 mmol) in THF (40 mL) was added NCS (1.11 g, 8.29 mmol) in one portion. The mixture was stirred at 45° C. for 1 h. Then triethylamine (1.58 mL, 11.31 mmol) and compound Int-11 (800.0 mg, 3.77 mmol) was added successively into the reaction mixture and stirred at 45° C. for 3 hours. After completion, the mixture was diluted with water (100 mL) and extracted with EtOAc (150 mL×3). The combined organic layer was washed with brine (100 mL×2), dried over Na₂SO₄ and concentrated under vacuum to give residue, which was purified by silica-gel chromatography (20% EA in PE) to give compound 026a (800 mg, 40.79% yield) as white solid. MS obsd. (ESI⁺) [(M+H)⁺]: 476.0.

Step 2: 4-[S-[4-[6-chloro-4-(trifluoromethyl)-2-pyridyl]piperazin-1-yl]-N-methyl-sulfonimidoyl]aniline

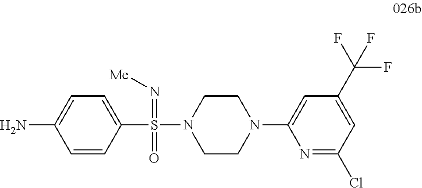 [SMILES: CN=S(=O)(c1ccc(N)cc1)N1CCN(c2cc(C(F)(F)F)cc(Cl)n2)CC1]

The mixture of compound 026a (700.0 mg, 1.47 mmol) in THF (15 mL) and concentrated hydrochloric acid (4.0 mL, 48 mmol) was stirred at 50° C. for 2 hours. After completion, the mixture was concentrated under vacuum to give residue, which was purified by prep-HPLC (NH₃·H₂O as additive) to give compound 026b (500 mg, 77.1% yield) as white solid. MS obsd. (ESI⁺) [(M+H)⁺]: 433.9.

Step 3: 3-(Chloromethyl)-N-[4-[S-[4-[6-chloro-4-(trifluoromethyl)-2-pyridyl]piperazin-1-yl]-N-methyl-sulfonimidoyl]phenyl]benzamide

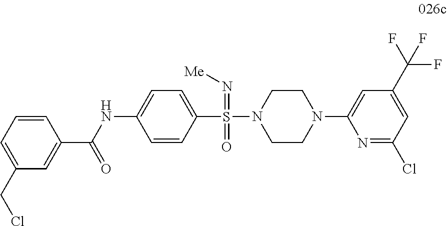 [SMILES: CN=S(=O)(c1ccc(NC(=O)c2cccc(CCl)c2)cc1)N1CCN(c2cc(C(F)(F)F)cc(Cl)n2)CC1]

To a mixture of compound 026b (150.0 mg, 0.350 mmol) and 3-(chloromethyl)benzoyl chloride (78.43 mg, 0.410 mmol) in DCM (4 mL) was added TEA (0.14 mL, 1.04 mmol) dropwise. The mixture was stirred at 20° C. for 2 hours. After completion, the mixture was diluted with water (50 mL) and extracted with EtOAc(100 mL×3). The combined organic layer was washed with brine (50 mL×2), dried with anhydrous Na₂SO₄ and concentrated under vacuum to give residue, which was purified by prep-HPLC (FA as additive) to afford compound 026c (150 mg, 73.25% yield) as white solid. MS obsd. (ESI⁺) [(M+H)⁺]: 586.3.

Step 4: 3-[2-(3-Aminopropylamino)ethyl]-N-[4-[S-[4-[6-chloro-4-(trifluoromethyl)-2-pyridyl]piperazin-1-yl]-N-cyano-sulfonimidoyl]phenyl]benzamide

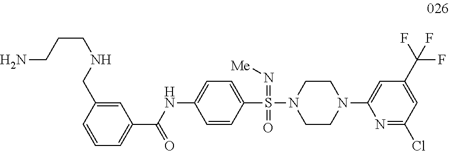 [SMILES: CN=S(=O)(c1ccc(NC(=O)c2cccc(CNCCCN)c2)cc1)N1CCN(c2cc(C(F)(F)F)cc(Cl)n2)CC1]

The mixture of compound 026c(100.0 mg, 0.170 mmol), 1,3-diaminopropane (101.11 mg, 1.36 mmol), K₂CO₃ (70.7 mg, 0.510 mmol) and KI (0.01 mL, 0.170 mmol) was stirred at 60° C. for 1 hour. After completion, the mixture was concentrated under vacuum to give residue, which was purified by prep-HPLC (HCl as additive) to give Example 026 (68.4 mg, 64.19% yield) as white solid. MS obsd. (ESI⁺) [(M+H)⁺]: 624.3.

### Example 026: ¹H NMR (400 MHz, DMSO-d6) δ ppm 10.95-11.12 (m, 1H), 9.63-9.87 (m, 2H), 8.31-8.40 (m, 1H), 8.15-8.27 (m, 4H), 7.98-8.08 (m, 1H), 7.87-7.94 (m, 2H), 7.76-7.87 (m, 1H), 7.54-7.64 (m, 1H), 7.07-7.19 (m, 1H), 6.92-7.01 (m, 1H), 4.15-4.30 (m, 2 H), 3.65-3.83 (m, 4H), 2.99-3.20 (m, 6H), 2.88-2.98 (m, 2H), 2.74-2.84 (m, 3H), 1.98-2.16 (m, 2H)

### Example 027: 3-[(3-Aminopropylamino)methyl]-N-[4-[4-[6-chloro-4-(trifluoromethyl)-2-pyridyl]piperazin-1-yl]sulfonyl-3-methoxy-phenyl]benzamide

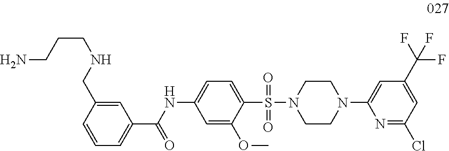 [SMILES: COc1cc(NC(=O)c2cccc(CNCCCN)c2)ccc1S(=O)(=O)N1CCN(c2cc(C(F)(F)F)cc(Cl)n2)CC1]

Step 1: 1-[6-Chloro-4-(trifluoromethyl)-2-pyridyl]-4-(2-methoxy-4-nitro-phenyl)sulfonyl-piperazine

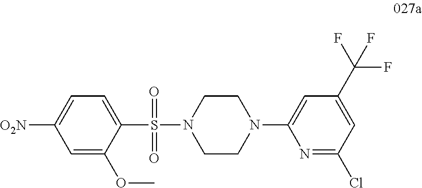 [SMILES: COc1cc([N+](=O)[O-])ccc1S(=O)(=O)N1CCN(c2cc(C(F)(F)F)cc(Cl)n2)CC1]

The title compound was prepared in analogy to the preparation of compound Int-1b by replacing 4-acetamidobenzenesulfonyl chloride with 2-methoxy-4-nitrobenzenesulfonyl chloride. Compound 027a (400 mg) was obtained as a light yellow solid. MS obsd. (ESI⁺) [(M+H)⁺]: 483.0.

Step 2: 4-[4-[6-Chloro-4-(trifluoromethyl)-2-pyridyl]piperazin-1-yl]sulfonyl-3-methoxy-aniline

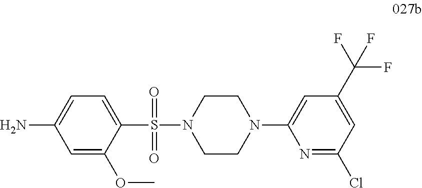 [SMILES: COc1cc(N)ccc1S(=O)(=O)N1CCN(c2cc(C(F)(F)F)cc(Cl)n2)CC1]

A mixture of compound 027a (400.0 mg, 0.830 mmol) and Fe (465.84 mg, 8.32 mmol) in methanol (15 mL) and NH₄Cl (sat., 3 mL) was heated at 65° C. for 1 hour. After completion, the mixture was filtered, the filtrate was poured into water (25 mL) and extracted by EtOAc (20 mL×2). The combined organic layer was washed with brine (20 mL×2), dried over Na₂SO₄ and concentrated under vacuum to give crude compound 027b (320 mg, 81.91% yield), which was used in the next step directly. MS obsd. (ESI⁺) [(M+H)⁺]: 451.1.

Step 3: N-[4-[4-[6-chloro-4-(trifluoromethyl)-2-pyridyl]piperazin-1-yl]sulfonyl-3-methoxy-phenyl]-4-formyl-benzamide

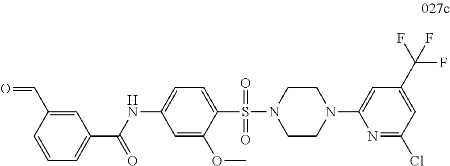 [SMILES: COc1cc(NC(=O)c2cccc(C=O)c2)ccc1S(=O)(=O)N1CCN(c2cc(C(F)(F)F)cc(Cl)n2)CC1]

To a mixture of 4-formylbenzoic acid (49.95 mg, 0.330 mmol) in DCM (12.86 mL) was added oxalyl chloride (42.23 mg, 0.330 mmol, 1 eq) and a drop of DMF. The resulted mixture was stirred at rt. After completion, the solvent was removed under vacuum to give a residue, which was added to a mixture of compound 027b (150.0 mg, 0.330 mmol) and TEA (0.2 mL), DMAP (25 mg) in DCM (12.86 mL). The mixture was stirred at rt for 1 hour. After completion, the solvent was removed under vacuum to give residue which was purified by silica-gel chromatography (elute with EtOAc:PE=50%) to give the desired product compound 027c (180 mg, 33.81% yield) as a colorless oil. MS obsd. (ESI⁺) [(M+H)⁺]: 583.0.

Step 4: Tert-butyl N-[3-[[3-[[4-[4-[6-chloro-4-(trifluoromethyl)-2-pyridyl]piperazin-1-yl]sulfonyl-3-methoxy-phenyl]carbamoyl]phenyl]methylamino]propyl]carbamate

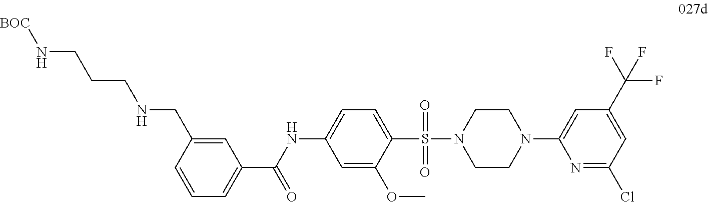 [SMILES: COc1cc(NC(=O)c2cccc(CNCCCNC(=O)OC(C)(C)C)c2)ccc1S(=O)(=O)N1CCN(c2cc(C(F)(F)F)cc(Cl)n2)CC1]

A mixture of N-BOC-1,3-diaminopropane (89.66 mg, 0.510 mmol), compound 027c (150.0 mg, 0.260 mmol) and Ti(OEt)₄ (58.69 mg, 0.260 mmol) in THF (15 mL) was stirred at 25° C. for 2 hours. And then, sodium cyanoborohydride (32.34 mg, 0.510 mmol, 2 eq) was added to this mixture and the resulted mixture was stirred at rt for another 1 hour. After completion, brine (20 mL) was added to this mixture. The mixture was extracted by EtOAc (25 mL×2), washed with brine (25 mL), dried over Na₂SO₄ and purified by silica-gel chromatography (elute with EtOAc:PE=50%) to afford the desired product compound 027d (40 mg, 19.3% yield) as a white solid. MS obsd. (ESI⁺) [(M+H)⁺]: 741.2.

Step 4: 3-[(3-Aminopropylamino)methyl]-N-[4-[4-[6-chloro-4-(trifluoromethyl)-2-pyridyl]piperazin-1-yl]sulfonyl-3-methoxy-phenyl]benzamide

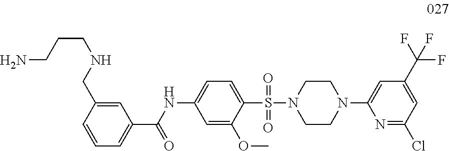 [SMILES: COc1cc(NC(=O)c2cccc(CNCCCN)c2)ccc1S(=O)(=O)N1CCN(c2cc(C(F)(F)F)cc(Cl)n2)CC1]

A mixture of compound 027d (60.0 mg, 0.080 mmol) in HCl-dioxane (5 mL, 4 M) was stirred at 25° C. for 1 hour. After completion, the solvent was removed under vacuum to give a residue, which was purified by prep-HPLC (ACN/water=35-45%) to give the desired product Example 027 (TFA as additive) as a white solid. MS obsd. (ESI⁺) [(M+H)⁺]: 641.2.

### Example 027: ¹H NMR (400 MHz, DMSO-d6) δ ppm 10.68 (s, 1H), 8.23 (s, 1H), 7.99 (s, 1H), 7.85 (d, J=7.8 Hz, 1H), 7.80 (d, J=1.6 Hz, 1H), 7.70 (d, J=8.7 Hz, 1H), 7.54-7.64 (m, 2H), 7.48 (t, J=7.7 Hz, 1H), 7.07 (s, 1H), 6.96 (s, 1H), 3.85 (s, 3H), 3.81 (s, 2H), 3.66-3.62 (m, 4H), 3.11-3.18 (m, 4H), 2.84 (t, J=7.3 Hz, 2H), 2.62 (t, J=6.7 Hz, 2H), 1.74 (dd, J=14.0, 6.9 Hz, 2H). ¹⁹F NMR (376 MHz, CDCl₃) δ ppm −58.72 (s, 3F)

### Example 028: 3-[(3-Aminopropylamino)methyl]-N-[4-[4-[6-chloro-4-(trifluoromethyl)-2-pyridyl]piperazin-1-yl]sulfonyl-3-hydroxy-phenyl]benzamide

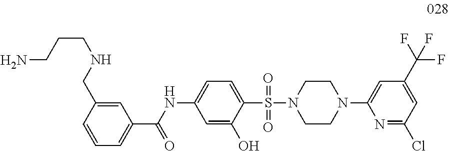 [SMILES: NCCCNCc1cccc(C(=O)Nc2ccc(S(=O)(=O)N3CCN(c4cc(C(F)(F)F)cc(Cl)n4)CC3)c(O)c2)c1]

To a mixture of Example 027 (40.0 mg, 0.060 mmol) in DCM (10 mL) was added BBr₃ (156.31 mg, 0.620 mmol) dropwise. The mixture was stirred at rt for 2 hours. After completion, the mixture was poured into 20 mL ice water, extracted with DCM (10 mL×2). The combined organic layer was washed with NaHCO₃ aq. (15 mL), dried over Na₂SO₄ and concentrated under vacuum to give residue, which was purified by prep-HPLC (TFA as additive) to afford Example 028 (2.6 mg, 6.03% yield) as a white solid. MS obsd. (ESI⁺) [(M+H)⁺]: 627.2.

### Example 028: ¹H NMR (400 MHz, CD₃OD) δ ppm 8.06 (s, 1H), 8.00 (d, J=7.4 Hz, 1H), 7.58-7.77 (m, 4H), 7.24 (d, J=8.4 Hz, 1H), 6.93 (s, 1H), 6.83 (s, 1H), 4.32 (s, 2H), 3.71 (s, 4H), 3.26-3.29 (m, 4H), 3.19 (s, 2H), 3.05 (s, 2H), 2.10 (s, 2H). ¹⁹F NMR (376 MHz, CD₃OD) δ ppm −66.50 (s, 3F)

### Example 029: 4-[(3-Aminopropylamino)methyl]-N-[5-[4-[6-chloro-4-(trifluoromethyl)-2-pyridyl]piperazin-1-yl]sulfonyl-2-pyridyl]benzamide

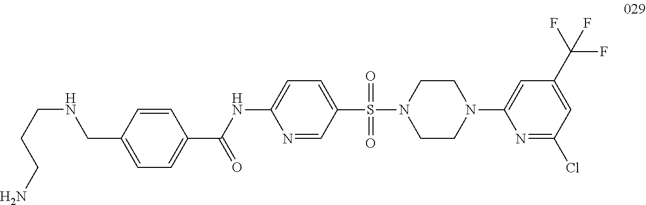 [SMILES: NCCCNCc1ccc(C(=O)Nc2ccc(S(=O)(=O)N3CCN(c4cc(C(F)(F)F)cc(Cl)n4)CC3)cn2)cc1]

Step 1: 1-[(6-Bromo-3-pyridyl)sulfonyl]-4-[6-chloro-4-(trifluoromethyl)-2-pyridyl]piperazine

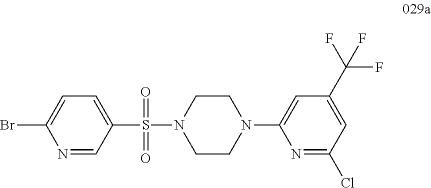 [SMILES: O=S(=O)(c1ccc(Br)nc1)N1CCN(c2cc(C(F)(F)F)cc(Cl)n2)CC1]

The title compound was prepared in analogy to the preparation of compound Int-1b by replacing 4-acetamidobenzenesulfonyl chloride with bromo-pyridine-3-sulfonyl chloride. Compound 029a (350 mg) was obtained as a white solid. MS obsd. (ESI⁺) [(M+H)⁺]: 484.9.

Step 2: N-[5-[4-[6-chloro-4-(trifluoromethyl)-2-pyridyl]piperazin-1-yl]sulfonyl-2-pyridyl]-1,1-diphenyl-methanimine

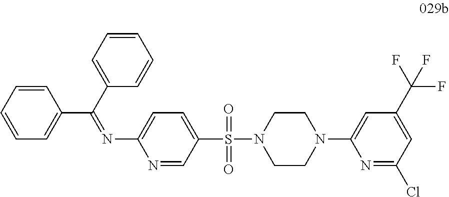 [SMILES: O=S(=O)(c1ccc(N=C(c2ccccc2)c2ccccc2)nc1)N1CCN(c2cc(C(F)(F)F)cc(Cl)n2)CC1]

A mixture of compound 029a (350.0 mg, 0.720 mmol), benzenemethanimine (200 mg, 1.10 mmol), tris(dibenzylideneacetone)dipalladium (0) (65.99 mg, 0.070 mmol), (R)-BINAP (44.87 mg, 0.070 mmol) and Cs₂CO₃ (352.18 mg, 1.08 mmol) in 1,4-dioxane (15 mL) was heated at 90° C. under N₂ for 16 hours. After being cooled to rt, the mixture was filtered and the filtrate was concentrated under vacuum to give a residue, which was purified by silica-gel chromatography (elute with EtOAc:PE=20%) to afford the desired product compound 029b (300 mg, 39.07% yield) as a light yellow solid. MS obsd. (ESI⁺) [(M+H)⁺]: 586.1.

Step 3: 5-[4-[6-Chloro-4-(trifluoromethyl)-2-pyridyl]piperazin-1-yl]sulfonylpyridin-2-amine

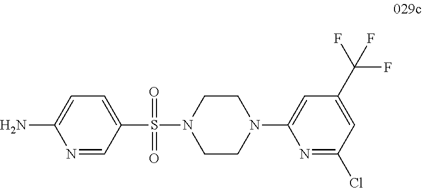 [SMILES: Nc1ccc(S(=O)(=O)N2CCN(c3cc(C(F)(F)F)cc(Cl)n3)CC2)cn1]

To a mixture of compound 029b (200.0 mg, 0.340 mmol) in THF (10 mL) was added hydrochloric acid (0.5 mL, 6 mmol). The mixture was stirred at 25° C. for 1 hour. After completion, the solvent was removed under vacuum and purified by silica-gel chromatography (elute with EtOAc:PE=70%) to afford the desired product compound 029c (120 mg, 80.02% yield) as a white solid. MS obsd. (ESI⁺) [(M+H)⁺]: 422.1.

Step 4: 4-(Chloromethyl)-N-[5-[4-[6-chloro-4-(trifluoromethyl)-2-pyridyl]piperazin-1-yl]sulfonyl-2-pyridyl]benzamide

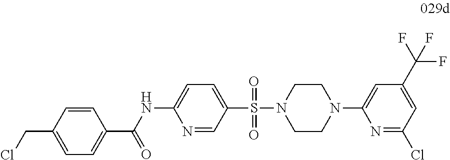 [SMILES: O=C(Nc1ccc(S(=O)(=O)N2CCN(c3cc(C(F)(F)F)cc(Cl)n3)CC2)cn1)c1ccc(CCl)cc1]

Compound 029d was prepared in analogy to the preparation of compound Int-2 by replacing compound Int-1 with compound 029c. Compound 029d (17 mg) was obtained as a white solid. MS obsd. (ESI⁺) [(M+H)⁺]: 574.0.

Step 5: Tert-butyl N-[3-[[4-[[5-[4-[6-chloro-4-(trifluoromethyl)-2-pyridyl]piperazin-1-yl]sulfonyl-2-pyridyl]carbamoyl]phenyl]methylamino]propyl]carbamate

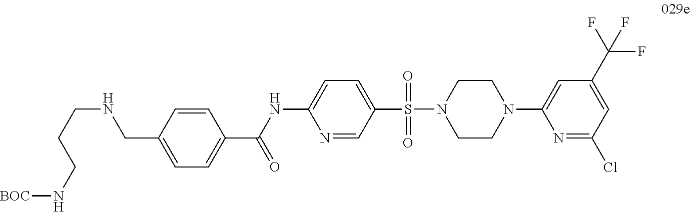 [SMILES: CC(C)(C)OC(=O)NCCCNCc1ccc(C(=O)Nc2ccc(S(=O)(=O)N3CCN(c4cc(C(F)(F)F)cc(Cl)n4)CC3)cn2)cc1]

Compound 029e was prepared in analogy to the preparation of compound 001a by replacing compound Int-2 with compound 029d in step 1. Compound 029e (40 mg) was obtained as a colorless oil. MS obsd. (ESI⁺) [(M+H)⁺]: 712.1.

Step 6: 4-[(3-Aminopropylamino)methyl]-N-[5-[4-[6-chloro-4-(trifluoromethyl)-2-pyridyl]piperazin-1-yl]sulfonyl-2-pyridyl]benzamide

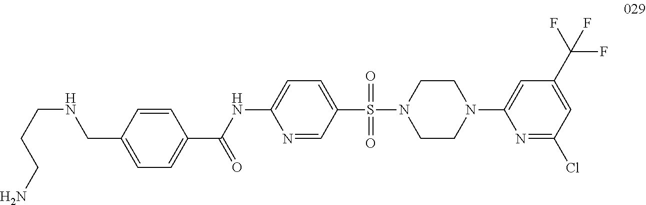 [SMILES: NCCCNCc1ccc(C(=O)Nc2ccc(S(=O)(=O)N3CCN(c4cc(C(F)(F)F)cc(Cl)n4)CC3)cn2)cc1]

The title compound was prepared in analogy to the preparation of Example 001 by replacing compound 001a with compound 029e in step 2. Example 029 (4 mg) was obtained as a white solid. MS obsd. (ESI⁺) [(M+H)⁺]: 612.2.

### Example 029: ¹H NMR (400 MHz, DMSO-d6) δ ppm 8.71 (d, J=2.3 Hz, 1H), 8.44 (d, J=8.9 Hz, 1H), 8.35 (s, 2H), 8.21 (dd, J=8.9, 2.3 Hz, 1H), 7.99 (d, J=8.0 Hz, 2H), 7.47 (d, J=8.0 Hz, 2H), 7.10 (s, 1H), 6.97 (s, 1H), 3.76 (s, 6H), 3.07 (s, 4H), 2.84 (s, 2H), 2.56 (d, J=6.2 Hz, 2H), 1.69 (s, 2H). ¹⁹F NMR (376 MHz, DMSO-d6) δ ppm −63.45 (s, 3F)

### Example 030: 3-[(3-Aminopropylamino)methyl]-N-[4-[4-[6-chloro-4-(trifluoromethyl)-2-pyridyl]piperazin-1-yl]sulfonyl-2-methoxy-phenyl]benzamide

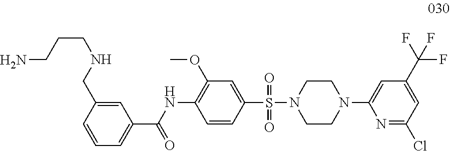 [SMILES: COc1cc(S(=O)(=O)N2CCN(c3cc(C(F)(F)F)cc(Cl)n3)CC2)ccc1NC(=O)c1cccc(CNCCCN)c1]

The title compound was prepared in analogy to the preparation of Example 027 by replacing 2-methoxy-4-nitrobenzenesulfonyl chloride with 3-methoxy-4-nitrobenzenesulfonyl chloride in step 1. Example 030 (4 mg) was obtained as a white solid. MS obsd. (ESI⁺) [(M+H)⁺]: 641.2.

### Example 030: ¹H NMR (400 MHz, DMSO-d6) δ ppm 9.61 (s, 1H), 8.23 (d, J=8.4 Hz, 1H), 7.92 (s, 1H), 7.82 (d, J=7.6 Hz, 1H), 7.58 (d, J=7.1 Hz, 1H), 7.49 (t, J=7.4 Hz, 1H), 7.41 (dd, J=8.4, 1.6 Hz, 1H), 7.32 (d, J=1.5 Hz, 1H), 7.10 (s, 1H), 6.97 (s, 1H), 3.97 (s, 3H), 3.75 (d, J=19.4 Hz, 6H), 3.05 (s, 4H), 2.84 (s, 2H), 2.60 (s, 2H), 1.71 (s, 2H). ¹⁹F NMR (376 MHz, DMSO-d6) δ ppm −63.46 (s, 3F)

### Example 031: 3-[(3-Aminopropylamino)methyl]-N-[4-[4-[6-chloro-4-(trifluoromethyl)-2-pyridyl]piperazin-1-yl]sulfonyl-2-hydroxy-phenyl]benzamide

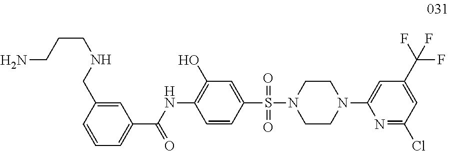 [SMILES: NCCCNCc1cccc(C(=O)Nc2ccc(S(=O)(=O)N3CCN(c4cc(C(F)(F)F)cc(Cl)n4)CC3)cc2O)c1]

The title compound was prepared in analogy to the preparation of Example 028 by replacing Example 027 with Example 030. Example 031 (6 mg) was obtained as a white solid. MS obsd. (ESI⁺) [(M+H)⁺]: 627.2.

### Example 031: ¹H NMR (400 MHz, DMSO-d6) δ ppm 8.27 (d, J=8.4 Hz, 1H), 7.95 (s, 1H), 7.81 (d, J=7.4 Hz, 1H), 7.56 (d, J=7.4 Hz, 1H), 7.49 (t, J=7.6 Hz, 1H), 7.16 (s, 1H), 7.07 (s, 1H), 6.98 (d, J=8.7 Hz, 2H), 3.80 (s, 2H), 3.66 (s, 4H), 2.87 (dd, J=19.4, 12.4 Hz, 6H), 2.61 (t, J=6.3 Hz, 2H), 1.65-1.81 (m, 2H). 1⁹F NMR (376 MHz, DMSO-d6) δ ppm −63.50 (s, 3F)

### Example 032 and 033: N-[4-[4-[6-chloro-4-(trifluoromethyl)-2-pyridyl]piperazin-1-yl]sulfonylphenyl]-2-methyl-4,6-dihydropyrrolo[3,4-c]pyrazole-5-carboxamide (Example 032) and N-[4-[4-[6-chloro-4-(trifluoromethyl)-2-pyridyl]piperazin-1-yl]sulfonylphenyl]-1-methyl-4,6-dihydropyrrolo[3,4-c]pyrazole-5-carboxamide (Example 033)

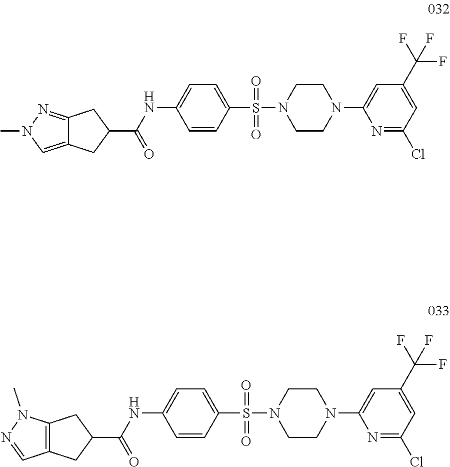 [SMILES: Cn1cc2c(n1)CC(C(=O)Nc1ccc(S(=O)(=O)N3CCN(c4cc(C(F)(F)F)cc(Cl)n4)CC3)cc1)C2 ; Cn1ncc2c1CC(C(=O)Nc1ccc(S(=O)(=O)N3CCN(c4cc(C(F)(F)F)cc(Cl)n4)CC3)cc1)C2]

To a mixture solution of compound Int-1 (130 mg, 0.309 mmol) in DCM (5 mL) was added DIEA (0.204 mL, 1.24 mmol), triphosgene (45.83 mg, 0.154 mmol) and stirred at 0° C. for 15 minutes. And then, mixture of compound Int-12A&12B (80.6, mg, 0.340 mmol) was added to the mixture and stirred at 0° C. for another 2 hours. After completion, the mixture was quenched with water (20 mL), extracted with DCM (10 mL×2). The combined organic layer was dried over anhydrous Na₂SO₄, concentrated under vacuum to give a residue, which was purified by prep-HPLC (NH₄HCO₃ as additive) to give 110 mg mixture of desired products. The further SFC purification to give pure Example 032 (19.2 mg, 10.73% yield) as a white solid and Example 033 (19.2 mg, 10.73% yield) as a white solid.

### Example 032: ¹H NMR (400 MHz, CDCl₃) δ ppm 7.55-7.61 (m, 4H), 6.70 (s, 1H), 6.59 (s, 1H), 6.53 (s, 1H), 4.59 (s, 2H), 4.52 (s, 2H), 3.78 (s, 3H), 3.65 (s, 4H), 3.01 (s, 4H). ¹⁹F NMR (376 MHz, CDCl₃) −65.03. MS obsd. (ESI⁺) [(M+H)⁺]: 570.1

### Example 033: ¹H NMR (400 MHz, CDCl₃) δ ppm 7.64-7.69 (m, 4H), 7.18 (s, 1H), 6.77 (s, 1H), 6.60 (s, 1H), 4.62 (d, J=6.9 Hz, 4H), 3.93 (s, 3H), 3.72 (s, 4H), 3.09 (s, 4H). ¹⁹F NMR (376 MHz, CDCl₃) −65.03. MS obsd. (ESI⁺) [(M+H)⁺]: 570.1

### Example 034: [5-[4-[6-Chloro-4-(trifluoromethyl)-2-pyridyl]piperazin-1-yl]sulfonylindolin-1-yl]-pyrazolo[1,5-a]pyrimidin-3-yl-methanone

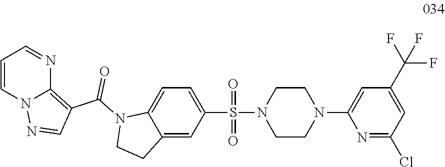 [SMILES: O=C(c1cnn2cccnc12)N1CCc2cc(S(=O)(=O)N3CCN(c4cc(C(F)(F)F)cc(Cl)n4)CC3)ccc21]

To a solution of pyrazolo[1,5-a]pyrimidine-3-carboxylic acid (84.44 mg, 0.520 mmol) in DCM (6 mL) was added oxalyl chloride (0.3 mL) and the reaction was stirred at rt for 3 hours. The reaction mixture was concentrated under vacuum to give crude residue, which was re-dissolved in 4 mL DCM. To a solution of compound Int-4 (50.0 mg, 0.110 mmol) and DIPEA (129 mg, 1.0 mmol) in DCM (4 mL) was added above solution dropwise. The reaction was stirred at rt for 4 hours. And then, the mixture was concentrated under vacuum and purified by silica-gel chromatography (elute with EtOAc:PE=50%) to give Example 034 (9 mg, 13.54% yield) as a white solid. MS obsd. (ESI⁺) [(M+H)⁺]: 592.1.

### Example 034: ¹H NMR (400 MHz, DMSO-d6) δ ppm 9.28 (dd, J=7.0, 1.7 Hz, 1H), 8.70-8.77 (m, 1H), 8.60 (d, J=4.5 Hz, 1H), 8.15 (d, J=7.8 Hz, 1H), 7.63 (s, 1H), 7.48 (dd, J=39.6, 7.9 Hz, 1H), 7.23-7.29 (m, 1H), 7.10 (s, 1H), 6.97 (s, 1H), 4.37 (dd, J=10.3, 6.5 Hz, 2H), 3.73 (s, 4H), 3.21 (t, J=7.0 Hz, 2H), 2.99 (s, 4H). ¹⁹F NMR (376 MHz, DMSO-d6): δ ppm −63.46 (s, 3F)

### Example 035: 4-[5-[4-[6-Chloro-4-(trifluoromethyl)-2-pyridyl]piperazin-1-yl]sulfonylindoline-1-carbonyl]-2-methyl-pyrazole-3-carboxylic acid

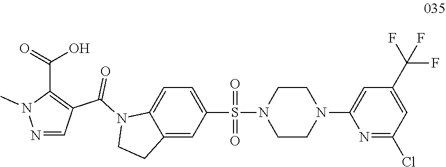 [SMILES: Cn1ncc(C(=O)N2CCc3cc(S(=O)(=O)N4CCN(c5cc(C(F)(F)F)cc(Cl)n5)CC4)ccc32)c1C(=O)O]

Step 1: Ethyl 4-[5-[4-[6-chloro-4-(trifluoromethyl)-2-pyridyl]piperazin-1-yl]sulfonylindoline-1-carbonyl]-2-methyl-pyrazole-3-carboxylate

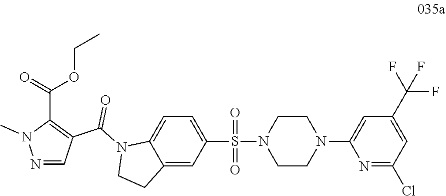 [SMILES: CCOC(=O)c1c(C(=O)N2CCc3cc(S(=O)(=O)N4CCN(c5cc(C(F)(F)F)cc(Cl)n5)CC4)ccc32)cnn1C]

Compound 035a was prepared in analogy to the preparation of Example 034 by replacing pyrazolo[1,5-a]pyrimidine-3-carboxylic acid with 5-ethoxycarbonyl-1-methyl-pyrazole-4-carboxylic acid. Compound 035a (50 mg) was obtained as a white solid. MS obsd. (ESI⁺) [(M+H)⁺]: 627.2.

Step 2: 4-[5-[4-[6-Chloro-4-(trifluoromethyl)-2-pyridyl]piperazin-1-yl]sulfonylindoline-1-carbonyl]-2-methyl-pyrazole-3-carboxylic acid

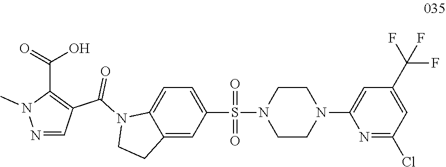 [SMILES: Cn1ncc(C(=O)N2CCc3cc(S(=O)(=O)N4CCN(c5cc(C(F)(F)F)cc(Cl)n5)CC4)ccc32)c1C(=O)O]

A solution of ethyl compound 035a (20 mg, 0.032 mmol) and LiGH (2.93 mg, 0.128 mmol) in THF/MeOH/water (4/4/1 mL). The resulting mixture was stirred at rt for 16 hours. To the mixture was added 0.1 mL 2 M HCl aq. The resulting mixture was concentrated under vacuum to give a residue, which was purified by prep-HPLC to give Example 035 (3.5 mg, 18.21% yield) as a white solid. MS obsd. (ESI⁺) [(M+H)⁺]: 599.1.

### Example 035: ¹H NMR (400 MHz, CD3OD) δ ppm 8.41 (d, J=8.4 Hz, 1H), 7.83 (s, 1H), 7.75-7.65 (m, 2H), 6.93 (s, 1H), 6.83 (s, 1H), 4.04 (s, 2H), 3.86 (s, 3H), 3.74 (dd, J=12.5, 7.6 Hz, 4H), 3.24 (t, J=7.7 Hz, 2H), 3.07 (dd, J=16.9, 12.0 Hz, 4H). ¹⁹F NMR (376 MHz, CD₃OD) δ ppm −66.46 (s, 3F)

### Example 036: [1-(Azetidin-3-yl)pyrazol-4-yl]-[5-[4-[6-chloro-4-(trifluoromethyl)-2-pyridyl]piperazin-1-yl]sulfonylindolin-1-yl]methanone

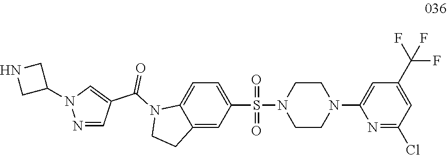 [SMILES: O=C(c1cnn(C2CNC2)c1)N1CCc2cc(S(=O)(=O)N3CCN(c4cc(C(F)(F)F)cc(Cl)n4)CC3)ccc21]

Step 1: Methyl 1-(1-tert-butoxycarbonylazetidin-3-yl)pyrazole-4-carboxylate

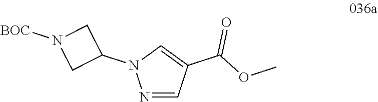 [SMILES: COC(=O)c1cnn(C2CN(C(=O)OC(C)(C)C)C2)c1]

To a solution of methyl 1H-pyrazole-4-carboxylate (63 mg, 0.500 mmol) in MeCN (10 mL) were added Cs₂CO₃ (325.56 mg, 0.999 mmol) and tert-butyl 3-bromoazetidine-1-carboxylate (129.75 mg, 0.999 mmol). The resulting suspension mixture was stirred at 90° C. for 16 hours. After completion, the mixture was concentrated under vacuum to give a residue, which was purified by silica-gel chromatography (elute with EtOAc:PE=50%) to give the desired crude compound 036a (50 mg, 71.32% yield) as a white solid. MS obsd. (ESI⁺) [(M+H)⁺]: 281.0.

Step 2: 1-(1-Tert-butoxycarbonylazetidin-3-yl)pyrazole-4-carboxylic acid

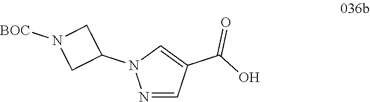 [SMILES: CC(C)(C)OC(=O)N1CC(n2cc(C(=O)O)cn2)C1]

A solution of compound 036a (50 mg, 0.178 mmol) and LiGH (8.18 mg, 0.355 mmol) in THF/MeOH/water (5/5/0.5 mL) was stirred at rt for 16 hours. After completion, the mixture was concentrated under vacuum to give a residue, which was purified by silica-gel chromatography (elute with EtOAc:PE=50%) to give the desired crude compound 036b (30 mg, 63.05% yield) as a white solid. MS obsd. (ESI⁺) [(M+H)⁺]: 267.1.

Step 3: Tert-butyl 3-[4-[5-[4-[6-chloro-4-(trifluoromethyl)-2-pyridyl]piperazin-1-yl]sulfonylindoline-1-carbonyl]pyrazol-1-yl]azetidine-1-carboxylate

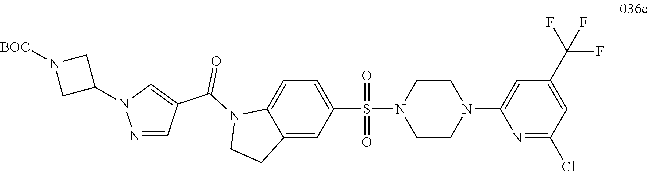 [SMILES: CC(C)(C)OC(=O)N1CC(n2cc(C(=O)N3CCc4cc(S(=O)(=O)N5CCN(c6cc(C(F)(F)F)cc(Cl)n6)CC5)ccc43)cn2)C1]

To a solution of compound 036b (30 mg, 0.112 mmol) in MeCN (45 mL) was added 1-methylimidazole (27.61 mg, 0.337 mmol), TCFH (62.86 mg, 0.224 mmol). The resulting mixture was stirred at rt for 15 minutes. Then a solution of compound Int-4 was added to the mixture and the mixture was stirred at 25° C. for 16 hours. After completion, the reaction was quenched with 10 mL water, extracted with DCM (10 mL×2). The combined organic layer was dried over anhydrous Na₂SO₄, concentrated under vacuum to give a residue, which was purified by prep-TLC (EtOAc:PE=50%) to give compound 036c (30 mg, 19.2% yield) as a colorless oil. MS obsd. (ESI⁺) [(M+H)⁺]: 696.1.

Step 4: [1-(Azetidin-3-yl)pyrazol-4-yl]-[5-[4-[6-chloro-4-(trifluoromethyl)-2-pyridyl]piperazin-1-yl]sulfonylindolin-1-yl]methanone

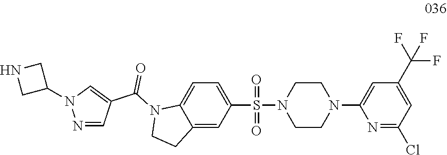 [SMILES: O=C(c1cnn(C2CNC2)c1)N1CCc2cc(S(=O)(=O)N3CCN(c4cc(C(F)(F)F)cc(Cl)n4)CC3)ccc21]

To a solution of compound 036 (30 mg, 0.034 mmol) in DCM (10 mL) was added TFA (2 mL). The mixture was stirred at 25° C. for 3 hours. Then the mixture was concentrated under vacuum to give a residue, which was purified by prep-HPLC to give Example 036 (5 mg, 11.08% yield) as a white solid. MS obsd. (ESI⁺) [(M+H)⁺]: 596.1.

### Example 036: ¹H NMR (400 MHz, CD₃OD) δ ppm 8.28 (d, J=7.2 Hz, 2H), 8.14 (s, 1H), 7.60 (dd, J=40.0, 31.3 Hz, 2H), 6.91 (s, 1H), 6.82 (s, 1H), 5.54-5.42 (m, 1H), 4.52 (d, J=6.8 Hz, 4H), 4.43 (t, J=8.4 Hz, 2H), 3.77-3.68 (m, 4H), 3.34 (d, J=8.4 Hz, 2H), 3.12-3.02 (m, 4H). 1⁹F NMR (376 MHz, CD3OD) δ ppm −63.46 (s, 3F)

### Example 037: [1-(3-Aminopropyl)pyrazol-4-yl]-[5-[4-[6-chloro-4-(trifluoromethyl)-2-pyridyl]piperazin-1-yl]sulfonylindolin-1-yl]methanone

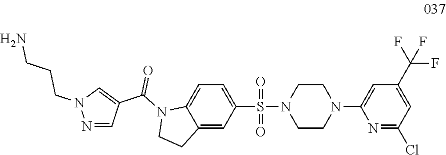 [SMILES: NCCCn1cc(C(=O)N2CCc3cc(S(=O)(=O)N4CCN(c5cc(C(F)(F)F)cc(Cl)n5)CC4)ccc32)cn1]

The title compound was prepared in analogy to the preparation of Example 036 by replacing tert-butyl 3-bromoazetidine-1-carboxylate with tert-butyl N-(3-aminopropyl)carbamate in step 1. Compound 037a (50 mg) was obtained as a white solid. MS obsd. (ESI⁺) [(M+H)⁺]: 597.2.

### Example 037: ¹H NMR (400 MHz, DMSO-d6) δ ppm 8.44 (d, J=22.7 Hz, 2H), 8.26 (d, J=8.5 Hz, 1H), 7.97 (s, 1H), 7.60 (d, J=11.2 Hz, 1H), 7.46 (dd, J=40.1, 7.7 Hz, 1H), 7.09 (s, 1H), 6.96 (s, 1H), 4.40 (s, 2H), 4.26 (s, 2H), 3.72 (s, 4H), 3.28 (d, J=6.2 Hz, 2H), 2.97 (s, 4H), 2.67 (s, 2H), 2.03 (s, 2H). 1⁹F NMR (376 MHz, DMSO) δ ppm −63.47 (s, 3F)

### Example 038: [5-[4-[6-Chloro-4-(trifluoromethyl)-2-pyridyl]piperazin-1-yl]sulfonylindolin-1-yl]-(4,5,6,7-tetrahydropyrazolo[1,5-a]pyrazin-3-yl)methanone

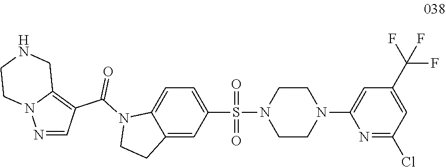 [SMILES: O=C(c1cnn2c1CNCC2)N1CCc2cc(S(=O)(=O)N3CCN(c4cc(C(F)(F)F)cc(Cl)n4)CC3)ccc21]

Step 1: Tert-butyl 3-[5-[4-[6-chloro-4-(trifluoromethyl)-2-pyridyl]piperazin-1-yl]sulfonylindoline-1-carbonyl]-6,7-dihydro-4H-pyrazolo[1,5-a]pyrazine-5-carboxylate

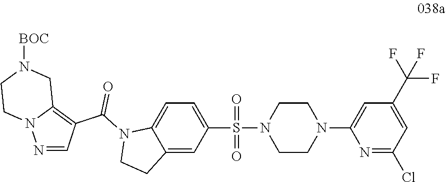 [SMILES: CC(C)(C)OC(=O)N1CCn2ncc(C(=O)N3CCc4cc(S(=O)(=O)N5CCN(c6cc(C(F)(F)F)cc(Cl)n6)CC5)ccc43)c2C1]

The title compound was prepared in analogy to the preparation of Example 034 by replacing pyrazolo[1,5-a]pyrimidine-3-carboxylic acid with 5-tert-butoxycarbonyl-6,7-dihydro-4H-pyrazolo[1,5-a]pyrazine-3-carboxylic acid. Compound 038a (95 mg) was obtained as a white solid. MS obsd. (ESI⁺) [(M+H)⁺]: 696.2.

Step 2: [5-[4-[6-Chloro-4-(trifluoromethyl)-2-pyridyl]piperazin-1-yl]sulfonylindolin-1-yl]-(4,5,6,7-tetrahydropyrazolo[1,5-a]pyrazin-3-yl)methanone

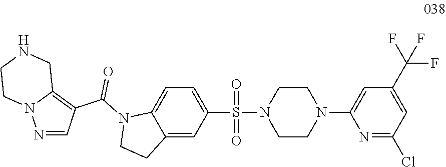 [SMILES: O=C(c1cnn2c1CNCC2)N1CCc2cc(S(=O)(=O)N3CCN(c4cc(C(F)(F)F)cc(Cl)n4)CC3)ccc21]

Compound 038a (95.0 mg, 0.140 mmol) in 2 M HCl/EA(10 mL) was stirred at rt for 4 hours. After completion, the solvent was removed under vacuum to give the crude product, which was purified by prep-HPLC (TFA as additive) to afford Example 038 (40 mg, 46.11% yield). MS obsd. (ESI⁺) [(M+H)⁺]: 596.2.

### Example 038: ¹H NMR (400 MHz, DMSO-d6) δ ppm 8.20 (d, J=8.5 Hz, 1H), 7.94 (s, 1H), 7.63-7.54 (m, 2H), 7.09 (s, 1H), 6.97 (s, 1H), 4.40 (t, J=8.4 Hz, 2H), 4.09 (d, J=11.5 Hz, 2H), 4.02 (s, 2H), 3.72 (s, 4H), 3.24 (t, J=8.3 Hz, 2H), 3.12 (s, 2H), 2.97 (d, J=4.2 Hz, 4H). ¹⁹F NMR (376 MHz, DMSO) δ ppm −63.47 (s, 3F)

### Example 039: [5-[4-[6-Chloro-4-(trifluoromethyl)-2-pyridyl]piperazin-1-yl]sulfonylindolin-1-yl]-(1-oxa-2,9-diazaspiro[4.51dec-2-en-3-yl)methanone

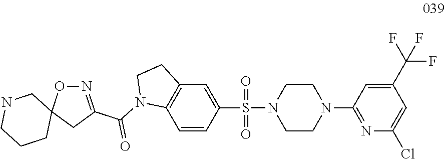 [SMILES: O=C(C1=NOC2(CCCNC2)C1)N1CCc2cc(S(=O)(=O)N3CCN(c4cc(C(F)(F)F)cc(Cl)n4)CC3)ccc21]

Step 1: Tert-butyl 3-[5-[4-[6-chloro-4-(trifluoromethyl)-2-pyridyl]piperazin-1-yl]sulfonylindoline-1-carbonyl]-1-oxa-2,9-diazaspiro[4.5]dec-2-ene-9-carboxylate

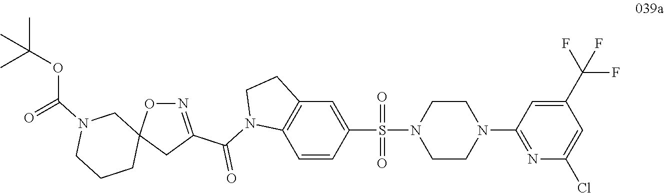 [SMILES: CC(C)(C)OC(=O)N1CCCC2(CC(C(=O)N3CCc4cc(S(=O)(=O)N5CCN(c6cc(C(F)(F)F)cc(Cl)n6)CC5)ccc43)=NO2)C1]

To a solution of 7-(tert-butoxycarbonyl)-1-oxa-2,7-diazaspiro[4.5]dec-2-ene-3-carboxylic acid (35 mg, 123 μmol) in MeCN (2.5 mL) was added 1-methylimidazole (30.3 mg, 369 μmol) and HATU (69.1 mg, 246 μmol). The resulting mixture was stirred at rt for 15 minutes. Then a solution of compound Int-4 (59 mg, 132 μmol) in MeCN was added. The mixture was stirred at 25° C. for 5 hours. After completion, the reaction was quenched with water (10 mL). The reaction mixture was extracted with EtOAc (10 mL×3). The combined organic layer was washed with brine (50 mL), dried over anhydrous Na₂SO₄ and concentrated under vacuum to afford crude compound 039a (80 mg, 91% yield) as yellow oil. MS obsd. (ESI⁺) [(M+Na)⁺]: 735.60.

Step 2: [5-[4-[6-Chloro-4-(trifluoromethyl)-2-pyridyl]piperazin-1-yl]sulfonylindolin-1-yl]-(1-oxa-2,9-diazaspiro[4.5]dec-2-en-3-yl)methanone

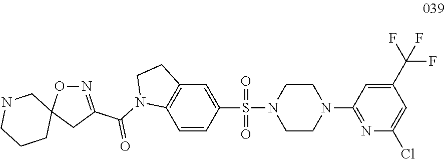 [SMILES: O=C(C1=NOC2(CCCNC2)C1)N1CCc2cc(S(=O)(=O)N3CCN(c4cc(C(F)(F)F)cc(Cl)n4)CC3)ccc21]

Compound 039a (70 mg, 98.2 μmol) was dissolved in the DCM (7.5 mL), followed by adding TFA (2 mL, 98.2 umol). The reaction was stirred at rt and went completed for 2 hours. After completion, the reaction was concentrated in vacuo to give crude product, which was purified by prep-HPLC to afford Example 039 (34.87 mg, 46.4% yield) as a white solid. MS obsd. (ESI⁺) [(M+H)⁺]: 613.3.

### Example 039: ¹H NMR (400 MHz, DMSO-d6) δ ppm 8.8-9.1 (m, 1H), 8.6-8.8 (m, 1H), 8.24 (br d, 1H, J=7.6 Hz), 7.5-7.8 (m, 2H), 7.09 (s, 1H), 6.98 (s, 1H), 4.42 (s, 2H), 3.7-3.8 (m, 4H), 3.1-3.3 (m, 6H), 2.9-3.0 (m, 5H), 1.7-2.1 (m, 4H). ¹⁹F NMR (376 MHz, DMSO-d₆) δ ppm −63.46 (m, 3F)

### Example 040: N-[4-[4-[6-chloro-4-(trifluoromethyl)-2-pyridyl]piperazin-1-yl]sulfonylphenyl]-2-piperazin-1-yl-acetamide

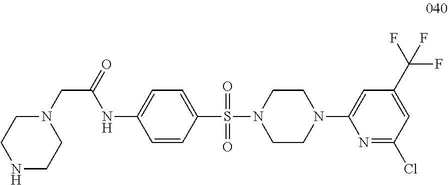 [SMILES: O=C(CN1CCNCC1)Nc1ccc(S(=O)(=O)N2CCN(c3cc(C(F)(F)F)cc(Cl)n3)CC2)cc1]

Step 1: 2-Chloro-N-[4-[4-[6-chloro-4-(trifluoromethyl)-2-pyridyl]piperazin-1-yl]sulfonylphenyl]acetamide

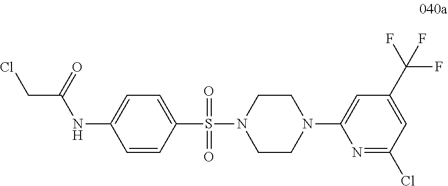 [SMILES: O=C(CCl)Nc1ccc(S(=O)(=O)N2CCN(c3cc(C(F)(F)F)cc(Cl)n3)CC2)cc1]

Compound Int-1 (100 mg, 238 μmol), chloroacetyl chloride (67.1 mg, 594 μmol) and DIPEA (468 mg, 3.62 mmol) was stirred at rt for 4 hours. After completion, the reaction mixture was extracted with EtOAc (50 mL×3). The combined organic layer was washed with brine (100 mL), dried over anhydrous Na₂SO₄ and concentrated under vacuum to afford crude product 040a (118 mg, 100% yield) as a yellow solid. MS obsd. (ESI⁺) [(M+H)⁺]: 496.8.

Step 2: Tert-butyl 4-[2-[4-[4-[6-chloro-4-(trifluoromethyl)-2-pyridyl]piperazin-1-yl]sulfonylanilino]-2-oxo-ethyl]piperazine-1-carboxylate

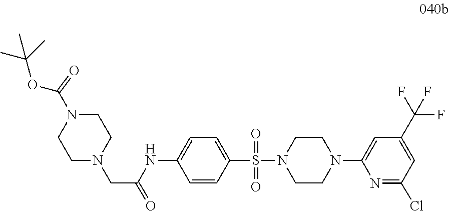 [SMILES: CC(C)(C)OC(=O)N1CCN(CC(=O)Nc2ccc(S(=O)(=O)N3CCN(c4cc(C(F)(F)F)cc(Cl)n4)CC3)cc2)CC1]

Compound 040b (60 mg, 121 μmol) and tert-butyl piperazine-1-carboxylate (44.9 mg, 241 μmol) were dissolved in the DCM (10 mL), followed by adding DIPEA (46.8 mg, 63.2 μL, 362 μmol). The reaction was stirred at rt for 1 hour. After completion, the reaction mixture was extracted with EtOAc (50 mL×3). The combined organic layer was washed with brine (50 mL), dried over anhydrous Na₂SO₄ and concentrated under vacuum to afford crude product 040b (75 mg, 96.1% yield) as yellow oil. MS obsd. (ESI⁺) [(M+H)⁺]: 647.8.

Step 3: N-[4-[4-[6-chloro-4-(trifluoromethyl)-2-pyridyl]piperazin-1-yl]sulfonylphenyl]-2-piperazin-1-yl-acetamide

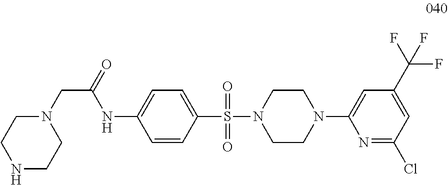 [SMILES: O=C(CN1CCNCC1)Nc1ccc(S(=O)(=O)N2CCN(c3cc(C(F)(F)F)cc(Cl)n3)CC2)cc1]

Compound 040b (70 mg, 108 μmol) in the DCM (5 mL) and TFA (2 mL, 108 μmol) was stirred at rt for 2 hours. After completion, the reaction mixture was concentrated under vacuum to give crude product, which was purified by prep-HPLC (TFA as additive) to afford Example 040 (29 mg, 38.5% yield) as a white solid. MS obsd. (ESI⁺) [(M+H)⁺]: 547.5.

### Example 040: ¹H NMR (400 MHz, DMSO-d₆) δ ppm 8.4-8.7 (m, 2H), 7.82 (d, 2H, J=8.7 Hz), 7.66 (d, 2H, J=8.8 Hz), 7.01 (s, 1H), 6.8-6.9 (m, 1H), 3.64 (br d, 4H, J=4.6 Hz), 3.10 (s, 6H), 2.89 (d, 4H, J=4.4 Hz), 2.7-2.8 (m, 4H). ¹⁹F NMR (376 MHz, DMSO-d₆) δ ppm −63.46 (m, 3F)

### Example 041: 1-[5-[4-[6-Chloro-4-(trifluoromethyl)-2-pyridyl]piperazin-1-yl]sulfonylindolin-1-yl]-2-(2-hydroxyethylamino)ethanone

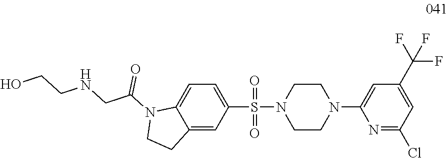 [SMILES: O=C(CNCCO)N1CCc2cc(S(=O)(=O)N3CCN(c4cc(C(F)(F)F)cc(Cl)n4)CC3)ccc21]

The title compound was prepared in analogy to the preparation of compound 040 by replacing compound Int-1 with compound Int-4 in Step 1 and tert-butyl piperazine-1-carboxylate with 2-aminoethanol in step 2. Example 041 (20 mg) was obtained as a white solid. MS obsd. (ESI⁺) [(M+H)⁺]: 548.5.

### Example 041: ¹H NMR (400 MHz, DMSO-d6) δ ppm 8.8-9.1 (m, 2H), 8.21 (d, 1H, J=8.3 Hz), 7.5-7.8 (m, 2H), 7.09 (s, 1H), 6.9-7.0 (m, 1H), 4.0-4.3 (m, 4H), 3.72 (br s, 6H), 3.08 (s, 2H), 2.97 (s, 4H). ¹⁹F NMR (376 MHz, DMSO-d₆) δ ppm −63.45 (m, 3F)

### Example 042: 2-[5-[4-[6-Chloro-4-(trifluoromethyl)-2-pyridyl]piperazin-1-yl]sulfonylindoline-1-carbonyl]benzoic acid

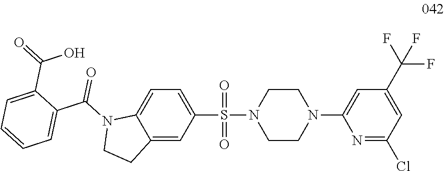 [SMILES: O=C(O)c1ccccc1C(=O)N1CCc2cc(S(=O)(=O)N3CCN(c4cc(C(F)(F)F)cc(Cl)n4)CC3)ccc21]

The title compound was prepared in analogy to the preparation of Example 035 by replacing 5-ethoxycarbonyl-1-methyl-pyrazole-4-carboxylic acid with 2-methoxycarbonylbenzoic acid in step 1. Example 042 (15.1 mg) was obtained as a white solid. MS obsd. (ESI⁺) [(M+H)⁺]: 595.2.

### Example 042: ¹H NMR (400 MHz, DMSO-d₆) δ ppm 8.30 (br d, J=8.4 Hz, 1H), 8.08-7.93 (m, 1H), 7.79-7.55 (m, 4H), 7.53-7.43 (m, 1H), 7.09 (s, 1H), 6.97 (s, 1H), 3.76-3.63 (m, 6H), 3.14 (br s, 1H), 3.06-2.82 (m, 5H)

### Example 043: [2-Acetoxy-4-[5-[4-[6-chloro-4-(trifluoromethyl)-2-pyridyl]piperazin-1-yl]sulfonylindoline-1-carbonyl]phenyl]acetate

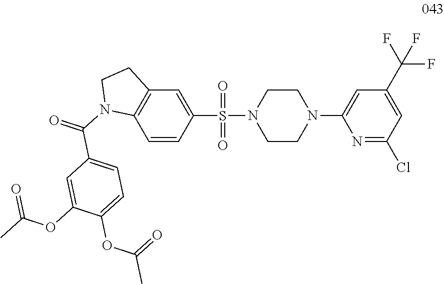 [SMILES: CC(=O)Oc1ccc(C(=O)N2CCc3cc(S(=O)(=O)N4CCN(c5cc(C(F)(F)F)cc(Cl)n5)CC4)ccc32)cc1OC(C)=O]

The title compound was prepared in analogy to the step of preparation of compound 039a by replacing (tert-butoxycarbonyl)-1-oxa-2,7-diazaspiro[4.5]dec-2-ene-3-carboxylic acid with 7-3,4-diacetoxybenzoic acid. Example 043 (9 mg) was obtained as a white solid. MS obsd. (ESI⁺) [(M+H)⁺]: 667.6.

### Example 043: ¹H NMR (400 MHz, DMSO-d₆) δ ppm 7.5-7.8 (m, 4H), 7.42 (s, 1H), 7.10 (s, 1H), 6.97 (s, 1H), 5.75 (s, 1H), 3.9-4.2 (m, 2H), 3.6-3.8 (m, 4H), 3.1-3.3 (m, 2H), 2.97 (br t, 4H, J=4.8 Hz), 2.30 (d, 6H, J=8.8 Hz). 1⁹F NMR (376 MHz, DMSO-d₆) δ ppm −63.46 (m, 3F)

### Example 044: N-[4-[4-[4-methyl-6-(trifluoromethyl)pyrimidin-2-yl]piperazin-1-yl]sulfonylphenyl]-6,7-dihydro-5H-pyrrolo[3,4-b]pyridine-3-carboxamide

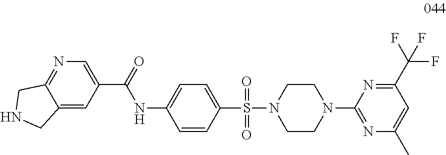 [SMILES: Cc1cc(C(F)(F)F)nc(N2CCN(S(=O)(=O)c3ccc(NC(=O)c4cnc5c(c4)CNC5)cc3)CC2)n1]

Step 1: Tert-butyl 3-[[4-[4-[4-methyl-6-(trifluoromethyl)pyrimidin-2-yl]piperazin-1-yl]sulfonylphenyl]carbamoyl]-5,7-dihydropyrrolo[3,4-b]pyridine-6-carboxylate

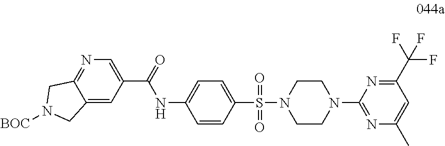 [SMILES: Cc1cc(C(F)(F)F)nc(N2CCN(S(=O)(=O)c3ccc(NC(=O)c4cnc5c(c4)CN(C(=O)OC(C)(C)C)C5)cc3)CC2)n1]

To a solution of 6-tert-butoxycarbonyl-5,7-dihydropyrrolo[3,4-b]pyridine-3-carboxylic acid (80.0 mg, 0.300 mmol) in ACN (1 mL) were added 3-methylpyridine (56.38 mg, 0.610 mmol), methanesulfonyl chloride (0.04 mL, 0.450 mmol, 1.5 eq) in one portion. The reaction was heated at 50 C for 1 hour and then compound Int-13 (133.66 mg, 0.330 mmol) was added to the mixture. The reaction was stirred at 50° C. for another 3 hours. After completion, the mixture was diluted with water (20 mL), extracted with DCM (20 mL×3). The combined organic layer was washed with brine (50 mL), dried over anhydrous Na₂SO₄, concentrated under vacuum to afford the crude product, which was purified by prep-HPLC (TFA as additive) to give Example 044a (85 mg, 43.36% yield) as a yellow solid. MS obsd. (ESI⁺) [(M+H)⁺]: 648.2.

Step 2: N-[4-[4-[4-methyl-6-(trifluoromethyl)pyrimidin-2-yl]piperazin-1-yl]sulfonylphenyl]-6,7-dihydro-5H-pyrrolo[3,4-b]pyridine-3-carboxamide hydrochloride

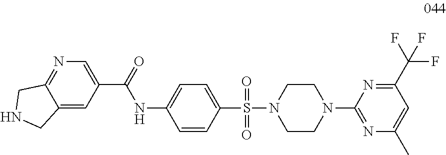 [SMILES: Cc1cc(C(F)(F)F)nc(N2CCN(S(=O)(=O)c3ccc(NC(=O)c4cnc5c(c4)CNC5)cc3)CC2)n1]

A mixture of compound 044a (85.0 mg, 0.130 mmol) in DCM (1.0 mL) and TFA (0.5 mL) was stirred at 25° C. for 1 hour. After completion, the reaction solution was concentrated under vacuum to give a residue, which was purified by prep-HPLC (HCl as additive) to give Example 044 (74 mg, 95.99% yield) as a white solid. MS obsd. (ESI⁺) [(M+H)⁺]: 548.2.

### Example 044: ¹H NMR (400 MHz, DMSO-d6) δ ppm 11.03-10.97 (m, 1H), 10.30-10.09 (m, 2H), 9.11-9.05 (m, 1H), 8.41-8.32 (m, 1H), 8.11-8.01 (m, 2H), 7.79-7.71 (m, 2H), 6.96 (s, 1H), 4.68-4.62 (m, 2H), 4.59-4.53 (m, 2H), 3.90-3.79 (m, 4H), 3.03-2.92 (m, 4H), 2.37 (s, 3H)

### Example 045: N-[4-[4-[6-methyl-4-(trifluoromethyl)-2-pyridyl]piperazin-1-yl]sulfonylphenyl]-6,7-dihydro-5H-pyrrolo[3,4-b]pyridine-3-carboxamide

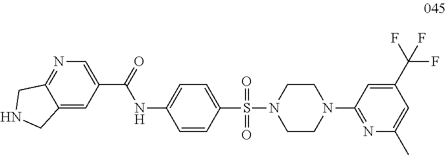 [SMILES: Cc1cc(C(F)(F)F)cc(N2CCN(S(=O)(=O)c3ccc(NC(=O)c4cnc5c(c4)CNC5)cc3)CC2)n1]

Step 1: Tert-butyl 3-[[4-[4-[6-chloro-4-(trifluoromethyl)-2-pyridyl]piperazin-1-yl]sulfonylphenyl]carbamoyl]-5,7-dihydropyrrolo[3,4-b]pyridine-6-carboxylate

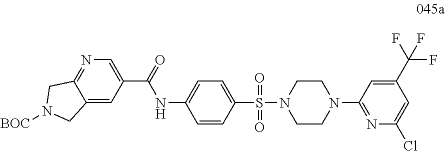 [SMILES: CC(C)(C)OC(=O)N1Cc2cc(C(=O)Nc3ccc(S(=O)(=O)N4CCN(c5cc(C(F)(F)F)cc(Cl)n5)CC4)cc3)cnc2C1]

Compound 045a was prepared in analogy to the step of preparation compound 044a by replacing compound Int-13 with compound Int-1. Compound 045a (34 mg) was obtained as a white solid. MS obsd. (ESI⁺) [(M+H)⁺]: 667.2.

Step 2: N-[4-[4-[6-methyl-4-(trifluoromethyl)-2-pyridyl]piperazin-1-yl]sulfonylphenyl]-6,7-dihydro-5H-pyrrolo[3,4-b]pyridine-3-carboxamide

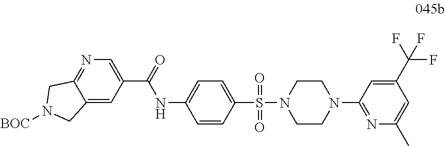 [SMILES: Cc1cc(C(F)(F)F)cc(N2CCN(S(=O)(=O)c3ccc(NC(=O)c4cnc5c(c4)CN(C(=O)OC(C)(C)C)C5)cc3)CC2)n1]

A mixture of trimethylboroxine (33.95 mg, 0.270 mmol), compound 045b (150.0 mg, 0.220 mmol), K₃PO₄ (67.9 mg, 0.320 mmol) and chloro[(di(1-adamantyl)-N-butylphosphine)-2-(2-aminobiphenyl)]palladium(II) (15.03 mg, 0.020 mmol) in THF (8 mL) and water (0.800 mL) was stirred at 70° C. for 3 h under Ar. After completion, the mixture was poured into water (100 mL), extracted with EA (100 mL×2). The combined organic layer was washed with brine (100 mL×2), dried over Na₂SO₄ and concentrated under vacuum to give residue, which was purified by prep-HPLC (FA as additive) to give compound 045b (105 mg, 85.44% yield) as a white solid. MS obsd. (ESI⁺) [(M+H)⁺]: 647.2.

Step 3: N-[4-[4-[6-methyl-4-(trifluoromethyl)-2-pyridyl]piperazin-1-yl]sulfonylphenyl]-6,7-dihydro-5H-pyrrolo[3,4-b]pyridine-3-carboxamide

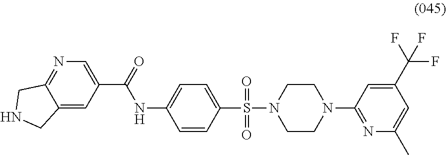 [SMILES: Cc1cc(C(F)(F)F)cc(N2CCN(S(=O)(=O)c3ccc(NC(=O)c4cnc5c(c4)CNC5)cc3)CC2)n1]

A solution of compound 045b (100.0 mg, 0.150 mmol) in DCM (2 mL) and TFA (1.0 mL) was stirred at 25° C. for 1 hour. After completion, the mixture was concentrated under vacuum to give a residue, which was purified by prep-HPLC (HCl as additive) to give Example 045 (48.3 mg, 53.55% yield) as a white solid. MS obsd. (ESI⁺) [(M+H)⁺]: 547.3.

### Example 045: ¹H NMR (400 MHz, DMSO-d6) δ ppm 11.09-11.24 (m, 1H), 10.38-10.62 (m, 2H), 9.06-9.14 (m, 1H), 8.36-8.42 (m, 1H), 8.04-8.16 (m, 2H), 7.67-7.86 (m, 2H), 6.90-6.98 (m, 1H), 6.76-6.85 (m, 1H), 4.57 (br t, J=5.50 Hz, 4H), 3.68-3.79 (m, 4H), 2.90-3.04 (m, 4H), 2.35-2.43 (m, 3H)

### Example 046: N-[4-[4-[6-chloro-4-(trifluoromethyl)-2-pyridyl]piperazin-1-yl]sulfonylphenyl]isoindoline-5-carboxamide

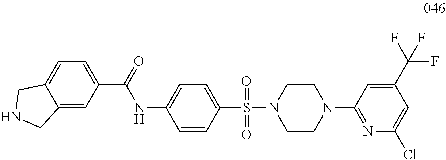 [SMILES: O=C(Nc1ccc(S(=O)(=O)N2CCN(c3cc(C(F)(F)F)cc(Cl)n3)CC2)cc1)c1ccc2c(c1)CNC2]

The title compound was prepared in analogy to the preparation of Example 044 by replacing 6-tert-butoxycarbonyl-5,7-dihydropyrrolo[3,4-b]pyridine-3-carboxylic acid with compound Int-14 and compound Int-13 with compound Int-1 in step1. Example 045 (22.85 mg) was obtained. MS obsd. (ESI⁺) [(M+H)⁺]: 566.1.

### Example 046: ¹H NMR (400 MHz, DMSO-d₆) δ ppm 10.87-10.64 (m, 1H), 10.11-9.70 (m, 2H), 8.12-8.03 (m, 2H), 8.01-7.93 (m, 2H), 7.82-7.68 (m, 2H), 7.61-7.52 (m, 1H), 7.13-7.02 (m, 1H), 6.95 (s, 1H), 4.75-4.42 (m, 4H), 3.78-3.64 (m, 4H), 2.98 (br t, J=4.6 Hz, 4H)

### Example 047: N-[4-[4-[6-chloro-4-(trifluoromethyl)-2-pyridyl]piperazin-1-yl]sulfonylphenyl]-3-(2,5-diazabicyclo[2.2.1]heptan-2-yl)benzamide

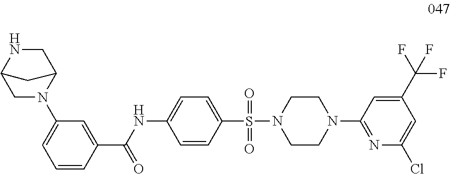 [SMILES: O=C(Nc1ccc(S(=O)(=O)N2CCN(c3cc(C(F)(F)F)cc(Cl)n3)CC2)cc1)c1cccc(N2CC3CC2CN3)c1]

Step 1: 3-Bromo-N-[4-[4-[6-chloro-4-(trifluoromethyl)-2-pyridyl]piperazin-1-yl]sulfonylphenyl]benzamide

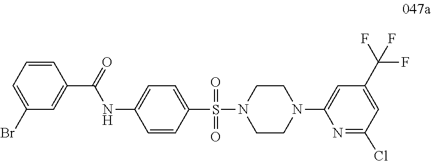 [SMILES: O=C(Nc1ccc(S(=O)(=O)N2CCN(c3cc(C(F)(F)F)cc(Cl)n3)CC2)cc1)c1cccc(Br)c1]

A mixture of compound Int-1 (0.5 g, 1.19 mmol), 3-bromobenzoyl chloride (0.3 g, 1.36 mmol) and TEA (331 μL, 2.38 mmol) in DCM (12 mL) was stirred at rt overnight. After completion, the mixture was quenched with water (20 mL), diluted with DCM (30 mL). The organic layer was washed with brine (25 mL), dried over anhydrous Na₂SO₄ and concentrated under vacuum to give crude compound 047a (700 mg, 95.7% yield) as a light yellow solid. MS obsd. (ESI⁺) [(M+H)⁺]: 604.8.

Step 2: N-[4-[4-[6-chloro-4-(trifluoromethyl)-2-pyridyl]piperazin-1-yl]sulfonylphenyl]-3-(4,4,5,5-tetramethyl-1,3,2-dioxaborolan-2-yl)benzamide

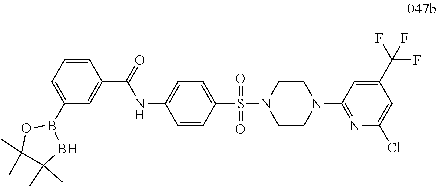

A mixture of compound 047a (1.0 g, 1.66 mmol), bis(pinacolato)diboron (463 mg, 1.82 mmol) and potassium acetate (406 mg, 4.14 mmol) in 1,4-dioxane (5 mL) was degassed for 5 minutes, then 1,1′-bis(diphenylphosphino)ferrocene-palladium(II)dichloride dichloromethane complex (67.6 mg, 82.8 μmol) was added. The reaction mixture was stirred in a sealed tube at 90° C. for 2 hours. After completion, the reaction was cooled to rt and quenched with water (100 mL) and extracted with EtOAc (50 mL×3). The combined organic layer was washed with brine (50 mL), dried over anhydrous Na₂SO₄ and concentrated under vacuum to afford crude product, which was purified by silica gel chromatography (elute with EtOAc:PE=30%) to afford compound 047b (0.6 g, 55.4% yield) as orange oil. MS obsd. (ESI⁺) [(M+H)⁺]: 651.3.

Step 3: [3-[[4-[4-[6-Chloro-4-(trifluoromethyl)-2-pyridyl]piperazin-1-yl]sulfonylphenyl]carbamoyl]phenyl]boronic acid

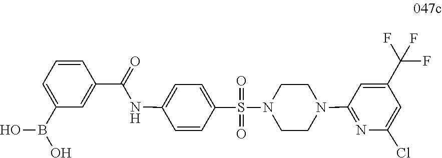 [SMILES: O=C(Nc1ccc(S(=O)(=O)N2CCN(c3cc(C(F)(F)F)cc(Cl)n3)CC2)cc1)c1cccc(B(O)O)c1]

Compound 047b (0.5 g, 768 μmol) was dissolved in the 1,4-dioxane (10 mL), followed by adding 6 N HCl (2 mL, 12 mmol). The reaction was heated at 90° C. for 4 hours. After completion, the reaction was concentrated under vacuum to give crude 047c (0.4 g, 91.6%) as a brown oil, which was used directly in the next step.

Step 4: Tert-butyl 5-[3-[[4-[4-[6-chloro-4-(trifluoromethyl)-2-pyridyl]piperazin-1-yl]sulfonylphenyl]carbamoyl]phenyl]-2,5-diazabicyclo[2.2.1]heptane-2-carboxylate

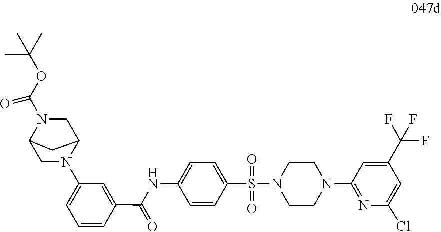 [SMILES: CC(C)(C)OC(=O)N1CC2CC1CN2c1cccc(C(=O)Nc2ccc(S(=O)(=O)N3CCN(c4cc(C(F)(F)F)cc(Cl)n4)CC3)cc2)c1]

A mixture of compound 047c (130 mg, 229 μmol), tert-butyl 2,5-diazabicyclo[2.2.1]heptane-2-carboxylate (45.3 mg, 229 μmol), copper (I) chloride (22.6 mg, 229 μmol), TEA (100 μL, 686 μmol) and molecular sieve 4A (0.2 g, 229 μmol) in MeCN (5 mL) was stirred for 12 h in air. Then the mixture was filtered and the filtrate was concentrated under vacuum to give a crude green residue, which was purified by silica gel chromatography (eluent with EtOAc:PE=40%) to afford compound 047d (90 mg, 54.6% yield) as yellow oil. MS obsd. (ESI⁺) [(M+H)⁺]: 721.2.

Step 5: N-[4-[4-[6-chloro-4-(trifluoromethyl)-2-pyridyl]piperazin-1-yl]sulfonylphenyl]-3-[2,5-diazabicyclo[2.2.11heptan-2-yl]benzamide

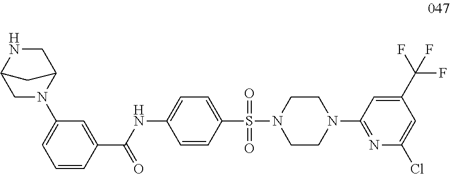 [SMILES: O=C(Nc1ccc(S(=O)(=O)N2CCN(c3cc(C(F)(F)F)cc(Cl)n3)CC2)cc1)c1cccc(N2CC3CC2CN3)c1]

Compound 047d (100 mg, 139 μmol) was dissolved in the DCM (5 mL), followed by adding TFA (2 mL, 139 μmol). The reaction was stirred at rt for 1 hour. After completion, the reaction was concentrated under vacuum and purified by prep-HPLC (TFA as additive) to give Example 047 (1.5 mg, 1.4% yield) as a white solid. MS obsd. (ESI⁺) [(M+H)⁺]: 621.2.

### Example 047: ¹H NMR (400 MHz, CD₃OD) δ ppm 8.00 (d, 2H, J=8.8 Hz), 7.82 (d, 2H, J=8.8 Hz), 7.3-7.5 (m, 2H), 7.23 (s, 1H), 6.95 (s, 2H), 6.85 (s, 1H), 4.78 (s, 1H), 4.53 (s, 1H), 3.82 (dd, 1H, J=2.4, 10.6 Hz), 3.7-3.8 (m, 4H), 3.41 (br s, 3H), 3.1-3.2 (m, 5H), 2.35 (br d, 1H, J=11.4 Hz), 2.0-2.2 (m, 1H). 1⁹F NMR (376 MHz, CD₃OD) δ ppm −66.49 (m, 3F)

### Example 048: 3-(2-Aminoethoxy)-N-[4-[4-[6-chloro-4-(trifluoromethyl)-2-pyridyl]piperazin-1-yl]sulfonylphenyl]benzamide

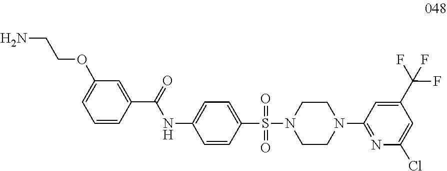 [SMILES: NCCOc1cccc(C(=O)Nc2ccc(S(=O)(=O)N3CCN(c4cc(C(F)(F)F)cc(Cl)n4)CC3)cc2)c1]

Step 1: N-[4-[4-[6-chloro-4-(trifluoromethyl)-2-pyridyl]piperazin-1-yl]sulfonylphenyl]-3-methoxy-benzamide

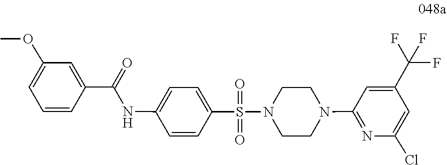 [SMILES: COc1cccc(C(=O)Nc2ccc(S(=O)(=O)N3CCN(c4cc(C(F)(F)F)cc(Cl)n4)CC3)cc2)c1]

To a solution of compound Int-1 (800.0 mg, 1.9 mmol, 1 eq) in DCM (8 mL) was added TEA (0.79 mL, 5.7 mmol, 3 eq), 3-methoxybenzoyl chloride (0.53 mL, 3.8 mmol, 2 eq) in sequence. The reaction was stirred at 20° C. for 1 hour. After completion, the mixture was diluted with DCM(100 mL), washed with water (50 mL) and brine (50 mL). The organic layer was dried over anhydrous Na₂SO₄ and concentrated under vacuum to give a residue, which was purified by prep-HPLC (TFA as additive) to give compound 048a (1000 mg, 94.79% yield) as a red solid. MS obsd. (ESI⁺) [(M+H)⁺]: 555.2.

Step 2: N-[4-[4-[6-chloro-4-(trifluoromethyl)-2-pyridyl]piperazin-1-yl]sulfonylphenyl]-3-hydroxy-benzamide

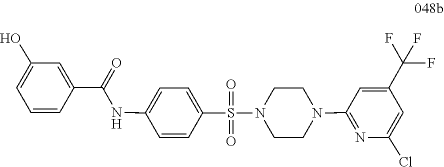 [SMILES: O=C(Nc1ccc(S(=O)(=O)N2CCN(c3cc(C(F)(F)F)cc(Cl)n3)CC2)cc1)c1cccc(O)c1]

A solution of compound 048a (980.0 mg, 1.77 mmol, 1 eq), BBr₃ (0.01 mL, 2.65 mmol) in in DCM (20 mL) was stirred at 20° C. for 2 hours. After completion, the mixture was quenched with MeOH (10 mL) and water (20 mL). The mixture was extracted with EtOAc (20 mL×3). The combined organic layer was dried over anhydrous Na₂SO₄ and concentrated under vacuum to give crude product, which was purified by silica-gel chromatography (elute with EtOAc:PE=20%) to give compound 048b (810 mg, 84.8% yield) as a red solid. MS obsd. (ESI⁺) [(M+H)⁺]: 563.2.

Step 3: Tert-butyl N-[2-[3-[[4-[4-[6-chloro-4-(trifluoromethyl)-2-pyridyl]piperazin-1-yl]sulfonylphenyl]carbamoyl]phenoxy]ethyl]carbamate

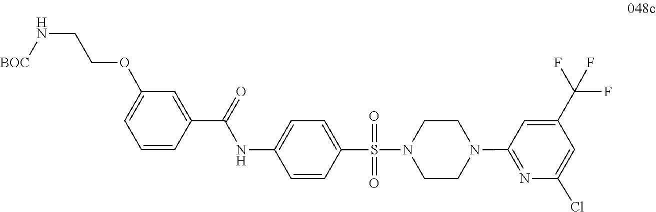 [SMILES: CC(C)(C)OC(=O)NCCOc1cccc(C(=O)Nc2ccc(S(=O)(=O)N3CCN(c4cc(C(F)(F)F)cc(Cl)n4)CC3)cc2)c1]

A mixture of compound 048b (150.0 mg, 0.280 mmol), tert-butyl N-(2-bromoethyl)carbamate (124.28 mg, 0.550 mmol) and K₂CO₃ (114.97 mg, 0.830 mmol) in DMF (1.25 mL) was stirred at 90° C. for 2 hours. After completion, the mixture was diluted with water (20 mL) and then extracted with EtOAc (20 mL×3). The combined organic layer was washed with water (50 mL), dried over anhydrous Na₂SO₄ and concentrated in vacuo to give a residue, which was purified by prep-HPLC (TFA as additive) to give compound 048c (110 mg, 57.98% yield) as a white solid. MS obsd. (ESI⁺) [(M+H)⁺]: 684.2.

Step 4: 3-(2-Aminoethoxy)-N-[4-[4-[6-chloro-4-(trifluoromethyl)-2-pyridyl]piperazin-1-yl]sulfonylphenyl]benzamide

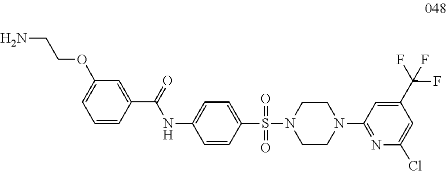 [SMILES: NCCOc1cccc(C(=O)Nc2ccc(S(=O)(=O)N3CCN(c4cc(C(F)(F)F)cc(Cl)n4)CC3)cc2)c1]

A mixture of compound 048c (110.0 mg, 0.160 mmol) in DCM (1 mL) and TFA (0.5 mL) was stirred at 20° C. for 1 hour. After completion, the mixture was concentrated in vacuo to give a residue, which was purified by prep-HPLC (HCl as additive) to afford Example 048 (45 mg, 45.1% yield) as a white solid. MS obsd. (ESI⁺) [(M+H)⁺]: 584.1.

### Example 048: ¹H NMR (400 MHz, DMSO-d6) δ ppm: 10.73 (s, 1H), 8.21-8.13 (m, 3H), 8.11-8.05 (m, 2H), 7.78-7.73 (m, 2H), 7.64-7.60 (m, 1H), 7.57-7.54 (m, 1H), 7.52-7.46 (m, 1H), 7.26-7.21 (m, 1H), 7.09 (s, 1H), 6.99-6.94 (m, 1H), 4.31-4.24 (m, 2H), 3.75-3.66 (m, 4H), 3.27-3.21 (m, 2H), 3.02-2.94 (m, 4H)

### Example 049: 3-(Azetidin-3-ylmethylamino)-N-[4-[4-[6-chloro-4-(trifluoromethyl)-2-pyridyl]piperazin-1-yl]sulfonylphenyl]benzamide

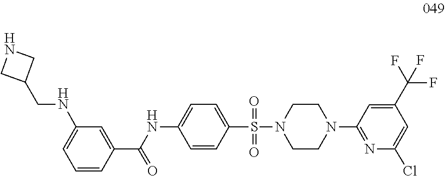 [SMILES: O=C(Nc1ccc(S(=O)(=O)N2CCN(c3cc(C(F)(F)F)cc(Cl)n3)CC2)cc1)c1cccc(NCC2CNC2)c1]

The title compound was prepared in analogy to the preparation of Example 047 by replacing tert-butyl 2,5-diazabicyclo[2.2.1]heptane-2-carboxylate with tert-butyl 3-(aminomethyl)azetidine-1-carboxylate in step 4. Example 049 (1 mg) was obtained as a white solid. MS obsd. (ESI⁺) [(M+H)⁺]: 609.4.

### Example 049: ¹H NMR (400 MHz, CD₃OD) δ ppm 7.8-7.9 (m, 2H), 7.69 (d, 2H, J=8.8 Hz), 7.3-7.5 (m, 1H), 7.0-7.2 (m, 3H), 6.7-6.9 (m, 3H), 5.25 (br, 2H, J=4.6 Hz), 4.06 (dd, 1H, J=9.4, 10.7 Hz), 3.82 (dd, 1H, J=7.2, 10.9 Hz), 3.6-3.7 (m, 4H), 3.34 (d, 2H, J=6.8 Hz), 2.9-3.1 (m, 4H), 2.10 (t, 2H, J=7.5 Hz). ¹⁹F NMR (376 MHz, CD₃OD) δ ppm −66.48 (m, 3F)

### Example 050: 3-(Azetidin-3-ylmethoxy)-N-[4-[4-[6-chloro-4-(trifluoromethyl)-2-pyridyl]piperazin-1-yl]sulfonylphenyl]benzamide

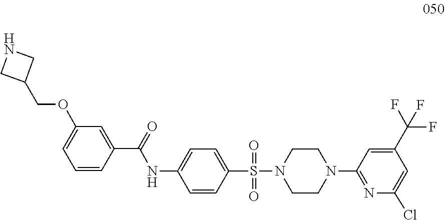 [SMILES: O=C(Nc1ccc(S(=O)(=O)N2CCN(c3cc(C(F)(F)F)cc(Cl)n3)CC2)cc1)c1cccc(OCC2CNC2)c1]

The title compound was prepared in analogy to the preparation of Example 048 by replacing tert-butyl N-(2-bromoethyl)carbamate with tert-butyl 3-(bromomethyl)azetidine-1-carboxylate in step 3. Example 050 (56 mg) was obtained as a white solid. MS obsd. (ESI⁺) [(M+H)⁺]: 610.4.

### Example 050: ¹H NMR (400 MHz, DMSO-d6) δ ppm 10.74-10.69 (m, 1H), 9.12-8.98 (m, 2H), 8.08 (d, J=8.9 Hz, 2H), 7.78-7.73 (m, 2H), 7.62-7.55 (m, 2H), 7.52-7.46 (m, 1H), 7.26-7.22 (m, 1H), 7.10-7.07 (m, 1H), 6.98-6.95 (m, 1H), 4.27-4.22 (m, 2H), 4.13-4.04 (m, 2H), 3.90-3.81 (m, 2H), 3.75-3.66 (m, 4H), 3.26-3.15 (m, 1H), 2.98 (br t, J=4.6 Hz, 4H)

### Example 051: N-[4-[4-[6-chloro-4-(trifluoromethyl)-2-pyridyl]piperazin-1-yl]sulfonylphenyl]-3-[(3R)-3-aminopyrrolidin-1-yl]benzamide

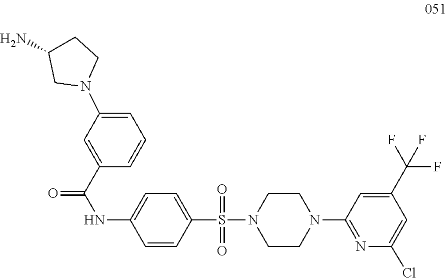 [SMILES: N[C@@H]1CCN(c2cccc(C(=O)Nc3ccc(S(=O)(=O)N4CCN(c5cc(C(F)(F)F)cc(Cl)n5)CC4)cc3)c2)C1]

The title compound was prepared in analogy to the preparation of Example 052 by replacing tert-butyl-3,6-diazabicyclo[3.2.0]heptane-6-carboxylate with tert-butyl (R)-pyrrolidin-3-ylcarbamate hydrochloride in step 2. Example 051 (18 mg) was obtained as a white solid. MS obsd. (ESI⁺) [(M+H)⁺]: 609.9.

### Example 051: ¹H NMR (400 MHz, DMSO-d6) δ ppm 10.56 (s, 1H), 8.0-8.2 (m, 5H), 7.75 (d, 2H, J=8.8 Hz), 7.3-7.4 (m, 1H), 7.28 (s, 1H), 7.0-7.2 (m, 2H), 6.97 (s, 1H), 6.82 (dd, 1H, J=1.9, 8.1 Hz), 3.99 (br s, 1H), 3.7-3.8 (m, 4H), 3.5-3.6 (m, 3H), 3.0-3.1 (m, 4H), 2.3-2.4 (m, 1H), 2.0-2.2 (m, 1H). 1⁹F NMR (376 MHz, DMSO-d6) δ ppm −63.46 (m, 3F)

### Example 052: N-[4-[4-[6-Chloro-4-(trifluoromethyl)-2-pyridyl]piperazin-1-yl]sulfonylphenyl]-3-[3,6-diazabicyclo[3.2.0]heptan-3-yl]benzamide

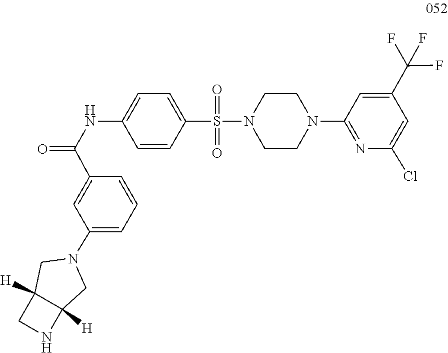 [SMILES: O=C(Nc1ccc(S(=O)(=O)N2CCN(c3cc(C(F)(F)F)cc(Cl)n3)CC2)cc1)c1cccc(N2C[C@H]3CN[C@H]3C2)c1]

Step 1: N-[4-[4-[6-Chloro-4-(trifluoromethyl)-2-pyridyl]piperazin-1-yl]sulfonylphenyl]-3-iodo-benzamide

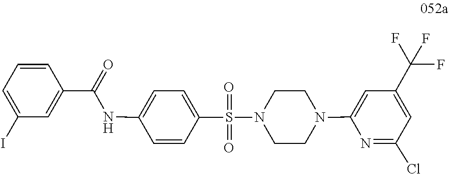 [SMILES: O=C(Nc1ccc(S(=O)(=O)N2CCN(c3cc(C(F)(F)F)cc(Cl)n3)CC2)cc1)c1cccc(I)c1]

Compound Int-1 (0.15 g, 356 μmol), 3-iodobenzoyl chloride (190 mg, 713 μmol) and TEA (100 μL, 713 μmol) was stirred at rt overnight. After completion, the mixture was quenched with water (10 mL), diluted with DCM (30 mL). The organic layer was washed with brine (25 mL), dried over anhydrous Na₂SO₄ and concentrated under vacuum to give the crude product, which was purified by silica-gel chromatography (eluent with EtOAc:PE=20%) to afford compound 052a (180 mg, 77.6% yield) as yellow oil. MS obsd. (ESI⁺) [(M+H)⁺]: 651.2.

Step 2: Tert-butyl-3-[3-[[4-[4-[6-chloro-4-(trifluoromethyl)-2-pyridyl]piperazin-1-yl]sulfonylphenyl]carbamoyl]phenyl]-3,6-diazabicyclo[3.2.0]heptane-6-carboxylate

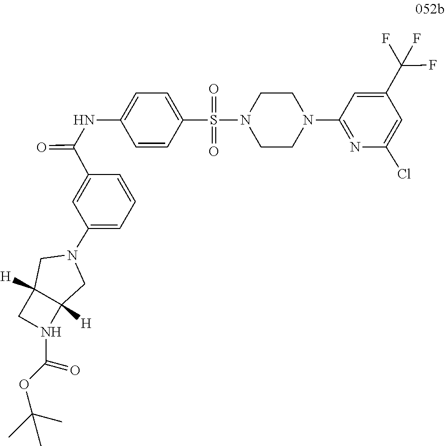

A degassed mixture of Cs₂CO₃ (150 mg, 461 μmol), chloro[(di(1-adamantyl)-N-butylphosphine)-2-(2-aminobiphenyl)]palladium(II) (15.4 mg, 23 μmol), tert-butyl-3,6-diazabicyclo[3.2.0]heptane-6-carboxylate (45.7 mg, 230 μmol), N-(4-((4-(6-chloro-4-(trifluoromethyl)pyridin-2-yl)piperazin-1-yl)sulfonyl)phenyl)-3-iodobenzamide (75 mg, 115 μmol) in 1,4-dioxane (2.5 mL) was heated at 75° C. in a microwave reactor for 1 hour. After completion, the mixture was poured into ice water (20 mL), extracted with DCM (10 mL×3), dried over anhydrous Na₂SO₄ and concentrated under vacuum to give the crude product, which was purified by silica-gel chromatography (eluent with EtOAc:PE=40%) to afford compound 052b (50 mg, 60.2% yield) as yellow oil. MS obsd. (ESI⁺) [(M+H)⁺]: 721.2.

Step 3: N-[4-[4-[6-Chloro-4-(trifluoromethyl)-2-pyridyl]piperazin-1-yl]sulfonylphenyl]-3-[3,6-diazabicyclo[3.2.0]heptan-3-yl]benzamide

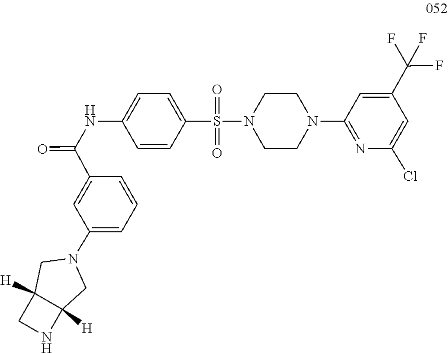 [SMILES: O=C(Nc1ccc(S(=O)(=O)N2CCN(c3cc(C(F)(F)F)cc(Cl)n3)CC2)cc1)c1cccc(N2C[C@H]3CN[C@H]3C2)c1]

Compound 052b (50 mg, 69.3 μmol) was dissolved in the DCM (5 mL), followed by adding TFA (2 mL, 69.3 μmol). The reaction was stirred at rt and went complete after 1 hour. After completion, the reaction was concentrated under vacuum and purified by prep-HPLC (TFA as additive) to afford Example 052 (2.7 mg, 5.2% yield) as a white solid. MS obsd. (ESI⁺) [(M+H)⁺]: 621.2.

### Example 052: ¹H NMR (400 MHz, DMSO-d6) δ ppm 8.01 (d, 2H, J=8.9 Hz), 7.82 (d, 2H, J=8.9 Hz), 7.46 (d, 3H, J=5.0 Hz), 7.1-7.3 (m, 1H), 6.95 (s, 1H), 6.85 (s, 1H), 4.60 (s, 3H), 4.19 (d, 1H, J=12.6 Hz), 3.98 (d, 1H, J=10.6 Hz), 3.7-3.8 (m, 4H), 3.5-3.6 (m, 2H), 3.1-3.2 (m, 4H), 3.0-3.1 (m, 2H). ¹⁹F NMR (376 MHz, DMSO-d6) δ ppm −66.49 (m, 3F)

### Example 053: 3-(2-Aminoethyl)-N-[4-[4-[6-chloro-4-(trifluoromethyl)-2-pyridyl]piperazin-1-yl]sulfonylphenyl]benzamide

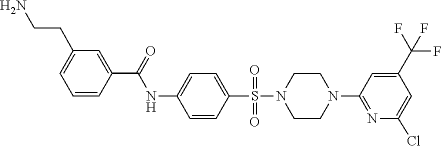 [SMILES: NCCc1cccc(C(=O)Nc2ccc(S(=O)(=O)N3CCN(c4cc(C(F)(F)F)cc(Cl)n4)CC3)cc2)c1]

The title compound was prepared in analogy to the preparation of Example 044 by replacing 6-tert-butoxycarbonyl-5,7-dihydropyrrolo[3,4-b]pyridine-3-carboxylic acid with compound Int-15 and compound Int-13 with compound Int-1 in step1. Example 053 (93.9 mg) was obtained as a white solid. MS obsd. (ESI⁺) [(M+H)⁺]: 568.3.

### Example 053: ¹H NMR (400 MHz, DMSO-d6) δ ppm 10.51-10.84 (m, 1H), 8.03-8.11 (m, 2H), 7.72-7.83 (m, 4H), 7.41-7.47 (m, 2H), 7.07-7.12 (m, 1H), 6.94-6.99 (m, 1H), 3.65-3.80 (m, 4H), 2.99 (br t, J=4.77 Hz, 4H), 2.70-2.87 (m, 4H)

### Example 054: N-[4-[4-[6-chloro-4-(trifluoromethyl)-2-pyridyl]piperazin-1-yl]sulfonylphenyl]-3-piperazin-1-yl-benzamide

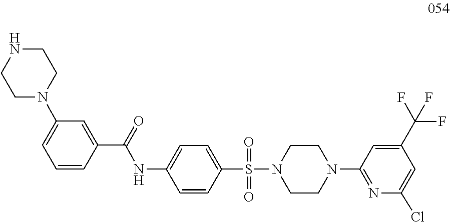 [SMILES: O=C(Nc1ccc(S(=O)(=O)N2CCN(c3cc(C(F)(F)F)cc(Cl)n3)CC2)cc1)c1cccc(N2CCNCC2)c1]

The title compound was prepared in analogy to the preparation of Example 044 by replacing 6-tert-butoxycarbonyl-5,7-dihydropyrrolo[3,4-b]pyridine-3-carboxylic acid with 3-(4-tert-butoxycarbonylpiperazin-1-yl)benzoic acid and compound Int-13 with compound Int-1 in step1. Example 054 (100.2 mg) was obtained as a white solid. MS obsd. (ESI⁺) [(M+H)⁺]: 609.3.

### Example 054: ¹H NMR (400 MHz, DMSO-d6) δ ppm 10.46-10.70 (m, 1H), 7.98-8.14 (m, 2H), 7.69-7.84 (m, 2H), 7.29-7.51 (m, 3H), 7.08-7.20 (m, 2H), 6.94-7.01 (m, 1H), 3.64-3.83 (m, 4H), 3.08-3.16 (m, 4H), 2.98 (br t, J=4.63 Hz, 4H), 2.86 (br d, J=4.50 Hz, 4H)

### Example 055: 2-Amino-N-[4-[4-[6-chloro-4-(trifluoromethyl)-2-pyridyl]piperazin-1-yl]sulfonylphenyl]indane-5-carboxamide

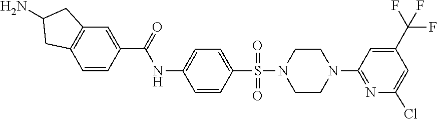 [SMILES: NC1Cc2ccc(C(=O)Nc3ccc(S(=O)(=O)N4CCN(c5cc(C(F)(F)F)cc(Cl)n5)CC4)cc3)cc2C1]

The title compound was prepared in analogy to the preparation of Example 044 by replacing 6-tert-butoxycarbonyl-5,7-dihydropyrrolo[3,4-b]pyridine-3-carboxylic acid with compound Int-16 and compound Int-13 with compound Int-1 in step1. Example 055 (49.6 mg) was obtained as a white solid. MS obsd. (ESI⁺) [(M+H)⁺]: 580.3.

### Example 055: ¹H NMR (400 MHz, DMSO-d6) δ ppm 10.47-10.64 (m, 1H), 8.04-8.14 (m, 2H), 7.70-7.87 (m, 4H), 7.30-7.40 (m, 1H), 7.08-7.15 (m, 1H), 6.93-6.99 (m, 1H), 3.65-3.83 (m, 5H), 3.09-3.15 (m, 2H), 2.99 (br d, J=4.16 Hz, 4H), 2.60-2.70 (m, 2H)

### Example 056: 5-(Aminomethyl)-N-[4-[4-[6-chloro-4-(trifluoromethyl)-2-pyridyl]piperazin-1-yl]sulfonylphenyl]-2-hydroxy-benzamide

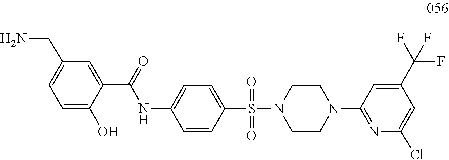 [SMILES: NCc1ccc(O)c(C(=O)Nc2ccc(S(=O)(=O)N3CCN(c4cc(C(F)(F)F)cc(Cl)n4)CC3)cc2)c1]

Step 1: N-(4-((4-(6-chloro-4-(trifluoromethyl)pyridin-2-yl)piperazin-1-yl)sulfonyl)phenyl)-5-(chloromethyl)-2-methoxybenzamide

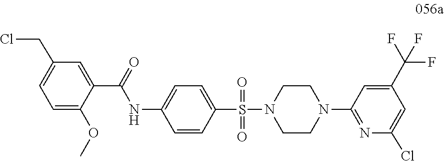 [SMILES: COc1ccc(CCl)cc1C(=O)Nc1ccc(S(=O)(=O)N2CCN(c3cc(C(F)(F)F)cc(Cl)n3)CC2)cc1]

To a solution of 5-(chloromethyl)-2-methoxy-benzoic acid (100.0 mg, 0.500 mmol) in DCM (1 mL) was added SOCl₂ (92.84 mg, 1 mmol) dropwise. The mixture was stirred at 20° C. for 30 minutes. And then, the reaction mixture was concentrated and the residue was re-dissolved in dry DCM (5 mL). Then compound Int-1 (0.21 g, 0.500 mmol) and TEA (0.10 g, 1.0 mmol) were added to the above solution. The mixture was stirred at 20° C. for 3 hours. After completion, the reaction mixture was concentrated under vacuum to give compound 056a (280 mg, 93.09% yield) as a yellow solid. MS obsd. (ESI⁺) [(M+H)⁺]: 603.2.

Step 2: 5-(Aminomethyl)-N-(4-((4-(6-chloro-4-(trifluoromethyl)pyridin-2-yl)piperazin-1-yl)sulfonyl)phenyl)-2-methoxybenzamide

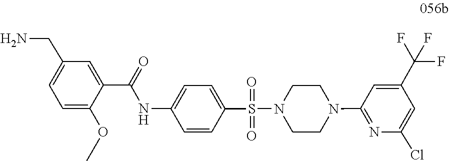 [SMILES: COc1ccc(CN)cc1C(=O)Nc1ccc(S(=O)(=O)N2CCN(c3cc(C(F)(F)F)cc(Cl)n3)CC2)cc1]

To a solution of compound 056a (200.0 mg, 0.330 mmol) in MeCN (5 mL) were added NH₃·H₂O (1.0 mL, 0.430 mmol) in one portion. The mixture was stirred in a sealed tube at 50° C. for 4 hours. After completion, the solution was poured into water (15 mL), extracted with DCM (20 mL×3). The combined organic layer was washed with brine, dried over Na₂SO₄ and concentrated under reduced pressure to give crude compound 056b (185 mg, 95.58% yield) as a yellow solid. MS obsd. (ESI⁺) [(M+H)⁺]: 584.1.

Step 3: 5-(Aminomethyl)-N-(4-((4-(6-chloro-4-(trifluoromethyl)pyridin-2-yl)piperazin-1-yl)sulfonyl)phenyl)-2-hydroxybenzamide

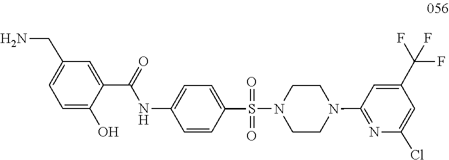 [SMILES: NCc1ccc(O)c(C(=O)Nc2ccc(S(=O)(=O)N3CCN(c4cc(C(F)(F)F)cc(Cl)n4)CC3)cc2)c1]

To a mixture of compound 056b (100.0 mg, 0.170 mmol, 1 eq) in DCM (5 mL) was added AlCl₃ (112.92 mg, 0.860 mmol), the mixture was stirred at 20° C. for 15 hours. After completion, solvent was concentrated under vacuum to give a residue which was purified by prep-HPLC (HCl as additive) to afford Example 056 (6.1 mg, 6.13% yield) as a blue solid. MS obsd. (ESI⁺) [(M+H)⁺]: 570.0.

### Example 056: ¹H NMR (400 MHz, DMSO-d₆) δ ppm 11.65-11.45 (m, 1H), 10.77 (s, 1H), 8.34-8.17 (m, 3H), 8.07-7.98 (m, 3H), 7.80-7.70 (m, 2H), 7.56-7.48 (m, 1H), 7.11-7.04 (m, 2H), 6.96 (s, 1H), 4.07-3.94 (m, 2H), 3.76-3.66 (m, 4H), 3.06-2.94 (m, 4H)

### Example 057: N-[4-[4-[6-chloro-4-(trifluoromethyl)-2-pyridyl]piperazin-1-yl]sulfonylphenyl]-3-(2,4-diaminobutanoylamino)benzamide

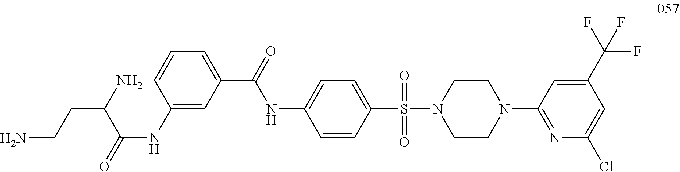 [SMILES: NCCC(N)C(=O)Nc1cccc(C(=O)Nc2ccc(S(=O)(=O)N3CCN(c4cc(C(F)(F)F)cc(Cl)n4)CC3)cc2)c1]

Step 1: Tert-butyl N-[3-[[4-[4-[6-chloro-4-(trifluoromethyl)-2-pyridyl]piperazin-1-yl]sulfonylphenyl]carbamoyl]phenyl]carbamate

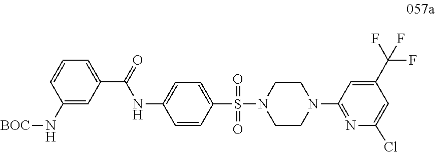 [SMILES: CC(C)(C)OC(=O)Nc1cccc(C(=O)Nc2ccc(S(=O)(=O)N3CCN(c4cc(C(F)(F)F)cc(Cl)n4)CC3)cc2)c1]

A mixture of 3-(tert-butoxycarbonylamino)benzoic acid (563.75 mg, 2.38 mmol) and SOCl₂ (2.83 g, 23.76 mmol) in dry DCM (10.0 mL) and THF (10.0 mL) was stirred at 25° C. for 6 hours. And then, the mixture was concentrated under vacuum to give residue, which was re-dissolved in dry DCM (10.0 mL) and added to the mixture of compound Int-1 (500.0 mg, 1.19 mmol) and TEA (0.5 mL, 3.56 mmol) in DCM (10 mL). The mixture was stirred at 15° C. for 2 hours. After completion, the mixture was diluted with EtOAc (200 mL), washed with brine (50 mL×3). The organic layer was dried over anhydrous Na₂SO₄ and concentrated under vacuum to give crude product, which was purified by prep-HPLC (FA as additive) to give compound 057a (620 mg, 81.53% yield) as light yellow solid. MS obsd. (ESI⁺) [(M+H)⁺]: 640.1.

Step 2: 3-Amino-N-[4-[4-[6-chloro-4-(trifluoromethyl)-2-pyridyl]piperazin-1-yl]sulfonylphenyl]benzamide

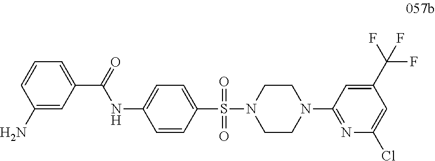 [SMILES: Nc1cccc(C(=O)Nc2ccc(S(=O)(=O)N3CCN(c4cc(C(F)(F)F)cc(Cl)n4)CC3)cc2)c1]

A mixture of compound 057a (620.0 mg, 0.970 mmol), hydrochloric acid in dioxane (20.0 mL, 80 mmol) was stirred at 25° C. for 2 hours. After completion, the mixture concentrated under vacuum to give residue, which was re-dissolved in EtOAc (300 mL) and basified with aq.K₂CO₃ to pH 8.0. The organic layer was washed with brine (50 mL×2) and dried over anhydrous Na₂SO₄ and concentrated in vacuo to give compound 057b (500 mg, 95.6% yield) as a light yellow solid. MS obsd. (ESI⁺) [(M+H)⁺]: 540.1.

Step 3: Tert-butyl N-[3-(tert-butoxycarbonylamino)-4-[3-[[4-[4-[6-chloro-4-(trifluoromethyl)-2-pyridyl]piperazin-1-yl]sulfonylphenyl]carbamoyl]anilino]-4-oxo-butyl]carbamate

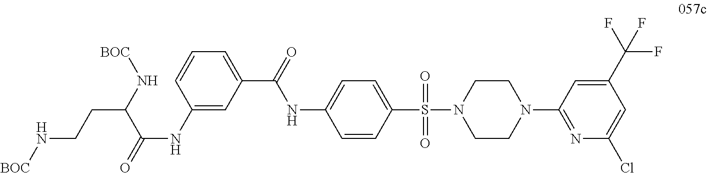 [SMILES: CC(C)(C)OC(=O)NCCC(NC(=O)OC(C)(C)C)C(=O)Nc1cccc(C(=O)Nc2ccc(S(=O)(=O)N3CCN(c4cc(C(F)(F)F)cc(Cl)n4)CC3)cc2)c1]

To a solution of 2,4-bis(tert-butoxycarbonylamino)butanoic acid (176.89 mg, 0.560 mmol) and 3-methylpyridine (137.98 mg, 1.48 mmol) in MeCN (3.03 mL) was added MsCl (0.05 mL, 0.590 mmol) and stirred at 15° C. for 4 hours. And then, compound 057b (200.0 mg, 0.370 mmol) was added to the above mixture which was stirred at 50° C. for 2 hours. After completion, the mixture was concentrated in vacuo to give crude compound 057c (200 mg, 64.26% yield) as a yellow oil, which was used in the next step directly. MS obsd. (ESI⁺) [(M+H)⁺]: 840.3.

Step 4: N-[4-[4-[6-chloro-4-(trifluoromethyl)-2-pyridyl]piperazin-1-yl]sulfonylphenyl]-3-(2,4-diaminobutanoylamino)benzamide

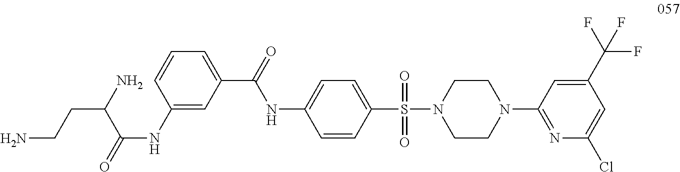 [SMILES: NCCC(N)C(=O)Nc1cccc(C(=O)Nc2ccc(S(=O)(=O)N3CCN(c4cc(C(F)(F)F)cc(Cl)n4)CC3)cc2)c1]

A mixture of compound 057c (200.0 mg, 0.240 mmol) in HCl in dioxane (5.0 mL, 20 mmol) was stirred at 25° C. for 2 hours. After completion, the mixture was concentrated under vacuum to give a residue, which was purified by prep-HPLC (NH₄HCO₃ as additive) to afford Example 057 (32.8 mg, 21.27% yield) as a white solid. MS obsd. (ESI⁺) [(M+H)⁺]: 640.2.

### Example 057: ¹H NMR (400 MHz, DMSO-d6) δ ppm 10.27-10.96 (m, 1H), 8.17-8.30 (m, 1 H), 8.04-8.11 (m, 2H), 7.90-7.96 (m, 1H), 7.73-7.81 (m, 2H), 7.60-7.69 (m, 1H), 7.44-7.53 (m, 1H), 7.07-7.11 (m, 1H), 6.94-6.99 (m, 1H), 3.70-3.80 (m, 4H), 3.29-3.44 (m, 5 H), 2.99 (br t, J=4.52 Hz, 4H)

### Example 058: 4-[(3-Aminopropylamino)methyl]-N-[4-[4-[6-chloro-4-(trifluoromethyl)-2-pyridyl]piperazin-1-yl]sulfonylphenyl]pyridine-2-carboxamide

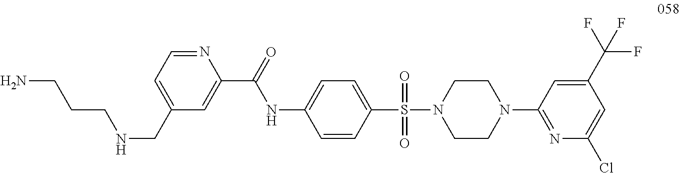 [SMILES: NCCCNCc1ccnc(C(=O)Nc2ccc(S(=O)(=O)N3CCN(c4cc(C(F)(F)F)cc(Cl)n4)CC3)cc2)c1]

Step 1: 4-(Chloromethyl)-N-[4-[4-[6-chloro-4-(trifluoromethyl)-2-pyridyl]piperazin-1-yl]sulfonylphenyl]pyridine-2-carboxamide

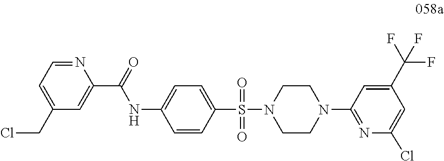 [SMILES: O=C(Nc1ccc(S(=O)(=O)N2CCN(c3cc(C(F)(F)F)cc(Cl)n3)CC2)cc1)c1cc(CCl)ccn1]

Compound 058a was prepared in analogy to the step of preparation compound 057a by replacing 3-(tert-butoxycarbonylamino)benzoic acid with 4-(chloromethyl)pyridine-2-carboxylic acid. Compound 058a (200 mg) was obtained as a white solid. MS obsd. (ESI⁺) [(M+H)⁺]: 574.2.

Step 2: Tert-butyl N-[3-[[2-[[4-[4-[6-chloro-4-(trifluoromethyl)-2-pyridyl]piperazin-1-yl]sulfonylphenyl]carbamoyl]-4-pyridyl]methylamino]propyl]carbamate

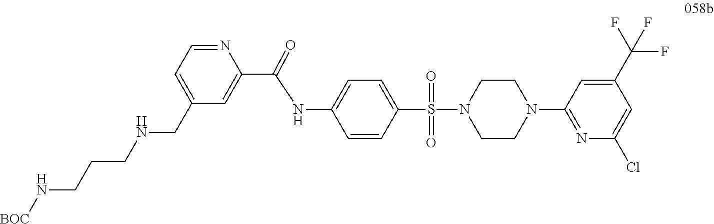 [SMILES: CC(C)(C)OC(=O)NCCCNCc1ccnc(C(=O)Nc2ccc(S(=O)(=O)N3CCN(c4cc(C(F)(F)F)cc(Cl)n4)CC3)cc2)c1]

A mixture of compound 058a (200.0 mg, 0.350 mmol), K₂CO₃ (144.37 mg, 1.04 mmol), KI (0.01 mL, 0.170 mmol) and tert-butyl N-(3-aminopropyl)carbamate (121.34 mg, 0.700 mmol) in MeCN (5 mL) was stirred at 70° C. for 2 hours. After completion, the mixture was concentrated in vacuo to give a crude compound 058b (200 mg, 80.65% yield) as yellow oil, which was used in the next step directly. MS obsd. (ESI⁺) [(M+H)⁺]: 712.2.

Step 3: 4-[(3-Aminopropylamino)methyl]-N-[4-[4-[6-chloro-4-(trifluoromethyl)-2-pyridyl]piperazin-1-yl]sulfonylphenyl]pyridine-2-carboxamide

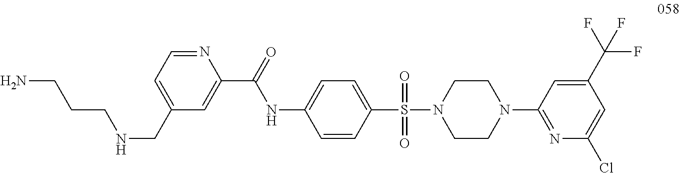 [SMILES: NCCCNCc1ccnc(C(=O)Nc2ccc(S(=O)(=O)N3CCN(c4cc(C(F)(F)F)cc(Cl)n4)CC3)cc2)c1]

A mixture of compound 058b (200.0 mg, 0.280 mmol) in DCM (4 mL) and TFA (3.2 g, 28.08 mmol) was stirred at 25° C. for 1 hour. After completion, the mixture was concentrated under vacuum to give crude, which was purified by prep-HPLC (NH₄HCO₃ as additive) to afford Example 058 (97.5 mg, 56.3% yield) as white solid. MS obsd. (ESI⁺) [(M+H)⁺]: 612.3.

### Example 058: ¹H NMR (400 MHz, DMSO-d6) δ ppm 11.25-10.84 (m, 1H), 8.66 (d, J=4.9 Hz, 1H), 8.30-8.08 (m, 3H), 7.76 (d, J=8.8 Hz, 2H), 7.64 (br d, J=4.6 Hz, 1H), 7.08 (s, 1H), 6.95 (s, 1H), 4.61-3.83 (s, 2H), 3.75-3.65 (m, 4H), 3.07-2.95 (m, 4H), 2.95-2.85 (m, 2H), 2.58-2.51 (m, 2H), 1.69-1.40 (m, 2H)

### Example 059: 6-[(3-Aminopropylamino)methyl]-N-[4-[4-[6-chloro-4-(trifluoromethyl)-2-pyridyl]piperazin-1-yl]sulfonylphenyl]pyridine-2-carboxamide

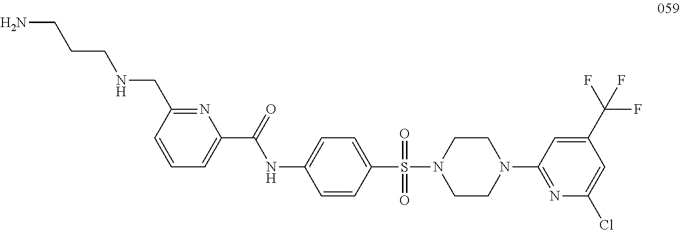 [SMILES: NCCCNCc1cccc(C(=O)Nc2ccc(S(=O)(=O)N3CCN(c4cc(C(F)(F)F)cc(Cl)n4)CC3)cc2)n1]

The title compound was prepared in analogy to the preparation of Example 044 by replacing 6-tert-butoxycarbonyl-5,7-dihydropyrrolo[3,4-b]pyridine-3-carboxylic acid with compound Int-17 and compound Int-13 with compound Int-1 in step1. Example 059 (51.3 mg) was obtained as a white solid. MS obsd. (ESI⁺) [(M+H)⁺]: 612.3.

### Example 059: ¹H NMR (400 MHz, DMSO-d6) δ ppm 8.20 (d, J=7.9 Hz, 2H), 8.02 (d, J=4.8 Hz, 2H), 7.79 (d, J=8.9 Hz, 2H), 7.69 (t, J=4.5 Hz, 1H), 7.09 (s, 1H), 6.97 (s, 1H), 3.92 (s, 2H), 3.80-3.69 (m, 5H), 3.01 (t, J=4.8 Hz, 5H), 2.62-2.57 (m, 2H), 1.55 (t, J=6.8 Hz, 2H)

### Example 060:5-[(3-Aminopropylamino)methyl]-N-[4-[4-[6-chloro-4-(trifluoromethyl)-2-pyridyl]piperazin-1-yl]sulfonylphenyl]pyridine-3-carboxamide

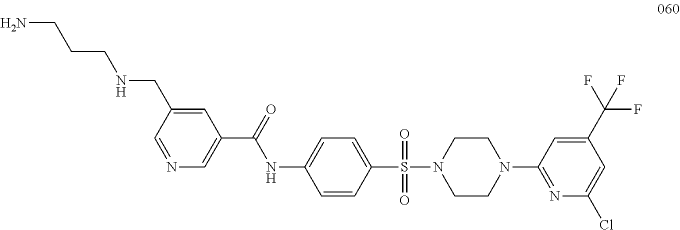 [SMILES: NCCCNCc1cncc(C(=O)Nc2ccc(S(=O)(=O)N3CCN(c4cc(C(F)(F)F)cc(Cl)n4)CC3)cc2)c1]

The title compound was prepared in analogy to the preparation of Example 058 by replacing 4-(chloromethyl)pyridine-2-carboxylic acid with 5-(bromomethyl)pyridine-3-carboxylic acid in step 1. Example 060 (38 mg) was obtained as a white solid. MS obsd. (ESI⁺) [(M+H)⁺]: 612.0.

### Example 060: ¹H NMR (400 MHz, DMSO-d₆) δ ppm: 8.95 (s, 1H), 8.75-8.64 (m, 1H), 8.31-8.17 (m, 1H), 8.10-8.03 (m, 2H), 7.80-7.74 (m, 2H), 7.09 (s, 1H), 6.98-6.91 (m, 1H), 3.77 (s, 2H), 3.74-3.68 (m, 4H), 2.99 (br s, 4H), 2.66-2.52 (m, 4H), 1.51 (s, 2H)

### Example 061: 2-(3-Aminopropylamino)-N-[4-[4-[6-chloro-4-(trifluoromethyl)-2-pyridyl]piperazin-1-yl]sulfonylphenyl]indane-5-carboxamide

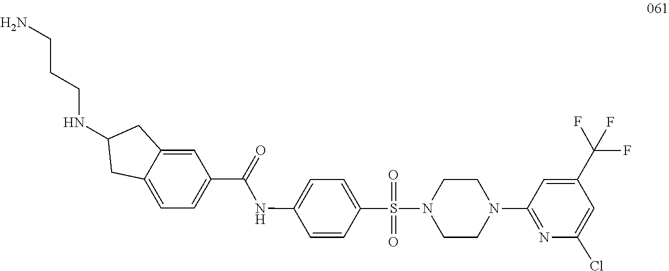 [SMILES: NCCCNC1Cc2ccc(C(=O)Nc3ccc(S(=O)(=O)N4CCN(c5cc(C(F)(F)F)cc(Cl)n5)CC4)cc3)cc2C1]

The title compound was prepared in analogy to the preparation of Example 044 by replacing 6-tert-butoxycarbonyl-5,7-dihydropyrrolo[3,4-b]pyridine-3-carboxylic acid with compound Int-18 and compound Int-13 with compound Int-1 in step1. Example 061 (87.9 mg) was obtained as a white solid. MS obsd. (ESI⁺) [(M+H)⁺]: 637.2.

### Example 061: ¹H NMR (400 MHz, DMSO-d6) δ ppm 10.66-10.82 (m, 1H), 9.71-9.97 (m, 2H), 8.07-8.37 (m, 5H), 7.82-7.96 (m, 2H), 7.70-7.78 (m, 2H), 7.40-7.51 (m, 1H), 7.05-7.15 (m, 1H), 6.87-7.00 (m, 1H), 3.98-4.15 (m, 1H), 3.63-3.79 (m, 4H), 3.22-3.37 (m, 4 H), 3.08-3.18 (m, 2H), 2.91-3.02 (m, 6H), 2.00-2.10 (m, 2H)

### Example 062: 3-(3-Aminopropylamino)-N-[4-[4-[6-chloro-4-(trifluoromethyl)-2-pyridyl]piperazin-1-yl]sulfonylphenyl]indane-5-carboxamide

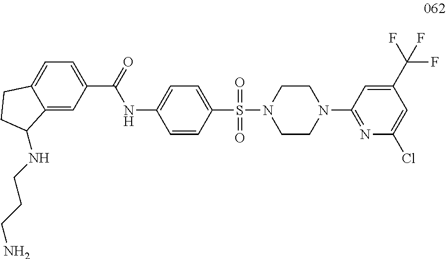 [SMILES: NCCCNC1CCc2ccc(C(=O)Nc3ccc(S(=O)(=O)N4CCN(c5cc(C(F)(F)F)cc(Cl)n5)CC4)cc3)cc21]

Step 1: N-[4-[4-[6-chloro-4-(trifluoromethyl)-2-pyridyl]piperazin-1-yl]sulfonylphenyl]-1-oxo-indane-5-carboxamide

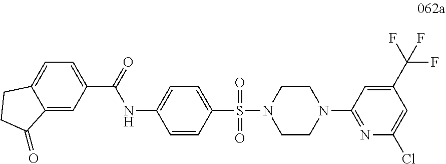 [SMILES: O=C(Nc1ccc(S(=O)(=O)N2CCN(c3cc(C(F)(F)F)cc(Cl)n3)CC2)cc1)c1ccc2c(c1)C(=O)CC2]

To a mixture of 3-oxoindane-5-carboxylic acid (200.0 mg, 1.14 mmol), compound Int-1 (477.77 mg, 1.14 mmol) and 3-methylpyridine (0.43 mL, 4.54 mmol) in MeCN (5 mL) was added MsCl (520.18 mg, 4.54 mmol) dropwise at rt. The mixture was heated to 45° C. and stirred for 2 hours. After completion, the mixture was poured in to water (20 mL), extracted with EtOAc (20 mL×3). The combined organic layer was washed with brine, dried over Na₂SO₄ and concentrated under vacuum to give crude product, which was purified by prep-HPLC (FA as additive) to give compound 062a (180 mg, 27.38% yield) as white solid. MS obsd. (ESI⁺) [(M+H)⁺]: 579.2.

Step 2: Tert-butyl N-[3-[[6-[[4-[4-[6-chloro-4-(trifluoromethyl)-2-pyridyl]piperazin-1-yl]sulfonylphenyl]carbamoyl]indan-1-yl]aminolpropyl]carbamate

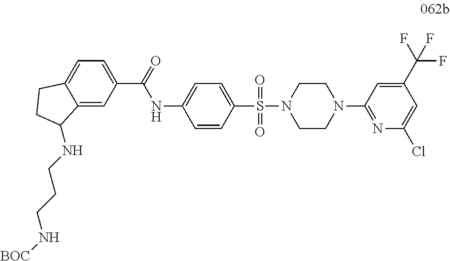 [SMILES: CC(C)(C)OC(=O)NCCCNC1CCc2ccc(C(=O)Nc3ccc(S(=O)(=O)N4CCN(c5cc(C(F)(F)F)cc(Cl)n5)CC4)cc3)cc21]

The mixture of compound 062a (100.0 mg, 0.170 mmol), tert-butyl N-(3-aminopropyl)carbamate (60.19 mg, 0.350 mmol), and titanium(IV)isopropoxide (98.18 mg, 0.350 mmol) in toluene (10 mL) was stirred at 50° C. for 15 hours. And then, the mixture was concentrated under vacuum to give a residue. Sodium cyanoborohydride (43.41 mg, 0.690 mmol) in MeOH was added to the above residue and the mixture was stirred for 4 hours at 50° C. After completion, the mixture was concentrated in vacuo to give a crude compound 062b (100 mg, 78.54% yield) as a yellow oil, which was used in the next step directly. MS obsd. (ESI⁺) [(M+H)⁺]: 737.4.

Step 3: 3-(3-Aminopropylamino)-N-[4-[4-[6-chloro-4-(trifluoromethyl)-2-pyridyl]piperazin-1-yl]sulfonylphenyl]indane-5-carboxamide

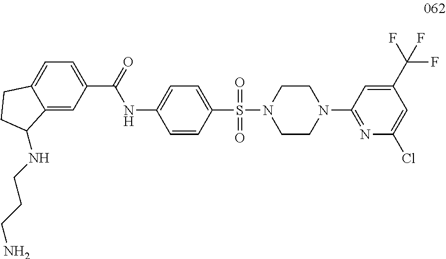 [SMILES: NCCCNC1CCc2ccc(C(=O)Nc3ccc(S(=O)(=O)N4CCN(c5cc(C(F)(F)F)cc(Cl)n5)CC4)cc3)cc21]

The mixture of compound 062b (100.0 mg, 0.140 mmol) in DCM (5 mL) and TFA (10.0 mL) was stirred at 25° C. for 2 hours. Solvent was evaporated and the residue was concentrated under vacuum to give crude product which was purified by and prep-HPLC (HCl as additive) to afford Example 062 (1.9 mg, 2.16% yield) as white solid. MS obsd. (ESI⁺) [(M+H)⁺]: 637.3.

### Example 062: ¹H NMR (400 MHz, DMSO-d6) δ ppm 10.85 (s, 1H), 9.66-9.23 (m, 2H), 8.37 (s, 1H), 8.20-8.05 (m, 4H), 8.00 (dd, J=1.2, 7.9 Hz, 3H), 7.76 (d, J=8.8 Hz, 2H), 7.09 (s, 1H), 6.97 (s, 1H), 4.80 (br d, J=1.8 Hz, 1H), 3.82-3.65 (m, 4H), 3.31-3.23 (m, 2H), 3.23-3.09 (m, 2H), 3.04-2.89 (m, 7H), 2.47-2.42 (m, 1H), 2.13-1.96 (m, 2H)

### Example 063: N-[4-[4-[6-chloro-4-(trifluoromethyl)-2-pyridyl]piperazin-1-yl]sulfonylphenyl]-2-methoxy-5-[(5-methyl-2,3,3a,4,6,6a-hexahydropyrrolo[2,3-c]pyrrol-1-yl)methyl]benzamide

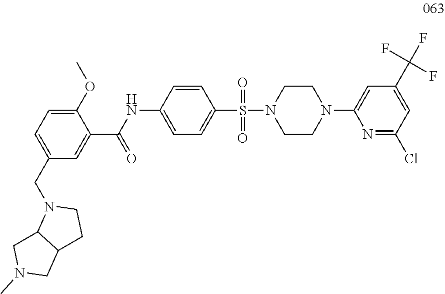 [SMILES: COc1ccc(CN2CCC3CN(C)CC32)cc1C(=O)Nc1ccc(S(=O)(=O)N2CCN(c3cc(C(F)(F)F)cc(Cl)n3)CC2)cc1]

Step 1: 5-(Chloromethyl)-N-[4-[4-[6-chloro-4-(trifluoromethyl)-2-pyridyl]piperazin-1-yl]sulfonylphenyl]-2-methoxy-benzamide

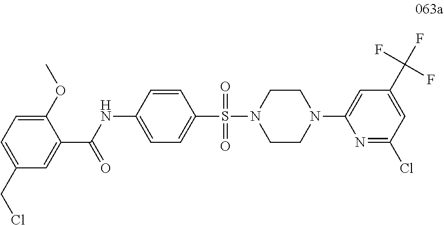 [SMILES: COc1ccc(CCl)cc1C(=O)Nc1ccc(S(=O)(=O)N2CCN(c3cc(C(F)(F)F)cc(Cl)n3)CC2)cc1]

A mixture of 5-(chloromethyl)-2-methoxybenzoyl chloride (687 mg, 3.14 mmol), compound Int-1 (0.660 g, 1.57 mmol) and TEA (794 mg, 7.84 mmol) in DCM (10 mL) was stirred at rt for 16 hours. After completion, the mixture was quenched with water (20 mL), extracted with DCM (10 mL×3). The combined organic layer was dried over anhydrous Na₂SO₄, and concentrated under vacuum to give crude compound 063a (946 mg, 100%), which was used in the next step directly. MS obsd. (ESI⁺) [(M+H)]⁺: 603.1 Step 2: N-[4-[4-[6-chloro-4-(trifluoromethyl)-2-pyridyl]piperazin-1-yl]sulfonylphenyl]-2-methoxy-5-[(5-methyl-2,3,3a,4,6,6a-hexahydropyrrolo[3,4-b]pyrrol-1-yl)methyl]benzamide

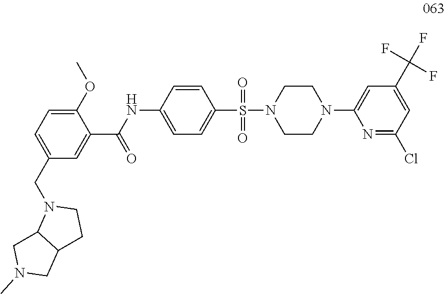 [SMILES: COc1ccc(CN2CCC3CN(C)CC32)cc1C(=O)Nc1ccc(S(=O)(=O)N2CCN(c3cc(C(F)(F)F)cc(Cl)n3)CC2)cc1]

A mixture of compound 063a (0.060 g, 99.4 μmol), DIPEA (38.6 mg, 298 μmol) and 5-methyloctahydropyrrolo[3,4-b]pyrrole (25.1 mg, 199 μmol) in DMF (10 mL) was stirred at 80° C. overnight. After completion, the mixture was diluted with EtOAc (20 mL), washed with water (20 mL). The organic layer was dried over Na₂SO₄, concentrated under vacuum to give a residue, which was purified by prep-HPLC (TFA as additive) to give product (1 mg, 1.41%) as a white solid. MS obsd. (ESI⁺) [(M+H)]⁺: 693.1

### Example 063: ¹H NMR (400 MHz, DMSO-d₆) δ ppm 10.53 (s, 1H), 8.00-7.96 (m, 2H), 7.76-7.71 (m, 2H), 7.60 (br s, 1H), 7.48 (br d, J=7.7 Hz, 1H), 7.14 (d, J=8.7 Hz, 1H), 7.09 (s, 1H), 6.97 (s, 1H), 3.93-3.90 (m, 1H), 3.88-3.86 (m, 3H), 3.80-3.75 (m, 1H), 3.73-3.70 (m, 4H), 3.43-3.37 (m, 3H), 3.00-2.95 (m, 6H), 2.80-2.72 (m, 4H), 2.64-2.58 (m, 1H), 2.20-2.09 (m, 1H), 2.06-1.96 (m, 1H), 1.56-1.46 (m, 1H). ¹⁹F NMR (376 MHz, DMSO-d6) δ ppm −68.55 (s, 3F)

### Example 064: 1-(3-Aminopropylamino)-N-[4-[4-[6-chloro-4-(trifluoromethyl)-2-pyridyl]piperazin-1-yl]sulfonylphenyl]indane-5-carboxamide

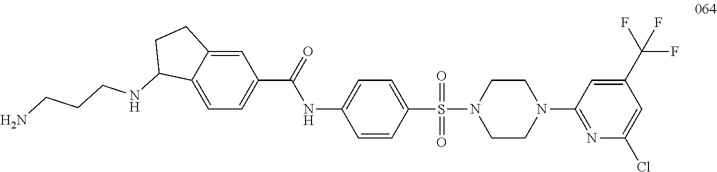 [SMILES: NCCCNC1CCc2cc(C(=O)Nc3ccc(S(=O)(=O)N4CCN(c5cc(C(F)(F)F)cc(Cl)n5)CC4)cc3)ccc21]

The title compound was prepared in analogy to the preparation of Example 062 by replacing 3-oxoindane-5-carboxylic acid with 1-oxoindane-5-carboxylic acid in step1. Example 064 (87.9 mg) was obtained as a white solid. MS obsd. (ESI⁺) [(M+H)⁺]: 637.3.

### Example 064: ¹H NMR (400 MHz, DMSO-d6) δ ppm 10.79 (s, 1H), 10.03-9.45 (m, 2H), 8.15 (br d, J=8.9 Hz, 4H), 8.04-7.92 (m, 3H), 7.80 (d, J=8.8 Hz, 2H), 7.12 (s, 1H), 6.97 (s, 1H), 5.92-5.67 (m, 1H), 4.87 (br s, 1H), 3.86-3.73 (m, 4H), 3.70-3.60 (m, 2H), 3.12 (br s, 2H), 3.07-2.93 (m, 7H), 2.18-2.06 (m, 1H), 1.86-1.73 (m, 2H)

### Example 065: 3,4-Bis(3-Aminopropoxy)-N-[4-[4-[6-chloro-4-(trifluoromethyl)-2-pyridyl]piperazin-1-yl]sulfonylphenyl]benzamide

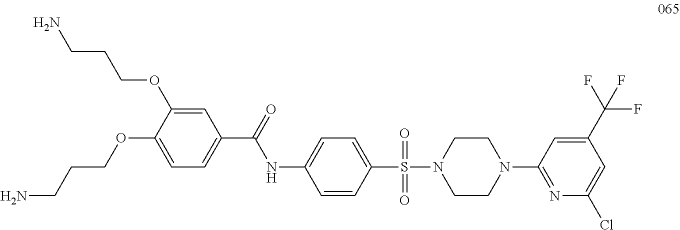 [SMILES: NCCCOc1ccc(C(=O)Nc2ccc(S(=O)(=O)N3CCN(c4cc(C(F)(F)F)cc(Cl)n4)CC3)cc2)cc1OCCCN]

Step 1: N-[4-[4-[6-chloro-4-(trifluoromethyl)-2-pyridyl]piperazin-1-yl]sulfonylphenyl]-3,4-dimethoxy-benzamide

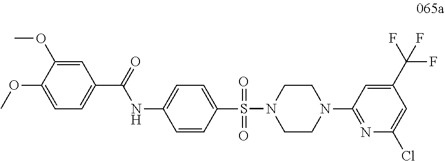 [SMILES: COc1ccc(C(=O)Nc2ccc(S(=O)(=O)N3CCN(c4cc(C(F)(F)F)cc(Cl)n4)CC3)cc2)cc1OC]

SOCl₂ (4.0 mL, 0.750 mmol, 1 eq) was added to a solution of 3,4-dimethoxybenzoic acid (274.44 mg, 1.51 mmol) in toluene (4 mL) at rt. And then, the mixture was heated to 100° C. for 3 hours. And then, the reaction mixture was concentrated and the residue was re-dissolved in 4 mL DCM, which was added dropwise to a mixture of compound Int-1 (317 mg, 0.8 mmol) and DIPEA (0.3 mL) in 10 mL DCM at rt. After completion, the mixture was concentrated under vacuum to give residue, which was purified by silica-gel chromatography (elute with EtOAc:PE=50%) to afford compound 065a (387 mg, 83.43% yield). MS obsd. (ESI⁺) [(M+H)⁺]: 585.1.

Step 2: N-[4-[4-[6-chloro-4-(trifluoromethyl)-2-pyridyl]piperazin-1-yl]sulfonylphenyl]-3,4-dihydroxy-benzamide

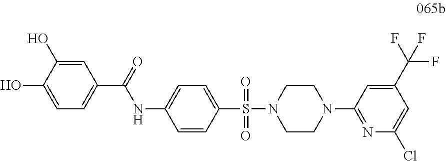 [SMILES: O=C(Nc1ccc(S(=O)(=O)N2CCN(c3cc(C(F)(F)F)cc(Cl)n3)CC2)cc1)c1ccc(O)c(O)c1]

To a solution of compound (187.0 mg, 0.320 mmol) in DCM (4 mL) was added BBr₃ (240.24 mg, 0.960 mmol) dropwise at 25° C. The resulted mixture was stirred for 2 hours at rt. After completion, MeOH (5 mL) was added to quench the reaction. And then, the reaction mixture was concentrated and the residue was purified by prep-HPLC (TFA as additive) to afford compound 065b (96 mg, 48.53% yield) as a light yellow solid. MS obsd. (ESI⁺) [(M+H)⁺]: 557.1.

Step 3: Tert-butyl N-[3-[2-[3-(tert-butoxycarbonylamino)propoxy]-4-[[4-[4-[6-chloro-4-(trifluoromethyl)-2-pyridyl]piperazin-1-yl]sulfonylphenyl]carbamoyl]phenoxy]propyl]carbamate

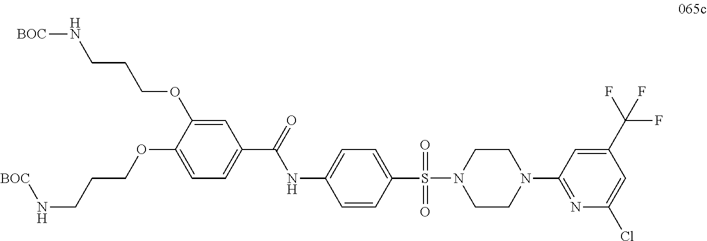 [SMILES: CC(C)(C)OC(=O)NCCCOc1ccc(C(=O)Nc2ccc(S(=O)(=O)N3CCN(c4cc(C(F)(F)F)cc(Cl)n4)CC3)cc2)cc1OCCCNC(=O)OC(C)(C)C]

A mixture of compound 065b (56.0 mg, 0.100 mmol), K₂CO₃ (20.0 mg) and tert-butyl N-(3-iodopropyl)carbamate (0.08 mL, 0.150 mmol) in DMF (1.4 mL) was stirred at 25° C. for 16 hours. And then, the mixture was poured into water (5 mL), extracted with EtOAc (8 mL×3). The combined organic layer was dried over Na₂SO₄ and concentrated under vacuum to give a residue, which was purified by prep-HPLC (TFA as additive) to afford the compound 065c (45 mg, 46.23% yield) as a white solid. MS obsd. (ESI⁺) [(M+H)⁺]: 871.3.

Step 4: 3,4-Bis(3-aminopropoxy)-N-[4-[4-[6-chloro-4-(trifluoromethyl)-2-pyridyl]piperazin-1-yl]sulfonylphenyl]benzamide

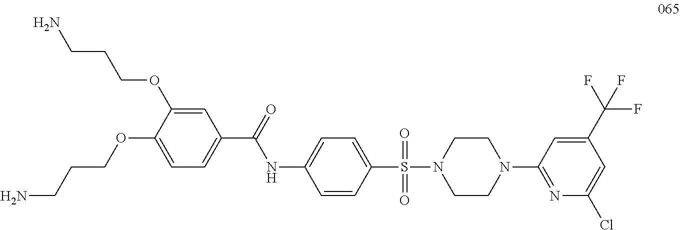 [SMILES: NCCCOc1ccc(C(=O)Nc2ccc(S(=O)(=O)N3CCN(c4cc(C(F)(F)F)cc(Cl)n4)CC3)cc2)cc1OCCCN]

Compound 065c (45.0 mg, 0.050 mmol) was dissolved in 4M TFA/dioxane (4 mL) and the mixture was stirred at rt for 4 hours. After completion, the reaction mixture was concentrated under vacuum to give residue, which was purified by prep-HPLC (TFA as additive) to afford

### Example 065 (23 mg, 65.69% yield) as white solid. MS obsd. (ESI⁺) [(M+H)⁺]: 671.3

### Example 065: ¹H NMR (400 MHz, DMSO-d6) δ ppm 10.67 (s, 1H), 8.08 (d, J=8.6 Hz, 2H), 7.74 (d, J=8.6 Hz, 2H), 7.70-7.59 (m, 2H), 7.16-7.05 (m, 2H), 6.97 (s, 1H), 4.16 (s, 4H), 3.72 (s, 4H), 2.99 (s, 8H), 2.04 (s, 4H). ¹⁹F NMR (376 MHz, DMSO) δ ppm −63.46 (s, 3F)

### Example 066: 3-[1-(3-Aminopropyl)pyrrolidin-2-yl]-N-[4-[4-[6-chloro-4-(trifluoromethyl)-2-pyridyl]piperazin-1-yl]sulfonylphenyl]benzamide

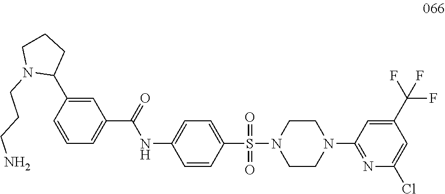 [SMILES: NCCCN1CCCC1c1cccc(C(=O)Nc2ccc(S(=O)(=O)N3CCN(c4cc(C(F)(F)F)cc(Cl)n4)CC3)cc2)c1]

The title compound was prepared in analogy to the preparation of example 044 by replacing 6-tert-butoxycarbonyl-5,7-dihydropyrrolo[3,4-b]pyridine-3-carboxylic acid with compound Int-19 and compound Int-13 with compound Int-1 in step1. Example 066 (22.85 mg) was obtained. MS obsd. (ESI⁺) [(M+H)⁺]: 651.2.

### Example 066: ¹H NMR (400 MHz, DMSO-d6) δ ppm 8.06 (d, J=8.8 Hz, 2H), 7.88 (s, 1H), 7.84 (br d, J=7.8 Hz, 1H), 7.76 (d, J=8.8 Hz, 2H), 7.61-7.55 (m, 1H), 7.53-7.46 (m, 1H), 7.09 (s, 1H), 6.97 (s, 1H), 3.78-3.69 (m, 4H), 3.00 (br t, J=4.5 Hz, 4H), 2.60-2.55 (m, 2H), 2.47-2.41 (m, 2H), 2.25-2.06 (m, 4H), 1.95-1.72 (m, 2H), 1.67-1.47 (m, 3H)

### Example 067:3-[1-(3-Aminopropyl)pyrrolidin-3-yl]-N-[4-[4-[6-chloro-4-(trifluoromethyl)-2-pyridyl]piperazin-1-yl]sulfonylphenyl]benzamide

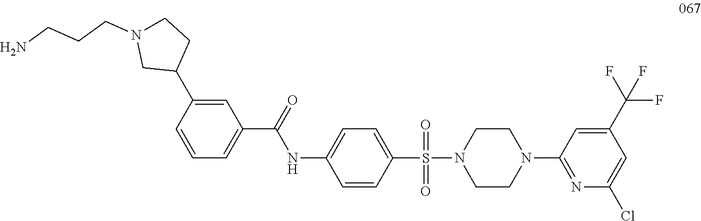 [SMILES: NCCCN1CCC(c2cccc(C(=O)Nc3ccc(S(=O)(=O)N4CCN(c5cc(C(F)(F)F)cc(Cl)n5)CC4)cc3)c2)C1]

The title compound was prepared in analogy to the preparation of example 044 by replacing 6-tert-butoxycarbonyl-5,7-dihydropyrrolo[3,4-b]pyridine-3-carboxylic acid with compound Int-20 and compound Int-13 with compound Int-1 in step1. Example 067 (22.85 mg) was obtained. MS obsd. (ESI⁺) [(M+H)⁺]: 651.2.

### Example 067: ¹H NMR (400 MHz, DMSO-d6) δ ppm 8.06 (d, J=8.9 Hz, 2H), 7.86-7.79 (m, 1H), 7.77 (s, 3H), 7.59-7.52 (m, 1H), 7.49-7.42 (m, 1H), 7.13-7.05 (m, 1H), 6.99-6.93 (m, 1H), 3.72 (br s, 4H), 3.02-2.96 (m, 4H), 2.92-2.86 (m, 1H), 2.75-2.60 (m, 4H), 2.32-2.23 (m, 1H), 1.87-1.72 (m, 1H), 1.68-1.50 (m, 2H)

### Example 068: 3-[4-(3-Aminopropyl)piperazin-1-yl]-N-[4-[4-[6-chloro-4-(trifluoromethyl)-2-pyridyl]piperazin-1-yl]sulfonylphenyl]benzamide

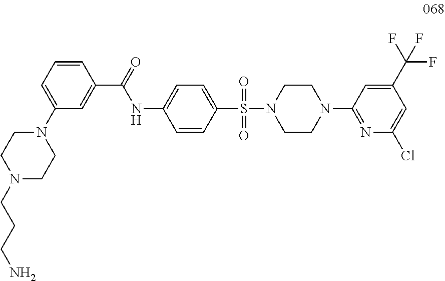 [SMILES: NCCCN1CCN(c2cccc(C(=O)Nc3ccc(S(=O)(=O)N4CCN(c5cc(C(F)(F)F)cc(Cl)n5)CC4)cc3)c2)CC1]

Step 1: Tert-butyl 4-[3-[[4-[4-[6-chloro-4-(trifluoromethyl)-2-pyridyl]piperazin-1-yl]sulfonylphenyl]carbamoyl]phenyl]piperazine-1-carboxylate

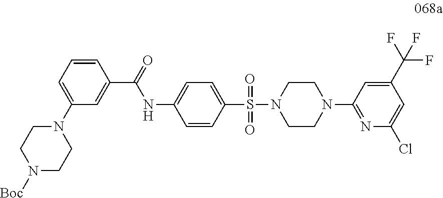 [SMILES: CC(C)(C)OC(=O)N1CCN(c2cccc(C(=O)Nc3ccc(S(=O)(=O)N4CCN(c5cc(C(F)(F)F)cc(Cl)n5)CC4)cc3)c2)CC1]

A mixture of 3-(4-tert-butoxycarbonylpiperazin-1-yl)benzoic acid (300.0 mg, 0.980 mmol), 3-methylpyridine (182.39 mg, 1.96 mmol) and MsCl (0.11 mL, 1.47 mmol) in MeCN (5 mL) was stirred at 25° C. for 1 hour. And then, compound Int-1 (412.1 mg, 0.980 mmol) was added to the mixture which was stirred at rt for another 2 hours. After completion, the mixture was diluted with water (10 mL) and extracted with EtOAc (20 mL×3). The combined organic layer was washed with brine (30 mL), dried with anhydrous Na₂SO₄ and concentrated under vacuum to give residue, which was purified by prep-HPLC(TFA as additive) to give compound 068a (210 mg, 30.24% yield) as yellow solid. MS obsd. (ESI⁺) [(M+H)⁺]: 709.2.

Step 2: N-[4-[4-[6-chloro-4-(trifluoromethyl)-2-pyridyl]piperazin-1-yl]sulfonylphenyl]-3-piperazin-1-yl-benzamide

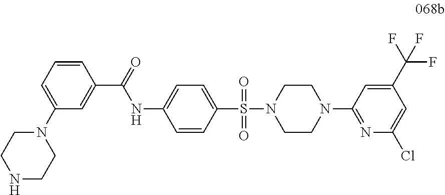 [SMILES: O=C(Nc1ccc(S(=O)(=O)N2CCN(c3cc(C(F)(F)F)cc(Cl)n3)CC2)cc1)c1cccc(N2CCNCC2)c1]

The mixture of compound 068a (210.0 mg, 0.300 mmol) in DCM (1 mL) and TFA (1.14 mL) was stirred at 25° C. for 1 hour. After completion, the mixture was concentrated under vacuum to give compound 068b (180 mg, 99.8% yield) as yellow oil, which was used directly in the next step. MS obsd. (ESI⁺) [(M+H)⁺]: 609.1.

Step 3: Tert-butyl N-[3-[4-[3-[[4-[4-[6-chloro-4-(trifluoromethyl)-2-pyridyl]piperazin-1-yl]sulfonylphenyl]carbamoyl]phenyl]piperazin-1-yl]propyl]carbamate

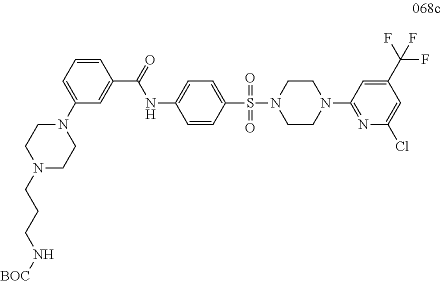 [SMILES: CC(C)(C)OC(=O)NCCCN1CCN(c2cccc(C(=O)Nc3ccc(S(=O)(=O)N4CCN(c5cc(C(F)(F)F)cc(Cl)n5)CC4)cc3)c2)CC1]

A mixture of compound 068b (190.0 mg, 0.310 mmol), KI (0.0 mL, 0.030 mmol), DIPEA (0.11 mL, 0.620 mmol) and tert-butyl N-(3-chloropropyl)carbamate (90.62 mg, 0.470 mmol) in in DMF (10 mL) was stirred at 80° C. for 16 hours. After completion, the mixture was diluted with water (20 mL) and extracted with EtOAc (20 mL×3). The combined organic layer was washed with brine (50 mL), dried with anhydrous Na₂SO₄ and concentrated under vacuum to give crude, which was purified by prep-TLC(DCM/MeOH=10/1) to give compound 068c (120 mg, 50.2% yield) as a yellow solid. MS obsd. (ESI⁺) [(M+H)⁺]: 766.2.

Step 4: 3-[4-(3-Aminopropyl)piperazin-1-yl]-N-[4-[4-[6-chloro-4-(trifluoromethyl)-2-pyridyl]piperazin-1-yl]sulfonylphenyl]benzamide

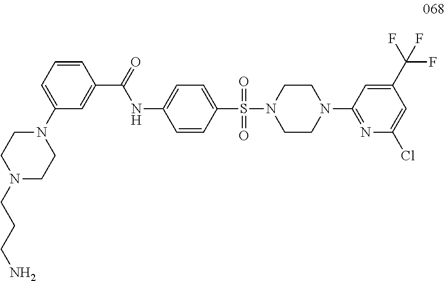 [SMILES: NCCCN1CCN(c2cccc(C(=O)Nc3ccc(S(=O)(=O)N4CCN(c5cc(C(F)(F)F)cc(Cl)n5)CC4)cc3)c2)CC1]

A mixture of compound 068c (120.0 mg, 0.160 mmol) in DCM (1 mL) and TFA (0.6 mL) was stirred at rt for 1 hour. After completion, the mixture was concentrated under vacuum to give a residue, which was purified by prep-HPLC (NH₄HCO₃ as additive) to afford Example 068 (65 mg, 62.01% yield) as a yellow solid. MS obsd. (ESI⁺) [(M+H)⁺]: 666.0.

### Example 067: ¹H NMR (400 MHz, DMSO-d₆) δ ppm: 8.07-8.01 (m, 2H), 7.75 (s, 2H), 7.43-7.39 (m, 1H), 7.39-7.30 (m, 2H), 7.19-7.14 (m, 1H), 7.10-7.06 (m, 1H), 6.96 (s, 1H), 3.74-3.67 (m, 4H), 3.23-3.18 (m, 4H), 2.97 (s, 4H), 2.37-2.32 (m, 2H), 1.60-1.50 (m, 2H)

### Example 069: 4-[2-(3-Aminopyrrolidin-1-yl)ethyl]-N-[4-[4-[6-chloro-4-(trifluoromethyl)-2-pyridyl]piperazin-1-yl]sulfonylphenyl]benzamide

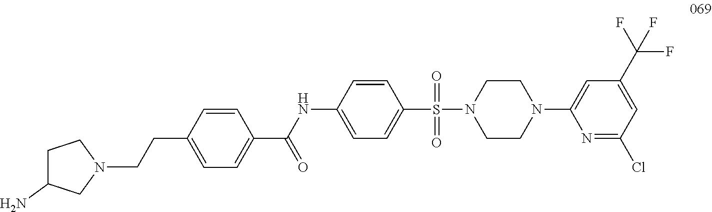 [SMILES: NC1CCN(CCc2ccc(C(=O)Nc3ccc(S(=O)(=O)N4CCN(c5cc(C(F)(F)F)cc(Cl)n5)CC4)cc3)cc2)C1]

Step1: 4-(2-Chloroethyl)-N-[4-[4-[6-chloro-4-(trifluoromethyl)-2-pyridyl]piperazin-1-yl]sulfonylphenyl]benzamide

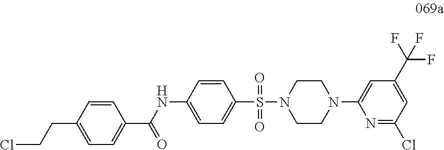 [SMILES: O=C(Nc1ccc(S(=O)(=O)N2CCN(c3cc(C(F)(F)F)cc(Cl)n3)CC2)cc1)c1ccc(CCCl)cc1]

To a mixture of compound Int-1 (1.26 g, 2.99 mmol) and TEA (1.51 g, 2.09 mL) in DCM (20 mL) was added a solution of 4-(2-chloroethyl)benzoyl chloride (1.03 g, 5.09 mmol) in DCM (3 mL) dropwise at 0° C. The mixture was slowly warmed up to rt and stirred for 40 minutes. After completion, the mixture was quenched with water (30 mL) and extracted with DCM (30 mL). The organic layer was dried over anhydrous Na₂SO₄ and concentrated under vacuum to give crude product, which was purified by silica-gel chromatography (elute with EtOAc:PE=10%) to afford compound 069a (0.850 g, 48%). MS obsd. (ESI⁺) [(M+H)]⁺: 587.1

Step2: Tert-butyl N-[1-[2-[4-[[4-[4-[6-chloro-4-(trifluoromethyl)-2-pyridyl]piperazin-1-yl]sulfonylphenyl]carbamoyl]phenyl]ethyl]pyrrolidin-3-yl]carbamate

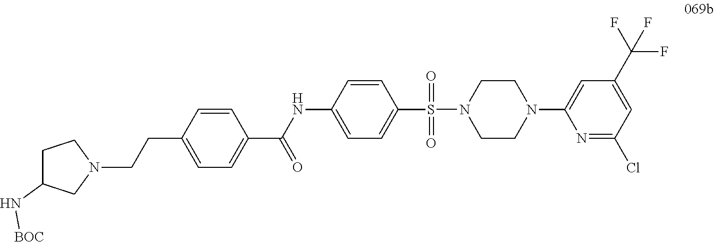 [SMILES: CC(C)(C)OC(=O)NC1CCN(CCc2ccc(C(=O)Nc3ccc(S(=O)(=O)N4CCN(c5cc(C(F)(F)F)cc(Cl)n5)CC4)cc3)cc2)C1]

A mixture of tert-butyl pyrrolidin-3-ylcarbamate (66.6 mg, 357 μmol), compound 069a (0.070 g, 119 μmol) and K₂CO₃ (49.4 mg, 357 μmol) in DMF (10 mL) was stirred at 80° C. for 2 hours. After completion, the mixture was diluted with EtOAc (10 mL), washed with water (10 mL). The organic was dried over anhydrous Na₂SO₄ and concentrated under vacuum to give crude compound 069b (87.9 mg, 100%) which was used directly in the next step. MS obsd. (ESI⁺) [(M+H)]⁺: 736.9.

Step 3: 4-[2-(3-Aminopyrrolidin-1-yl)ethyl]-N-[4-[4-[6-chloro-4-(trifluoromethyl)-2-pyridyl]piperazin-1-yl]sulfonylphenyl]benzamide

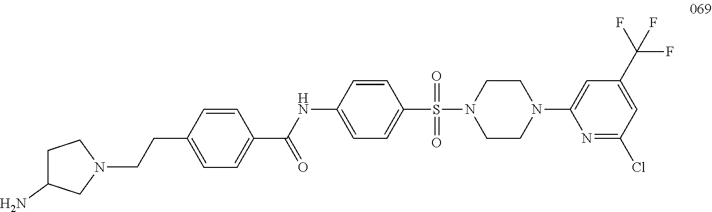 [SMILES: NC1CCN(CCc2ccc(C(=O)Nc3ccc(S(=O)(=O)N4CCN(c5cc(C(F)(F)F)cc(Cl)n5)CC4)cc3)cc2)C1]

A mixture of compound 069b (0.0879 g, 119 μmol) in TFA (1 mL) was stirred at rt for one hour. And then, the mixture was concentrated under vacuum to give a residue, which was purified by prep-HPLC (TFA as additive) to provide Example 069 (8 mg, 10.5%) as a white solid. MS obsd. (ESI⁺) [(M+H)]⁺: 637.2

### Example 069: ¹H NMR (400 MHz, DMSO-d6) δ ppm 10.59 (s, 1H), 8.08-8.03 (m, 2H), 7.92-7.87 (m, 2H), 7.78-7.72 (m, 2H), 7.44-7.39 (m, 2H), 7.09 (s, 1H), 6.96 (s, 1H), 3.75-3.69 (m, 4H), 3.67-3.60 (m, 1H), 3.01-2.95 (m, 4H), 2.86-2.81 (m, 2H), 2.78 (br dd, J=3.0, 8.6 Hz, 1H), 2.72-2.67 (m, 3H), 2.61-2.58 (m, 1H), 2.48-2.41 (m, 1H), 2.20-2.04 (m, 1H), 1.65 (br d, J=4.2 Hz, 1H). ¹⁹F NMR (376 MHz, DMSO-d6) δ ppm −68.53 (s, 3F)

### Example 070: 4-[2-(7-Amino-2-azaspiro[3.3]heptan-2-yl)ethyl]-N-[4-[4-[6-chloro-4-(trifluoromethyl)-2-pyridyl]piperazin-1-yl]sulfonylphenyl]benzamide

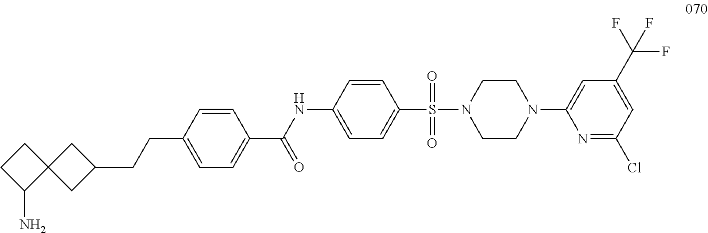 [SMILES: NC1CCC12CC(CCc1ccc(C(=O)Nc3ccc(S(=O)(=O)N4CCN(c5cc(C(F)(F)F)cc(Cl)n5)CC4)cc3)cc1)C2]

The title compound was prepared in analogy to the preparation of Example 069 by replacing tert-butyl pyrrolidin-3-ylcarbamate with tert-butyl (2-azaspiro[3.3]heptan-5-yl)carbamate in

step2. Example 070 (3 mg) was obtained as a white solid. MS obsd. (ESI⁺) [(M+H)⁺]: 663.1.

### Example 070: ¹H NMR (400 MHz, DMSO-d6) δ ppm 10.62 (s, 1H), 8.07 (s, 1H), 8.05 (s, 1H), 8.00-7.95 (m, J=8.1 Hz, 2H), 7.76 (d, J=8.9 Hz, 2H), 7.48-7.40 (m, 2H), 7.09 (s, 1H), 6.97 (s, 1H), 4.14-3.92 (m, 4H), 3.75-3.70 (m, 4H), 3.45 (m, 3H), 3.01-2.96 (m, 4H), 2.94-2.86 (m, 2H), 2.22 (br d, J=7.6 Hz, 1H), 2.16-2.01 (m, 2H), 1.90 (br d, J=6.6 Hz, 1H). ¹⁹F NMR (376 MHz, DMSO-d6) δ ppm −68.44 (s, 3F)

### Example 071: N-[4-[4-[6-chloro-4-(trifluoromethyl)-2-pyridyl]piperazin-1-yl]sulfonylphenyl]-4-[2-(5-methyl-2,3,3a,4,6,6a-hexahydropyrrolo[2,3-c]pyrrol-1-yl)ethyl]benzamide

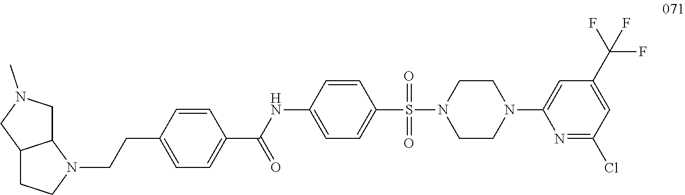 [SMILES: CN1CC2CCN(CCc3ccc(C(=O)Nc4ccc(S(=O)(=O)N5CCN(c6cc(C(F)(F)F)cc(Cl)n6)CC5)cc4)cc3)C2C1]

The title compound was prepared in analogy to the preparation of compound 069b by replacing tert-butyl pyrrolidin-3-ylcarbamate with 5-methyloctahydropyrrolo[3,4-b]pyrrole in step 2. Example 071 (4 mg) was obtained as a white solid. MS obsd. (ESI⁺) [(M+H)⁺]: 676.8.

### Example 071: ¹H NMR (400 MHz, DMSO-d6) δ ppm 10.59 (s, 1H), 8.09-8.04 (m, J=8.9 Hz, 2H), 7.94-7.88 (m, J=8.2 Hz, 2H), 7.77-7.73 (m, J=8.9 Hz, 2H), 7.46-7.39 (m, J=8.3 Hz, 2H), 7.09 (s, 1H), 6.96 (s, 1H), 3.75-3.69 (m, 4H), 3.24-3.17 (m, 2H), 3.14-3.04 (m, 2H), 3.01-2.97 (m, 4H), 2.93-2.80 (m, 5H), 2.70-2.63 (m, 2H), 2.52 (br s, 3H), 2.44-2.33 (m, 1H), 2.02 (br s, 1H), 1.56 (br t, J=11.3 Hz, 1H). ¹⁹F NMR (376 MHz, DMSO-d6) δ ppm −68.55 (s, 2F)

### Example 072: N-[4-[4-[6-chloro-4-(trifluoromethyl)-2-pyridyl]piperazin-1-yl]sulfonylphenyl]-4-[[(2S)-2-(2-aminoethyl)pyrrolidin-1-yl]methyl]benzamide

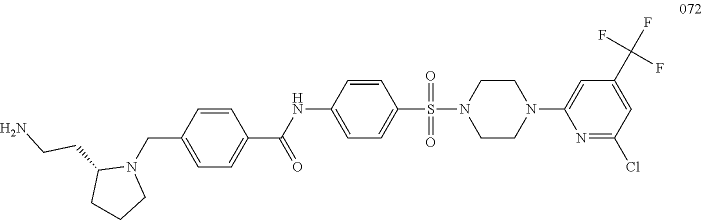 [SMILES: NCC[C@H]1CCCN1Cc1ccc(C(=O)Nc2ccc(S(=O)(=O)N3CCN(c4cc(C(F)(F)F)cc(Cl)n4)CC3)cc2)cc1]

The title compound was prepared in analogy to the preparation of Example 001 by replacing compound tert-butyl N-(3-aminopropyl)carbamate with tert-butyl N-[2-[(2R)-pyrrolidin-2-yl]ethyl]carbamate in step 1. Example 072 (31 mg) was obtained as a pink solid. MS obsd. (ESI⁺) [(M+H)⁺]: 651.1.

### Example 072: ¹H NMR (400 MHz, DMSO-d₆) δ ppm 8.09-8.01 (m, 2H), 7.95-7.88 (m, 2H), 7.77-7.70 (m, 2H), 7.50-7.42 (m, 2H), 7.11-7.05 (m, 1H), 6.95 (s, 1H), 4.08-4.02 (m, 1H), 3.75-3.70 (m, 4H), 3.26-3.21 (m, 1H), 3.03-2.95 (m, 4H), 2.82-2.68 (m, 4H), 2.11-2.00 (m, 1H), 1.95-1.75 (m, 2H), 1.61 (br s, 2H), 1.52-1.39 (m, 2H)

### Example 073: N-[4-[4-[6-chloro-4-(trifluoromethyl)-2-pyridyl]piperazin-1-yl]sulfonylphenyl]-4-[2-(3-morpholinopropylamino)ethyl]benzamide

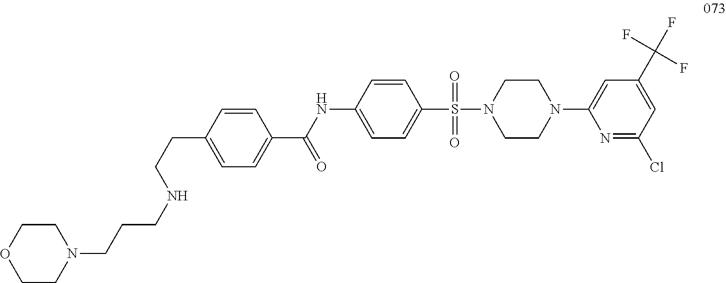 [SMILES: O=C(Nc1ccc(S(=O)(=O)N2CCN(c3cc(C(F)(F)F)cc(Cl)n3)CC2)cc1)c1ccc(CCNCCCN2CCOCC2)cc1]

The title compound was prepared in analogy to the preparation of compound 069b by replacing tert-butyl pyrrolidin-3-ylcarbamate with 3-morpholinopropan-1-amine in step2. Example 073 (10 mg) was obtained as a white solid. MS obsd. (ESI⁺) [(M+H)⁺]: 695.2.

### Example 073: ¹H NMR (400 MHz, DMSO-d6) δ ppm 10.62 (s, 1H), 8.10-8.03 (m, J=8.9 Hz, 2H), 7.97 (d, J=8.3 Hz, 2H), 7.79-7.73 (m, J=8.9 Hz, 2H), 7.48-7.40 (m, 2H), 7.11-7.07 (m, 1H), 6.97 (s, 1H), 4.00 (br s, 2H), 3.82-3.61 (m, 8H), 3.24 (br s, 4H), 3.07-2.95 (m, 10H), 2.00 (br s, 2H). ¹⁹F NMR (376 MHz, DMSO-d6) δ ppm −68.35 (s, 3F)

### Example 074: 4-[2-(2-Amino-5-azaspiro[2.4]heptan-5-yl)ethyl]-N-[4-[4-[6-chloro-4-(trifluoromethyl)-2-pyridyl]piperazin-1-yl]sulfonylphenyl]benzamide

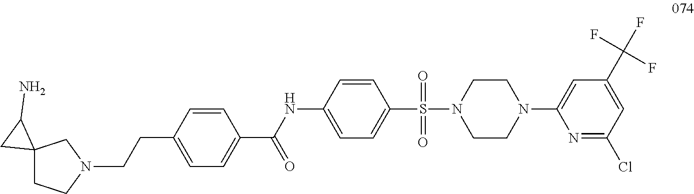 [SMILES: NC1CC12CCN(CCc1ccc(C(=O)Nc3ccc(S(=O)(=O)N4CCN(c5cc(C(F)(F)F)cc(Cl)n5)CC4)cc3)cc1)C2]

The title compound was prepared in analogy to the preparation of Example 069 by replacing tert-butyl pyrrolidin-3-ylcarbamate with tert-butyl (5-azaspiro[2.4]heptan-1-yl)carbamate hydrochloride in step 2. Example 074 (5.5 mg) was obtained as a white solid. MS obsd. (ESI⁺) [(M+H)⁺]: 663.1.

### Example 074: ¹H NMR (400 MHz, DMSO-d6) δ ppm 10.62 (s, 1H), 8.09-8.04 (m, J=8.8 Hz, 2H), 8.00-7.95 (m, 2H), 7.78-7.74 (m, J=8.8 Hz, 2H), 7.49-7.43 (m, 2H), 7.09 (s, 1H), 6.97 (s, 1H), 3.74-3.69 (m, 4H), 3.47-3.43 (m, 2H), 3.25-3.15 (m, 2H), 3.13-3.03 (m, 2H), 2.99 (br t, J=4.6 Hz, 4H), 2.88-2.68 (m, 2H), 2.59-2.52 (m, 1H), 2.03 (br s, 2H), 1.26-1.13 (m, 1H), 1.11-0.97 (m, 1H). ¹⁹F NMR (376 MHz, DMSO-d6) δ ppm −68.42 (s, 3F)

### Example 075: N-[4-[4-[6-chloro-4-(trifluoromethyl)-2-pyridyl]piperazin-1-yl]sulfonylphenyl]-4-[2-(3,6-diazabicyclo[3.1.1]heptan-6-yl)ethyl]benzamide

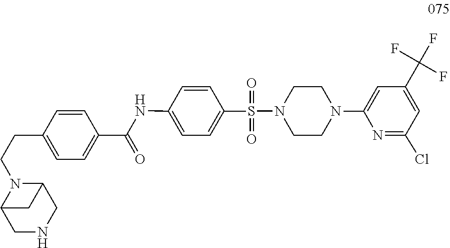 [SMILES: O=C(Nc1ccc(S(=O)(=O)N2CCN(c3cc(C(F)(F)F)cc(Cl)n3)CC2)cc1)c1ccc(CCN2C3CNCC2C3)cc1]

The title compound was prepared in analogy to the preparation of Example 069 by replacing tert-butyl pyrrolidin-3-ylcarbamate with tert-butyl 3,6-diazabicyclo[3.1.1]heptane-3-carboxylate in step2. Example 075 (3 mg) was obtained as a white solid. MS obsd. (ESI⁺) [(M+H)⁺]: 649.1.

### Example 075: ¹H NMR (400 MHz, DMSO-d6) δ ppm 10.62 (s, 1H), 8.09-8.04 (m, J=8.8 Hz, 2H), 7.99-7.94 (m, J=8.2 Hz, 2H), 7.78-7.74 (m, J=8.8 Hz, 2H), 7.50-7.44 (m, J=8.3 Hz, 2H), 7.11-7.08 (m, 1H), 6.98-6.96 (m, 1H), 3.74-3.70 (m, 4H), 3.50-3.38 (m, 8H), 3.01-2.97 (m, 4H), 2.60-2.52 (m, 4H). ¹⁹F NMR (376 MHz, DMSO-d6) δ ppm −68.31 (s, 3F)

### Example 076: N-[4-[4-[6-chloro-4-(trifluoromethyl)-2-pyridyl]piperazin-1-yl]sulfonylphenyl]-3-[(4-piperidylmethylamino)methyl]benzamide

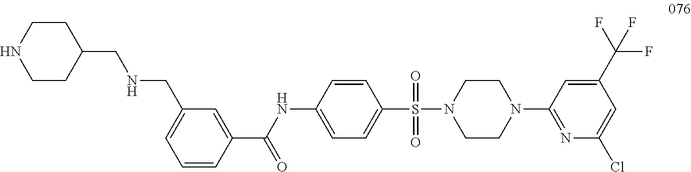 [SMILES: O=C(Nc1ccc(S(=O)(=O)N2CCN(c3cc(C(F)(F)F)cc(Cl)n3)CC2)cc1)c1cccc(CNCC2CCNCC2)c1]

The title compound was prepared in analogy to the preparation of Example 001 by replacing compound tert-butyl N-(3-aminopropyl)carbamate with tert-butyl 4-(aminomethyl)piperidine-1-carboxylate and Int-2 with Int-3 in step 1. Example 076 (10 mg) was obtained as a white solid. MS obsd. (ESI⁺) [(M+H)⁺]: 651.1.

### Example 076: ¹H NMR (CD₃OD, 400 MHz) δ ppm 8.01 (s, 1H), 7.9-8.0 (m, 3H), 7.7-7.7 (m, 2H), 7.65 (d, 1H, J=7.8 Hz), 7.5-7.6 (m, 1H), 6.82 (s, 1H), 6.73 (s, 1H), 4.24 (s, 2H), 3.6-3.7 (m, 4H), 3.34 (br d, 2H, J=13.0 Hz), 2.9-3.0 (m, 8H), 1.9-2.1 (m, 3H), 1.3-1.5 (m, 2H). ¹⁹F NMR (376 MHz, CD₃OD) δ ppm −66.48 (s, 3F)

### Example 077: 3-[[(3-Aminocyclobutyl)methylamino]methyl]-N-[4-[4-[6-chloro-4-(trifluoromethyl)-2-pyridyl]piperazin-1-yl]sulfonylphenyl]benzamide

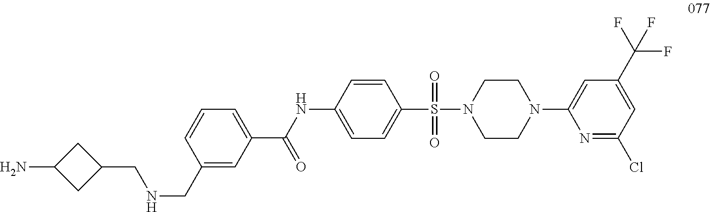 [SMILES: NC1CC(CNCc2cccc(C(=O)Nc3ccc(S(=O)(=O)N4CCN(c5cc(C(F)(F)F)cc(Cl)n5)CC4)cc3)c2)C1]

The title compound was prepared in analogy to the preparation of Example 001 by replacing compound tert-butyl N-(3-aminopropyl)carbamate with tert-butyl N-[(3-aminocyclobutyl)methyl]carbamate and Int-2 with Int-3 in step 1. Example 077 (10 mg) was obtained as a white solid. MS obsd. (ESI⁺) [(M+H)⁺]: 637.1.

### Example 077: ¹H NMR (CD₃OD, 400 MHz) δ ppm 8.0-8.0 (m, 1H), 7.8-8.0 (m, 3H), 7.7-7.8 (m, 2H), 7.6-7.7 (m, 1H), 7.5-7.6 (m, 1H), 6.82 (s, 1H), 6.73 (s, 1H), 4.1-4.3 (m, 2H), 3.6-3.8 (m, 5H), 3.1-3.2 (m, 1H), 3.09 (d, 1H, J=6.8 Hz), 3.0-3.0 (m, 4H), 2.4-2.6 (m, 2H), 2.2-2.4 (m, 2H), 1.8-2.0 (m, 1H). ¹⁹F NMR (376 MHz, CD₃OD) δ ppm −66.48 (s, 3F)

### Example 078: N-[4-[4-[6-chloro-4-(trifluoromethyl)-2-pyridyl]piperazin-1-yl]sulfonylphenyl]-3-[(3-piperazin-1-ylpropylamino)methyl]benzamide

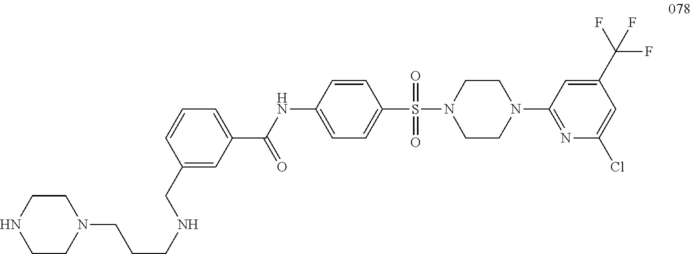 [SMILES: O=C(Nc1ccc(S(=O)(=O)N2CCN(c3cc(C(F)(F)F)cc(Cl)n3)CC2)cc1)c1cccc(CNCCCN2CCNCC2)c1]

The title compound was prepared in analogy to the preparation of Example 001 by replacing compound tert-butyl N-(3-aminopropyl)carbamate with tert-butyl 4-(3-aminopropyl)piperazine-1-carboxylate and Int-2 with Int-3 in step 1. Example 078 (12 mg) was obtained as a white solid. MS obsd. (ESI⁺) [(M+H)⁺]: 680.2.

### Example 078: ¹H NMR (CD₃OD, 400 MHz) δ 8.0-8.0 (m, 1H), 7.9-8.0 (m, 3H), 7.7-7.7 (m, 2H), 7.6-7.7 (m, 1H), 7.5-7.6 (m, 1H), 6.83 (s, 1H), 6.73 (s, 1H), 4.23 (s, 2H), 3.6-3.7 (m, 4H), 3.1-3.2 (m, 4H), 3.1-3.1 (m, 2H), 3.0-3.0 (m, 4H), 2.62 (br s, 4H), 2.49 (t, 2H, J=6.6 Hz), 1.8-1.9 (m, 2H). ¹⁹F NMR (376 MHz, CD₃OD) δ ppm −66.49 (s, 3F)

### Example 079: 3-[[3-(1-Aminocyclopropyl)pyrrolidin-1-yl]methyl]-N-[4-[4-[6-chloro-4-(trifluoromethyl)-2-pyridyl]piperazin-1-yl]sulfonylphenyl]benzamide

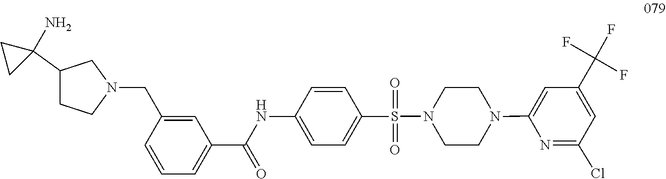 [SMILES: NC1(C2CCN(Cc3cccc(C(=O)Nc4ccc(S(=O)(=O)N5CCN(c6cc(C(F)(F)F)cc(Cl)n6)CC5)cc4)c3)C2)CC1]

The title compound was prepared in analogy to the preparation of Example 001 by replacing tert-butyl N-(3-aminopropyl)carbamate with tert-butyl N-(1-pyrrolidin-3-ylcyclopropyl)carbamate and compound Int-2 with compound Int-3 in step 1. Example 079 (46 mg) was obtained as a white solid. MS obsd. (ESI⁺) [(M+H)⁺]: 663.1.

### Example 079: ¹H NMR (CD3OD, 400 MHz) δ ppm 8.02 (s, 1H), 7.9-8.0 (m, 3H), 7.6-7.8 (m, 3H), 7.5-7.6 (m, 1H), 6.82 (s, 1H), 6.73 (s, 1H), 4.4-4.5 (m, 2H), 3.6-3.7 (m, 4H), 3.5-3.6 (m, 1H), 3.3-3.5 (m, 2H), 3.0-3.0 (m, 4H), 2.9-3.1 (m, 1H), 2.7-2.9 (m, 1H), 2.17 (br dd, 1H, J=5.1, 11.7 Hz), 1.6-1.8 (m, 1H), 0.89 (s, 4H). ¹⁹F NMR (376 MHz, CD₃OD) δ ppm −66.47 (s, 3F)

### Example 080: 3-[[3-[Bis(2-hydroxyethyl)amino]propylamino]methyl]-N-[4-[4-[6-chloro-4-(trifluoromethyl)-2-pyridyl]piperazin-1-yl]sulfonylphenyl]benzamide

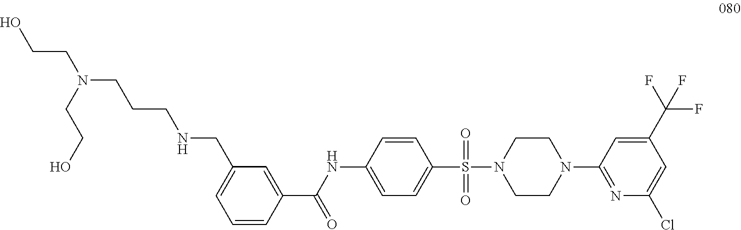 [SMILES: O=C(Nc1ccc(S(=O)(=O)N2CCN(c3cc(C(F)(F)F)cc(Cl)n3)CC2)cc1)c1cccc(CNCCCN(CCO)CCO)c1]

The title compound was prepared in analogy to the step of preparation compound 001a by replacing compound tert-butyl N-(3-aminopropyl)carbamate with 2-[3-aminopropyl(2-hydroxyethyl)amino]ethanol and Int-2 with Int-3. Example 080 (18 mg) was obtained as a white solid. MS obsd. (ESI⁺) [(M+H)⁺]: 699.1.

### Example 080: ¹H NMR (CD₃OD, 400 MHz) δ ppm 8.0-8.0 (m, 1H), 7.9-8.0 (m, 3H), 7.7-7.7 (m, 2H), 7.6-7.7 (m, 1H), 7.5-7.6 (m, 1H), 6.83 (s, 1H), 6.73 (s, 1H), 4.23 (s, 2H), 3.6-3.7 (m, 4H), 3.1-3.2 (m, 4H), 3.1-3.1 (m, 2H), 3.0-3.0 (m, 4H), 2.64-2.60 (m, 4H), 2.49 (t, 2H, J=6.6 Hz), 1.8-1.9 (m, 2H). ¹⁹F NMR (376 MHz, CD₃OD) δ ppm −66.49 (s, 3F)

### Example 081: N-[4-[4-[6-chloro-4-(trifluoromethyl)-2-pyridyl]piperazin-1-yl]sulfonylphenyl]-2-methoxy-5-[[3-(4-piperidyl)propylamino]methyl]benzamide

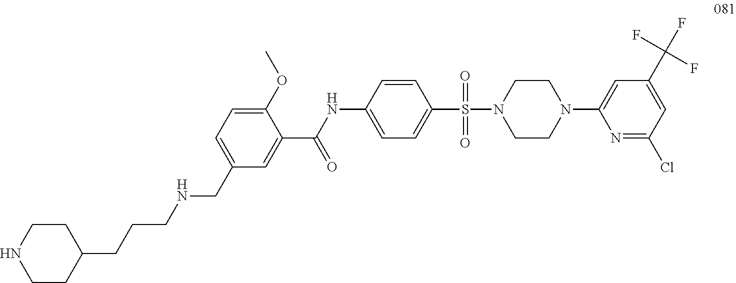 [SMILES: COc1ccc(CNCCCC2CCNCC2)cc1C(=O)Nc1ccc(S(=O)(=O)N2CCN(c3cc(C(F)(F)F)cc(Cl)n3)CC2)cc1]

Step 1: Tert-butyl 4-[3-[[3-[[4-[4-[6-chloro-4-(trifluoromethyl)-2-pyridyl]piperazin-1-yl]sulfonylphenyl]carbamoyl]-4-methoxy-phenyl]methylamino]propyl]piperidine-1-carboxylate

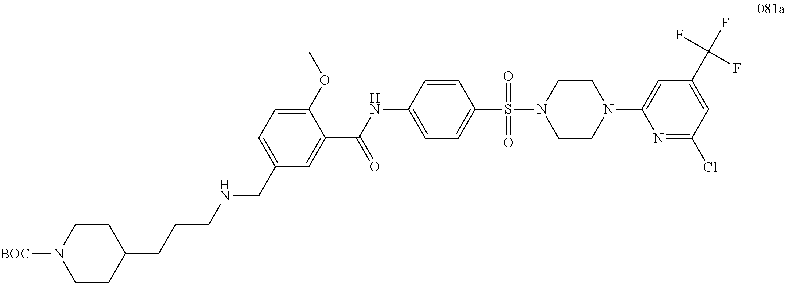 [SMILES: COc1ccc(CNCCCC2CCN(C(=O)OC(C)(C)C)CC2)cc1C(=O)Nc1ccc(S(=O)(=O)N2CCN(c3cc(C(F)(F)F)cc(Cl)n3)CC2)cc1]

A mixture of compound 056a (60 mg, 99.4 μmol), DIPEA (38.6 mg, 298 μmol) and tert-butyl 4-(3-aminopropyl)piperidine-1-carboxylate (48.2 mg, 199 μmol) in THF (5 mL) was stirred at 80° C. overnight. The mixture was diluted with EtOAc (20), washed with water (10 mL). The organic phase was dried over anhydrous Na₂SO₄ and concentrated under vacuum to give crude compound 081a (80 mg, 100%) which was used directly in next step. MS obsd. (ESI⁺) [(M+H)]⁺: 809.0

Step 2: N-[4-[4-[6-chloro-4-(trifluoromethyl)-2-pyridyl]piperazin-1-yl]sulfonylphenyl]-2-methoxy-5-[[3-(4-piperidyl)propylamino]methyl]benzamide

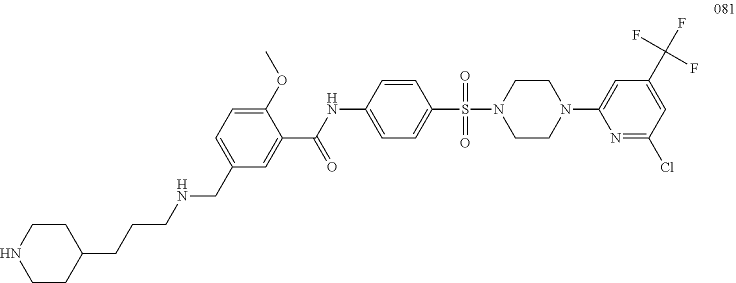 [SMILES: COc1ccc(CNCCCC2CCNCC2)cc1C(=O)Nc1ccc(S(=O)(=O)N2CCN(c3cc(C(F)(F)F)cc(Cl)n3)CC2)cc1]

A mixture of compound 081a (80 mg, 98.8 μmol) in TFA (1 mL) was stirred at rt for 2 hours. The mixture was concentrated under vacuum to give residue which was purified by HPLC (TFA as additive) to give Example 081 (3 mg, 4%) as white solid. MS obsd. (ESI⁺) [(M+H)]⁺: 709.1.

### Example 081: ¹H NMR (400 MHz, DMSO-d6) δ ppm 10.57-10.53 (m, 1H), 8.73-8.69 (m, 1H), 8.01-7.95 (m, 2H), 7.78-7.74 (m, 4H), 7.11-7.07 (m, 1H), 6.99-6.96 (m, 1H), 4.17-4.14 (m, 1H), 3.92-3.91 (m, 3H), 3.73-3.71 (m, 4H), 3.00-2.99 (m, 4H), 2.89-2.86 (m, 4H), 1.79-1.76 (m, 2H), 1.62-1.56 (m, 5H), 1.27-1.23 (m, 5H). ¹⁹F NMR (376 MHz, DMSO-d6) δ ppm −68.46 (s, 3F)

### Example 082: 3-[[1-(Aminomethyl)-3-azabicyclo[3.1.0]hexan-3-yl]methyl]-N-[4-[4-[6-chloro-4-(trifluoromethyl)-2-pyridyl]piperazin-1-yl]sulfonylphenyl]benzamide

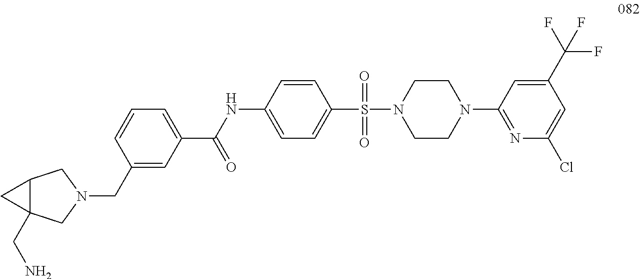 [SMILES: NCC12CC1CN(Cc1cccc(C(=O)Nc3ccc(S(=O)(=O)N4CCN(c5cc(C(F)(F)F)cc(Cl)n5)CC4)cc3)c1)C2]

The title compound was prepared in analogy to the preparation of Example 001 by replacing compound tert-butyl N-(3-aminopropyl)carbamate with tert-butyl N-(3-azabicyclo[3.1.0]hexan-1-ylmethyl)carbamate and compound Int-2 with compound Int-3 in step 1. Example 082 (26.1 mg) was obtained as a white solid. MS obsd. (ESI⁺) [(M+H)⁺]: 649.2.

### Example 082: ¹H NMR (CD₃OD, 400 MHz) δ 8.0-8.0 (m, 1H), 7.96 (d, 1H, J=7.8 Hz), 7.9-7.9 (m, 2H), 7.7-7.7 (m, 2H), 7.64 (d, 1H, J=7.9 Hz), 7.5-7.6 (m, 1H), 6.82 (s, 1H), 6.73 (s, 1H), 4.3-4.5 (m, 2H), 3.6-3.7 (m, 4H), 3.4-3.6 (m, 4H), 3.32 (d, 1H, J=13.8 Hz), 2.9-3.1 (m, 5H), 1.89 (br s, 1H), 0.9-1.3 (m, 2H). ¹⁹F NMR (376 MHz, CD₃OD) δ ppm −66.48 (s, 3F)

### Example 083: (2R)-2-Amino-4-[[3-[[4-[4-[6-chloro-4-(trifluoromethyl)-2-pyridyl]piperazin-1-yl]sulfonylphenyl]carbamoyl]phenyl]methylamino]butanoic acid

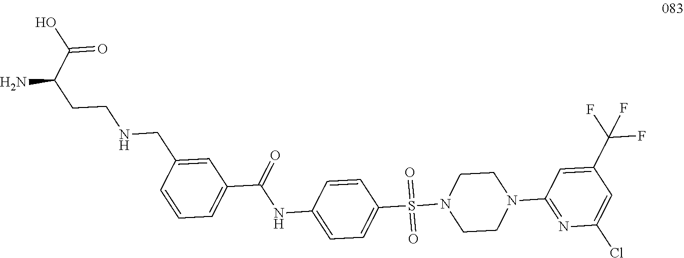 [SMILES: N[C@H](CCNCc1cccc(C(=O)Nc2ccc(S(=O)(=O)N3CCN(c4cc(C(F)(F)F)cc(Cl)n4)CC3)cc2)c1)C(=O)O]

The title compound was prepared in analogy to the preparation of Example 001 by replacing compound tert-butyl N-(3-aminopropyl)carbamate with (2R)-4-amino-2-(tert-butoxycarbonylamino)butanoic acid and compound Int-2 with compound Int-3 in step 1. Example 084 (2 mg) was obtained as a white solid. MS obsd. (ESI⁺) [(M+H)⁺]: 655.1.

### Example 083: ¹H NMR (CD₃OD, 400 MHz) δ ppm 8.11 (t, 1H, J=1.5 Hz), 8.0-8.1 (m, 3H), 7.8-7.9 (m, 2H), 7.7-7.8 (m, 1H), 7.6-7.7 (m, 1H), 6.94 (s, 1H), 6.85 (s, 1H), 4.36 (s, 2H), 4.09 (dd, 1H, J=5.2, 8.4 Hz), 3.7-3.8 (m, 4H), 3.3-3.5 (m, 2H), 3.1-3.2 (m, 4H), 2.2-2.5 (m, 2H). ¹⁹F NMR (376 MHz, CD₃OD) δ ppm −66.49 (s, 3F)

### Example 084: N-[4-[4-[6-chloro-4-(trifluoromethyl)-2-pyridyl]piperazin-1-yl]sulfonylphenyl]-3-[[(2-oxooxazolidin-4-yl)methylamino]methyl]benzamide

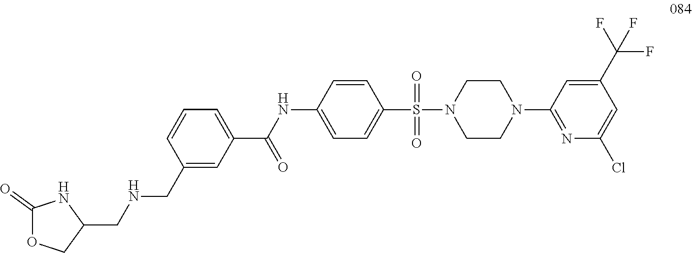 [SMILES: O=C1NC(CNCc2cccc(C(=O)Nc3ccc(S(=O)(=O)N4CCN(c5cc(C(F)(F)F)cc(Cl)n5)CC4)cc3)c2)CO1]

The title compound was prepared in analogy to the step of preparation compound 001a by replacing tert-butyl N-(3-aminopropyl)carbamate with 4-(aminomethyl)oxazolidin-2-one and compound Int-2 with compound Int-3 in step 1. Example 084 (20 mg) was obtained as a white solid. MS obsd. (ESI⁺) [(M+H)⁺]: 653.1.

### Example 084: ¹H NMR (CD₃OD, 400 MHz) δ ppm 7.8-8.0 (m, 2H), 7.7-7.8 (m, 2H), 7.7-7.7 (m, 2H), 7.3-7.5 (m, 2H), 6.83 (s, 1H), 6.73 (s, 1H), 4.3-4.4 (m, 2H), 3.6-3.7 (m, 5H), 3.3-3.4 (m, 3H), 3.10 (dd, 1H, J=5.7, 9.1 Hz), 3.0-3.0 (m, 4H). ¹⁹F NMR (376 MHz, CD₃OD) δ ppm −66.49 (s, 3F)

### Example 085: N-[4-[4-[6-chloro-4-(trifluoromethyl)-2-pyridyl]piperazin-1-yl]sulfonylphenyl]-3-[[(5-oxopyrrolidin-3-yl)methylamino]methyl]benzamide

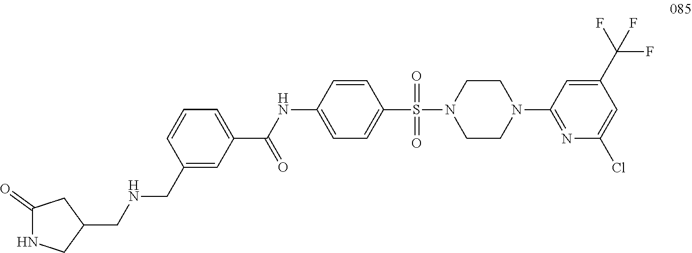 [SMILES: O=C1CC(CNCc2cccc(C(=O)Nc3ccc(S(=O)(=O)N4CCN(c5cc(C(F)(F)F)cc(Cl)n5)CC4)cc3)c2)CN1]

The title compound was prepared in analogy to the preparation of compound 001a in step 1 by replacing compound tert-butyl N-(3-aminopropyl)carbamate with 4-(aminomethyl)pyrrolidin-2-one and compound Int-2 with compound Int-3. Example 085 (32 mg) was obtained as a white solid. MS obsd. (ESI⁺) [(M+H)⁺]: 651.2.

### Example 085: ¹H NMR (CD₃OD, 400 MHz) δ ppm 8.01 (s, 1H), 7.9-8.0 (m, 1H), 7.9-7.9 (m, 2H), 7.7-7.8 (m, 2H), 7.65 (d, 1H, J=7.8 Hz), 7.5-7.6 (m, 1H), 6.83 (s, 1H), 6.73 (s, 1H), 4.24 (s, 2H), 3.6-3.7 (m, 4H), 3.50 (dd, 1H, J=7.9, 10.1 Hz), 3.15 (dd, 2H, J=3.6, 7.3 Hz), 3.08 (dd, 1H, J=6.6, 10.1 Hz), 3.0-3.0 (m, 4H), 2.79 (quind, 1H, J=7.7, 15.3 Hz), 2.44 (dd, 1H, J=8.8, 16.9 Hz), 2.12 (dd, 1H, J=7.7, 16.9 Hz). ¹⁹F NMR (376 MHz, CD₃OD) δ ppm −66.49 (s, 3F)

### Example 086: N-[4-[4-[6-chloro-4-(trifluoromethyl)-2-pyridyl]piperazin-1-yl]sulfonylphenyl]-3-[[[3-(dimethylamino)-3-oxo-propyl]amino]methyl]benzamide

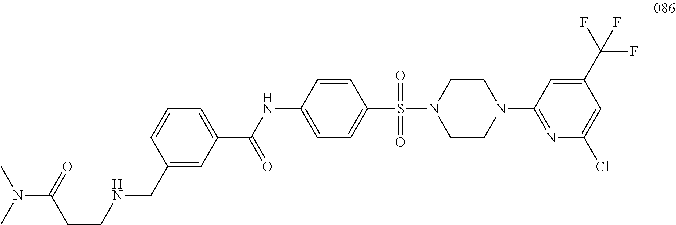 [SMILES: CN(C)C(=O)CCNCc1cccc(C(=O)Nc2ccc(S(=O)(=O)N3CCN(c4cc(C(F)(F)F)cc(Cl)n4)CC3)cc2)c1]

The title compound was prepared in analogy to the preparation compound 001a by replacing compound tert-butyl N-(3-aminopropyl)carbamate with 3-amino-N,N-dimethyl-propanamide and compound Int-2 with compound Int-3. Example 086 (32 mg) was obtained as a white solid. MS obsd. (ESI⁺) [(M+H)⁺]: 653.1.

### Example 086: ¹H NMR (CD₃OD, 400 MHz) δ ppm 8.00 (s, 1H), 7.9-8.0 (m, 3H), 7.71 (d, 2H, J=8.8 Hz), 7.65 (d, 1H, J=7.8 Hz), 7.5-7.6 (m, 1H), 6.82 (s, 1H), 6.73 (s, 1H), 4.26 (s, 2H), 3.6-3.7 (m, 4H), 3.0-3.0 (m, 4H), 2.94 (s, 3H), 2.86 (s, 3H), 2.8-2.8 (m, 2H), 2.73 (t, 2H, J=6.2 Hz). ¹⁹F NMR (376 MHz, CD₃OD) δ ppm −66.48 (s, 3F)

### Example 087: 5-(3,3a,4,5,6,6a-Hexahydro-2H-pyrrolo[3,4-b]pyrrol-1-ylmethyl)-N-[4-[4-[6-chloro-4-(trifluoromethyl)-2-pyridyl]piperazin-1-yl]sulfonylphenyl]-2-methoxy-benzamide

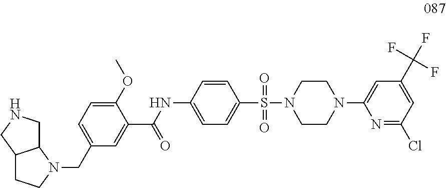 [SMILES: COc1ccc(CN2CCC3CNCC32)cc1C(=O)Nc1ccc(S(=O)(=O)N2CCN(c3cc(C(F)(F)F)cc(Cl)n3)CC2)cc1]

The title compound was prepared in analogy to the preparation of Example 081 by replacing tert-butyl 4-(3-aminopropyl)piperidine-1-carboxylate with tert-butyl 2,3,3a,4,6,6a-hexahydro-1H-pyrrolo[2,3-c]pyrrole-5-carboxylate in step 1. Example 087 (20 mg) was obtained as a white solid. MS obsd. (ESI⁺) [(M+H)⁺]: 679.0.

### Example 087: ¹H NMR (400 MHz, DMSO-d6) δ ppm 10.46 (s, 1H), 7.91 (d, J=8.7 Hz, 2H), 7.66 (d, J=8.9 Hz, 2H), 7.49 (d, J=2.1 Hz, 1H), 7.38 (dd, J=2.2, 8.4 Hz, 1H), 7.06 (d, J=8.6 Hz, 1H), 7.02 (s, 1H), 6.90 (s, 1H), 3.83-3.76 (m, 4H), 3.68-3.61 (m, 4H), 3.38-3.36 (m, 2H), 3.13-3.02 (m, 3H), 2.93-2.89 (m, 4H), 2.79-2.63 (m, 3H), 2.12-1.99 (m, 1H), 1.96-1.85 (m, 1H), 1.43-1.32 (m, 1H). ¹⁹F NMR (376 MHz, DMSO-d6) δ ppm −68.53 (s, 3F)

### Example 088: 2-[(3-Aminopropylamino)methyl]-N-[4-[4-[6-chloro-4-(trifluoromethyl)-2-pyridyl]piperazin-1-yl]sulfonylphenyl]benzamide

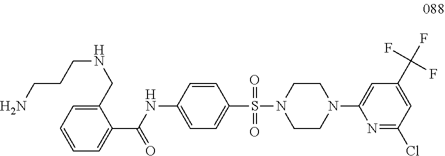 [SMILES: NCCCNCc1ccccc1C(=O)Nc1ccc(S(=O)(=O)N2CCN(c3cc(C(F)(F)F)cc(Cl)n3)CC2)cc1]

Step 1: 2-(Chloromethyl)-N-[2-(chloromethyl)benzoyl]-N-[4-[4-[6-chloro-4-(trifluoromethyl)-2-pyridyl]piperazin-1-yl]sulfonylphenyl]benzamide

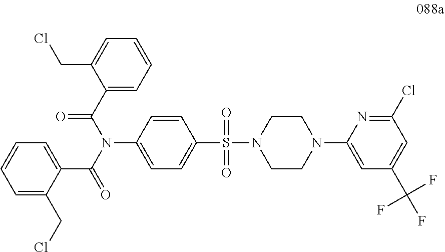 [SMILES: O=C(c1ccccc1CCl)N(C(=O)c1ccccc1CCl)c1ccc(S(=O)(=O)N2CCN(c3cc(C(F)(F)F)cc(Cl)n3)CC2)cc1]

To a mixture of compound Int-1 (400 mg, 950 μmol) and TEA (288 mg, 2.85 mmol) in DCM (5 mL) was added 2-(chloromethyl)benzoyl chloride (216 mg, 1.14 mmol) dropwise at rt and the mixture was stirred at rt for 2 hours. After completion, the mixture was quenched with saturated NaHCO₃ aq. (40 mL), extracted with DCM (30 mL×3). The combined organic layer was concentrated under vacuum to give crude, which was purified by silica-gel chromatography (elute with EtOAc:PE=10%) to afford compound 088a (200 mg, 29% yield) as white solid. MS obsd. (ESI⁺) [(M+H)⁺]: 725.2.

Step 2: Tert-butyl N-[3-[[2-[[4-[4-[6-chloro-4-(trifluoromethyl)-2-pyridyl]piperazin-1-yl]sulfonylphenyl]carbamoyl]phenyl]methylamino]propyl]carbamate

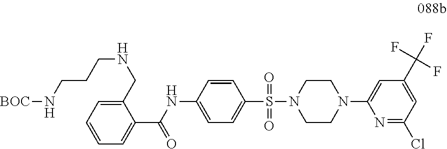 [SMILES: CC(C)(C)OC(=O)NCCCNCc1ccccc1C(=O)Nc1ccc(S(=O)(=O)N2CCN(c3cc(C(F)(F)F)cc(Cl)n3)CC2)cc1]

A mixture of compound 089a (120 mg, 165 μmol), tert-butyl (3-aminopropyl)carbamate (43.2 mg, 248 μmol) and DIEA (64 mg, 496 μmol) in THF (5 mL) was heated at 70° C. for 10 hours. After completion, the mixture was diluted with DCM (20 mL), washed with NaHCO₃ aq. (20 mL). The organic layer was dried over Na₂SO₄ and concentrated under vacuum to give crude compound 088b (118 mg, 100% yield), which was used in the next step directly. MS obsd. (ESI⁺) [(M+H)⁺]: 711.2.

Step 3: 2-[(3-Aminopropylamino)methyl]-N-[4-[4-[6-chloro-4-(trifluoromethyl)-2-pyridyl]piperazin-1-yl]sulfonylphenyl]benzamide

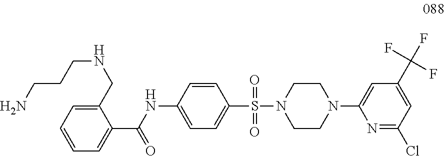 [SMILES: NCCCNCc1ccccc1C(=O)Nc1ccc(S(=O)(=O)N2CCN(c3cc(C(F)(F)F)cc(Cl)n3)CC2)cc1]

A crude solution of compound 088 (70 mg, 98.4 μmol) in TFA (10 mL) was stirred at rt for 2 hour. And then, the reaction mixture was concentrated and the residue was purified by prep-HPLC (TFA as additive) to afford Example 088 (10 mg, 15.8% yield). MS obsd. (ESI⁺) [(M+H)⁺]: 611.1.

### Example 088: ¹H NMR (CD₃OD, 400 MHz) δ ppm 7.9-7.9 (m, 2H), 7.8-7.9 (m, 1H), 7.7-7.7 (m, 2H), 7.5-7.6 (m, 3H), 6.83 (s, 1H), 6.74 (s, 1H), 4.20 (s, 2H), 3.6-3.7 (m, 4H), 3.1-3.2 (m, 2H), 2.9-3.0 (m, 6H), 2.05 (quin, 2H, J=7.7 Hz). ¹⁹F NMR (376 MHz, CD₃OD) δ ppm −66.48 (s, 3F)

Compound F-1, disclosed in the literature (Bioorganic Chemistry 102 (2020) 104055) and reported having LPS synthesis pathway inhibition activity and displaying antibiotic activity against efflux-deficient E. coli strains, was chosen as reference compound in present invention.

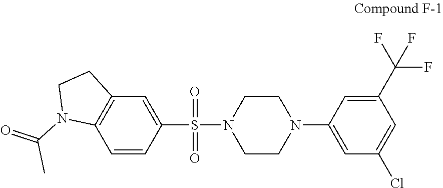 [SMILES: CC(=O)N1CCc2cc(S(=O)(=O)N3CCN(c4cc(Cl)cc(C(F)(F)F)c4)CC3)ccc21]

### BIOLOGICAL EXAMPLES

### Example 089: Minimal Inhibitory Concentration Protocol (MIC) Assay

The antibacterial activity of the compounds of the present invention was evaluated against the commonly used quality control strain Escherichia coli ATCC 25922 and the rifampin-resistant mutant strain Klebsiella pneumonia ATCC 43816. Both were originally derived from human clinical samples and are available from ATCC (American Type Culture Collection). The in vitro potency of compounds to inhibit Escherichia coli (ATCC 25922) and Klebsiella pneumonia (ATCC 43816) growth was assessed by the MIC (Minimal Inhibitory Concentration) broth dilution method. Specifically compound dilutions were prepared from 10 mM DMSO stock solutions as follows: i) serial 2-fold dilution in 20 μL DMSO were prepared in a master plate (Greiner, Cat No: 651201), ii) 180 μL sterile distilled water was added to each aliquot and iii) 10 μL diluted compounds were transferred into a new assay plate (Costar, 3599).

Vials of each test microorganisms were maintained frozen in the vapor phase of a liquid nitrogen freezer. Single-use frozen vials of the two strains Escherichia coli ATCC 25922 (KWIKSTIK, 0335K) and Klebsiella pneumonia ATCC 43816, with predetermined CFU/mL, were taken out from the freezer, thawed at room temperature, and diluted in Cation-Adjusted Mueller Hinton Broth (CAMHB) to achieve a final inoculum of 5×10⁵ CFU/mL. 90 μL bacteria containing broth was dispensed to the assay plate containing the pre-dispensed compound dilutions and mixed by pipetting 5 times.

Then the assay plates were incubated for 20 hours at 35° C. in ambient air. Following incubation, the MIC (pag/mL), the lowest concentration of drug that inhibits visible growth of the microorganism was read with the help of a magnification mirror and recorded.

TABLE 1
MIC values of the compounds of this invention
against E. coli and K. pneumonia

MIC (μg/mL)

| | Escherichia | Klebsiella |
|---|---|---|
| Example | coli ATCC | pneumoniae |
| No. | 25922 | ATCC43816 |
| F-1 | >24 | >24 |
| 001 | 0.51 | 1.5 |
| 002 | 1.8 | 5.4 |
| 003 | 1.4 | 3.8 |
| 004 | 1.9 | 3.8 |
| 005 | 1.9 | 7.7 |
| 006 | 3.7 | 15 |
| 007 | 0.97 | 0.97 |
| 008 | N.A. | 7.7 |
| 009 | 5.4 | 11 |
| 010 | 5.3 | 11 |
| 011 | 3.8 | 7.6 |
| 012 | 1.8 | 3.6 |
| 013 | 0.94 | N.A. |
| 014 | 15 | 30 |
| 015 | 1.9 | 3.8 |
| 016 | 1.9 | 3.8 |
| 017 | 0.97 | 0.97 |
| 018 | 0.64 | 2.6 |
| 019 | 2.6 | 5.2 |
| 020 | 1.2 | 2.5 |
| 021 | 3.9 | 7.8 |
| 022 | 3.9 | 3.9 |
| 023 | 4.2 | 4.2 |
| 024 | 0.48 | 0.96 |
| 025 | 1.3 | 5.1 |
| 026 | 3.9 | 7.8 |
| 027 | 4 | 4 |
| 028 | 4.6 | 9.3 |
| 029 | 1.9 | 3.8 |
| 030 | 4 | 8 |
| 031 | 3.9 | 3.9 |
| 032 | 7.1 | 7.1 |
| 033 | 3.6 | 3.6 |
| 034 | 0.92 | 0.92 |
| 035 | 3.7 | 7.5 |
| 036 | 3.7 | 7.4 |
| 037 | 3.7 | 7.5 |
| 038 | 3.7 | 7.4 |
| 040 | 2.1 | 8.3 |
| 041 | 1 | 8.3 |
| 042 | 0.99 | 2 |
| 043 | 4.2 | 2.1 |
| 044 | 0.91 | 1.8 |
| 045 | 0.91 | 3.6 |
| 046 | 0.47 | 0.47 |
| 047 | 1.1 | 2.3 |
| 048 | 0.48 | 0.97 |
| 050 | 1 | 1 |
| 051 | 0.56 | 1.1 |
| 052 | 1.1 | 2.3 |
| 053 | 0.44 | 0.89 |
| 054 | 0.48 | 0.95 |
| 055 | 0.45 | 0.91 |
| 056 | 3.8 | 3.8 |
| 057 | 1 | 2 |
| 058 | 3.8 | 3.8 |
| 059 | 1.9 | 3.8 |
| 060 | 0.48 | 1.9 |
| 061 | 0.53 | 1.1 |
| 062 | 2.1 | 4.2 |
| 063 | 4.3 | 4.3 |
| 064 | 2.1 | 4.2 |
| 065 | 0.26 | 0.52 |
| 066 | 0.51 | 1 |
| 067 | 0.51 | 1 |
| 068 | 1 | 2.1 |
| 069 | 1.2 | 2.3 |
| 070 | 1.2 | 2.4 |
| 071 | 4.2 | 4.2 |
| 072 | 1 | 2 |
| 073 | 1.4 | 2.9 |
| 074 | 2.8 | 2.8 |
| 075 | 2.7 | 5.5 |
| 076 | 0.69 | 2.7 |
| 077 | 0.68 | 2.7 |
| 078 | 1.4 | 5.7 |
| 079 | 1.1 | 2.2 |
| 080 | 1.4 | 2.9 |
| 081 | 1.5 | 2.9 |
| 082 | 0.69 | 1.4 |
| 084 | 4.8 | 4.8 |
| 085 | 1.2 | N.A. |
| 086 | 2.4 | N.A. |
| 087 | 1.2 | 2.5 |
| 088 | 1.3 | 2.6 |

### Example 090: Human Microsome Stability Assay

The human microsomal stability assay is used for early assessment of metabolic stability of a test compound in human liver microsomes.

Human liver microsomes (Cat.NO.: 452117, Corning, USA) were pre-incubated with test compound for 10 minutes at 37° C. in 100 mM potassium phosphate buffer, pH 7.4. The reactions were initiated by adding NADPH regenerating system. The final incubation mixtures contained 1 μM test compound, 0.5 mg/mL liver microsomal protein, 1 mM MgCl₂, 1 mM NADP, 1 unit/mL isocitric dehydrogenase and 6 mM isocitric acid in 100 mM potassium phosphate buffer, pH 7.4. After incubation times of 0, 3, 6, 9, 15 and 30 minutes at 37° C., 300 μL of cold acetonitrile (including internal standard) was added to 100 μL incubation mixture to terminate the reaction. Following precipitation and centrifugation, the amount of compound remaining in the samples were determined by LC-MS/MS. Controls of no NADPH regenerating system at zero and 30 minutes were also prepared and analyzed. The compounds of present invention showed good human liver microsome stability determined in the above assay, results are shown in Table 2 below. (Human microsome clearance: <7 mL/min/kg (low); 7-16.2 mL/min/kg (medium); >16.2 mL/min/kg (high))

TABLE 2
Human microsome clearance values of
the compounds of this invention

| | Clearance of |
|---|---|
| Example | Human microsome |
| No. | (mL/min/kg) |
| F-1 | 14 |
| 001 | 6.2 |
| 012 | 6.2 |
| 020 | 8.8 |
| 034 | 6.2 |
| 039 | 9.4 |
| 041 | 6.2 |
| 043 | 6.2 |
| 068 | 6.2 |
| 078 | 6.2 |

1. A compound of formula (I), 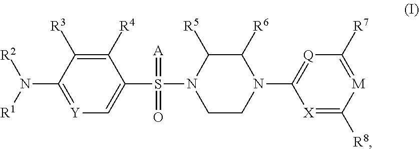
wherein
R¹ is COR⁹; wherein R⁹ is selected from the group consisting of
((hydroxyC₁₋₆alkyl)amino)C₁₋₆alkyl,
1-oxa-2,9-diazaspiro[4.5]dec-2-enyl,
4,5,6,7-tetrahydropyrazolo[1,5-a]pyrazinyl,
4,6-dihydropyrrolo[3,4-c]pyrazolyl substituted by C₁₋₆alkyl,
6,7-dihydro-5H-pyrrolo[3,4-b]pyridinyl,
indanyl substituted by amino or aminoC₁₋₆alkylamino,
isoindolinyl,
piperazinylC₁₋₆alkyl,
pyrazolo[1,5-a]pyrimidinyl,
pyrazolyl substituted once or twice by the substituents independently selected from aminoC₁₋₆alkyl, azetidinyl, C₁₋₆alkyl and carboxy,
pyridinyl substituted by (aminoC₁₋₆alkylamino)C₁₋₆alkyl, and
phenyl substituted once or twice by the substituents independently selected from ((amino(carboxy)C₁₋₆alkyl)amino)C₁₋₆alkyl, ((aminoC₁₋₆alkyl)-3-azabicyclo[3.1.0]hexanyl)C₁₋₆alkyl, ((aminoC₁₋₆alkyl)pyrrolidinyl)C₁₋₆alkyl, ((aminoC₃₋₇ cycloalkyl)C₁₋₆alkylamino)C₁₋₆alkyl, ((aminoC₃₋₇cycloalkyl)pyrrolidinyl)C₁₋₆alkyl, ((oxooxazolidinyl)C₁₋₆alkylamino)C₁₋₆alkyl, ((oxopyrrolidinyl)C₁₋₆alkylamino)C₁₋₆alkyl, ((piperidinyl)C₁₋₆alkylamino)C₁₋₆alkyl, (3,3a,4,5,6,6a-hexahydro-2H-pyrrolo[3,4-b]pyrrolyl)C₁₋₆alkyl, (3,6-diazabicyclo[3.1.1]heptanyl)C₁₋₆alkyl, (amino-2-azaspiro[3.3]heptanyl)C₁₋₆alkyl, (amino-5-azaspiro[2.4]heptanyl)C₁₋₆alkyl, (aminoC₁₋₆alkyl)piperazinyl, (aminoC₁₋₆alkyl)pyrrolidinyl, (aminoC₁₋₆alkylamino)C₁₋₆alkyl, (aminopyrrolidinyl)C₁₋₆alkyl, (C₁₋₆alkyl-2,3,3a,4,6,6a-hexahydropyrrolo[2,3-c]pyrrolyl)C₁₋₆alkyl, (diaminoC₁₋₆alkylcarbonyl)amino, (morpholinylC₁₋₆alkylamino)C₁₋₆alkyl, (piperazinylC₁₋₆alkylamino)C₁₋₆alkyl, (piperidinylC₁₋₆alkylamino)C₁₋₆alkyl, [((C₁₋₆alkyl)₂aminocarbonylC₁₋₆alkyl)amino]C₁₋₆alkyl, [((hydroxyC₁₋₆alkyl)₂amino)C₁₋₆alkylamino]C₁₋₆alkyl, 2,5-diazabicyclo[2.2.1]heptanyl, 3,6-diazabicyclo[3.2.0]heptanyl, aminoC₁₋₆alkoxy, aminoC₁₋₆alkyl, aminopyrrolidinyl, azetidinylC₁₋₆alkoxy, azetidinylC₁₋₆alkylamino, C₁₋₆alkylcarbonyloxy, C₁₋₆alkoxy, carboxy, hydroxy and piperazinyl;
R² is H;
R³ is H, hydroxy or C₁₋₆alkoxy, or
R² and R³ together with the atoms they are attached to form a pyrrolidinyl;
R⁴ is H, hydroxy or C₁₋₆alkoxy;
R⁵ is H or C₁₋₆alkyl,
R⁶ is H or C₁₋₆alkyl, or
R⁵ and R⁶ together with the atoms they are attached to form a C₃₋₇cycloalkyl;
R⁷ is halogen, cyano, haloC₁₋₆alkyl or C₁₋₆alkoxycarbonyl;
R⁸ is H, halogen, cyano, amino, C₃₋₇cycloalkoxy, C₁₋₆alkyl, C₁₋₆alkoxy or haloC₁₋₆alkyl;
Y is CH or N;
A is O, N(CN), or N(C₁₋₆alkyl),
Q is CH or N;
X is CH or N; and
M is N or CR¹⁰; wherein R¹⁰ is H, halogen, C₁₋₆alkyl or C₃₋₇cycloalkyl;
with the proviso that Q, X and M are not N simultaneously;
or a pharmaceutically acceptable salt thereof.

2. A compound according to claim 1, wherein
R¹ is COR⁹; wherein R⁹ is selected from the group consisting of
6,7-dihydro-5H-pyrrolo[3,4-b]pyridinyl,
indanyl substituted by amino or aminoC₁₋₆alkylamino,
isoindolinyl,
pyrazolo[1,5-a]pyrimidinyl,
pyridinyl substituted by (aminoC₁₋₆alkylamino)C₁₋₆alkyl, and
phenyl substituted once or twice by the substituents independently selected from ((aminoC₁₋₆alkyl)-3-azabicyclo[3.1.0]hexanyl)C₁₋₆alkyl, (aminoC₁₋₆alkyl)pyrrolidinyl, (aminoC₁₋₆alkylamino)C₁₋₆alkyl, aminoC₁₋₆alkoxy, aminoC₁₋₆alkyl, aminopyrrolidinyl, azetidinylC₁₋₆alkoxy, carboxy and piperazinyl.

3. A compound according to claim 1, wherein
R¹ is COR⁹, wherein R⁹ is selected from the group consisting of
6,7-dihydro-5H-pyrrolo[3,4-b]pyridinyl, aminoindanyl, (3-aminopropylamino)indanyl, isoindolinyl, pyrazolo[1,5-a]pyrimidinyl, ((3-aminopropylamino)methyl)pyridinyl, ((3-aminopropylamino)methyl)phenyl, ((3-aminopropylamino)ethyl)phenyl, carboxyphenyl, (aminoethoxy)phenyl, (azetidin-3-ylmethoxy)phenyl, (aminopyrrolidin-1-yl)phenyl, (aminoethyl)phenyl, (piperazin-1-yl)phenyl, (3-aminopropoxy)₂phenyl, (1-(3-aminopropyl)pyrrolidin-2-yl)phenyl, (1-(3-aminopropyl)pyrrolidin-3-yl)phenyl; and [[1-(aminomethyl)-3-azabicyclo[3.1.0]hexan-3-yl)methyl]phenyl.

4. A compound according to claim 1, wherein R² is H; R³ is H; or R² and R³ together with the atoms they are attached to form a pyrrolidinyl.

5. A compound according to claim 1, wherein R⁴ is H; R⁵ is H; and R⁶ is H.

6. A compound according to claim 1, wherein R⁷ is halogen or haloC₁₋₆alkyl.

7. A compound according to claim 1, wherein R⁷ is bromo, trifluoromethyl or difluoroethyl.

8. A compound according to claim 1, wherein R⁸ is halogen or C₁₋₆alkyl.

9. A compound according to claim 1, wherein R⁸ is chloro or methyl.

10. A compound according to claim 1, wherein Y is CH.

11. A compound according to claim 1, wherein A is O.

12. A compound according to claim 1, wherein X is N.

13. A compound according to claim 1, wherein M is CH.

14. A compound according to claim 1, wherein
R¹ is COR⁹; wherein R⁹ is selected from the group consisting of
6,7-dihydro-5H-pyrrolo[3,4-b]pyridinyl,
indanyl substituted by amino or aminoC₁₋₆alkylamino,
isoindolinyl,
pyrazolo[1,5-a]pyrimidinyl,
pyridinyl substituted by (aminoC₁₋₆alkylamino)C₁₋₆alkyl, and
phenyl substituted once or twice by the substituents independently selected from ((aminoC₁₋₆alkyl)-3-azabicyclo[3.1.0]hexanyl)C₁₋₆alkyl, (aminoC₁₋₆alkyl)pyrrolidinyl, (aminoC₁₋₆alkylamino)C₁₋₆alkyl, aminoC₁₋₆alkoxy, aminoC₁₋₆alkyl, aminopyrrolidinyl, azetidinylC₁₋₆alkoxy, carboxy and piperazinyl;
R² is H,
R³ is H, or
R² and R³ together with the atoms they are attached to form a pyrrolidinyl;
R⁴ is H;
R⁵ is H;
R⁶ is H;
R⁷ is halogen or haloC₁₋₆alkyl;
R⁸ is halogen or C₁₋₆alkyl;
Y is CH;
A is O;
Q is CH or N;
X is N;
M is CH;
or a pharmaceutically acceptable salt thereof.

15. A compound according to claim 14, wherein
R¹ is COR⁹, wherein R⁹ is 6,7-dihydro-5H-pyrrolo[3,4-b]pyridinyl, aminoindanyl, (3-aminopropylamino)indanyl, isoindolinyl, pyrazolo[1,5-a]pyrimidinyl, ((3-aminopropylamino)methyl)pyridinyl, ((3-aminopropylamino)methyl)phenyl, ((3-aminopropylamino)ethyl)phenyl, carboxyphenyl, (aminoethoxy)phenyl, (azetidin-3-ylmethoxy)phenyl, (aminopyrrolidin-1-yl)phenyl, (aminoethyl)phenyl, (piperazin-1-yl)phenyl, (3-aminopropoxy)₂phenyl, (1-(3-aminopropyl)pyrrolidin-2-yl)phenyl, (1-(3-aminopropyl)pyrrolidin-3-yl)phenyl or [[1-(aminomethyl)-3-azabicyclo[3.1.0]hexan-3-yl)methyl]phenyl;
R² is H,
R³ is H, or
R² and R³ together with the atoms they are attached to form a pyrrolidinyl;
R⁴ is H;
R⁵ is H;
R⁶ is H;
R⁷ is bromo, trifluoromethyl or difluoroethyl;
R⁸ is chloro or methyl;
Y is CH;
A is O;
Q is CH or N;
X is N;
M is CH;
or a pharmaceutically acceptable salt thereof.

16. A compound selected from:
4-[(3-Aminopropylamino)methyl]-N-[4-[4-[6-chloro-4-(trifluoromethyl)-2-pyridyl]piperazin-1-yl]sulfonylphenyl]benzamide;
4-[(3-Aminopropylamino)methyl]-N-[4-[4-[4-(trifluoromethyl)-2-pyridyl]piperazin-1-yl]sulfonylphenyl]benzamide;
4-[(3-Aminopropylamino)methyl]-N-[4-[4-[5-chloro-4-(trifluoromethyl)-2-pyridyl]piperazin-1-yl]sulfonylphenyl]benzamide;
4-[(3-Aminopropylamino)methyl]-N-[4-[4-[3-chloro-5-(trifluoromethyl) phenyl]piperazin-1-yl]sulfonylphenyl]benzamide;
4-[(3-Aminopropylamino)methyl]-N-[4-[4-[2-chloro-6-(trifluoromethyl)pyrimidin-4-yl]piperazin-1-yl]sulfonylphenyl]benzamide;
4-[(3-Aminopropylamino)methyl]-N-[4-[4-[2-amino-6-(trifluoromethyl)pyrimidin-4-yl]piperazin-1-yl]sulfonylphenyl]benzamide;
4-[(3-Aminopropylamino)methyl]-N-[4-[4-(4-bromo-6-chloro-2-pyridyl)piperazin-1-yl]sulfonylphenyl]benzamide;
4-[(3-Aminopropylamino)methyl]-N-[4-[4-[5-cyclopropyl-4-(trifluoromethyl)-2-pyridyl]piperazin-1-yl]sulfonylphenyl]benzamide;
4-[(3-Aminopropylamino)methyl]-N-[4-[4-[6-(cyclopropoxy)-4-(trifluoromethyl)-2-pyridyl]piperazin-1-yl]sulfonylphenyl]benzamide;
4-[(3-Aminopropylamino)methyl]-N-[4-[4-[6-ethoxy-4-(trifluoromethyl)-2-pyridyl]piperazin-1-yl]sulfonylphenyl]benzamide;
4-[(3-Aminopropylamino)methyl]-N-[4-[4-[6-ethyl-4-(trifluoromethyl)-2-pyridyl]piperazin-1-yl]sulfonylphenyl]benzamide;
4-[(3-Aminopropylamino)methyl]-N-[4-[4-[4-(trifluoromethyl)pyrimidin-2-yl]piperazin-1-yl]sulfonylphenyl]benzamide;
4-[2-(3-Aminopropylamino)ethyl]-N-[4-[4-[5-methyl-4-(trifluoromethyl)-2-pyridyl]piperazin-1-yl]sulfonylphenyl]benzamide;
4-[2-(3-Aminopropylamino)ethyl]-N-[4-[4-(4,5-dichloro-2-pyridyl)piperazin-1-yl]sulfonylphenyl]benzamide;
4-[2-(3-Aminopropylamino)ethyl]-N-[4-[4-[4-cyano-6-(trifluoromethyl)-2-pyridyl]piperazin-1-yl]sulfonylphenyl]benzamide;
4-[2-(3-Aminopropylamino)ethyl]-N-[4-[4-[6-cyano-4-(trifluoromethyl)-2-pyridyl]piperazin-1-yl]sulfonylphenyl]benzamide;
4-[2-(3-Aminopropylamino)ethyl]-N-[4-[4-[6-chloro-4-(1,1-difluoroethyl)-2-pyridyl]piperazin-1-yl]sulfonylphenyl]benzamide;
3-[(3-Aminopropylamino)methyl]-N-[4-[4-[6-methyl-4-(trifluoromethyl)-2-pyridyl]piperazin-1-yl]sulfonylphenyl]benzamide;
Methyl 2-[4-[4-[[3-[(3-aminopropylamino)methyl]benzoyl]amino]phenyl]sulfonylpiperazin-1-yl]-6-chloro-pyridine-4-carboxylate;
3-[(3-Aminopropylamino)methyl]-N-[4-[4-(6-chloro-4-cyano-2-pyridyl)piperazin-1-yl]sulfonylphenyl]benzamide;
3-[(3-Aminopropylamino)methyl]-N-[4-[4-[6-chloro-4-(trifluoromethyl)-2-pyridyl]-2-methyl-piperazin-1-yl]sulfonylphenyl]benzamide;
3-[(3-Aminopropylamino)methyl]-N-[4-[4-[6-chloro-4-(trifluoromethyl)-2-pyridyl]-3-methyl-piperazin-1-yl]sulfonylphenyl]benzamide;
3-[(3-Aminopropylamino)methyl]-N-[4-[[5-[6-chloro-4-(trifluoromethyl)-2-pyridyl]-2,5-diazabicyclo[4.2.0]octan-2-yl]sulfonyl]phenyl]benzamide;
3-[(3-Aminopropylamino)methyl]-N-[4-[4-[6-chloro-4-(trifluoromethyl)-2-pyridyl]piperazin-1-yl]sulfonylphenyl]benzamide;
3-[2-(3-Aminopropylamino)ethyl]-N-[4-[S-[4-[6-chloro-4-(trifluoromethyl)-2-pyridyl]piperazin-1-yl]-N-cyano-sulfonimidoyl]phenyl]benzamide;
3-[(3-aminopropylamino)methyl]-N-[4-[S-[4-[6-chloro-4-(trifluoromethyl)-2-pyridyl]piperazin-1-yl]-N-methyl-sulfonimidoyl]phenyl]benzamide;
3-[(3-Aminopropylamino)methyl]-N-[4-[4-[6-chloro-4-(trifluoromethyl)-2-pyridyl]piperazin-1-yl]sulfonyl-3-methoxy-phenyl]benzamide;
3-[(3-Aminopropylamino)methyl]-N-[4-[4-[6-chloro-4-(trifluoromethyl)-2-pyridyl]piperazin-1-yl]sulfonyl-3-hydroxy-phenyl]benzamide;
4-[(3-Aminopropylamino)methyl]-N-[5-[4-[6-chloro-4-(trifluoromethyl)-2-pyridyl]piperazin-1-yl]sulfonyl-2-pyridyl]benzamide;
3-[(3-Aminopropylamino)methyl]-N-[4-[4-[6-chloro-4-(trifluoromethyl)-2-pyridyl]piperazin-1-yl]sulfonyl-2-methoxy-phenyl]benzamide;
3-[(3-Aminopropylamino)methyl]-N-[4-[4-[6-chloro-4-(trifluoromethyl)-2-pyridyl]piperazin-1-yl]sulfonyl-2-hydroxy-phenyl]benzamide;
N-[4-[4-[6-chloro-4-(trifluoromethyl)-2-pyridyl]piperazin-1-yl]sulfonylphenyl]-2-methyl-4,6-dihydropyrrolo[3,4-c]pyrazole-5-carboxamide;
N-[4-[4-[6-chloro-4-(trifluoromethyl)-2-pyridyl]piperazin-1-yl]sulfonylphenyl]-1-methyl-4,6-dihydropyrrolo[3,4-c]pyrazole-5-carboxamide;
[5-[4-[6-Chloro-4-(trifluoromethyl)-2-pyridyl]piperazin-1-yl]sulfonylindolin-1-yl]-pyrazolo[1,5-a]pyrimidin-3-yl-methanone;
4-[5-[4-[6-Chloro-4-(trifluoromethyl)-2-pyridyl]piperazin-1-yl]sulfonylindoline-1-carbonyl]-2-methyl-pyrazole-3-carboxylic acid;
[1-(Azetidin-3-yl)pyrazol-4-yl]-[5-[4-[6-chloro-4-(trifluoromethyl)-2-pyridyl]piperazin-1-yl]sulfonylindolin-1-yl]methanone;
[1-(3-Aminopropyl)pyrazol-4-yl]-[5-[4-[6-chloro-4-(trifluoromethyl)-2-pyridyl]piperazin-1-yl]sulfonylindolin-1-yl]methanone;
[5-[4-[6-Chloro-4-(trifluoromethyl)-2-pyridyl]piperazin-1-yl]sulfonylindolin-1-yl]-(4,5,6,7-tetrahydropyrazolo[1,5-a]pyrazin-3-yl)methanone;
[5-[4-[6-Chloro-4-(trifluoromethyl)-2-pyridyl]piperazin-1-yl]sulfonylindolin-1-yl]-(1-oxa-2,9-diazaspiro[4.5]dec-2-en-3-yl)methanone;
N-[4-[4-[6-chloro-4-(trifluoromethyl)-2-pyridyl]piperazin-1-yl]sulfonylphenyl]-2-piperazin-1-yl-acetamide;
1-[5-[4-[6-Chloro-4-(trifluoromethyl)-2-pyridyl]piperazin-1-yl]sulfonylindolin-1-yl]-2-(2-hydroxyethylamino)ethanone;
2-[5-[4-[6-Chloro-4-(trifluoromethyl)-2-pyridyl]piperazin-1-yl]sulfonylindoline-1-carbonyl]benzoic acid;
[2-Acetoxy-4-[5-[4-[6-chloro-4-(trifluoromethyl)-2-pyridyl]piperazin-1-yl]sulfonylindoline-1-carbonyl]phenyl]acetate;
N-[4-[4-[4-methyl-6-(trifluoromethyl)pyrimidin-2-yl]piperazin-1-yl]sulfonylphenyl]-6,7-dihydro-5H-pyrrolo[3,4-b]pyridine-3-carboxamide;
N-[4-[4-[6-methyl-4-(trifluoromethyl)-2-pyridyl]piperazin-1-yl]sulfonylphenyl]-6,7-dihydro-5H-pyrrolo[3,4-b]pyridine-3-carboxamide;
N-[4-[4-[6-chloro-4-(trifluoromethyl)-2-pyridyl]piperazin-1-yl]sulfonylphenyl]isoindoline-5-carboxamide;
N-[4-[4-[6-chloro-4-(trifluoromethyl)-2-pyridyl]piperazin-1-yl]sulfonylphenyl]-3-(2,5-diazabicyclo[2.2.1]heptan-2-yl)benzamide;
3-(2-Aminoethoxy)-N-[4-[4-[6-chloro-4-(trifluoromethyl)-2-pyridyl]piperazin-1-yl]sulfonylphenyl]benzamide;
3-(Azetidin-3-ylmethylamino)-N-[4-[4-[6-chloro-4-(trifluoromethyl)-2-pyridyl]piperazin-1-yl]sulfonylphenyl]benzamide;
3-(Azetidin-3-ylmethoxy)-N-[4-[4-[6-chloro-4-(trifluoromethyl)-2-pyridyl]piperazin-1-yl]sulfonylphenyl]benzamide;
N-[4-[4-[6-chloro-4-(trifluoromethyl)-2-pyridyl]piperazin-1-yl]sulfonylphenyl]-3-[(3R)-3-aminopyrrolidin-1-yl]benzamide;
N-[4-[4-[6-Chloro-4-(trifluoromethyl)-2-pyridyl]piperazin-1-yl]sulfonylphenyl]-3-[3,6-diazabicyclo[3.2.0]heptan-3-yl]benzamide;
3-(2-Aminoethyl)-N-[4-[4-[6-chloro-4-(trifluoromethyl)-2-pyridyl]piperazin-1-yl]sulfonylphenyl]benzamide;
N-[4-[4-[6-chloro-4-(trifluoromethyl)-2-pyridyl]piperazin-1-yl]sulfonylphenyl]-3-piperazin-1-yl-benzamide;
2-Amino-N-[4-[4-[6-chloro-4-(trifluoromethyl)-2-pyridyl]piperazin-1-yl]sulfonylphenyl]indane-5-carboxamide;
5-(Aminomethyl)-N-[4-[4-[6-chloro-4-(trifluoromethyl)-2-pyridyl]piperazin-1-yl]sulfonylphenyl]-2-hydroxy-benzamide;
N-[4-[4-[6-chloro-4-(trifluoromethyl)-2-pyridyl]piperazin-1-yl]sulfonylphenyl]-3-(2,4-diaminobutanoylamino)benzamide;
4-[(3-Aminopropylamino)methyl]-N-[4-[4-[6-chloro-4-(trifluoromethyl)-2-pyridyl]piperazin-1-yl]sulfonylphenyl]pyridine-2-carboxamide;
6-[(3-Aminopropylamino)methyl]-N-[4-[4-[6-chloro-4-(trifluoromethyl)-2-pyridyl]piperazin-1-yl]sulfonylphenyl]pyridine-2-carboxamide;
5-[(3-Aminopropylamino)methyl]-N-[4-[4-[6-chloro-4-(trifluoromethyl)-2-pyridyl]piperazin-1-yl]sulfonylphenyl]pyridine-3-carboxamide;
2-(3-Aminopropylamino)-N-[4-[4-[6-chloro-4-(trifluoromethyl)-2-pyridyl]piperazin-1-yl]sulfonylphenyl]indane-5-carboxamide;
3-(3-Aminopropylamino)-N-[4-[4-[6-chloro-4-(trifluoromethyl)-2-pyridyl]piperazin-1-yl]sulfonylphenyl]indane-5-carboxamide;
N-[4-[4-[6-chloro-4-(trifluoromethyl)-2-pyridyl]piperazin-1-yl]sulfonylphenyl]-2-methoxy-5-[(5-methyl-2,3,3a,4,6,6a-hexahydropyrrolo[2,3-c]pyrrol-1-yl)methyl]benzamide;
1-(3-Aminopropylamino)-N-[4-[4-[6-chloro-4-(trifluoromethyl)-2-pyridyl]piperazin-1-yl]sulfonylphenyl]indane-5-carboxamide;
3,4-bis(3-Aminopropoxy)-N-[4-[4-[6-chloro-4-(trifluoromethyl)-2-pyridyl]piperazin-1-yl]sulfonylphenyl]benzamide;
3-[1-(3-Aminopropyl)pyrrolidin-2-yl]-N-[4-[4-[6-chloro-4-(trifluoromethyl)-2-pyridyl]piperazin-1-yl]sulfonylphenyl]benzamide;
3-[1-(3-Aminopropyl)pyrrolidin-3-yl]-N-[4-[4-[6-chloro-4-(trifluoromethyl)-2-pyridyl]piperazin-1-yl]sulfonylphenyl]benzamide;
3-[4-(3-Aminopropyl)piperazin-1-yl]-N-[4-[4-[6-chloro-4-(trifluoromethyl)-2-pyridyl]piperazin-1-yl]sulfonylphenyl]benzamide;
4-[2-(3-Aminopyrrolidin-1-yl)ethyl]-N-[4-[4-[6-chloro-4-(trifluoromethyl)-2-pyridyl]piperazin-1-yl]sulfonylphenyl]benzamide;
4-[2-(7-Amino-2-azaspiro[3.3]heptan-2-yl)ethyl]-N-[4-[4-[6-chloro-4-(trifluoromethyl)-2-pyridyl]piperazin-1-yl]sulfonylphenyl]benzamide;
N-[4-[4-[6-chloro-4-(trifluoromethyl)-2-pyridyl]piperazin-1-yl]sulfonylphenyl]-4-[2-(5-methyl-2,3,3a,4,6,6a-hexahydropyrrolo[2,3-c]pyrrol-1-yl)ethyl]benzamide;
N-[4-[4-[6-chloro-4-(trifluoromethyl)-2-pyridyl]piperazin-1-yl]sulfonylphenyl]-4-[[(2S)-2-(2-aminoethyl)pyrrolidin-1-yl]methyl]benzamide;
N-[4-[4-[6-chloro-4-(trifluoromethyl)-2-pyridyl]piperazin-1-yl]sulfonylphenyl]-4-[2-(3-morpholinopropylamino)ethyl]benzamide;
4-[2-(2-Amino-5-azaspiro[2.4]heptan-5-yl)ethyl]-N-[4-[4-[6-chloro-4-(trifluoromethyl)-2-pyridyl]piperazin-1-yl]sulfonylphenyl]benzamide;
N-[4-[4-[6-chloro-4-(trifluoromethyl)-2-pyridyl]piperazin-1-yl]sulfonylphenyl]-4-[2-(3,6-diazabicyclo[3.1.1]heptan-6-yl)ethyl]benzamide;
N-[4-[4-[6-chloro-4-(trifluoromethyl)-2-pyridyl]piperazin-1-yl]sulfonylphenyl]-3-[(4-piperidylmethylamino)methyl]benzamide;
3-[[(3-Aminocyclobutyl)methylamino]methyl]-N-[4-[4-[6-chloro-4-(trifluoromethyl)-2-pyridyl]piperazin-1-yl]sulfonylphenyl]benzamide;
N-[4-[4-[6-chloro-4-(trifluoromethyl)-2-pyridyl]piperazin-1-yl]sulfonylphenyl]-3-[(3-piperazin-1-ylpropylamino)methyl]benzamide;
3-[[3-(1-Aminocyclopropyl)pyrrolidin-1-yl]methyl]-N-[4-[4-[6-chloro-4-(trifluoromethyl)-2-pyridyl]piperazin-1-yl]sulfonylphenyl]benzamide;
3-[[3-[Bis(2-hydroxyethyl)amino]propylamino]methyl]-N-[4-[4-[6-chloro-4-(trifluoromethyl)-2-pyridyl]piperazin-1-yl]sulfonylphenyl]benzamide;
N-[4-[4-[6-chloro-4-(trifluoromethyl)-2-pyridyl]piperazin-1-yl]sulfonylphenyl]-2-methoxy-5-[[3-(4-piperidyl)propylamino]methyl]benzamide;
3-[[1-(Aminomethyl)-3-azabicyclo[3.1.0]hexan-3-yl]methyl]-N-[4-[4-[6-chloro-4-(trifluoromethyl)-2-pyridyl]piperazin-1-yl]sulfonylphenyl]benzamide;
(2R)-2-Amino-4-[[3-[[4-[4-[6-chloro-4-(trifluoromethyl)-2-pyridyl]piperazin-1-yl]sulfonylphenyl]carbamoyl]phenyl]methylamino]butanoic acid;
N-[4-[4-[6-chloro-4-(trifluoromethyl)-2-pyridyl]piperazin-1-yl]sulfonylphenyl]-3-[[(2-oxooxazolidin-4-yl)methylamino]methyl]benzamide;
N-[4-[4-[6-chloro-4-(trifluoromethyl)-2-pyridyl]piperazin-1-yl]sulfonylphenyl]-3-[[(5-oxopyrrolidin-3-yl)methylamino]methyl]benzamide;
N-[4-[4-[6-chloro-4-(trifluoromethyl)-2-pyridyl]piperazin-1-yl]sulfonylphenyl]-3-[[[3-(dimethylamino)-3-oxo-propyl]amino]methyl]benzamide;
5-(3,3a,4,5,6,6a-Hexahydro-2H-pyrrolo[3,4-b]pyrrol-1-ylmethyl)-N-[4-[4-[6-chloro-4-(trifluoromethyl)-2-pyridyl]piperazin-1-yl]sulfonylphenyl]-2-methoxy-benzamide; and
2-[(3-Aminopropylamino)methyl]-N-[4-[4-[6-chloro-4-(trifluoromethyl)-2-pyridyl]piperazin-1-yl]sulfonylphenyl]benzamide;
or a pharmaceutically acceptable salt thereof.

17. A process for the preparation of a compound according to claim 1 comprising any of the following steps:
a) formation of compound of formula (Ia), 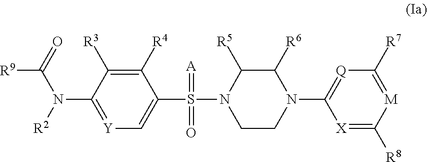
via coupling reaction or acylation reaction between compound of formula (IX), 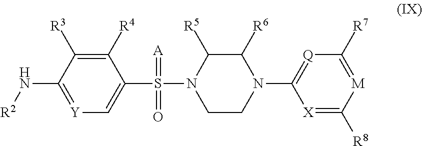
and compound of formula (XII), 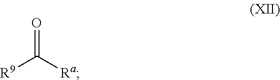
b) formation of compound of formula (Ib), 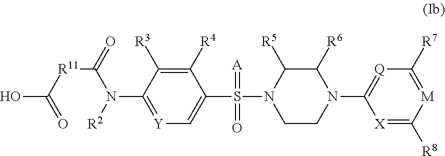
via hydrolysis of compound of formula (XIV), 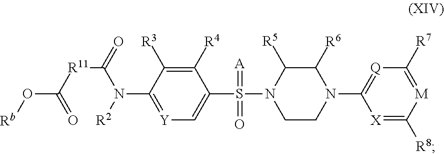
c) formation of compound of formula (Ia), 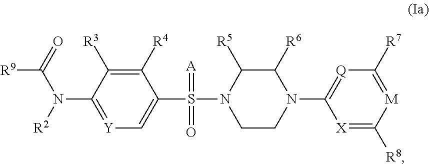
via nucleophilic substitution between compound of formula (XVIII), 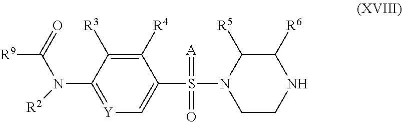
and compound of formula (II), 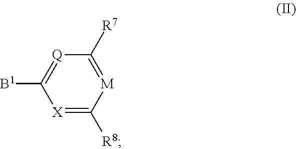
wherein
in step a), coupling reaction was conducted in the presence of a condensation reagent; and the acylation reaction was conducted in the presence of a base;
in step b), the hydrolysis reaction was conducted in the presence of a base;
in step c), the nucleophilic substitution reaction was conducted in the presence of a base;
wherein B¹ is halogen; PG¹ and PG² are protecting group; R^{a} is halogen or OH; R^{b} is C₁₋₆alkyl; R¹¹ is pyrazolylene, phenylene or aminoC₁₋₆alkylaminoC₁₋₆alkylene; and R² to R⁹, Y, A, Q, X and M are defined as in claim 1.

18. (canceled)

19. A pharmaceutical composition comprising a compound of claim 1; and a therapeutically inert carrier.

20. A method for the inhibition of LpxH in a patient in need thereof said method comprising administering to said patient a compound of claim 1.

21. The method of claim 20, wherein the bacterial infection is caused by gram-negative bacteria.

22. (canceled)

23. The method of claim 21, wherein the gram-negative bacteria is selected from Enterobacteriaceae, Neisseria gonorrhoeae, Haemophilus influenzae, Helicobacter pylorus, Acinetobacter baumannii and Pseudomonas aeruginosa.

24. The method of claim 23, wherein the gram-negative bacteria is Enterobacteriaceae.

25. The method of claim 29, wherein the bacterial infection is caused by gram-negative bacteria.

26. A method according to claim 25, wherein the gram-negative bacteria is selected from Enterobacteriaceae, Neisseria gonorrhoeae, Haemophilus influenzae, Helicobacter pylorus, Acinetobacter baumannii and Pseudomonas aeruginosa.

27. A method according to claim 26, wherein the gram-negative bacteria is Enterobacteriaceae.

28. A compound or pharmaceutically acceptable salt according to claim 1, said compound or salt manufactured according to the process of claim 17.

29. A method for the treatment or prophylaxis of bacterial infection, which method comprises administering a therapeutically effective amount of a compound as defined in claim 1, to a patient in need thereof.

30. The method of claim 24, wherein Enterobacteriaceae is Klebsiella pneumoniae or Escherichia coli

31. The method of claim 27, wherein Enterobacteriaceae is Klebsiella pneumoniae or Escherichia coli

32. The process of claim 17, wherein the condensation reagent in the condensation reaction of step a) is TCFH.

33. The process of claim 17, wherein the base in the acylation reaction of step a) is TEA.

34. The process of claim 17, wherein the base in the hydrolysis reaction of step b) is LiGH.

35. The process of claim 17, wherein the base in the nucleophilic substitution reaction of step c) is Cs₂CO₃.

36. The process of claim 17, wherein PG¹ is tert-butoxycarbonyl or benzyloxycarbonyl

37. The process of claim 17, wherein PG² is an acetyl group.

38. The process of claim 17, wherein R^{b} is methyl or ethyl.
